(19) United States
(12) Patent Application Publication
(10) Pub. No.: US 2021/0189392 A1
(43) Pub. Date: Jun. 24, 2021

(54) S-ANTIGEN TRANSPORT INHIBITING OLIGONUCLEOTIDE POLYMERS AND METHODS

(71) Applicant: Aligos Therapeutics, Inc., South San Francisco, CA (US)

(72) Inventors: Leonid Beigelman, San Mateo, CA (US); Rajendra Pandey, South San Francisco, CA (US); Vivek Kumar Rajwanshi, Cupertino, CA (US); David Bernard Smith, San Mateo, CA (US); Lawrence M. Blatt, Healdsburg, CA (US); Jin Hong, Brisbane, CA (US); Megan Elizabeth Fitzgerald, San Francisco, CA (US)

(21) Appl. No.: 17/117,494

(22) Filed: Dec. 10, 2020

Related U.S. Application Data
(60) Provisional application No. 63/054,380, filed on Jul. 21, 2020.
(60) Provisional application No. 63/020,923, filed on May 6, 2020.
(60) Provisional application No. 62/947,401, filed on Dec. 12, 2019.

(51) Int. Cl. C12N 15/113

(52) U.S. Cl. CPC C12N 15/113; C12N 2310/315; C12N 2310/346; C12N 2310/334; C12N 2310/3233; C12N 2310/333

(57) ABSTRACT

Various embodiments provide STOPS™ polymers that are S-antigen transport inhibiting oligonucleotide polymers, processes for making them and methods of using them to treat diseases and conditions. In some embodiments the STOPS™ modified oligonucleotides include an at least partially phosphorothioated sequence of alternating A and C units having modifications as described herein. The sequence independent antiviral activity against hepatitis B of embodiments of STOPS™ modified oligonucleotides, as determined by HBsAg Secretion Assay, is an EC₅₀ that is less than 100 nM.

### INCORPORATION BY REFERENCE TO PRIORITY APPLICATION

This application claims priority to U.S. Provisional Application Ser. No. 62/947,401, filed Dec. 12, 2019; Ser. No. 63/020,923, filed May 6, 2020; and Ser. No. 63/054,380, filed Jul. 21, 2020; all of which are hereby incorporated herein by reference in their entireties.

### BACKGROUND

### Field

This application relates to STOPS™ antiviral compounds that are S-antigen transport inhibiting oligonucleotide polymers, processes for making them and methods of using them to treat diseases and conditions.

### Description

The STOPS™ compounds described herein are antiviral oligonucleotides that can be at least partially phosphorothioated and exert their antiviral activity by a non-sequence dependent mode of action. See A. Vaillant, “Nucleic acid polymers: Broad spectrum antiviral activity, antiviral mechanisms and optimization for the treatment of hepatitis B and hepatitis D infection”, Antiviral Research 133, 32-40 (2016). The term “Nucleic Acid Polymer” (NAP) has been used in the literature to refer to such oligonucleotides, although that term does not necessarily connotate antiviral activity. A number of patent applications filed in the early 2000s disclosed the structures of certain specific compounds and identified various structural options as potential areas for future experimentation. See, e.g., U.S. Pat. Nos. 7,358,068; 8,008,269; 8,008,270 and 8,067,385. These efforts resulted in the identification of the compound known to those skilled in the art as REP 2139, a phosphorothioated 40-mer having repeating adenosine-cytidine (AC) units with 5-methylation of all cytosines and 2′-O methyl modification of all ribose, along with the compound known as its clinical progenitor, REP 2055. See I. Roehl et al., “Nucleic Acid Polymers with Accelerated Plasma and Tissue Clearance for Chronic Hepatitis B Therapy”, Molecular Therapy: Nucleic Acids Vol. 8, 1-12 (2017). The authors of that publication indicated that the structural features of these compounds had been optimized for the treatment of hepatitis B (HBV) and hepatitis D (HBD). See also A. Vaillant, “Nucleic acid polymers: Broad spectrum antiviral activity, antiviral mechanisms and optimization for the treatment of hepatitis B and hepatitis D infection”, Antiviral Research 133 (2016) 32-40. According to these authors and related literature, such compounds preserve antiviral activity against HBV while preventing recognition by the innate immune response to allow their safe use with immunotherapies such as pegylated interferon. However, there remains a long-felt need for more effective compounds in this class.

### SUMMARY

It has now been discovered that, contrary to the teachings in the art regarding the optimum combination of desirable structural features for antiviral compounds, significantly improved properties can be obtained by modifying them to provide STOPS™ compounds as described herein. For example, in some embodiments the sequence independent antiviral activity of the new STOPS™ compounds against HBV, as determined by HBsAg Secretion Assay, is evidenced by an EC₅₀ that is less than 100 nM. In view of the many years of research culminating in the art-recognized optimized structure of REP 2139, there had been little expectation by those skilled in the art that embodiments of the modified STOPS™ compounds described herein would be reasonably likely to display such improvements in potency. Thus, the structures of the new STOPS™ compounds and methods of using them to treat HBV and HBD are surprising and unexpected.

Some embodiments described herein relate to a modified oligonucleotide or complex thereof having sequence independent antiviral activity against hepatitis B, that can include an at least partially phosphorothioated sequence of modified nucleoside units that comprise modified A units, modified C units and/or other modified nucleoside units, wherein:

the modified A units comprise one or more selected from:

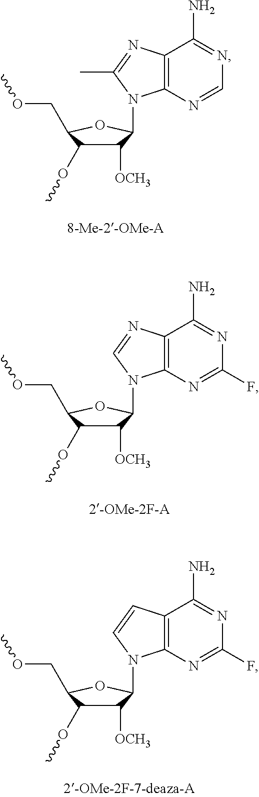

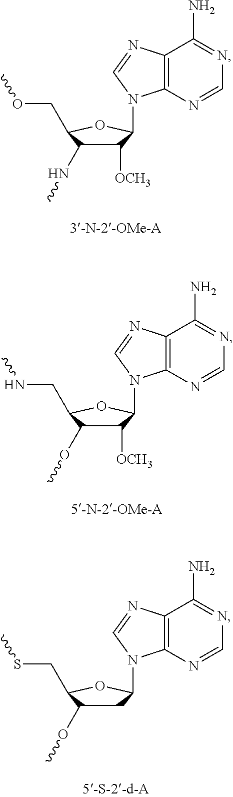

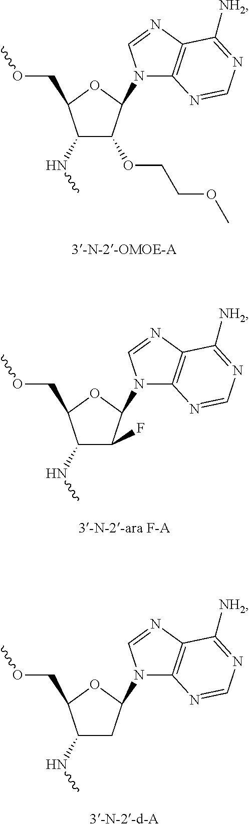

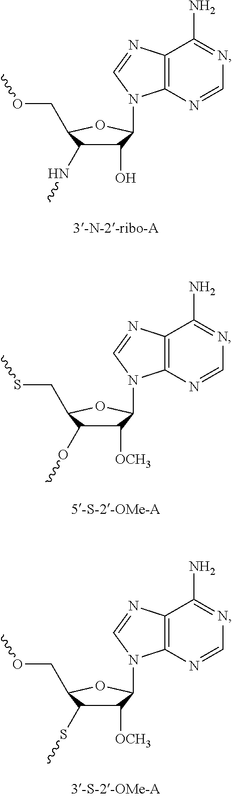

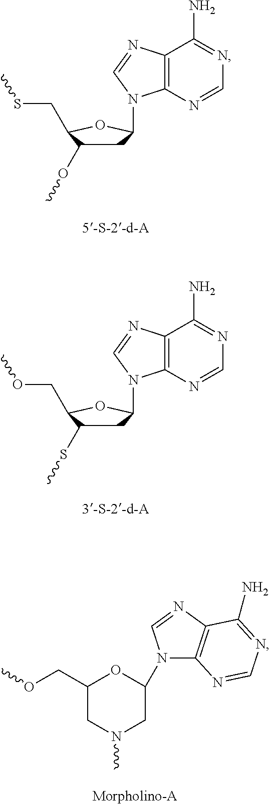

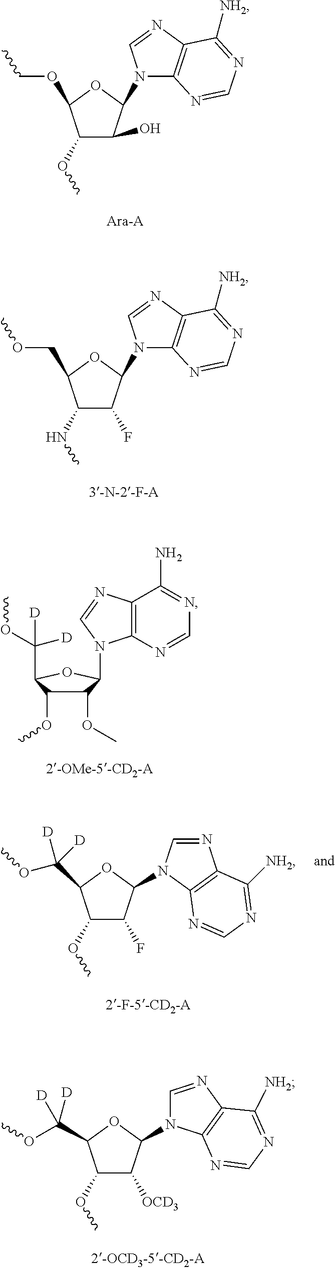

the modified C units comprise one or more selected from:

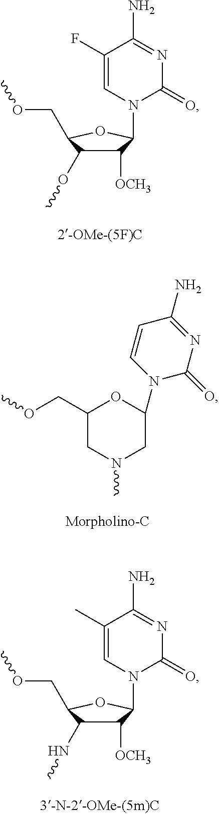

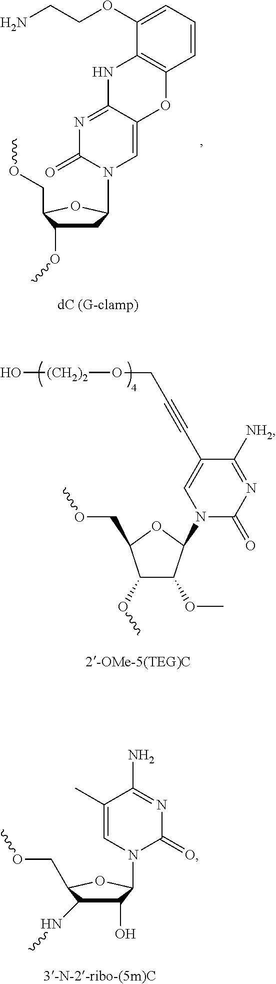

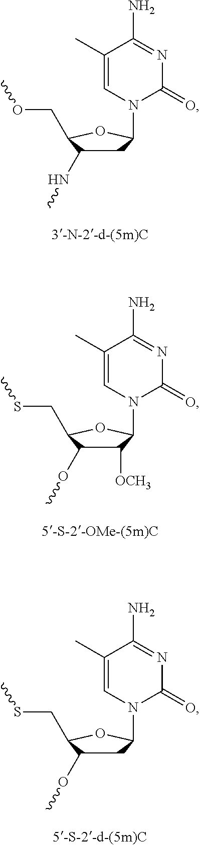

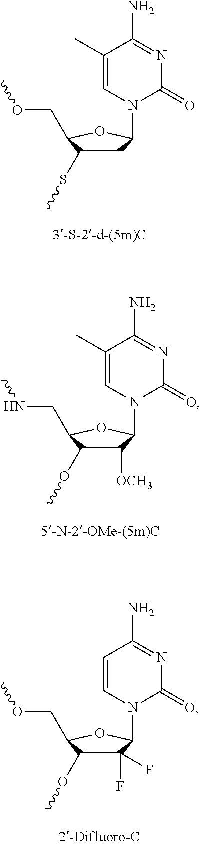

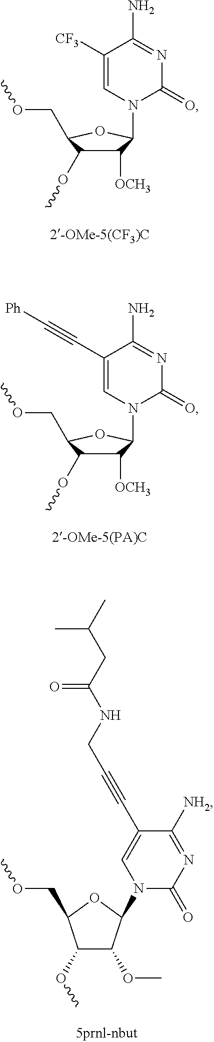

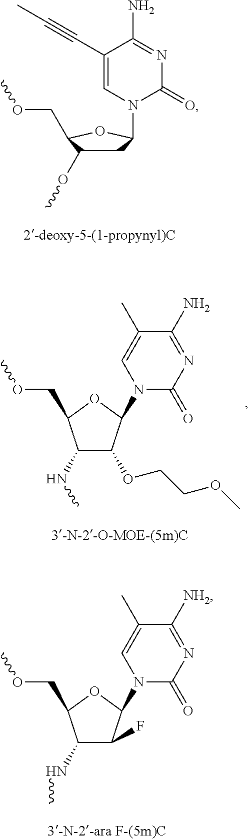

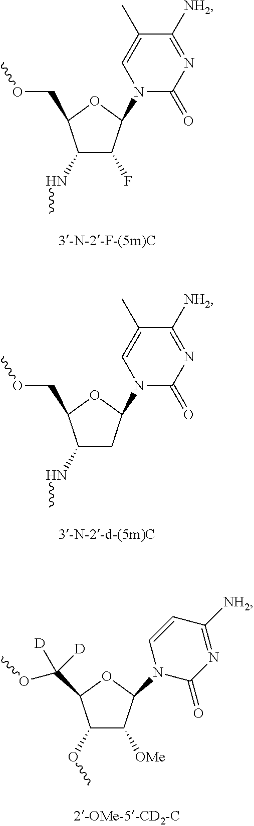

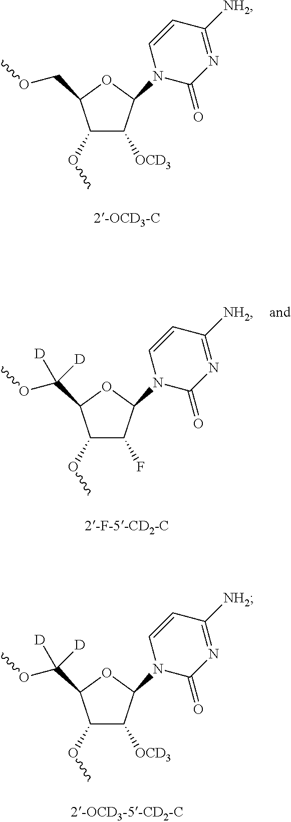

the other modified nucleoside units comprise one or more selected from:

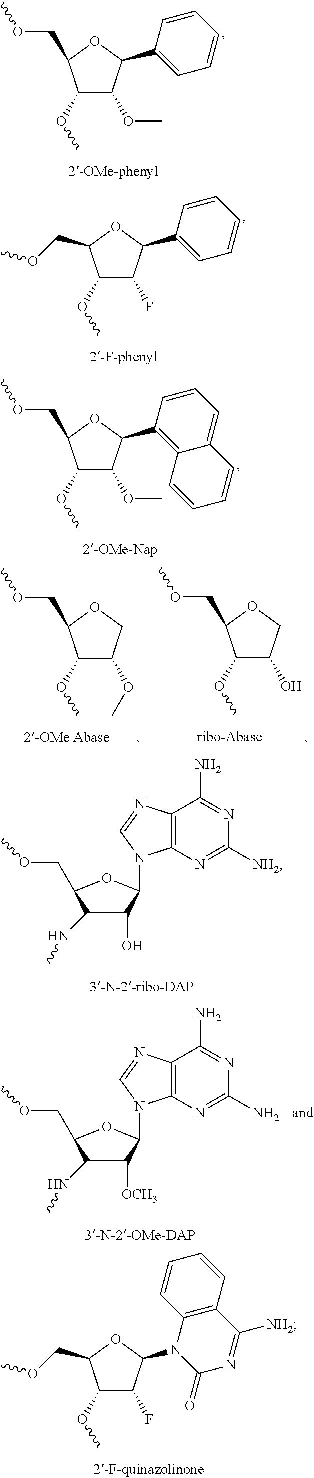

each terminal

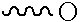

is independently hydroxyl, an O,O-dihydrogen phosphorothioate, a dihydrogen phosphate, an endcap or a linking group;

each terminal

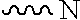

is independently an amine, a C₁₋₆ alkylamine, a di-C₁₋₆alkylamine, an endcap or a linking group;

each terminal

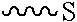

is independently a thiol, an O,O-dihydrogen phosphorothioate, a dihydrogen phosphate, an endcap or a linking group;

each internal

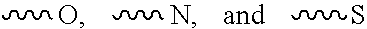

is joined to the

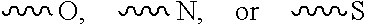

of a neighboring nucleoside unit to form a phosphorus-containing internucleoside linkage of the formula

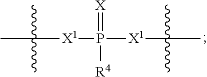

each X is individually S or O, with the proviso that at least one X is S;

each X¹ is individually O, NR^{b}, or S;

each R⁴ is individually OH, SH, optionally substituted C₁₋₆ alkyl, optionally substituted C₁₋₆ alkoxy, or optionally substituted amino;

each R^{b} is individually hours or C₁₋₆ alkyl; and

the sequence independent antiviral activity against hepatitis B, as determined by HBsAg Secretion Assay, is an EC₅₀ that is less than 100 nM.

Some embodiments described herein relate to a method of treating a HBV and/or HDV infection that can include administering to a subject identified as suffering from the HBV and/or HDV infection an effective amount of a modified oligonucleotide modified oligonucleotide as described herein, or a pharmaceutical composition that includes an effective amount of a modified oligonucleotide as described herein.

Some embodiments disclosed herein relate to a method of inhibiting replication of HBV and/or HDV that can include contacting a cell infected with the HBV and/or HDV with an effective amount of a modified oligonucleotide modified oligonucleotide as described herein, or a pharmaceutical composition that includes an effective amount of a modified oligonucleotide as described herein.

These are other embodiments are described in greater detail below.

### BRIEF DESCRIPTION OF THE DRAWINGS

FIG. 1 illustrates an embodiment of a modified oligonucleotide that comprises a C₂₋₆ alkylene linkage.

FIG. 2 illustrates an embodiment of a modified oligonucleotide that comprises a TREB-ps-c3-O—C3 linkage.

FIG. 3A illustrates an embodiment of a modified oligonucleotide having cholesterol attached via a 5′ tetraethylene glycol (TEG) linkage.

FIG. 3B illustrates an embodiment of a modified oligonucleotide having cholesterol attached via a 3′ TEG linkage.

FIG. 3C illustrates an embodiment of a modified oligonucleotide having a tocopherol (Vitamin E) attached via a 5′ TEG linkage.

FIG. 3D illustrates an embodiment of a modified oligonucleotide having a tocopherol (Vitamin E) attached via a 3′ TEG linkage.

FIGS. 4A and 4B illustrate embodiments of modified oligonucleotides having GalNac attached via a linking group.

FIG. 5 illustrates an embodiment of a reaction scheme for preparing a 5′-EP building block.

FIG. 6 illustrates embodiments of modified oligonucleotides and corresponding values of sequence independent antiviral activity against hepatitis B (as determined by HBsAg Secretion Assay) and cytotoxicity and abbreviations used in Table A.

FIG. 7 illustrates an embodiment of a reaction scheme for preparing compound 5′-VP.

FIG. 8 illustrates an embodiment of a reaction scheme for preparing compounds 8-5 and 8-6.

FIG. 9A illustrates an embodiment of a reaction scheme for preparing compound 9R.

FIG. 9B illustrates an embodiment of a reaction scheme for preparing compound 9S.

FIG. 10 illustrates an embodiment of a reaction scheme for preparing compounds 10-5 and 10-6.

FIG. 11A illustrates an embodiment of a reaction scheme for preparing compound 11R.

FIG. 11B illustrates an embodiment of a reaction scheme for preparing compound 11S.

FIG. 12 illustrates an embodiment of a reaction scheme for preparing compounds 12-5 and 12-6.

FIG. 13A illustrates an embodiment of a reaction scheme for preparing compound 13R.

FIG. 13B illustrates an embodiment of a reaction scheme for preparing compound 13S.

FIG. 14 illustrates an embodiment of a reaction scheme for preparing compound 14-8.

FIG. 15A illustrates an embodiment of a reaction scheme for preparing compound 15-9.

FIG. 15B illustrates an embodiment of a reaction scheme for preparing compound 15-19.

FIG. 15C illustrates an embodiment of a reaction scheme for preparing compound 15-22.

FIG. 16 illustrates an embodiment of a reaction scheme for preparing compound 16-6.

FIG. 17 illustrates an embodiment of a reaction scheme for preparing compound 17-4.

FIG. 18 illustrates an embodiment of a reaction scheme for preparing compound 18-7.

FIG. 19 illustrates an embodiment of a reaction scheme for preparing compound 19-5.

FIG. 20 illustrates an embodiment of a reaction scheme for preparing compound 20-9.

FIG. 21 illustrates an embodiment of a reaction scheme for preparing compound 21-13.

FIG. 22 illustrates an embodiment of a reaction scheme for preparing compound 22-7.

FIG. 23 illustrates an embodiment of a reaction scheme for preparing compound 23-8.

FIG. 24 illustrates an embodiment of a reaction scheme for preparing compound 24-8.

FIG. 25 illustrates an embodiment of a reaction scheme for preparing compound 25-10.

FIG. 26 illustrates an embodiment of a reaction scheme for preparing compound 26-11.

FIG. 27 illustrates an embodiment of a reaction scheme for preparing compound 27-15.

FIG. 28 illustrates an embodiment of a reaction scheme for preparing compound 28-16.

FIG. 29 illustrates an embodiment of a reaction scheme for preparing compound 29-6.

FIG. 30 illustrates an embodiment of a reaction scheme for preparing compound 30-15.

FIG. 31 illustrates an embodiment of a reaction scheme for preparing compound 31-10.

FIG. 32 illustrates an embodiment of a reaction scheme for preparing compound 32-14.

FIG. 33 illustrates an embodiment of a reaction scheme for preparing compound 33-10.

FIG. 34 illustrates an embodiment of a reaction scheme for preparing compound 34-11.

FIG. 35 illustrates an embodiment of a reaction scheme for preparing compound 35-10.

FIG. 36 illustrates an embodiment of a reaction scheme for preparing compound 36-8.

FIG. 37 illustrates an embodiment of a reaction scheme for preparing compound 37-8.

FIG. 38 illustrates an embodiment of a reaction scheme for preparing compound 38-12.

FIG. 39 illustrates an embodiment of a reaction scheme for preparing compound 39-14.

FIG. 40 illustrates an embodiment of a reaction scheme for preparing compound 40-9.

FIG. 41 illustrates an embodiment of a reaction scheme for preparing compound 41-10.

FIG. 42 illustrates an embodiment of a reaction scheme for preparing compound 42-8.

FIG. 43 illustrates an embodiment of a reaction scheme for preparing compound 43-10.

FIG. 44 illustrates an embodiment of a reaction scheme for preparing compound 44-10.

FIG. 45 illustrates an embodiment of a reaction scheme for preparing compound 45-7.

FIG. 46 illustrates an embodiment of a reaction scheme for preparing compound 46-7.

FIG. 47 illustrates an embodiment of a reaction scheme for preparing compound 47-7.

FIG. 48 illustrates an embodiment of a reaction scheme for preparing compound 48-7.

FIG. 49 illustrates an embodiment of a reaction scheme for preparing compound 49-7.

FIG. 50 illustrates an embodiment of a reaction scheme for preparing compound 50-11.

FIG. 51 illustrates an embodiment of a reaction scheme for preparing compound 51-12.

FIG. 52 illustrates an embodiment of a reaction scheme for preparing compound 52-12.

FIG. 53 illustrates an embodiment of a reaction scheme for preparing compound 53-13.

FIG. 54 illustrates an embodiment of a reaction scheme for preparing compound 54-6.

### DETAILED DESCRIPTION

The hepatitis B virus (HBV) is a DNA virus and a member of the Hepadnaviridae family. HBV infects more than 300 million worldwide and is a causative agent of liver cancer and liver disease such as chronic hepatitis, cirrhosis, and hepatocellular carcinoma. HBV can be acute and/or chronic. Acute HBV infection can be either asymptomatic or present with symptomatic acute hepatitis. HBV is classified into eight genotypes, A to hours.

HBV is a partially double-stranded circular DNA of about 3.2 kilobase (kb) pairs. The HBV replication pathway has been studied in great detail. T. J. Liang, Hepataology (2009) 49(5 Suppl): S13-S21. One part of replication includes the formation of the covalently closed circular (cccDNA) form. The presence of the cccDNA gives rise to the risk of viral reemergence throughout the life of the host organism. HBV carriers can transm/z the disease for many years. An estimated 257 million people are living with hepatitis B virus infection, and it is estimated that over 750,000 people worldwide die of hepatitis B each year. In addition, immunosuppressed individuals or individuals undergoing chemotherapy are especially at risk for reactivation of an HBV infection.

HBV can be transmitted by blood, semen, and/or another body fluid. This can occur through direct blood-to-blood contact, unprotected sex, sharing of needles, and from an infected mother to her baby during the delivery process. The HBV surface antigen (HBsAg) is most frequently used to screen for the presence of this infection. Currently available medications do not cure an HBV and/or HDV infection. Rather, the medications suppress replication of the virus.

The hepatitis D virus (HDV) is a DNA virus, also in the Hepadnaviridae family of viruses. HDV can propagate only in the presence of HBV. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all the hepatitis infections, at 20%. There is currently no cure or vaccine for hepatitis D.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein in the context of oligonucleotides or other materials, the term “antiviral” has its usual meaning as understood by those skilled in the art and thus includes an effect of the presence of the oligonucleotides or other material that inhibits production of viral particles, typically by reducing the number of infectious viral particles formed in a system otherwise suitable for formation of infectious viral particles for at least one virus. In certain embodiments, the antiviral oligonucleotide has antiviral activity against multiple different virus, e.g., both HBV and HDV.

As used herein the term “oligonucleotide” (or “oligo”) has its usual meaning as understood by those skilled in the art and thus refers to a class of compounds that includes oligodeoxynucleotides, oligodeoxyribonucleotides and oligoribonucleotides. Thus, “oligonucleotide” refers to an oligomer or polymer of ribonucleic acid (RNA) or deoxyribonucleic acid (DNA) or mimetics thereof, including reference to oligonucleotides composed of naturally-occurring nucleobases, sugars and phosphodiester (PO) internucleoside (backbone) linkages as well as “modified” or substituted oligonucleotides having non-naturally-occurring portions which function similarly. Thus, the term “modified” (or “substituted”) oligonucleotide has its usual meaning as understood by those skilled in the art and includes oligonucleotides having one or more of various modifications, e.g., stabilizing modifications, and thus can include at least one modification in the internucleoside linkage and/or on the ribose, and/or on the base. For example, a modified oligonucleotide can include modifications at the 2′-position of the ribose, acyclic nucleotide analogs, methylation of the base, phosphorothioated (PS) linkages, phosphorodithioate linkages, methylphosphonate linkages, diphosphorothioate linkages, 5′-phosphoramidate linkages, 3′,5′-phosphordiamidate linkages, 5′-thiophosphoramidate linkages, 3′,5′-thiophosphordiamidate linkages, diphosphodiester linkages, 3′-S-phosphorothiolate linkages, other modified linkages that connect to the sugar ring via oxygen, sulfur or nitrogen, and/or other modifications as described elsewhere herein. Thus, a modified oligonucleotide can include one or more phosphorothioated (PS) linkages, instead of or in addition to PO linkages.

As used herein in the context of modified oligonucleotides, the term “phosphorothioated” oligonucleotide has its usual meaning as understood by those skilled in the art and thus refers to a modified oligonucleotide in which all of the phosphodiester internucleoside linkages have been replaced by phosphorothioate linkages. Those skilled in the art thus understand that the term “phosphorothioated” oligonucleotide is synonymous with “fully phosphorothioated” oligonucleotide. A phosphorothioated oligonucleotide (or a sequence of phosphorothioated oligonucleotides within a partially phosphorothioated oligonucleotide) can be modified analogously, including (for example) by replacing one or more phosphorothioated internucleoside linkages by phosphodiester linkages. Thus, the term “modified phosphorothioated” oligonucleotide refers to a phosphorothioated oligonucleotide that has been modified in the manner analogous to that described herein with respect to oligonucleotides, e.g., by replacing a phosphorothioated linkage with a modified linkage such as phosphodiester, phosphorodithioate, methylphosphonate, diphosphorothioate, 5′-phosphoramidate, 3′,5′-phosphordiamidate, 5′-thiophosphoramidate, 3′,5′-thiophosphordiamidate, diphosphodiester or 3′-S-phosphorothiolate. An at least partially phosphorothioated sequence of a modified oligonucleotide can be modified similarly, and thus, for example, can be modified to contain a non-phosphorothioated linkage such as phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate, diphosphodiester or 3′-S-phosphorothiolate. In the context of describing modifications to a phosphorothioated oligonucleotide, or to an at least partially phosphorothioated sequence of a modified oligonucleotide, modification by inclusion of a phosphodiester linkage may be considered to result in a modified phosphorothioated oligonucleotide, or to a modified phosphorothioated sequence, respectively. Analogously, in the context of describing modifications to an oligonucleotide, or to an at least partially phosphodiesterified sequence of a modified oligonucleotide, the inclusion of a phosphorothioated linkage may be considered to result in a modified oligonucleotide or a modified phosphodiesterified sequence, respectively.

As used herein in the context of dinucleotides or oligonucleotides, the terms “stereochemically defined linkage” or “stereochemically defined phosphorothioate linkage” has its usual meaning as understood by those skilled in the art and thus refers to a linkage (e.g., a phosphorothioate linkage) having a phosphorus stereocenter with a selected chirality (R or S configuration). A composition containing such a dinucleotide or oligonucleotide can be enriched in molecules having the selected chirality. The stereopurity of such a composition can vary over a broad range, e.g. from about 51% to about 100% stereopure. In various embodiments, the stereopurity is greater than 55%, 65%, 75%, 85%, 90%, 91%, 92%, 93%, 94%, 95%, 96%, 97%, 98%, or 99%; or in a range defined as having any two of the foregoing stereopurity values as endpoints.

The term “sequence independent” antiviral activity has its usual meaning as understood by those skilled in the art and thus refers to an antiviral activity of an oligonucleotide (e.g., a modified oligonucleotide) that is independent of the sequence of the oligonucleotide. Methods for determining whether the antiviral activity of an oligonucleotide is sequence independent are known to those skilled in the art and include the tests for determining if an oligonucleotide acts predominantly by a non-sequence complementary mode of action as disclosed in Example 10 of U.S. Pat. Nos. 7,358,068; 8,008,269; 8,008,270 and 8,067,385, which is hereby incorporated herein by reference and particularly for the purpose of describing such tests.

In the context of describing modified oligonucleotides having sequence independent antiviral activity and comprising a sequence (e.g., an at least partially phosphorothioated sequence) of modified nucleoside units (e.g., modified A, modified C units, and/or other modified units), the terms “A” and “C” refer to the modified adenosine-containing (A) units and modified cytosine-containing (C) units set forth in Tables 1 and 2 below, respectively, unless the context indicates that the units are unmodified. Other examples of modified nucleoside units are set forth in Table 3 below.

TABLE 1
MODIFIED “A” UNITS

| Abbreviation (A Unit) | Structure (A Unit) |
|---|---|
| 8-Me-2′-OMe-A | 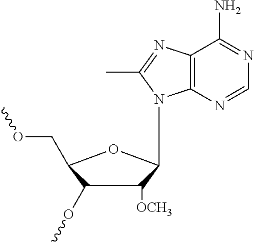 |
| 2′-OMe-2F-A | 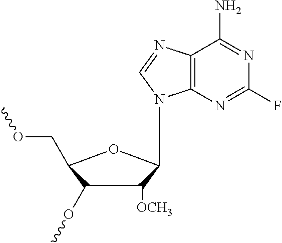 |
| 2′-OMe-2F-7-deaza-A | 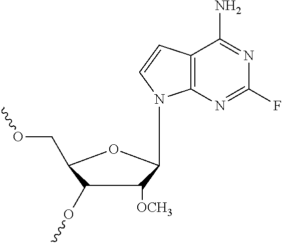 |
| 3′-N-2′-OMe-A | 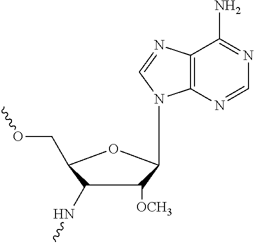 |
| 5′-N-2′-OMe-A | 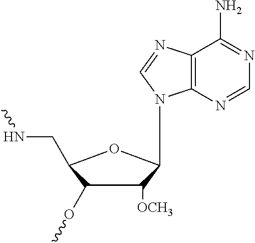 |
| 5′-S-2′-d-A | 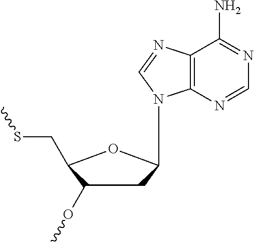 |
| 3′-N-2′-O-MOE-A | 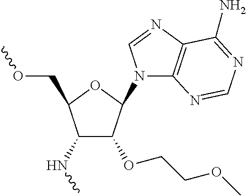 |
| 3′-N-2′-ara-F-A | 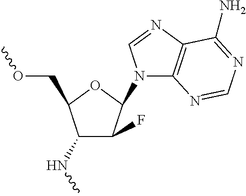 |
| 3′-N-2′-d-A | 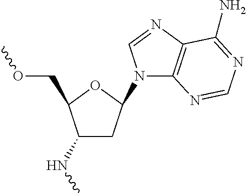 |
| 3′-N-2′-ribo-A | 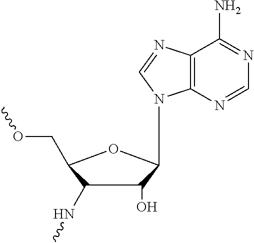 |
| 5′-S-2′-OMe-A | 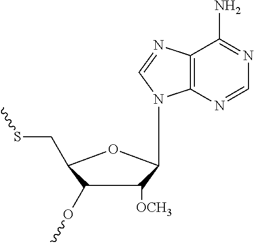 |
| 3′-S-2′-OMe-A | 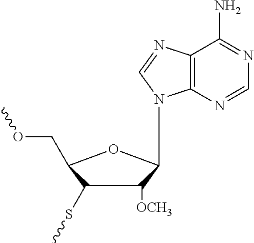 |
| 5′-S-2′-d-A | 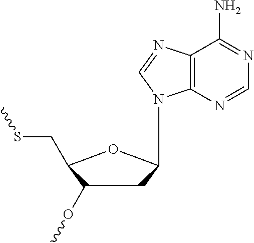 |
| 3′-S-2′-d-A | 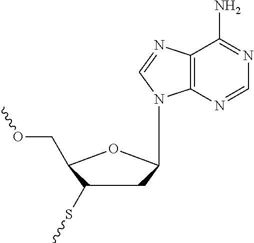 |
| Morpholino-A | 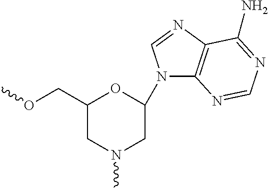 |
| Ara-A | 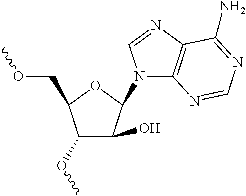 |
| 3′-N-2′-F-A | 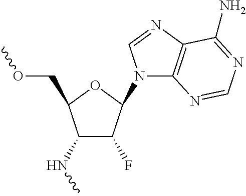 |
| 2′-OMe-5′-CD₂-A | 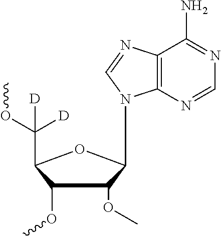 |
| 2′-F-5′-CD₂-A | 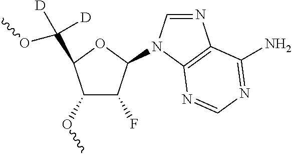 |
| 2′-OCD₃-5′-CD₂-A | 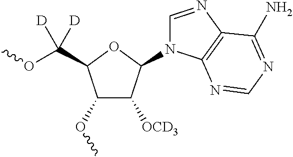 |

TABLE 2
MODIFIED “C” UNITS

| Abbreviation (C Unit) | Structure (C Unit) |
|---|---|
| 2′-OMe-(5F)C | 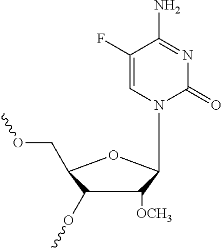 |
| Morpholino-C | 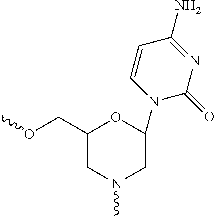 |
| 3′-N-2′-OMe-(5m)C | 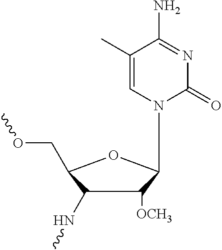 |
| dC (G-clamp) | 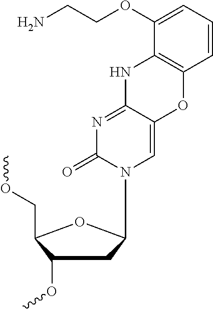 |
| 2′-OMe-5(TEG)C | 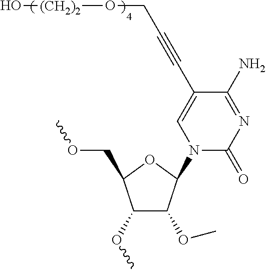 |
| 3′-N-2′-ribo-(5m)C | 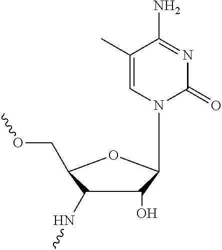 |
| 3′-N-2′-d-(5m)C | 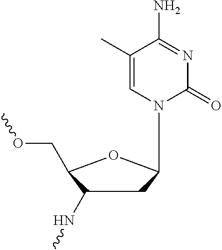 |
| 5′-S-2′-OMe-(5m)C | 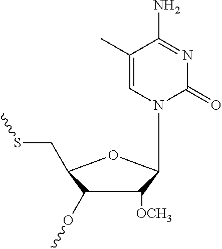 |
| 5′-S-2′-d-(5m)C | 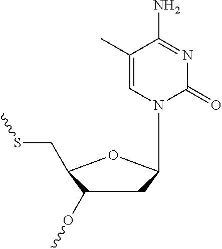 |
| 3′-S-2′-d-(5m)C | 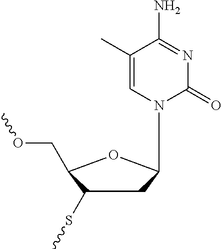 |
| 5′-N-2′-OMe-(5m)C | 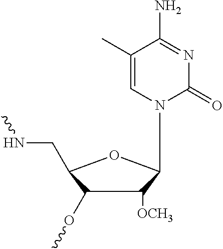 |
| 2′-Difluoro-C | 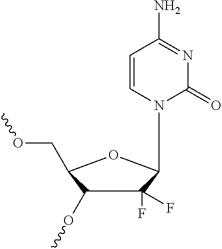 |
| 2′-OMe-5(CF₃)C | 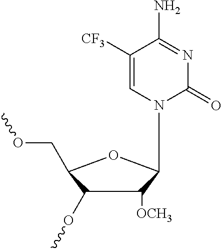 |
| 2′-OMe-5(PA)C | 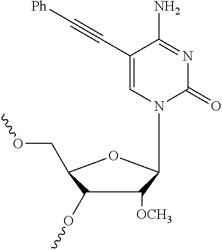 |
| 5prnl-nbut (2′-OMe-5(N- propargyl-2- methylpropanamide)C) | 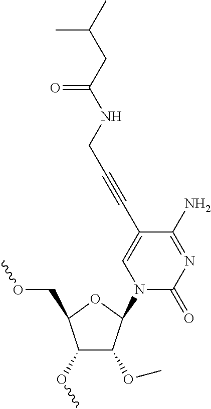 |
| 2′-Deoxy-5-(1- propynyl)C | 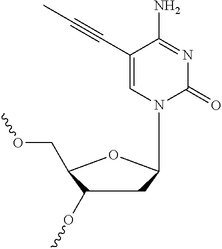 |
| 3′-N-2′-O-MOE-(5m)C | 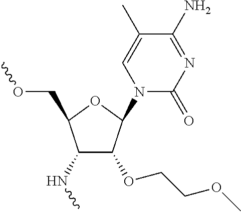 |
| 3′-N-2′-ara F-(5m)C | 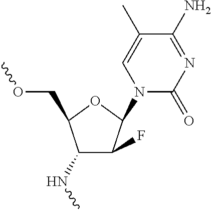 |
| 3′-N-2′-F-(5m)C | 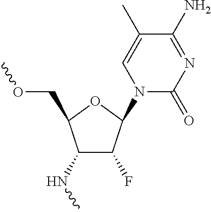 |
| 3′-N-2′-d-(5m)C | 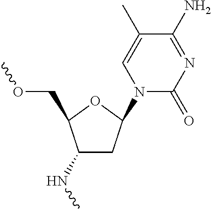 |
| 2′-OMe-5′-CD₂-C | 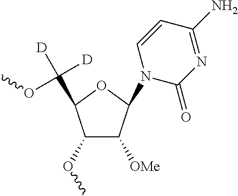 |
| 2′-OCD₃-C | 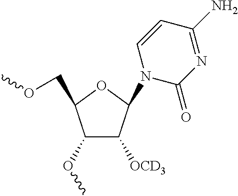 |
| 2′-F-5′-CD₂-C | 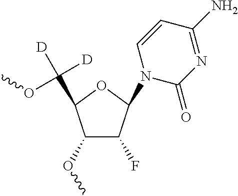 |
| 2′-OCD₃-5′-CD₂-C | 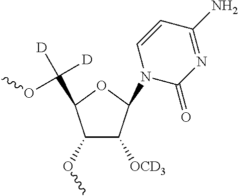 |

TABLE 3
OTHER MODIFIED NUCLEOSIDE UNITS

| Abbreviation | |
|---|---|
| (Other Unit) | Structure (Other Unit) |
| 2′-OMe-phenyl | 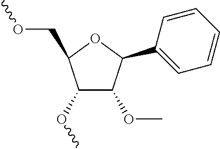 |
| 2′-F-phenyl | 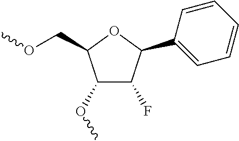 |
| 2′-OMe-Nap | 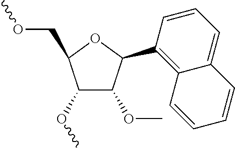 |
| 2′-OMe Abase | 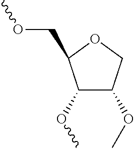 |
| Ribo-Abase | 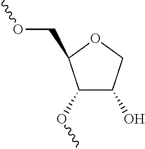 |
| 3′-N-2′-ribo-DAP | 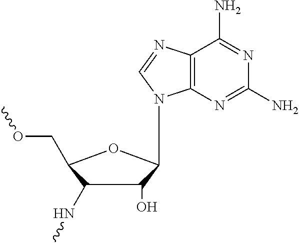 |
| 3′-N-2′-OMe-DAP | 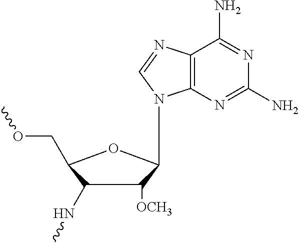 |
| 2′-F-quinazolinone | 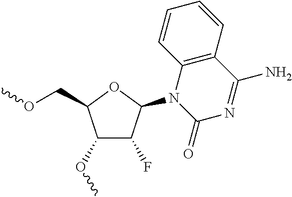 |

### Modified Oligonucleotide Compounds

An embodiment provides a STOPS™ modified oligonucleotide compound having sequence independent antiviral activity against hepatitis B, comprising an at least partially phosphorothioated sequence of modified nucleoside units, wherein the modified nucleoside units are any one or more selected from those set forth in Tables 1, 2 and 3 herein. In various embodiments, the STOPS™ modified oligonucleotide compound can further comprise one or more additional modified or unmodified nucleoside units, including but not limited to the A and C units described in Table 4 below.

TABLE 4
EXAMPLES OF A AND C UNITS

| Unit | Abbreviation | Structure |
|---|---|---|
| A | 2′-OMe-A | 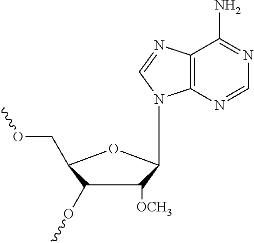 |
| A | 2′-O-MOE-A | 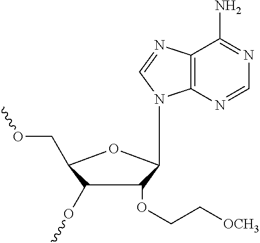 |
| A | LNA-A | 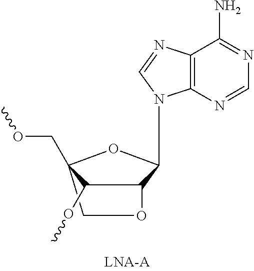 |
| A | 2′-O-Propargyl-A | 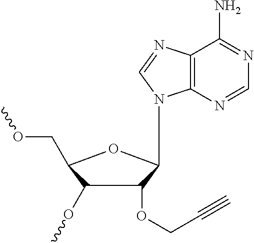 |
| A | 2′-F-A | 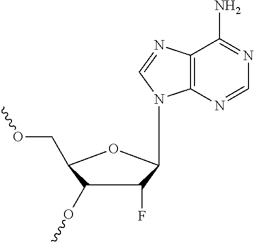 |
| A | 2′-araF-A | 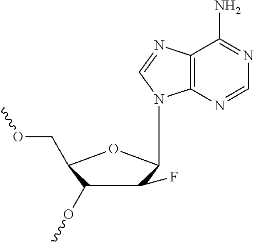 |
| A | 3′-OMe-A | 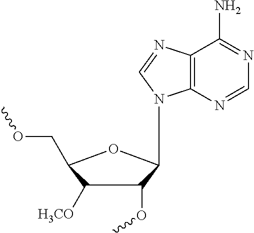 |
| A | UNA-A | 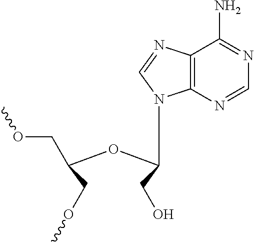 |
| A | 2′-NH₂-A | 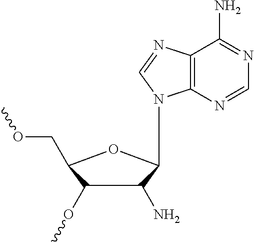 |
| A | GNA-A | 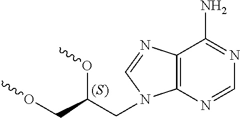 |
| A | ENA-A | 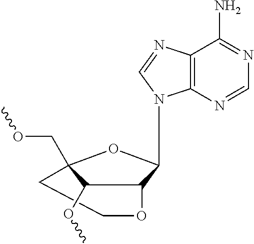 |
| A | 2′-O-Butynyl-A | 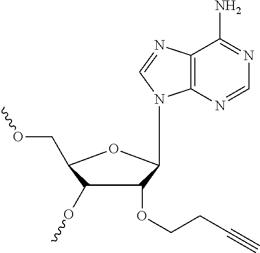 |
| A | scp-BNA-A | 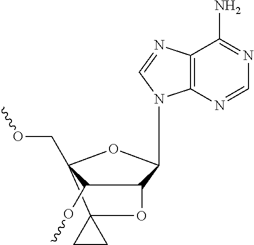 |
| A | AmNA(NMe)-A | 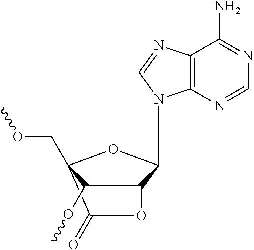 |
| A | nmLNA-A | 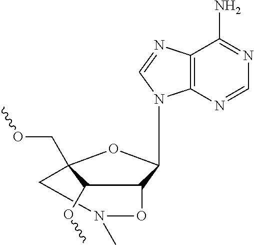 |
| A | 4etl-A | 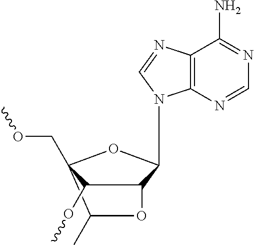 |
| A | Ribo-A | 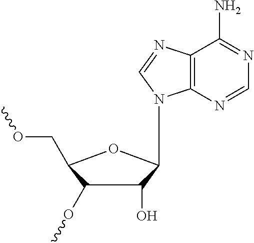 |
| C | 2′-OMe-(5m)C | 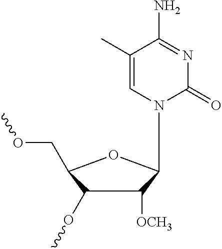 |
| C | 2′-O-MOE-(5m)C | 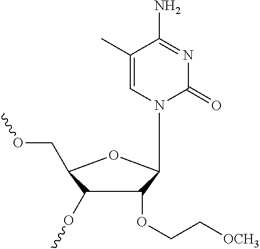 |
| C | LNA-(5m)C | 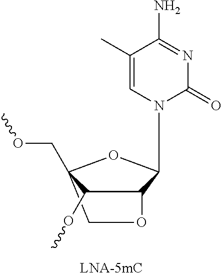 |
| C | 2′-O-Propargyl-(5m)C | 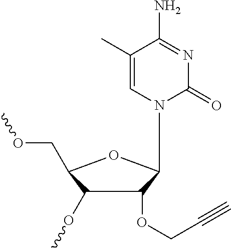 |
| C | 2′-F-(5m)C | 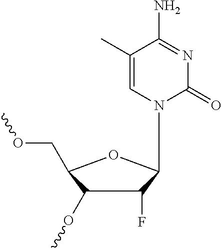 |
| C | 2′-araF-(5m)C | 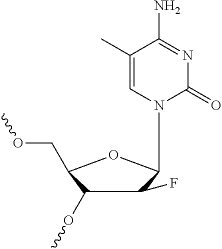 |
| C | 3′-OMe-(5m)C | 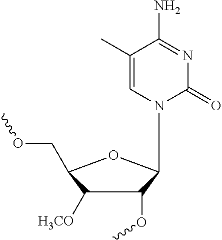 |
| C | UNA-(5m)C | 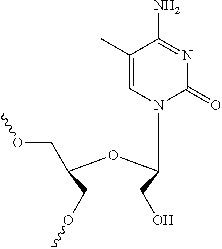 |
| C | 2′-NH₂-(5m)C | 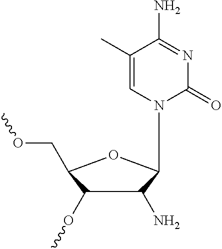 |
| C | GNA-(5m)C | 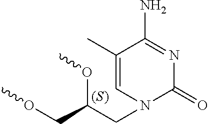 |
| C | ENA-(5m)C | 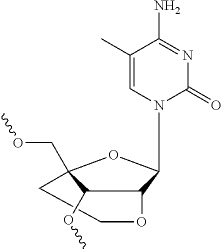 |
| C | 2′-O-Butynyl-(5m)C | 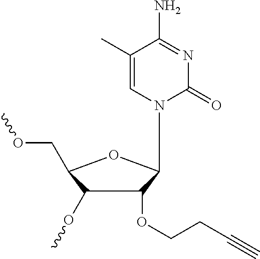 |
| C | scp-BNA-(5m)C | 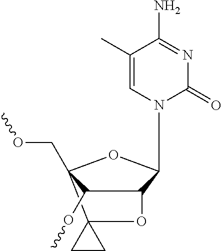 |
| C | AmNA-(NMe)-(5m)C | 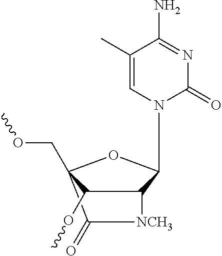 |
| C | 4etl-(5m)C | 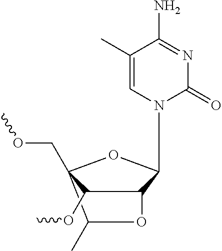 |
| C | nmLNA-(5m)C | 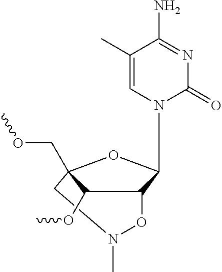 |
| C | Ribo-C | 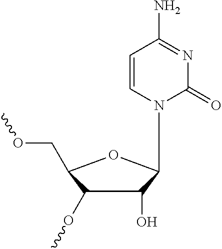 |
| C | Ribo-(5m)C | 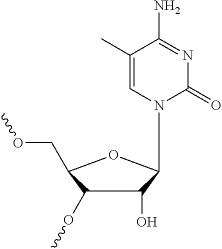 |

The length of a modified oligonucleotide as described herein can vary over a broad range. In various embodiments, a modified oligonucleotide as described herein comprises an at least partially phosphorothioated sequence of modified nucleoside units that has a sequence length of about 8 units, about 10 units, about 12 units, about 14 units, about 16 units, about 18 units, about 20 units, about 24 units, about 30 units, about 34 units, about 36 units, about 38 units, about 40 units, about 44 units, about 50 units, about 60 units, about 72 units, about 76 units, about 100 units, about 122 units, about 124 units, about 150 units, about 172 units, about 200 units, or a sequence length in a range between any two of the aforementioned values. For example, in an embodiment, the at least partially phosphorothioated sequence of modified nucleoside units has a sequence length in the range of 8 units to 200 units. In another embodiment, the at least partially phosphorothioated sequence of modified nucleoside units has a sequence length that is in any one or more (as applicable) of the following ranges: about 8 units to about 72 units; about 16 units to about 64 units; 20 units to 60 units; 24 units to 56 units; 30 units to 50 units; 34 units to 46 units, 36 units to 44 units; 38 units to 40 units; or about 40 units.

As described elsewhere herein, a modified oligonucleotide can comprise a single at least partially phosphorothioated sequence of modified nucleoside units in some embodiments, or in other embodiments the modified oligonucleotide can comprise a plurality of at least partially phosphorothioated sequences of modified nucleoside units that are linked together. Thus, a modified oligonucleotide that contains a single at least partially phosphorothioated sequence of modified nucleoside units can have the same sequence length as that sequence. Examples of such sequence lengths are described elsewhere herein. Similarly, a modified oligonucleotide that contains a plurality of at least partially phosphorothioated sequences of modified nucleoside units can have sequence length that is the result of linking those sequences as described elsewhere herein. Examples of sequence lengths for a modified oligonucleotide that contains a plurality of at least partially phosphorothioated sequences of modified nucleoside units are expressed elsewhere herein in terms of the lengths of the individual sequences, and also taking into account the length of the linking group.

A modified oligonucleotide as described herein can comprises a plurality of at least partially phosphorothioated sequences of modified nucleoside units. In an embodiment, the modified oligonucleotide can contain one or more of various nucleoside units (known to those skilled in the art, e.g., thymine (T), uracil (U), cytosine (C), adenine (A), guanine (G) and modified versions thereof) that are not modified nucleoside units as described in Tables 1-3, e.g., as an end group(s) and/or as a linking group(s) between two or more at least partially phosphorothioated sequences of modified nucleoside units. For example, in an embodiment, the modified oligonucleotide comprises one or more A, C, G, U and/or T units that link together at least two or more of the at least partially phosphorothioated sequences of modified nucleoside units as described in Tables 1-3. In an embodiment, the two or more at least partially phosphorothioated sequences of modified nucleoside units, which are linked together by A, C, G, U and/or T linking groups, are identical to one another. An example of such a modified oligonucleotide is (AC)₈-cytosine-(AC)₈, where in this context A and C are modified nucleoside units as described in Tables 1 and 2, respectively. Such a modified oligonucleotide that comprises a plurality of identical sequences that are joined together may be referred to herein as a concatemer. The two or more at least partially phosphorothioated sequences of modified nucleoside units that are linked together can also be different from one another. An example of such a modified oligonucleotide is (AC)₈-cytosine-(AC)₁₆, where in this context A and C are modified nucleoside units as described in Tables 1 and 2, respectively.

In various embodiments the modified oligonucleotide can contain two or more different modified A groups and/or two or more different modified C groups. In some embodiments such groups are arranged in an alternating fashion which may be expressed herein as (AC)ₙ, where n is an integer in the range of about 4 to about 100. When a modified A group or modified C group in such an (AC)ₙ sequence is replaced by a different modified

A group or modified C group, such a replacement is not ordinarily considered to interrupt the sequence of alternating modified A and C units. For example, in an embodiment, at least some of the modified A units are not 2′O-methylated on the ribose ring and/or at least some of the modified C units are not 2′O-methylated on the ribose ring. However, in some embodiments the group linking the two at least partially phosphorothioated sequences of modified A and C units is itself a modified A or C unit that interrupts the otherwise alternating sequence of modified A and C units. For example, an at least partially phosphorothioated 16-mer of modified A and C units may be linked by a modified A unit to another such 16-mer to form (AC)₈-A-(AC)₈. Similarly, such a 16-mer may be linked by a modified C unit to another such 16-mer to form (AC)₈-C-(AC)₈. As noted above, when a plurality of at least partially phosphorothioated sequences of modified A and C units that are identical to one another are joined together by a linking group, the modified oligonucleotide may be referred to herein as a concatemer. Also as noted above, the two or more at least partially phosphorothioated sequences of modified A and C units that are linked together can also be different from one another. Examples of such modified oligonucleotides include (AC)₈-A-(AC)₁₆ and (AC)₈-C-(AC)₁₆.

In an embodiment, the modified oligonucleotide comprises a 5′ endcap. In various embodiments, the 5′ endcap is selected from

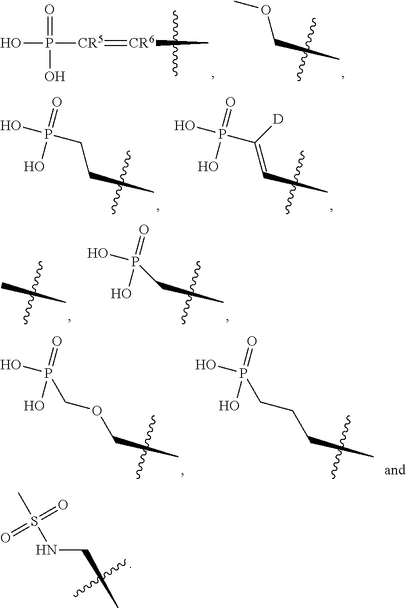

In an embodiment, R⁵ and R⁶ are each individually selected from hydrogen, deuterium, phosphate, thio C₁₋₆ alkyl, and cyano. For example, in an embodiment, R⁵ and R⁶ are both hydrogen and the modified oligonucleotide comprises a vinyl phosphonate endcap. In other embodiments, R⁵ and R⁶ are not both hydrogen. In some embodiments, the 5′ endcap is selected from

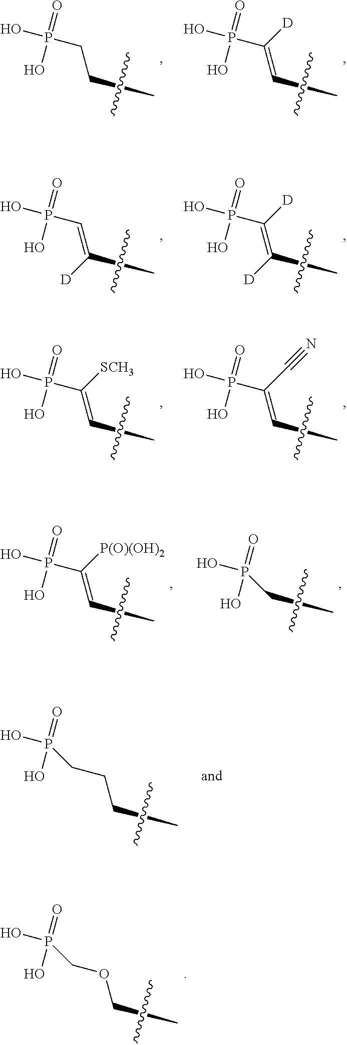

In an embodiment, the 5′ endcap is

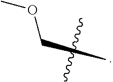

In an embodiment, the 5′ endcap is a methyl group, which may be depicted herein as

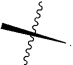

In another embodiment, the endcap is a C₁₋₃ alkylsulfonamide, such as

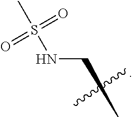

The 5′ endcap may be attached to the modified oligonucleotide in various ways. For example, in an embodiment, a vinyl phosphonate (VP) endcap may be incorporated into the modified oligonucleotide in the form of a 2′-OMe-4′-VP-phenyl end

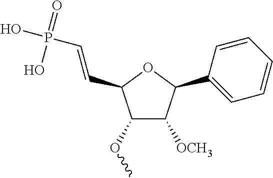

unit: 2′-OMe-4′-VP-phenyl or a 2′-OMe-4′-VP-Nap end unit:

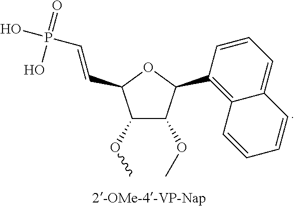

In another embodiment, a

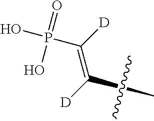

endcap may be incorporated into the modified oligonucleotide in the form of a 2′-OMe-DD VP-A end unit:

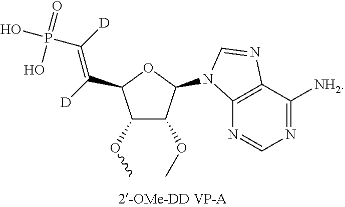

In another embodiment, a

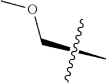

endcap may be incorporated into the modified oligonucleotide in the form of a 2′,5′-Di-OMe-A end unit:

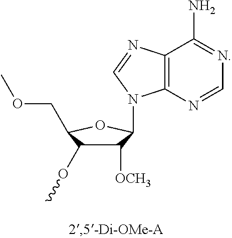

In another embodiment, a methylsulfonamide endcap may be incorporated into the modified oligonucleotide in the form of a 2′-OMe-5′-NH—SO₂ (CH₃)-A unit:

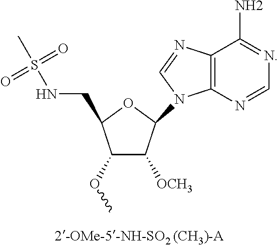

In other embodiments, the modified oligonucleotide comprises a 3′ and/or 5′ linking group. For example, with respect to modified oligonucleotide compounds comprising modified nucleoside units as described herein, such as the modified nucleoside units of Tables 1-3, at least one terminal

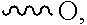

at least one terminal

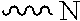

and/or at least one terminal

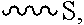

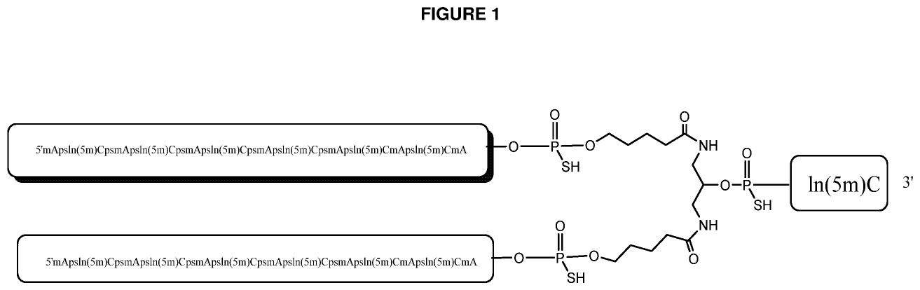

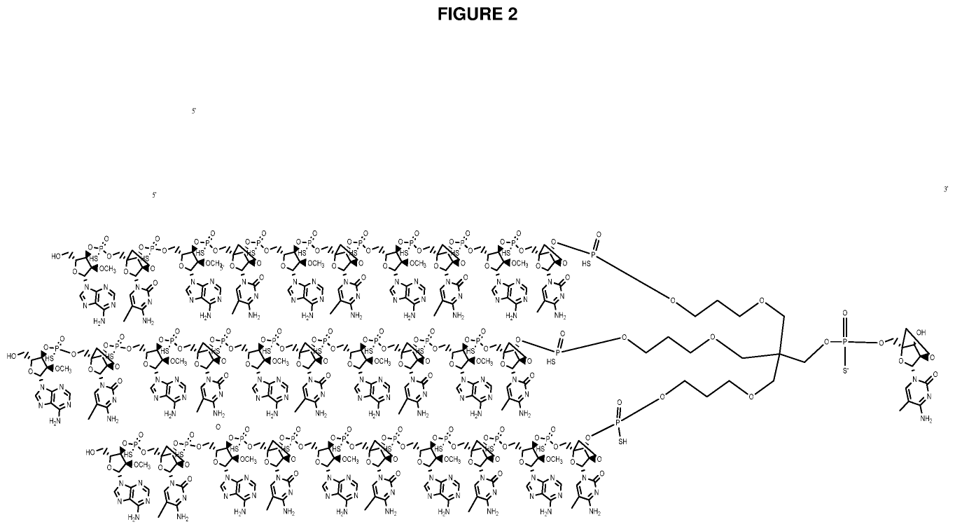

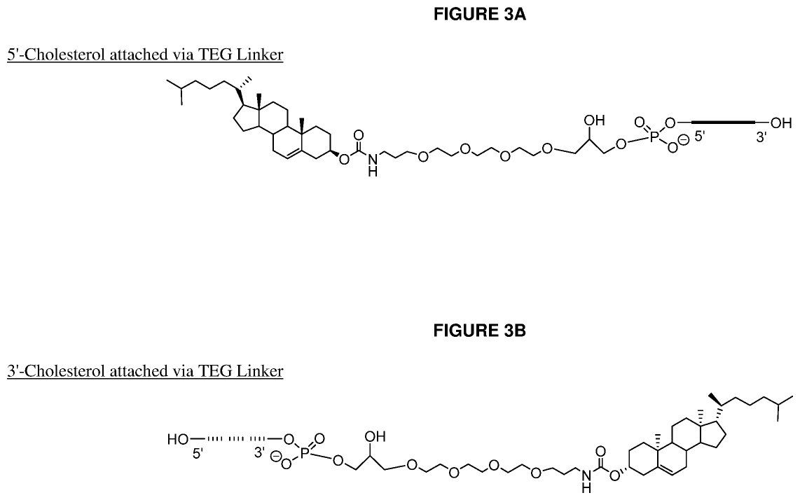

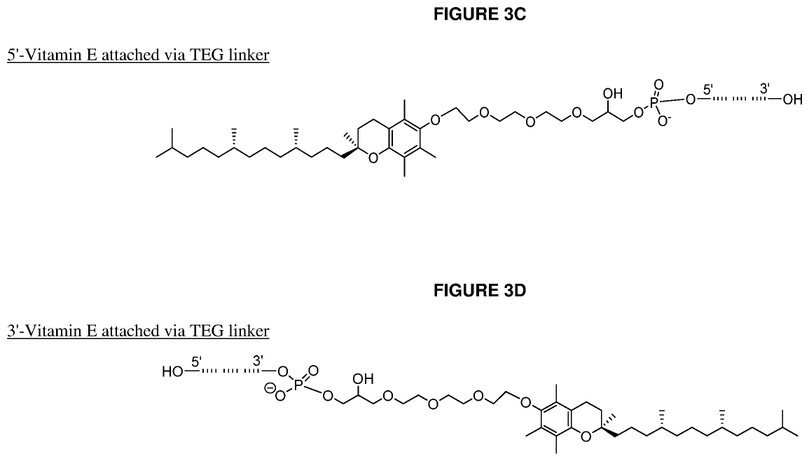

can be a linking group. Various linking groups known to those skilled in the art can be used to link the modified oligonucleotide to another moiety (such as one or more second oligonucleotides and/or targeting ligands). In an embodiment, the linking group comprises an A, C, G, U and/or T linking group or other unmodified unit that interrupts the sequence of modified nucleoside units as discussed above. In another embodiment, the linking group comprises a C₂₋₆ alkylene linkage (FIG. 1), a C₂₋₆alkylene oxide linkage, such as a propylene oxide linkage (FIG. 2), or a tetraethylene glycol (TEG) linkage (FIGS. 3A-D).

In various embodiments, two, three, four or more of the modified oligonucleotides can be connected to each other in various ways. For example, the modified oligonucleotides can be connected end-to-end via 3′ and/or 5′ linking groups, and/or a linking group can be connected to a one 3′ or 5′ end of multiple modified oligonucleotides, e.g., as illustrated in FIGS. 1 and 2.

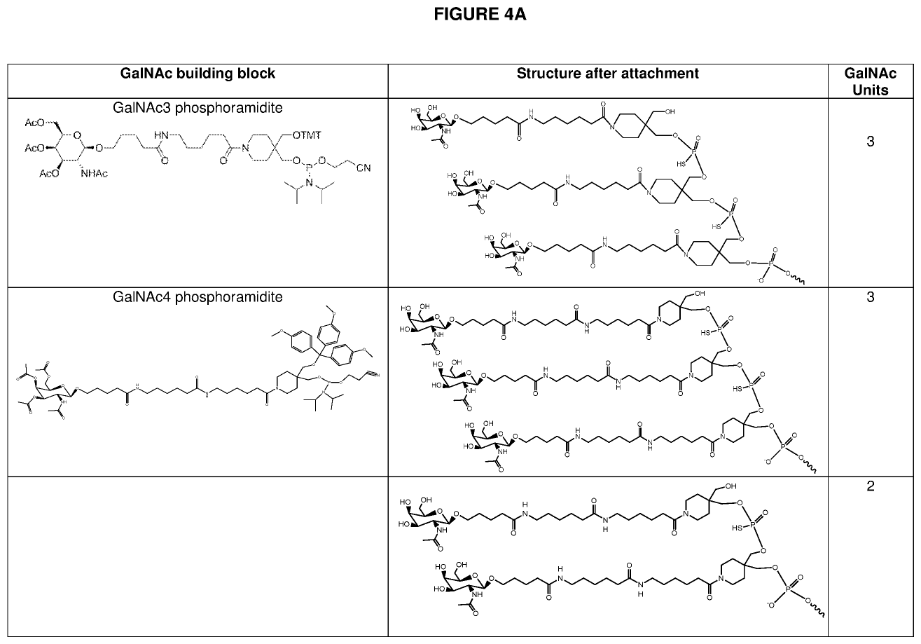

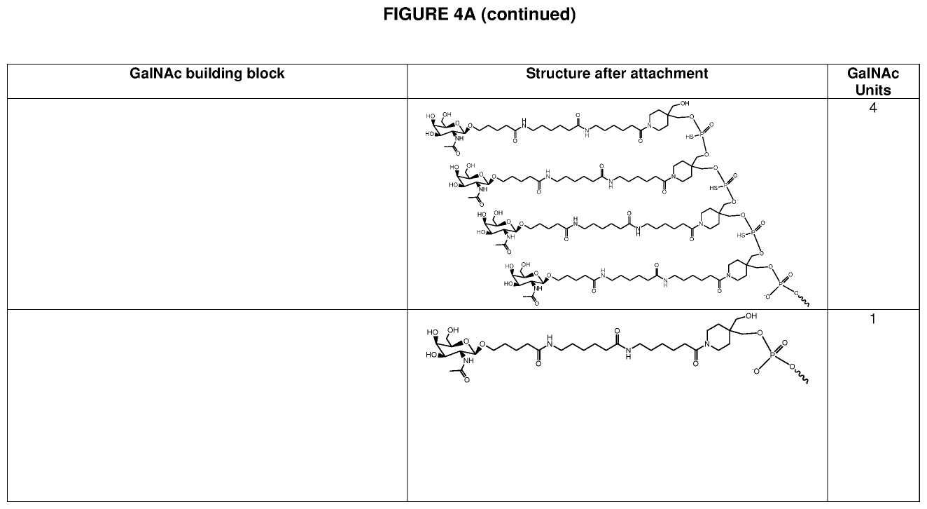

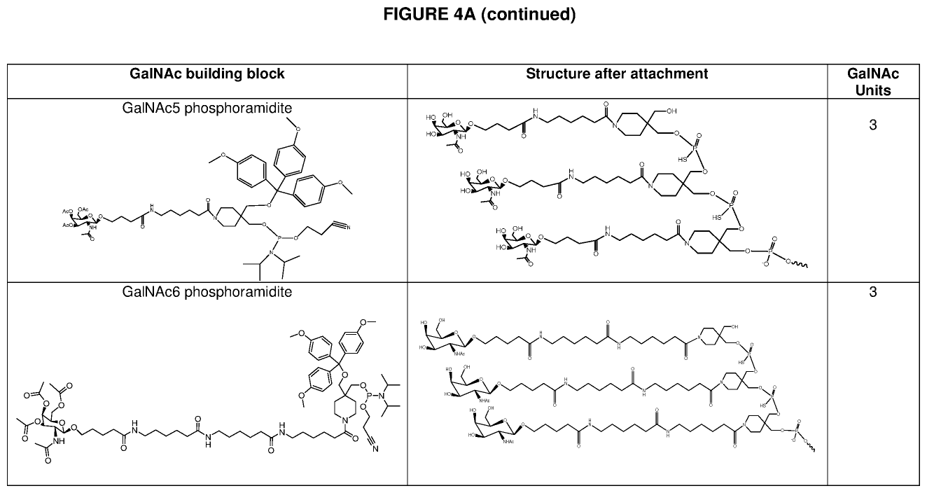

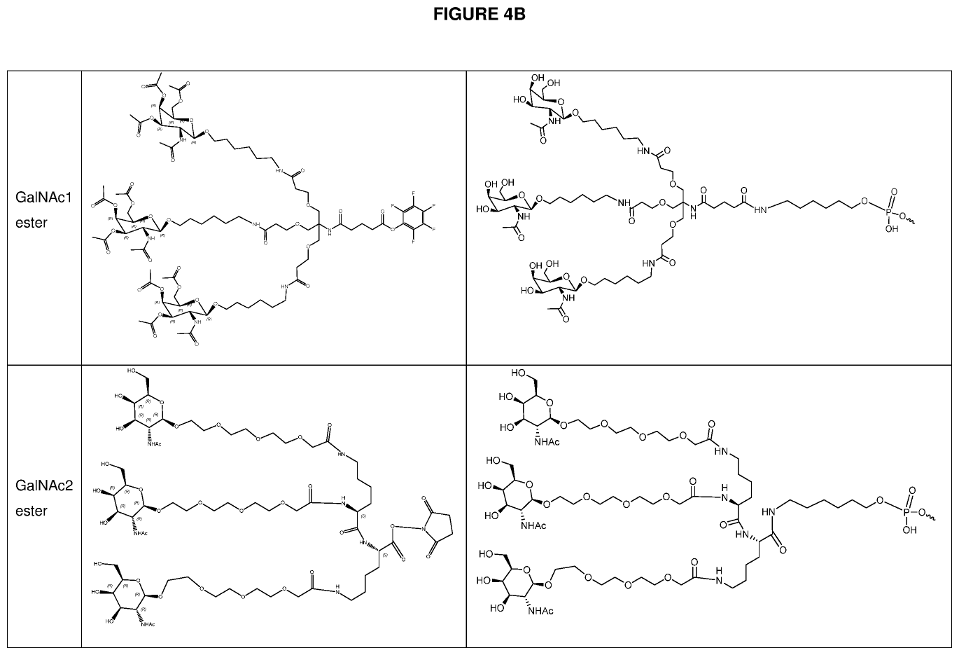

In various embodiments, the modified oligonucleotide further comprises a targeting ligand that is attached to the modified oligonucleotide via the linking group. For example, in various embodiments the targeting ligand is, or comprises, an N-acetylgalactosamine (GalNAc) (e.g., triantennary-GalNAc), a tocopherol or cholesterol. FIGS. 3A and 3B illustrate embodiments of modified oligonucleotides having cholesterol attached via a 5′ TEG linking group and a 3′TEG linking group, respectively. FIGS. 3C and 3D illustrate embodiments of modified oligonucleotides having a tocopherol (Vitamin E) attached via a 5′ TEG linking group and a 3′TEG linking group, respectively. FIGS. 4A and 4B illustrate embodiments of modified oligonucleotides having GalNAc attached via a linking group. In an embodiment, the GalNAc is a triantennary GalNAc. In various embodiments, the targeting ligand comprises GalNAc. For example, the targeting ligand may be a GalNAc targeting ligand that comprises 1, 2, 3, 4, 5 or 6 GalNAc units. In another embodiment, the targeting ligand is GalNAc2, GalNAc3, GalNAc4, GalNAc5 or GalNAc6.

In various embodiments, the at least partially phosphorothioated sequence of modified nucleoside units can include modification(s) to one or more phosphorothioated linkages. The inclusion of such a modified linkage is not ordinarily considered to interrupt the sequence of modified nucleoside units because those skilled in the art understand that such a sequence may be only partially phosphorothioated and thus may comprise one or more modifications to a phosphorothioate linkage. In various embodiments, the modification to the phosphorothioate linkage is a modified linkage selected from phosphodiester, phosphorodithioate, methylphosphonate, diphosphorothioate, 5′-phosphoramidate, 3′,5′-phosphordiamidate, 5′-thiophosphoramidate, 3′,5′-thiophosphordiamidate, 3′-S-phosphorothiolate and diphosphodiester. For example, in an embodiment, the modified linkage is a phosphodiester linkage.

In various embodiments, the at least partially phosphorothioated sequence of modified nucleoside units can have various degrees of phosphorothioation. For example, in an embodiment, the at least partially phosphorothioated sequence of modified nucleoside units is at least 50%, 55%, 60%, 65%, 70%, 75%, 80%, 85%, 90%, or 95% phosphorothioated. In an embodiment, the at least partially phosphorothioated sequence of modified nucleoside units is at least 85% phosphorothioated. In an embodiment, the at least partially phosphorothioated sequence of modified nucleoside units is fully phosphorothioated.

In various embodiments, the at least partially phosphorothioated sequence of modified nucleoside units can include stereochemical modification(s) to one or more phosphorothioated linkages. In an embodiment, the modified oligonucleotides described herein can comprise at least one stereochemically defined phosphorothioate linkage. In various embodiments, the stereochemically defined phosphorothioate linkage has an R configuration. In various embodiments, the stereochemically defined phosphorothioate linkage has an S configuration.

Those skilled in the art will recognize that modified oligonucleotide compounds comprising modified nucleoside units as described herein, such as the modified nucleoside units of Tables 1-3, contain internal linkages between the modified nucleoside units as well as terminal groups at the 3′ and 5′ ends. Thus, with respect to the modified nucleoside units described herein, such as the modified nucleoside units of Tables 1-3, each

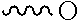

represents an internal

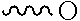

or a terminal

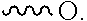

In various embodiments, each terminal

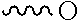

is independently hydroxyl, an O,O-dihydrogen phosphorothioate, a dihydrogen phosphate, an endcap or a linking group. A terminal

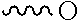

need not include an O atom, and thus may be an endcap such as a methyl group, which may be depicted herein as

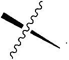

In various embodiments of the modified nucleoside units of Tables 1-3, each

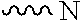

represents an internal

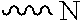

or a terminal

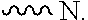

In various embodiments, each terminal

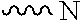

is independently an amine, a C₁₋₆ alkylamine, a di-C₁₋₆ alkylamine, an endcap or a linking group. A terminal

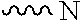

need not include an N atom, and thus may be an endcap such as a methyl group, which may be depicted herein as

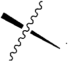

In various embodiments of the modified nucleoside units of Tables 1-3, each

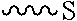

represents an internal

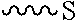

or a terminal

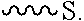

In various embodiments, each terminal

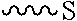

is independently a thiol, an O,O-dihydrogen phosphorothioate, a dihydrogen phosphate, an endcap or a linking group. A terminal

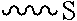

need not include an S atom, and thus may be an endcap such as a methyl group, which may be depicted herein as

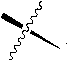

In various embodiments, each internal

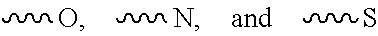

is joined together with the internal

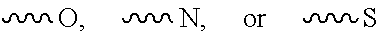

of a neighboring nucleoside unit to form a phosphorus-containing internucleoside linkage to the neighboring nucleoside unit, the phosphorus-containing linkage being of the formula

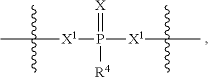

wherein each X is individually S or O; each X¹ is individually O, NR^{b}, or S; each R⁴ is individually OH, SH, optionally substituted C₁₋₆ alkyl, optionally substituted C₁₋₆ alkoxy, or optionally substituted amino; and each R^{b} is individually hours or C₁₋₆ alkyl. In an embodiment, at least one X is S. For example, in various embodiments the phosphorus-containing linkage of the formula

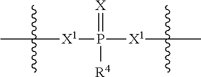

is selected from

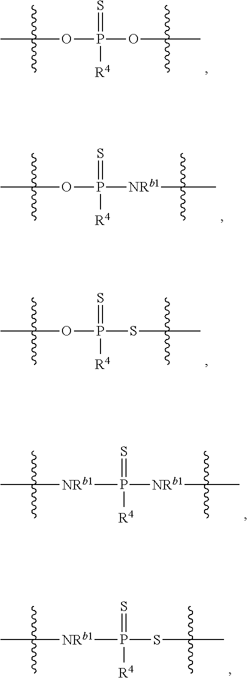

and

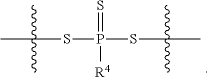

In some embodiments, examples of such phosphorus-containing linkages include

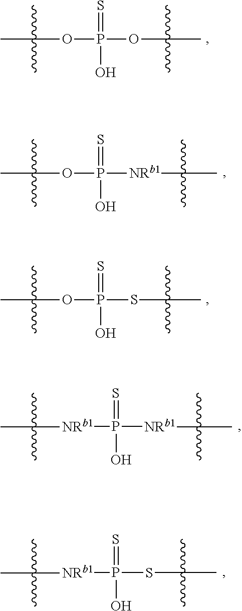

and

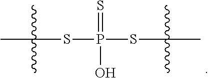 [SMILES: *SP(O)(=S)S*]

In various embodiments, the phosphorus-containing internucleoside linkage is a stereochemically defined linkage. Examples of such stereochemically defined linkages include the linkages of the formula (B1) and (B2) described below.

In various embodiments, each internal

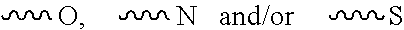

in the modified oligonucleotide is joined to the internal

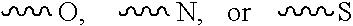

of a neighboring nucleoside unit to form an internucleoside phosphorothioate linkage or modified linkage as described elsewhere herein. For example, in an embodiment, the linkage is selected from phosphorothioate, phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate, 3′-S-phosphorothiolate or diphosphodiester.

In various embodiments, the modified nucleoside units of a modified oligonucleotide or complex thereof as described herein, can be arranged in various ways. In some embodiments, the at least partially phosphorothioated sequence of modified nucleoside units comprise alternating modified A units and modified C units, an arrangement that may be indicated herein as (AC)ₙ, where n is the number of such AC units. For example, in various embodiments n is an integer in the range of about 4 to about 100, about 9 to about 30, about 15 to about 25, about 17 to about 23, or about 18 to about 22.

In some embodiments, the modified nucleoside units of the modified oligonucleotide can be arranged in the form of blocks. For example, in an embodiment, the sequence of modified nucleoside units can comprise an A block that consists of 4, 5, 6, 7, 8, 9 or 10 consecutive modified A units selected from Table 1, or any range defined by any two such numbers of modified A units. In another embodiment, the sequence of modified nucleoside units can comprise a C block that consists of 4, 5, 6, 7, 8, 9 or 10 consecutive modified C units selected from Table 2, or any range defined by any two such numbers of modified C units. In another embodiment, the sequence of modified nucleoside units can comprise an other modified nucleoside block that consists of 4, 5, 6, 7, 8, 9 or 10 consecutive other modified nucleoside units selected from Table 3, or any range defined by any two such numbers of other modified nucleoside units.

Such A blocks, C blocks and/or other modified nucleoside blocks can themselves be arranged in various ways. In an embodiment, a modified oligonucleotide or complex thereof as described herein can comprise an A block at a first end position at a 3′ or 5′ end of the sequence of modified nucleoside units. In another embodiment, such a modified oligonucleotide or complex thereof can further comprise a second A block that is at a second end position at the opposite 5′ or 3′ end of the sequence of modified nucleoside units from the first end position. In another embodiment, a modified oligonucleotide or complex thereof as described herein can comprise a C block at a first end position at a 3′ or 5′ end of the sequence of modified nucleoside units. In another embodiment, such a modified oligonucleotide or complex thereof can further comprise a second C block that is at a second end position at the opposite 5′ or 3′ end of the sequence of modified nucleoside units from the first end position. In another embodiment, a modified oligonucleotide or complex thereof as described herein can comprise an A block at a first end position at a 3′ or 5′ end of the sequence of modified nucleoside units, and can further comprise a C block that is at a second end position at the opposite 5′ or 3′ end of the sequence of modified nucleoside units from the first end position.

In various embodiments, a modified oligonucleotide as described herein, comprising an at least partially phosphorothioated sequence of modified nucleoside units, has sequence independent antiviral activity against hepatitis B, as determined by HBsAg Secretion Assay, that is in an “A” activity range of less than 30 nanomolar (nM); in a “B” activity range of 30 nM to less than 100 nM; in a “C” activity range of 100 nM to less than 300 nM; or in a “D” activity range of greater than 300 nM. In some embodiments, a modified oligonucleotide as described herein, comprising an at least partially phosphorothioated sequence of modified nucleoside units, has sequence independent antiviral activity against hepatitis B, as determined by HBsAg Secretion Assay, that is less than 50 nM.

The modified oligonucleotides described herein can be prepared in the form of various complexes. Thus, an embodiment provides a chelate complex of a modified oligonucleotide as described herein. For example, in an embodiment such a chelate complex comprises a calcium, magnesium or zinc chelate complex of the modified oligonucleotide. The modified oligonucleotides described herein can also be prepared in the form of various monovalent counterion complexes. For example, in an embodiment such a counterion complex comprises a lithium, sodium or potassium complex of the modified oligonucleotide.

### Synthesis

The modified oligonucleotides described herein can be prepared in various ways. In an embodiment, the building block monomers described in Tables 5-7 are employed to make the modified oligonucleotides described herein by applying standard phosphoramidite chemistry. The building blocks described in Tables 5-7 and other building block phosphoramidite monomers can be prepared by known methods or obtained from commercial sources (Thermo Fischer Scientific US, Hongene Biotechnology USA Inc., Chemgenes Corporation). Exemplary procedures for making modified oligonucleotides are set forth in the Examples below.

TABLE 5
BUILDING BLOCKS FOR “A” AND MODIFIED “A” UNITS

| Abbreviation | Structure |
|---|---|
| 8-Me-2′- OMe-A PHOSPHOR- AMIDITE | 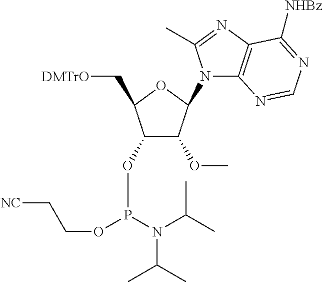 |
| 2′-OMe- 2F-A PHOSPHOR- AMIDITE | 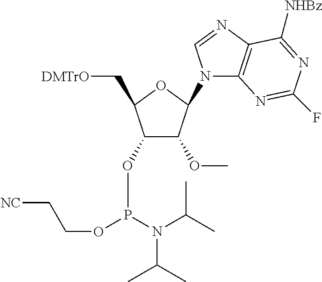 |
| 2′-OMe-2F- 7-deaza A PHOSPHOR- AMIDITE | 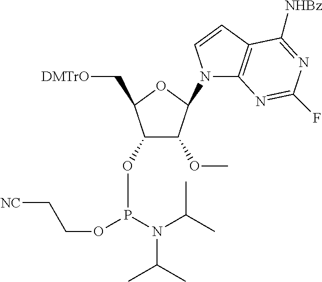 |
| 3′-N-2′- OMe-A PHOSPHOR- AMIDITE | 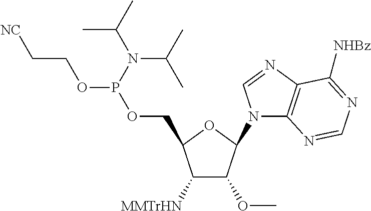 |
| 5′-N-2′- OMe-A PHOSPHOR- AMIDITE | 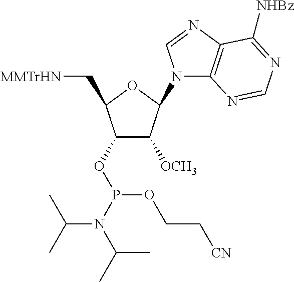 |
| 5′-S- 2′-d-A PHOSPHOR- AMIDITE | 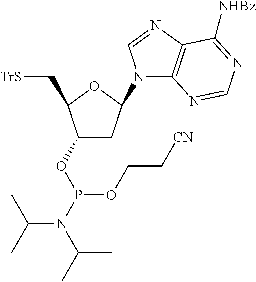 |
| 3′-N- 2′-d-A PHOSPHOR- AMIDITE | 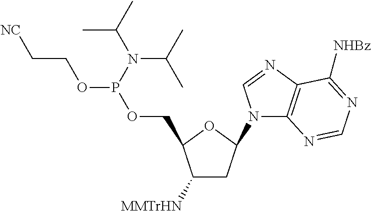 |
| 5′-DMTrO- 2′-OMe-A- PS-5′-N-2′- OMe-(5m)C dimer PHOSPHOR- AMIDITE | 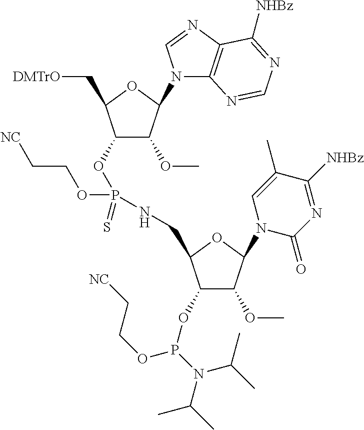 |
| 5′-DMTrO- 2′-OMe-A- 3′-S-PS-5′- S-2′-OMe- (5m)C dimer PHOSPHOR- AMIDITE | 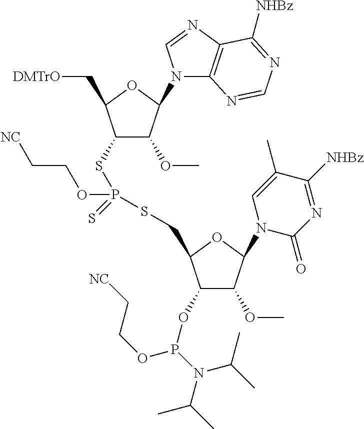 |
| 5′-DMTrO- 2′-OMe-A- 3′-S-PS-2′- OMe-(5m)C dimer PHOSPHOR- AMIDITE | 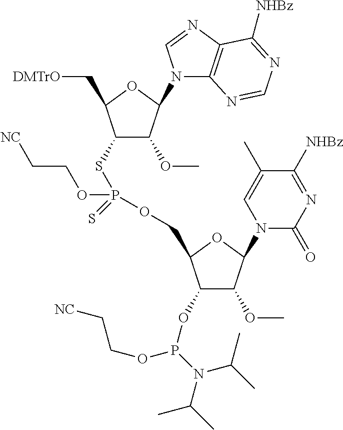 |
| Morpho- lino-A PHOSPHOR- AMIDITE | 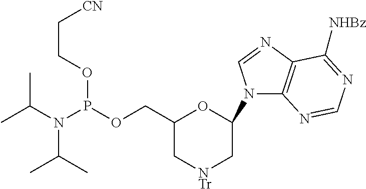 |
| Ara-A PHOSPHOR- AMIDITE | 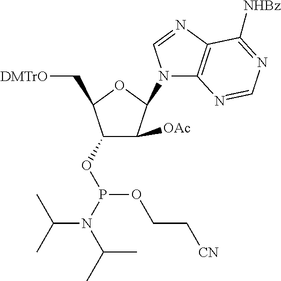 |
| 2′,5′-Di- OMe-A PHOSPHOR- AMIDITE | 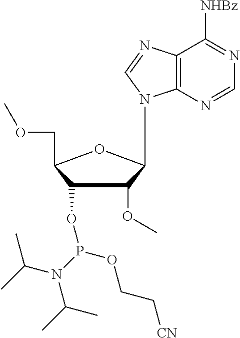 |
| 3′-N-2′- ara F-A PHOSPHOR- AMIDITE | 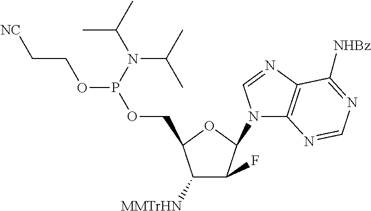 |
| 3′-N- 2′-F-A PHOSPHOR- AMIDITE | 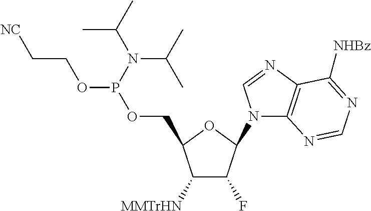 |
| 3′-N-2′- OMOE-A PHOSPHOR- AMIDITE | 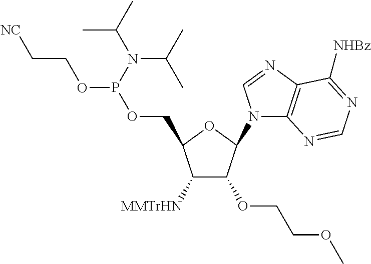 |
| 3′-N-2′- ribo-A | 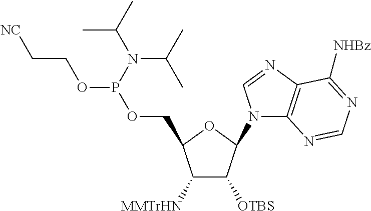 |
| 5′-S-2′- OMe-A PHOSPHOR- AMIDITE | 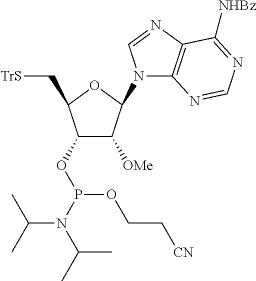 |
| 3′-S-2′- OMe-A PHOSPHOR- AMIDITE | 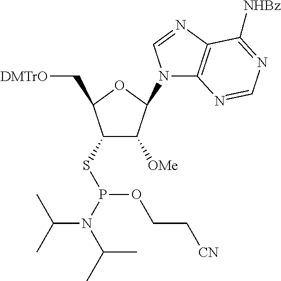 |
| 3′-S- 2′-d-A PHOSPHOR- AMIDITE | 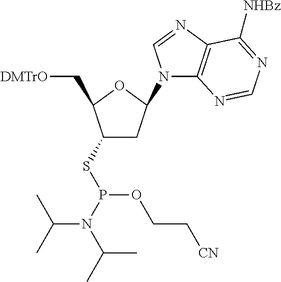 |
| 2′-OMe-A PHOSPHOR- AMIDITE | 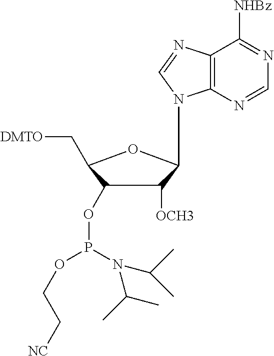 |
| 2′-F-A PHOSPHOR- AMIDITE | 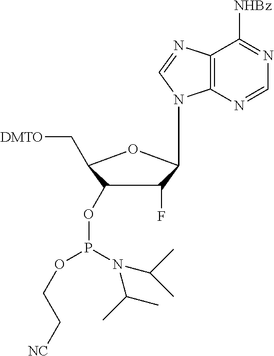 |
| 2′-O- MOE-A PHOSPHOR- AMIDITE | 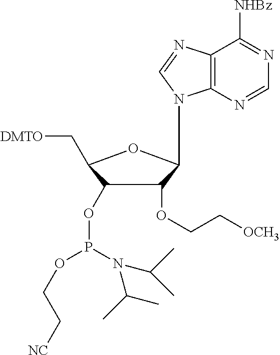 |
| LNA-A PHOSPHOR- AMIDITE | 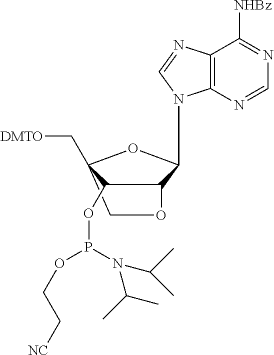 |
| ENA-A PHOSPHOR- AMIDITE | 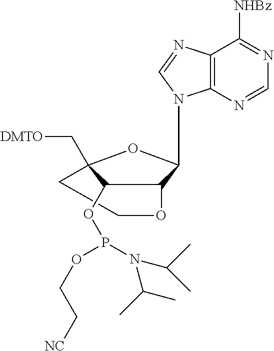 |
| 2′-O- Butyne-A PHOSPHOR- AMIDITE | 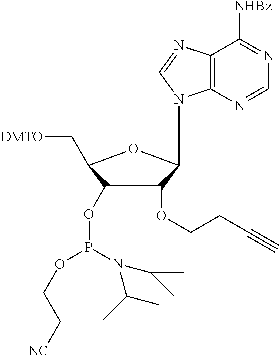 |
| 2′-NH₂-A PHOSPHOR- AMIDITE | 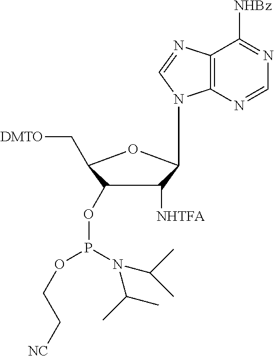 |
| 2′-F- Ara-A PHOSPHOR- AMIDITE | 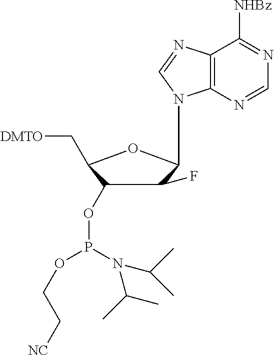 |
| 2′-O- Propargyl-A PHOSPHOR- AMIDITE | 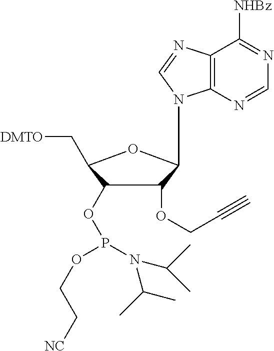 |
| UNA-A PHOSPHOR- AMIDITE | 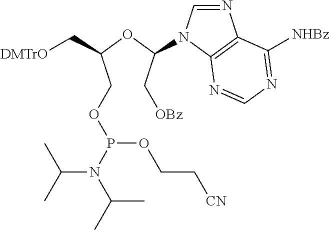 |
| GNA-A PHOSPHOR- AMIDITE | 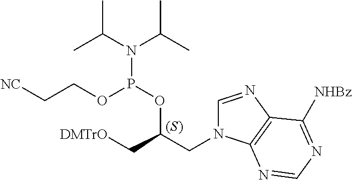 |
| 3′-O- Methyl-A PHOSPHOR- AMIDITE | 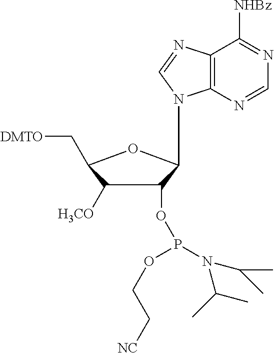 |
| scp- BNA-A PHOSPHOR- AMIDITE | 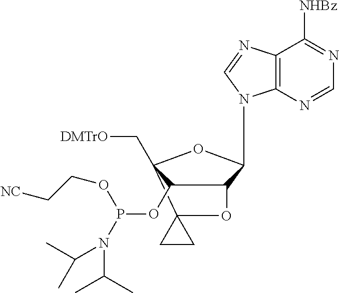 |
| AmNA- (N-Me)-A PHOSPHOR- AMIDITE | 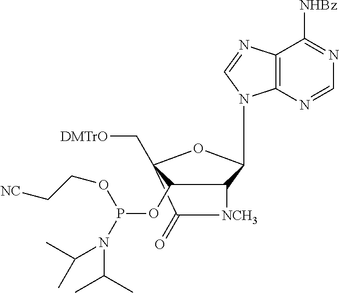 |
| nmLNA-A PHOSPHOR- AMIDITE | 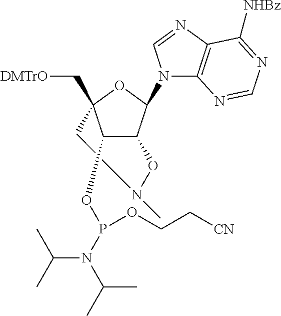 |
| 4etl-A PHOSPHOR- AMIDITE | 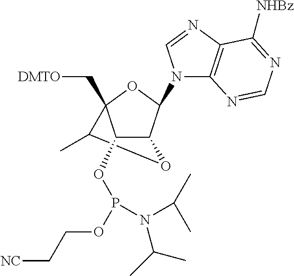 |
| Ribo-A PHOSPHOR- AMIDITE | 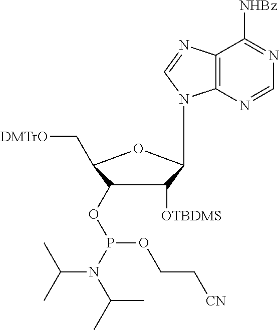 |
| 2′-OMe- 5′-CD₂-A PHOSPHOR- AMIDITE | 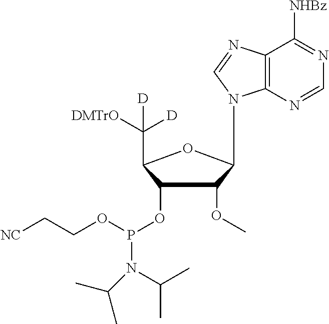 |
| 2′-F-5′- CD₂-A PHOSPHOR- AMIDITE | 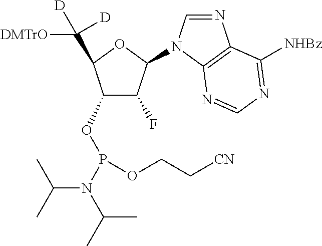 |
| 2′-OCD₃- 5′-CD₂-A PHOSPHOR- AMIDITE | 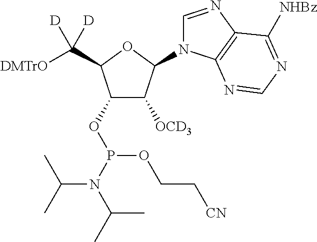 |
| 2′-OMe- DD VP-A PHOSPHOR- AMIDITE | 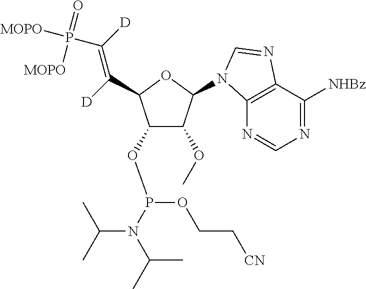 |
| 2′-OMe-5′- NH-SO₂ (CH₃)-A PHOSPHOR- AMIDITE | 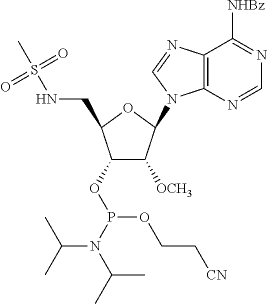 |

TABLE 6
BUILDING BLOCKS FOR “C” AND MODIFIED “C” UNITS

| Abbreviation | Structure |
|---|---|
| 2′-OMe-(5F)C PHOSPHORAMIDITE | 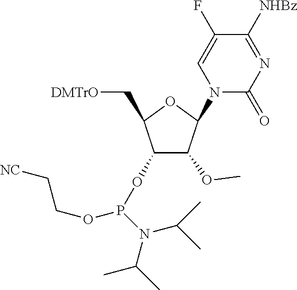 |
| Morpholino-C PHOSPHORAMIDITE | 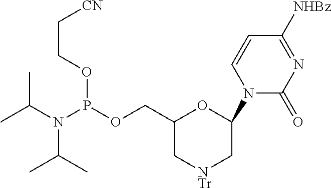 |
| 2′-Difluoro-C PHOSPHORAMIDITE | 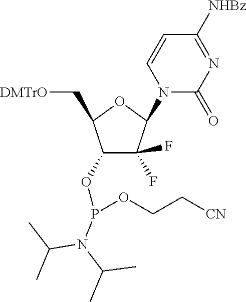 |
| 2′-OMe-5(CF₃)C PHOSPHORAMIDITE | 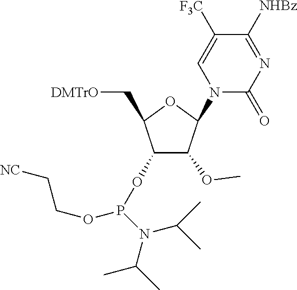 |
| 2′-OMe-5(PA)C PHOSPHORAMIDITE | 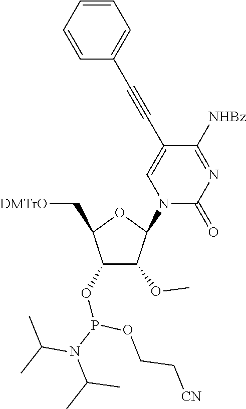 |
| 2′-OMe-5(N-propargyl-2- methylpropanamide)C PHOSPHORAMIDITE | 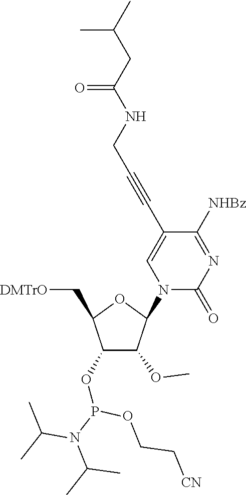 |
| Aminoethyl-Phenoxazine 2′-deoxyCytidine (dC) PHOSPHORAMIDITE (G clamp phosphoramidite) | 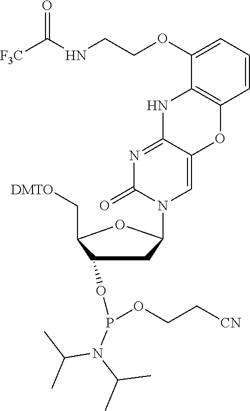 |
| 5-(1-Propynyl)-2′- deoxyCytidine PHOSPHORAMIDITE | 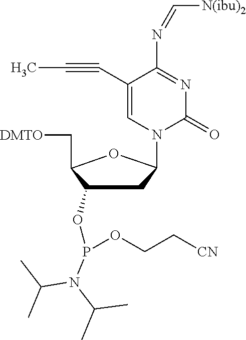 |
| 3′-N-2′-ara F-(5m)C PHOSPHORAMIDITE | 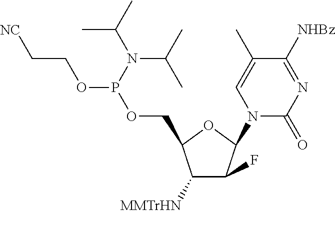 |
| 3′-N-2′-F-(5m)C PHOSPHORAMIDITE | 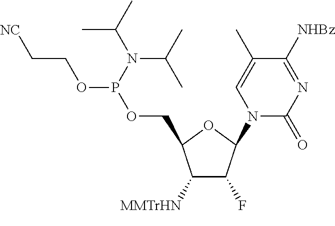 |
| 3′-N-2′-d-(5m)C PHOSPHORAMIDITE | 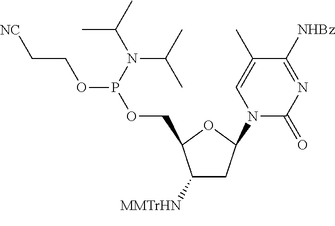 |
| 3′-N-2′-O-MOE-(5m)C PHOSPHORAMIDITE | 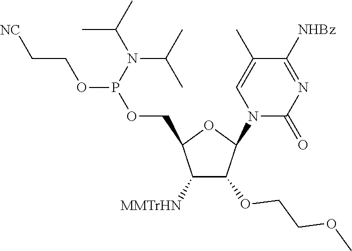 |
| 3′-N-2′-OME-(5m)C PHOSPHORAMIDITE | 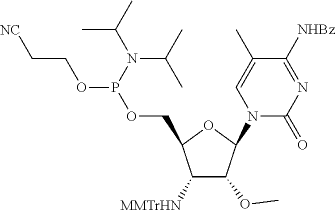 |
| 2′-OMe-(5- tetraoxapentadec-14-yn-1- ol)C (2′-OMe-5(TEG)C) PHOSPHORAMIDITE | 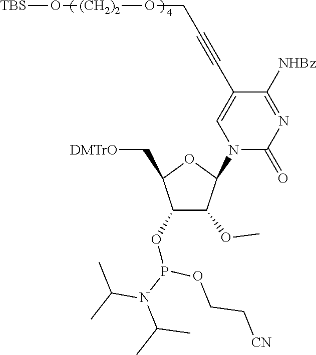 |
| 3′-N-2′-ribo-5(m)C PHOSPHORAMIDITE | 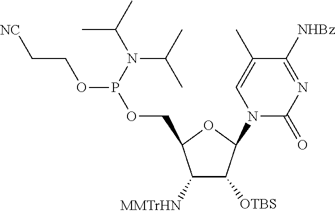 |
| 5′-S-2′-OMe-(5m)C PHOSPHORAMIDITE | 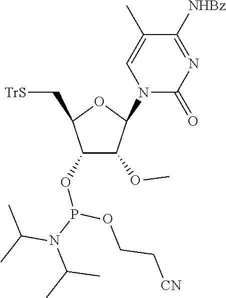 |
| 5′-S-2′-d-(5m)C PHOSPHORAMIDITE | 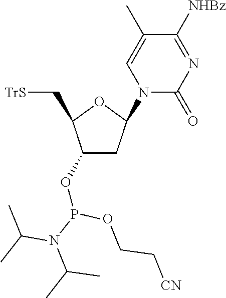 |
| 3′-S-2′-d-(5m)C PHOSPHORAMIDITE | 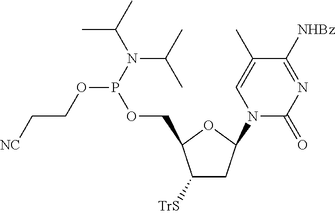 |
| 5′-N-2′-OMe-(5m)C PHOSPHORAMIDITE | 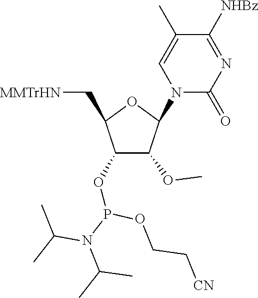 |
| 2′-OMe-(5m)C PHOSPHORAMIDITE | 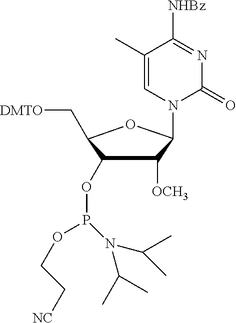 |
| 2′-F-(5m)C PHOSPHORAMIDITE | 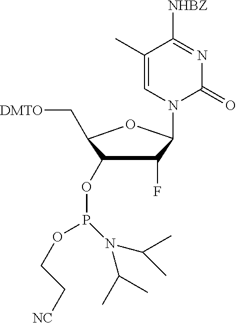 |
| 2′-O-MOE-(5m)C PHOSPHORAMIDITE | 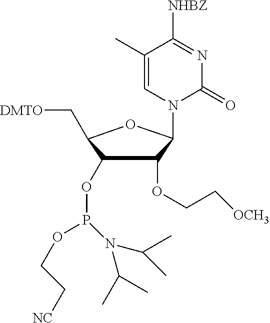 |
| LNA-(5m)C PHOSPHORAMIDITE | 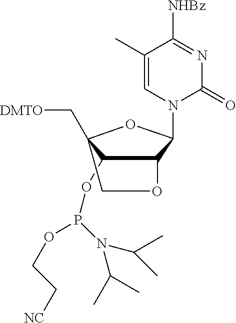 |
| ENA-(5m)C PHOSPHORAMIDITE | 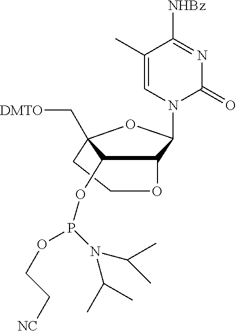 |
| 2′-O-Butyne-(5m)C PHOSPHORAMIDITE | 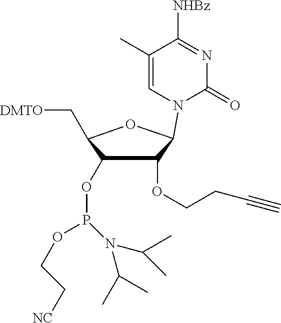 |
| 2′-NH₂-(5m)C PHOSPHORAMIDITE | 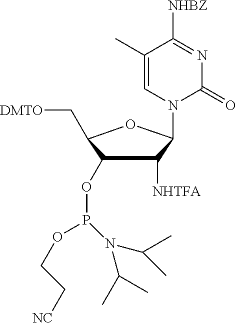 |
| 2′-F-Ara-(5m)C PHOSPHORAMIDITE | 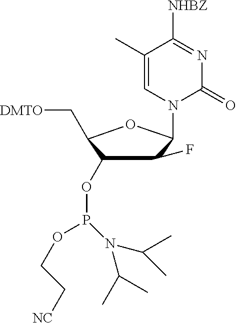 |
| 2′-O-Propargyl-(5m)C PHOSPHORAMIDITE | 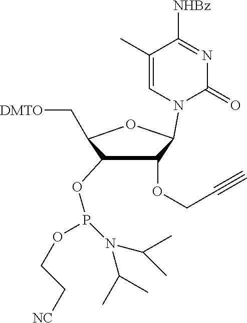 |
| UNA-(5m)C PHOSPHORAMIDITE | 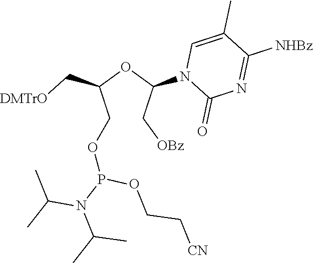 |
| GNA-(5m)C PHOSPHORAMIDITE | 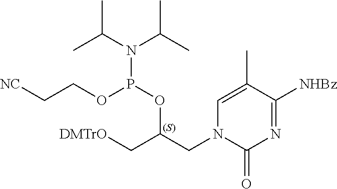 |
| 3′-O-Methyl-(5m)C PHOSPHORAMIDITE | 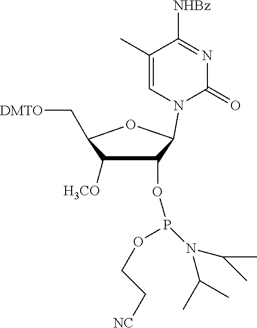 |
| scp-BNA-(5m)C PHOSPHORAMIDITE | 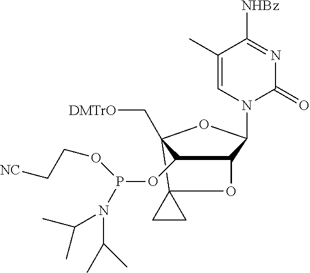 |
| AmNA-(NMe)-(5m)C PHOSPHORAMIDITE | 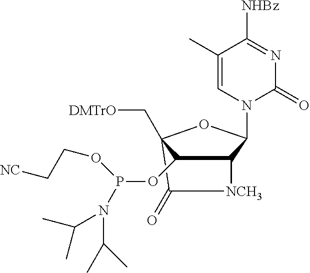 |
| 4etl-(5m)C PHOSPHORAMIDITE | 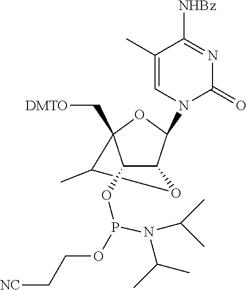 |
| nmLNA-(5m)C PHOSPHORAMIDITE | 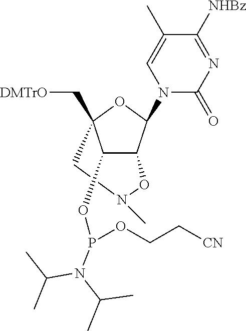 |
| Ribo-C PHOSPHORAMIDITE | 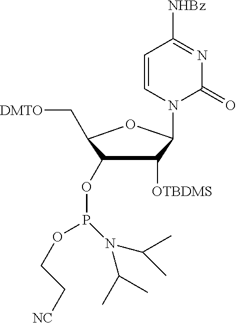 |
| Ribo-(5m)C PHOSPHORAMIDITE | 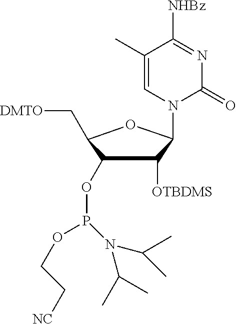 |
| 2′-OMe-5′-CD₂-C PHOSPHORAMIDITE | 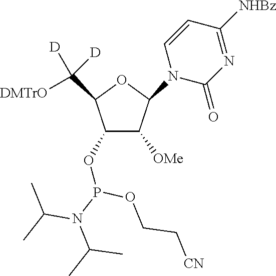 |
| 2′-OCD₃-C PHOSPHORAMIDITE | 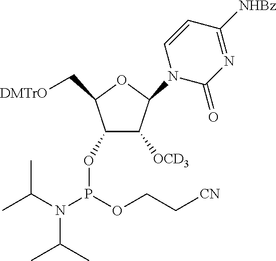 |
| 2′-F-5′-CD₂-C PHOSPHORAMIDITE | 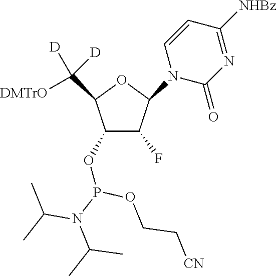 |
| 2′-OCD₃-5′-CD₂-C PHOSPHORAMIDITE | 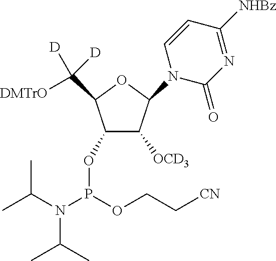 |
| 2′-F-quinazolinone PHOSPHORAMIDITE | 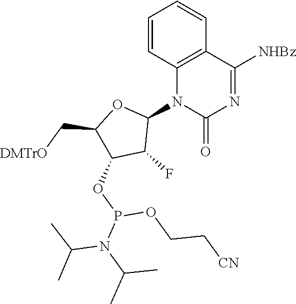 |

TABLE 7
BUILDING BLOCKS FOR OTHER MODIFIED NUCLEOSIDE UNITS

| Abbreviation | Structure |
|---|---|
| 2′-OMe-phenyl PHOSPHORAMIDITE | 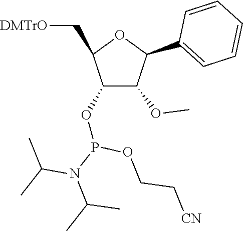 |
| 2′-OMe-4′-VP-phenyl PHOSPHORAMIDITE | 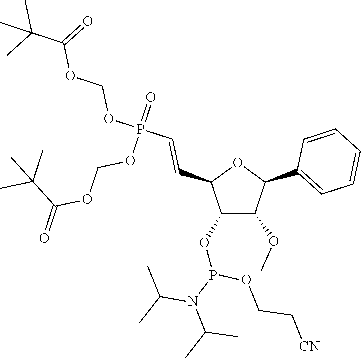 [SMILES: CO[C@@H]1[C@H](OP(OCCC#N)N(C(C)C)C(C)C)[C@@H](/C=C/P(=O)(OCOC(=O)C(C)(C)C)OCOC(=O)C(C)(C)C)O[C@H]1c1ccccc1] |
| 2′-F-phenyl PHOSPHORAMIDITE | 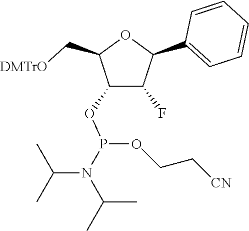 |
| 2′-OMe-Nap PHOSPHORAMIDITE | 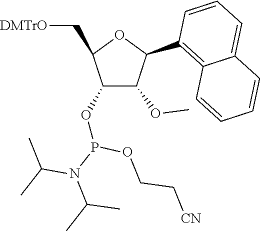 |
| 2′-OMe-4′-VP-naphthyl PHOSPHORAMIDITE | 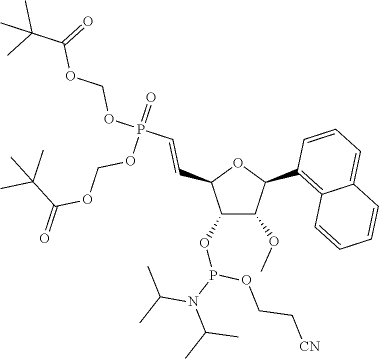 [SMILES: CO[C@@H]1[C@H](OP(OCCC#N)N(C(C)C)C(C)C)[C@@H](/C=C/P(=O)(OCOC(=O)C(C)(C)C)OCOC(=O)C(C)(C)C)O[C@H]1c1cccc2ccccc12] |
| 2′-OMe Abase PHOSPHORAMIDITE | 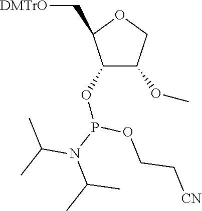 |
| Ribo-Abase PHOSPHORAMIDITE | 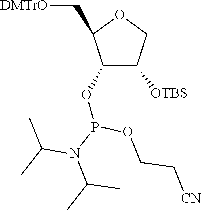 |
| 2′-OMe-4′-VP- PHOSPHORAMIDITE | 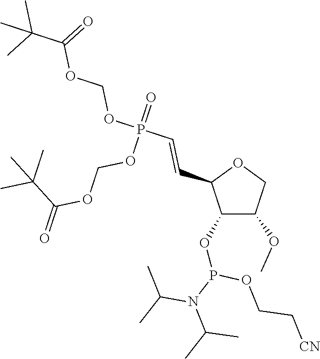 [SMILES: CO[C@H]1CO[C@H](/C=C/P(=O)(OCOC(=O)C(C)(C)C)OCOC(=O)C(C)(C)C)[C@H]1OP(OCCC#N)N(C(C)C)C(C)C] |
| 3′-N-2′-ribo-DAP PHOSPHORAMIDITE | 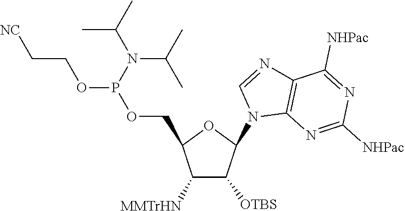 |
| 3′-N-2′-OMe-DAP PHOSPHORAMIDITE | 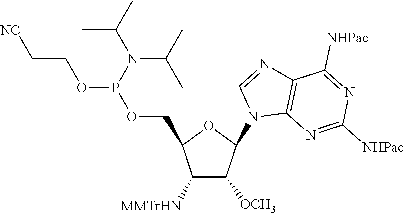 |

In various embodiments, the STOPS™ modified oligonucleotides described herein can also be prepared using dinucleotides that comprise or consist of the product obtainable or obtained by coupling any two of the building block monomers described in Tables 5-7. In various embodiments, the dinucleotides contain a stereochemically defined linkage that is also incorporated into the STOPS™ modified oligonucleotide that is formed by a process that comprises coupling one or more such dinucleotides. Accordingly, various embodiments provide a STOPS™ modified oligonucleotide as described herein, wherein such oligonucleotide comprises a stereochemically defined linkage as described herein. Exemplary procedures for making dinucleotides and the corresponding modified oligonucleotides are set forth in the Examples below.

An embodiment provides a dinucleotide comprising, or consisting of, two modified nucleoside units connected by a stereochemically defined linkage that is obtainable by coupling any two of the building block monomers described in Tables 5-7. In various embodiments, such dinucleotides comprise, or consist of, any two modified nucleoside units having a structure as described in Tables 1-3, in which two internal

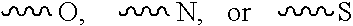

groups are joined together to form the stereochemically defined linkage of the dinucleotide; and in which each terminal

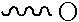

is independently hydroxyl, an O,O-dihydrogen phosphorothioate, an O,O-dihydrogen phosphate, a phosphoramidite, a trityl ether (TrO), a methoxytrityl ether (MMTrO), or a dimethoxytrityl ether (DMTO or DMTrO); each terminal

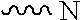

is independently an amine, a phosphoramidate, a thiophosphoramdiate, a phosphorodiamidate, a phosphorothiodiamidate, a tritylamino (TrNH), a methoxytritylamino (MMTrNH), or a dimethoxytrityl amino (DMTNH or DMTrNH); and/or each terminal

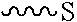

is independently a phosphoramidate, a S-phosphoramidite, a thiol, a thiolate, a phosphothioate, a phosphodithiolate, a trityl thioether (TrS), a methoxytrityl thioether (MMTrS), or a dimethoxytrityl thioether (DMTS or DMTrS). In an embodiment, the stereochemically defined linkage of the dinucleotide is a phosphorus-containing stereochemically defined linkage such as the stereochemically defined linkage of the formula (B1) or (B2) below. In an embodiment, one or more of the terminal

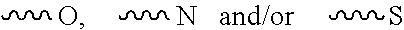

groups of the dinucleotide or the STOPS™ modified oligonucleotide is a phosphoramidite of the following formula (A):

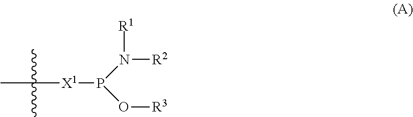

In various embodiments R¹ and R² of formula (A) are each individually a C₁₋₆ alkyl, X¹ is O, NR^{b} or S; R^{b} is hours or C₁₋₆ alkyl; and R³ is a C₁₋₆ alkyl or a cyano C₁₋₆ alkyl. For example, in an embodiment the phosphoramidite of the formula (A) is a phosphoramidite of the following formula (A1) in which X¹ is O, NR^{b} or S:

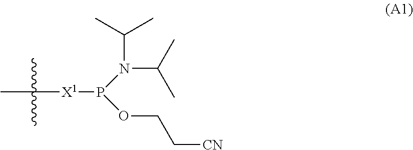

In various embodiments, two internal

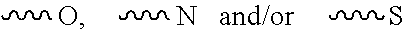

groups of the dinucleotide or the STOPS™ modified oligonucleotides are joined together to form a stereochemically defined linkage. In an embodiment, the stereochemically defined linkage is a phosphorus-containing stereochemically defined linkage. For example, in an embodiment, the stereochemically defined linkage is a linkage of the following formula (B1) or (B2):

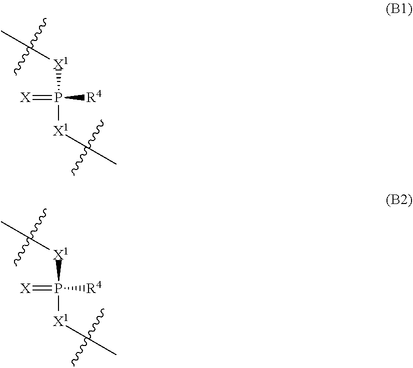

In various embodiments of formulae (B1) and (B2), X is S or O; each X¹ is independently O, NR^{b} or S; each R^{b} is independently hours or C₁₋₆ alkyl; and R⁴ is OH, SH, optionally substituted C₁₋₆ alkyl, optionally substituted C₁₋₆ alkoxy, or optionally substituted amino.

In some embodiments, an internal

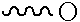

is joined together with another internal

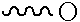

to form a stereochemically defined linkage of the dinucleotide or the STOPS™ modified oligonucleotide that is a phosphorothioate linkage. For example, in an embodiment, the stereochemically defined linkage is a phosphorothioate linkage of the following formula (B3) or (B4):

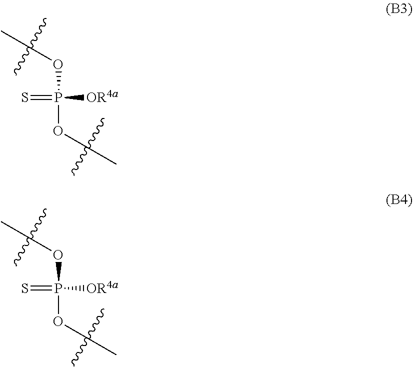

In various embodiments, R^{4a} of formulae (B3) and (B4) is a C₁₋₆ alkyl or a cyanoC₁₋₆ alkyl. For example, in an embodiment, the phosphorothioates of the formulae (B3) and (B4) are phosphorothioates of the following formulae (B5) or (B6), respectively:

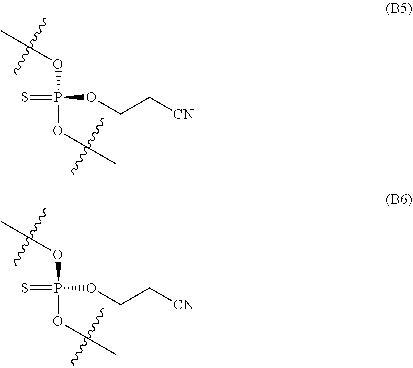

Various embodiments provide methods of making a modified oligonucleotide as described herein, comprising coupling one or more dinucleotides as described herein. Exemplary methods of carrying out such coupling are illustrated in the Examples below.

### Pharmaceutical Compositions

Some embodiments described herein relate to a pharmaceutical composition, that can include an effective amount of a compound described herein (e.g., a STOPS™ modified oligonucleotide compound or complex thereof as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, optionally in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a HBV and/or HDV infection that can include administering to a subject identified as suffering from the HBV and/or HDV infection an effective amount of a modified oligonucleotide or complex thereof as described herein, or a pharmaceutical composition that includes an effective amount of a modified oligonucleotide or complex thereof as described herein. Other embodiments described herein relate to using a modified oligonucleotide or complex thereof as described herein in the manufacture of a medicament for treating a HBV and/or HDV infection. Still other embodiments described herein relate to the use of a modified oligonucleotide or complex thereof as described herein or a pharmaceutical composition that includes a modified oligonucleotide as described herein for treating a HBV and/or HDV infection.

Various embodiments provide a treatment for hepatitis B, hepatitis D or both, comprising an effective amount of the modified oligonucleotide or complex thereof as described herein. Some embodiments provide a cross genotypic treatment for hepatitis B, hepatitis D or both, comprising an effective amount of the modified oligonucleotide or complex thereof as described herein. For example, in an embodiment, the modified oligonucleotide or complex thereof is effective to treat viral infections caused by two or more hepatitis B genotypes selected from genotype A, genotype B, genotype C, genotype D, genotype E, genotype F, genotype G, genotype H, genotype I and genotype J. In another embodiment, the modified oligonucleotide or complex thereof is effective to treat viral infections caused by two or more hepatitis B genotypes selected from genotype A, genotype B, genotype C and genotype D. In other embodiments, the modified oligonucleotide or complex thereof is effective to treat viral infections caused by two or more hepatitis D genotypes selected from genotype 1, genotype 2, genotype 3, genotype 4, genotype 5, genotype 6, genotype 7 and genotype 8.

Various routes may be used to administer a modified oligonucleotide or complex thereof to a subject in need thereof as indicated elsewhere herein. In an embodiment, the modified oligonucleotide or complex thereof is administered to the subject by a parenteral route. For example, in an embodiment, the modified oligonucleotide or complex thereof is administered to the subject intravenously. In another embodiment, the modified oligonucleotide or complex thereof is administered to the subject subcutaneously. In various embodiments, a modified oligonucleotide or complex thereof as described herein can be subcutaneously administered to a primate in an amount that is both safe and effective for treatment. Previously, subcutaneous administration of a modified oligonucleotide or complex thereof (such as REP 2139, REP 2055 or those described in U.S. Pat. Nos. 7,358,068; 8,008,269; 8,008,270 and 8,067,385) to a primate was considered unlikely to be safe and effective because of the relatively high dosages believed required to achieve efficacy and the concomitant increase in the potential risk of safety concerns such as undesirable injection site reactions. Thus, for example, prior clinical studies involving the administration of REP 2139 to humans are believed to have utilized only intravenous routes. At the dosage levels that were believed to be necessary for efficacy, it is believed that safety concerns such as undesirable injection site reactions would have precluded subcutaneous administration.

Some embodiments disclosed herein relate to a method of treating a HBV and/or HDV infection that can include contacting a cell infected with the HBV and/or HDV with an effective amount of a modified oligonucleotide or complex thereof as described herein, or a pharmaceutical composition that includes an effective amount of a modified oligonucleotide or complex thereof as described herein. In an embodiment, such a method of treating a HBV and/or HDV infection comprises safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

Other embodiments described herein relate to using a modified oligonucleotide or complex thereof as described herein in the manufacture of a medicament for treating a HBV and/or HDV infection. Still other embodiments described herein relate to the use of a modified oligonucleotide or complex thereof as described herein, or a pharmaceutical composition that includes an effective amount of a modified oligonucleotide or complex thereof as described herein for treating a HBV and/or HDV infection. In an embodiment, such uses comprise safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

Some embodiments disclosed herein relate to a method of inhibiting replication of HBV and/or HDV that can include contacting a cell infected with the HBV and/or HDV with an effective amount of a modified oligonucleotide or complex thereof as described herein, or a pharmaceutical composition that includes an effective amount of a modified oligonucleotide or complex thereof as described herein. In an embodiment, such a method of inhibiting replication of HBV and/or HDV comprises safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

Other embodiments described herein relate to using a modified oligonucleotide or complex thereof as described herein in the manufacture of a medicament for inhibiting replication of HBV and/or HDV. Still other embodiments described herein relate to the use of a modified oligonucleotide or complex thereof as described herein, or a pharmaceutical composition that includes an effective amount of a modified oligonucleotide or complex thereof as described herein, for inhibiting replication of HBV and/or HDV. In an embodiment, such uses for inhibiting replication of HBV and/or HDV comprise safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

In some embodiments, the HBV infection can be an acute HBV infection. In some embodiments, the HBV infection can be a chronic HBV infection.

Some embodiments disclosed herein relate to a method of treating liver cirrhosis that is developed because of a HBV and/or HDV infection that can include administering to a subject suffering from liver cirrhosis and/or contacting a cell infected with the HBV and/or HDV in a subject suffering from liver cirrhosis with an effective amount of a modified oligonucleotide or complex thereof as described herein, or a pharmaceutical composition that includes an effective amount of a modified oligonucleotide or complex thereof as described herein. In an embodiment, such a method of treating liver cirrhosis that is developed because of a HBV and/or HDV infection comprises safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

Other embodiments described herein relate to using a modified oligonucleotide or complex thereof as described herein in the manufacture of a medicament for treating liver cirrhosis that is developed because of a HBV and/or HDV infection, with an effective amount of the modified oligonucleotide(s). Still other embodiments described herein relate to the use of a modified oligonucleotide or complex thereof as described herein, or a pharmaceutical composition that includes an effective amount of a modified oligonucleotide or complex thereof as described herein for treating liver cirrhosis that is developed because of a HBV and/or HDV infection. In an embodiment, such uses for treating liver cirrhosis comprise safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein units, having sequence independent antiviral activity against hepatitis B, as determined by HBsAg Secretion Assay, that is in an “A” activity range of less than 30 nM.

Some embodiments disclosed herein relate to a method of treating liver cancer (such as hepatocellular carcinoma) that is developed because of a HBV and/or HDV infection that can include administering to a subject suffering from the liver cancer and/or contacting a cell infected with the HBV and/or HDV in a subject suffering from the liver cancer with an effective amount of a modified oligonucleotide or complex thereof as described herein, or a pharmaceutical composition that includes an effective amount of a modified oligonucleotide or complex thereof as described herein. In an embodiment, such a method of treating liver cancer (such as hepatocellular carcinoma) that is developed because of a HBV and/or HDV infection comprises safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein units, having sequence independent antiviral activity against hepatitis B, as determined by HBsAg Secretion Assay, that is in an “A” activity range of less than 30 nM.

Other embodiments described herein relate to using a modified oligonucleotide or complex thereof as described herein in the manufacture of a medicament for treating liver cancer (such as hepatocellular carcinoma) that is developed because of a HBV and/or HDV infection. Still other embodiments described herein relate to the use of a modified oligonucleotide or complex thereof as described herein, or a pharmaceutical composition that includes an effective amount of a modified oligonucleotide or complex thereof as described herein for treating liver cancer (such as hepatocellular carcinoma) that is developed because of a HBV and/or HDV infection. In an embodiment, such uses for treating liver cancer (such as hepatocellular carcinoma) comprise safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein units, having sequence independent antiviral activity against hepatitis B, as determined by HBsAg Secretion Assay, that is in an “A” activity range of less than 30 nM.

Some embodiments disclosed herein relate to a method of treating liver failure that is developed because of a HBV and/or HDV infection that can include administering to a subject suffering from liver failure and/or contacting a cell infected with the HBV and/or HDV in a subject suffering from liver failure with an effective amount of a modified oligonucleotide or complex thereof as described herein, or a pharmaceutical composition that includes an effective amount of a modified oligonucleotide or complex thereof as described herein. In an embodiment, such a method of treating liver failure that is developed because of a HBV and/or HDV infection comprises safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein units, having sequence independent antiviral activity against hepatitis B, as determined by HBsAg Secretion Assay, that is in an “A” activity range of less than 30 nM.

Other embodiments described herein relate to using a modified oligonucleotide or complex thereof as described herein in the manufacture of a medicament for treating liver failure that is developed because of a HBV and/or HDV infection. Still other embodiments described herein relate to the use of a modified oligonucleotide or complex thereof as described herein, or a pharmaceutical composition that includes an effective amount of a modified oligonucleotide or complex thereof as described herein for treating liver failure that is developed because of a HBV and/or HDV infection. In an embodiment, such uses for treating liver failure comprise safe and effective subcutaneous administration of the modified oligonucleotide or complex thereof to a human at a dosage lower than otherwise expected based on liver levels observed following otherwise comparable intravenous administration. For example, in an embodiment, the modified oligonucleotide or complex thereof comprises a highly potent STOPS™ compound or complex thereof as described herein. For example, in an embodiment, the STOPS™ compound or complex thereof is a modified oligonucleotide or complex thereof as described herein, comprising an at least partially phosphorothioated sequence of modified nucleoside units as described herein, having sequence independent antiviral activity against hepatitis B, as determined by HBsAg Secretion Assay, that is in an “A” activity range of less than 30 nM.

Various indicators for determining the effectiveness of a method for treating an HBV and/or HDV infection are also known to those skilled in the art. Examples of suitable indicators include, but are not limited to, a reduction in viral load indicated by reduction in HBV DNA (or load), HBV surface antigen (HBsAg) and HBV e-antigen (HBeAg), a reduction in plasma viral load, a reduction in viral replication, a reduction in time to seroconversion (virus undetectable in patient serum), an increase in the rate of sustained viral response to therapy, an improvement in hepatic function, and/or a reduction of morbidity or mortality in clinical outcomes.

In some embodiments, an effective amount of a modified oligonucleotide or complex thereof as described herein is an amount that is effective to achieve a sustained virologic response, for example, a sustained viral response 12 month after completion of treatment.

Subjects who are clinically diagnosed with an HBV and/or HDV infection include “naïve” subjects (e.g., subjects not previously treated for HBV and/or HDV) and subjects who have failed prior treatment for HBV and/or HDV (“treatment failure” subjects). Treatment failure subjects include “non-responders” (subjects who did not achieve sufficient reduction in ALT levels, for example, subject who failed to achieve more than 1 log 10 decrease from base-line within 6 months of starting an anti-HBV and/or anti-HDV therapy) and “relapsers” (subjects who were previously treated for HBV and/or HDV whose ALT levels have increased, for example, ALT>twice the upper normal limit and detectable serum HBV DNA by hybridization assays). Further examples of subjects include subjects with a HBV and/or HDV infection who are asymptomatic.

In some embodiments, a modified oligonucleotide or complex thereof as described herein can be provided to a treatment failure subject suffering from HBV and/or HDV. In some embodiments, a modified oligonucleotide or complex thereof as described herein can be provided to a non-responder subject suffering from HBV and/or HDV. In some embodiments, a modified oligonucleotide or complex thereof as described herein can be provided to a relapser subject suffering from HBV and/or HDV. In some embodiments, the subject can have HBeAg positive chronic hepatitis B. In some embodiments, the subject can have HBeAg negative chronic hepatitis B. In some embodiments, the subject can have liver cirrhosis. In some embodiments, the subject can be asymptomatic, for example, the subject can be infected with HBV and/or HDV but does not exhibit any symptoms of the viral infection. In some embodiments, the subject can be immunocompromised. In some embodiments, the subject can be undergoing chemotherapy.

Examples of agents that have been used to treat HBV and/or HDV include interferons (such as IFN-α and pegylated interferons that include PEG-IFN-α-2a), and nucleosides/nucleotides (such as lamivudine, telbivudine, adefovir dipivoxil, clevudine, entecavir, tenofovir alafenamide and tenofovir disoproxil). However, some of the drawbacks associated with interferon treatment are the adverse side effects, the need for subcutaneous administration and high cost. A drawback with nucleoside/nucleotide treatment can be the development of resistance.

Resistance can be a cause for treatment failure. The term “resistance” as used herein refers to a viral strain displaying a delayed, lessened and/or null response to an anti-viral agent. In some embodiments, a modified oligonucleotide or complex thereof as described herein can be provided to a subject infected with an HBV and/or HDV strain that is resistant to one or more anti-HBV and/or anti-HDV agents. Examples of anti-viral agents wherein resistance can develop include lamivudine, telbivudine, adefovir clevudine, entecavir, tenofovir alafenamide and tenofovir disoproxil. In some embodiments, development of resistant HBV and/or HDV strains is delayed when a subject is treated with a modified oligonucleotide as described herein compared to the development of HBV and/or HDV strains resistant to other HBV and/or HDV anti-viral agents, such as those described.

### Combination Therapies

In some embodiments, a modified oligonucleotide or complex thereof as described herein can be used in combination with one or more additional agent(s) for treating and/or inhibiting replication HBV and/or HDV. Additional agents include, but are not limited to, an interferon, nucleoside/nucleotide analogs, a capsid assembly modulator, a sequence specific oligonucleotide (such as anti-sense oligonucleotide and siRNA), an entry inhibitor and/or a small molecule immunomodulator. Examples of additional agents include recombinant interferon alpha 2b, IFN-α, PEG-IFN-α-2a, lamivudine, telbivudine, adefovir dipivoxil, clevudine, entecavir, tenofovir alafenamide, tenofovir disoproxil, JNJ-3989 (ARO-HBV), RG6004, GSK3228836, AB-729, VIR-2218, DCR-HBVS, JNJ-6379, GLS4, ABI-HO731, JNJ-440, NZ-4, RG7907, AB-423, AB-506, ABI-H2158, ALG-000184 and ALG-020572. In an embodiment, the additional agent is a capsid assembly modulator (CAM). In an embodiment, the additional agent is an anti-sense oligonucleotide (ASO).

In some embodiments, a modified oligonucleotide or complex thereof as described herein can be administered with one or more additional agent(s) together in a single pharmaceutical composition. In some embodiments, a modified oligonucleotide or complex thereof as described herein can be administered with one or more additional agent(s) as two or more separate pharmaceutical compositions. Further, the order of administration of a modified oligonucleotide or complex thereof as described herein with one or more additional agent(s) can vary.

### EXAMPLES

Additional embodiments are disclosed in further detail in the following examples, which are not in any way intended to limit the scope of the claims.

### Example A1

Embodiments of various end capped oligonucleotides described herein were made by using a 5′-ethyl phosphonate (5′-EP) building block to incorporate 5′-ethyl phosphonate endcaps:

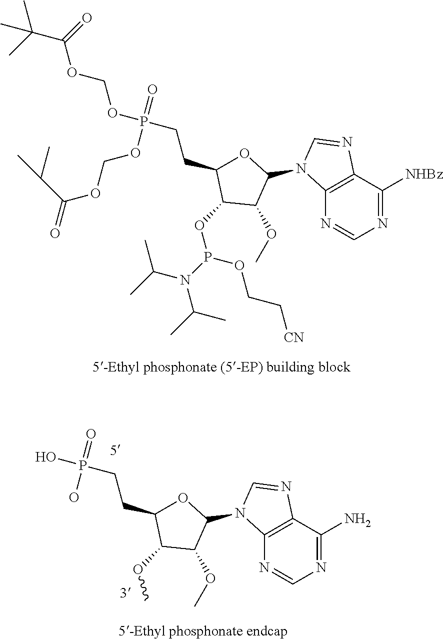

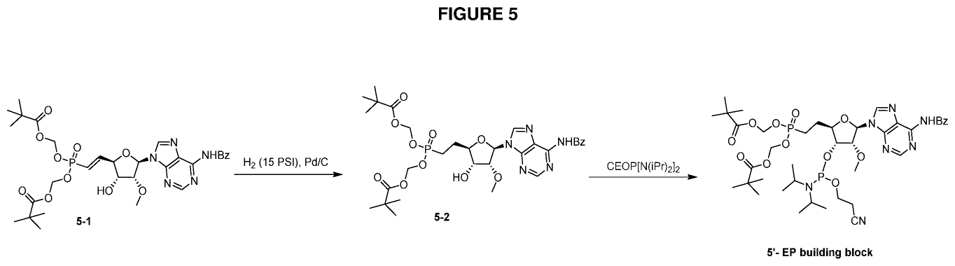

With reference to FIG. 5, the 5′-Ethyl phosphonate building block was prepared as follows:

Compound 5-1 was prepared according the procedure described in Journal of Medicinal Chemistry, 2018, vol. 61(3), 734-744. To a mixture of 5-1 (3.0 g, 4.35 mmol, 1 eq) in MeOH (5 mL) was added Pd/C (900 mg, 72.50 mol, 10% purity) under N₂. The suspension was degassed under vacuum and purged with H₂ for several times. The mixture was stirred under H₂ (15 psi) at 20° C. for 12 hour. ¹H NMR and ³¹P NMR showed 5-1 was consumed completely to form desired product. The reaction mixture was filtered and concentrated to give [2-[(2R,3R,4R,5R)-5-(6-benzamidopurin-9-yl)-3-hydroxy-4-methoxy-tetrahydrofuran-2-yl]ethyl-(2,2-dimethylpropanoyloxymethoxy)phosphoryl]oxymethyl 2,2-dimethylpropanoate, compound 5-2, (2.8 g, 4.05 mmol, 93.06% yield) as a white solid. ¹H NMR (400 MHz, CD₃OD) δ=8.75 (s, 1H), 8.53 (s, 1H), 8.08 (d, J=7.5 Hz, 2H), 7.68-7.61 (m, 1H), 7.59-7.50 (m, 2H), 7.23-7.17 (m, 1H), 7.15-7.10 (m, 1H), 6.15 (d, J=4.2 Hz, 1H), 5.71-5.61 (m, 4H), 4.57 (t, J=4.7 Hz, 1H), 4.41 (t, J=5.3 Hz, 1H), 4.09-3.99 (m, 1H), 3.49 (s, 3H), 2.16-1.97 (m, 4H), 1.17 (d, J=3.5 Hz, 18H); ³¹P NMR (162 MHz, CD₃CN) δ=32.91 (s, 1P).

To a solution of 5-2 (2.3 g, 3.33 mmol, 1 eq) in DCM (30 mL) was added 1H-imidazole-4,5-dicarbonitrile (589.06 mg, 4.99 mmol, 1.5 eq) followed by 3-bis(diisopropylamino)phosphanyloxypropanenitrile (2.00 g, 6.65 mmol, 2.11 mL, 2.0 eq), and the mixture was stirred at 25° C. for 2 hour. TLC indicated that majority of 5-2 was consumed and one major new spot was formed. The reaction mixture was washed with H₂O (50 mL*2) and brine (50 mL*2), dried over Na₂SO₄, and concentrated to give a residue. The residue was purified by Flash-C-18 column using the following conditions: Column, C18 silica gel; mobile phase, water and acetonitrile (0%-70% acetonitrile) to give 5′-EP building block, (1.4 g, 1.53 mmol, 45.88% yield, 97.2% purity) as a light yellow solid. LCMS (ESI): m/z calcd. for C₄₀H₆₀N₇O₁₂P₂ 892.37 [M+H]⁺, found 892.0. HPLC: Mobile Phase: 10 mMol NH₄Ac in water (solvent C) and acetonitrile (solvent D), using the elution gradient 80%-100% (solvent D) over 10 minutes and holding at 100% for 5 minutes at a flow rate of 1.0 mL/minute; Column30: Waters Xbridge C18 3.5 um, 150*4.6 mm; ¹H NMR (400 MHz, CD₃CN) δ=δ=9.40 (s, 1H), 8.67 (s, 1H), 8.27 (d, J=1.8 Hz, 1H), 8.01 (d, J=7.5 Hz, 2H), 7.68-7.60 (m, 1H), 7.58-7.52 (m, 2H), 6.05 (dd, J=5.1, 8.4 Hz, 1H), 5.62-5.54 (m, 4H), 4.68 (t, J=1.8, 5.0 Hz, 1H), 4.64-4.55 (m, 1H), 4.25-4.11 (m, 1H), 3.93-3.66 (m, 4H), 3.40 (d, J=19.2 Hz, 3H), 2.75-2.67 (m, 2H), 2.14-1.95 (m, 4H), 1.25-1.20 (m, 12H), 1.15-1.11 (m, 18H); ³¹P NMR (162 MHz, CD₃CN) δ=149.95, 149.27, 32.29, 32.05.

### Example A2

Embodiments of various end capped oligonucleotides described herein were made by using a 5′-vinyl phosphonate building block to incorporate 5′-vinyl phosphonate endcaps:

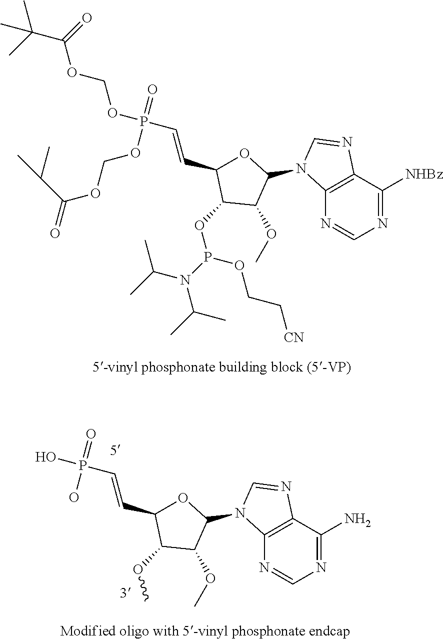

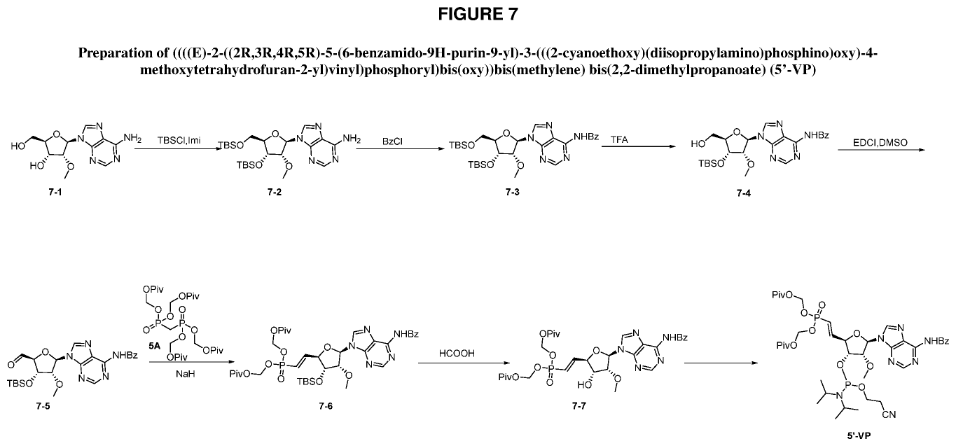

With reference to FIG. 7, the 5′-vinyl phosphonate building block (5′-VP) was prepared as follows:

Preparation of compound 7-2: To a solution of 7-1 (15.0 g, 53.3 mmol) in dry pyridine (150 mL) was added TBSCl (20.0 g, 133.3 mmol) and imidazole (10.8 g, 159.9 mmol). The mixture was stirred at room temperature for 15 h. TLC showed 7-1 was consumed completely. The reaction mixture was concentrated in vacuo to give residue. The residue was quenched with DCM (500 mL). The DCM layer was washed with H₂O (1 L*2) 2 times and brine. The DCM layer concentrated in vacuo to give crude 7-2 (27.2 g, 53.3 mmol) as a yellow oil. The crude 7-2 was used in next step directly. ESI-LCMS m/z 510.5 [M+H]⁺.

Preparation of compound 7-3: To a solution of 7-2 (26.2 g, 51.3 mmol) in pyridine (183 mL) was added dropwise the benzoyl chloride (15.8 g, 113.0 mmol) at 5° C. The reaction mixture was stirred at room temperature for 2 hours. TLC showed the 7-2 was consumed completely. The reaction mixture was quenched with H₂O (4 mL). Then NH₃.H₂O (20 mL) was added to the reaction mixture and stirred at room temperature for 30 min. Then the pyridine was removed from the mixture by concentration under reduced pressure. The residue was added to H₂O (100 mL) and extracted with EA (150 mL*3) and the EA layers combined. The EA layer was washed with brine and dried over Na₂SO₄. Filtered and concentrated to give the crude 7-3 (45.0 g). ESI-LCMS m/z=614.5 [M+H]⁺.

Preparation of compound 7-4: To a mixture solution of 7-3 (44.0 g, crude) in THF (440 mL) was added the H₂O (220 mL) and TFA (220 mL) at 0° C. Then the reaction mixture was stirred at 0° C. for 1.5 hours. TLC showed the 7-3 was consumed completely. The reaction mixture pH was adjusted to 7-8 with NH₃.H₂O. Then the mixture was extracted with EA (300 mL*7). The combined EA layer was washed with brine and concentrated in vacuo to give crude. The crude was purified by column chromatography (EA:PE=1:5-1:1) to give compound 7-4 (15.8 g) as a white solid. ¹H-NMR (400 MHz, DMSO-d₆): δ=11.24 (s, 1H, exchanged with D₂O), 8.77 (s, 2H), 8.04-8.06 (m, 2H), 7.64-7.66 (m, 2H), 7.54-7.58 (m, 2H), 6.14-6.16 (d, J=5.9 Hz, 1H), 5.20-5.23 (m, 1H), 4.58-4.60 (m, 1H), 4.52-4.55 (m, 1H), 3.99-4.01 (m, 1H), 3.69-3.75 (m, 1H), 3.57-3.61 (m, 1H), 3.34 (s, 4H), 0.93 (s, 9H), 0.14-0.15 (d, J=1.44 Hz, 6H). ESI-LCMS m/z=500.3 [M+H]⁺.

Preparation of compound 7-5: To a 500 mL round-bottom flask was added the DMSO (132 mL) and 7-4 (13.2 g, 26.4 mmol), EDCI (15.19 g, 79.2 mmol) in turn at room temperature Then the pyridine (2.09 g, 26.4 mmol, 2.1 mL) was added to the reaction mixture. After stirring 5 min, the TFA (1.51 g, 13.2 mmol) was added to the reaction mixture. Then reaction mixture was stirred at room temperature for 3 hours. LC-MS showed the 7-4 was consumed completely. The reaction mixture was added to the ice water (500 mL) and extracted with EA (300 mL*3) 3 times. The combined EA layer was washed with H₂O×2 and brine×1. Dried over Na₂SO₄ and filtered. The filtrate was concentrated to get crude 7-5 (14.6 g) as a white solid. ESI-LCMS m/z=516.3 [M+H]⁺.

Preparation of compound 7-6: The 5A (24.4 g, 38.5 mmol) was added to a mixture solution of NaH (2.5 g, 64.3 mmol, 60% purity) in THF (50 mL) at 0° C. After stirring 15 min, the 7-5 (16.0 g, 32.1 mmol) in THF (60 mL) was added to the reaction mixture. Then the reaction mixture was stirred at room temperature for 1 hour. LC-MS showed the 7-5 was consumed completely. Then the reaction mixture was quenched with sat. NH₄Cl (500 mL) and extracted with EA (400 mL*3) 3 times. The combined EA layer was washed with brine, dried over Na₂SO₄ and filtered. The filtrate was concentrated in vacuo to get crude. The crude was purified by c.c (EA:PE=1:5-1:1) to give 7-6 (10.0 g, 12.4 mmol, 38.6% yield) as a white solid. ESI-LCMS m/z=804.4 [M+H]⁺; ³¹P NMR (162 MHz, DMSO-d₆) δ 17.01.

Preparation of compound 7-7: To a 500 mL round-bottom flask was added the 7-6 (9.0 g, 11.2 mmol) and H₂O (225 mL), HCOOH (225 mL) in turn. The reaction mixture was stirred at 26° C. for 15 hours. LC-MS showed the 7-6 was consumed completely. The reaction mixture was adjusted the pH=6-7 with NH₃.H₂O. Then the mixture was extracted with EA (300 mL*3) 3 times. The combined EA layer was dried over Na₂SO₄, filtered and filtrate was concentrated to get crude. The crude was purified by column chromatography (DCM/MeOH=100:1-60:1) to give product 7-7 (7.0 g, 10.1 mmol, 90.6% yield). ¹H-NMR (400 MHz, DMSO-d₆): δ=11.11 (s, 1H, exchanged with D₂O), 8.71-8.75 (d, J=14.4, 2H), 8.04-8.06 (m, 2H), 7.64-7.65 (m, 1H), 7.54-7.58 (m, 2H), 6.88-7.00 (m, 1H), 6.20-6.22 (d, J=5.4, 2H), 6.06-6.16 (m, 1H), 5.74-5.75 (d, J=5.72, 2H), 5.56-5.64 (m, 4H), 4.64-4.67 (m, 1H), 4.58-4.59 (m, 1H), 4.49-4.52 (m, 1H), 3.37 (s, 3H), 1.09-1.10 (d, J=1.96, 18H). ³¹P NMR (162 MHz, DMS O-d₆) δ 17.45. ESI-LCMS m/z=690.4 [M+H]⁺.

Preparation of compound 5′-VP: To a solution of 7-7 (5.5 g, 7.9 mmol) in DCM (55 mL) was added the DCI (750 mg, 6.3 mmol), then CEP[N(iPr)₂]₂ (3.1 g, 10.3 mmol) was added. The mixture was stirred at room temperature for 2 hours. TLC showed 3.5% of 7.7 remained. The reaction mixture was washed with H₂O (40 mL*2) and brine (50 mL*2), dried over Na₂SO₄ and concentrated to give crude. The residue was purified by Flash-Prep-HPLC with the following conditions (IntelFlash-1): Column, C18 silica gel; mobile phase, CH₃CN/H₂O (0.5% NH₄HCO₃)=1/5 increasing to CH₃CN/H₂O (0.5% NH₄HCO₃)=1/0 within 30 min, the eluted product was collected at CH₃CN/H₂O (0.5% NH₄HCO₃)=3/1; Detector, UV 254 nm. The product was concentrated and extracted with EA (50 mL*3). The combined EA layer was washed with brine and dried over Na₂SO₄, filtered and filtrate was concentrated to get resulting 5′-VP (6.0 g) as a white solid. ¹H-NMR (400 MHz, DMSO-d₆): δ=11.27 (s, 1H, exchanged with D₂O), 8.72-8.75 (m, 2H), 8.04-8.06 (m, 2H), 7.54-7.68 (m, 3H), 6.85-7.05 (m, 1H), 6.09-6.26 (m, 2H), 5.57-5.64 (m, 4H), 4.70-4.87 (m, 3H), 3.66-3.88 (m, 4H), 3.37-3.41 (m, 3H), 2.82-2.86 (m, 2H), 1.20-1.21 (m, 12H), 1.08-1.09 (m, 18H). ³¹PNMR (162 MHz, DMSO-d₆): 149.99, 149.16, 17.05, 16.81. ESI-LCMS m/z=890.8 [M+H]⁺.

### Example A3

Embodiments of various oligonucleotides described herein were prepared by a modified method using a dinucleotide building block consisting of an A unit and a C unit connected by a stereochemically defined phosphorothioate linkage as follows:

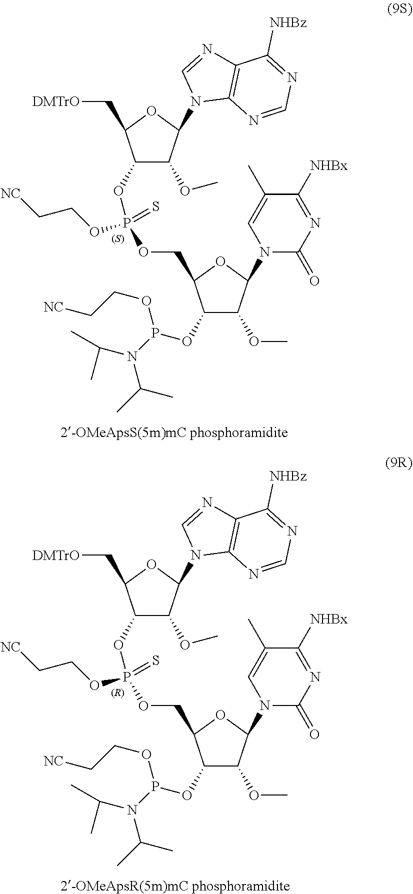

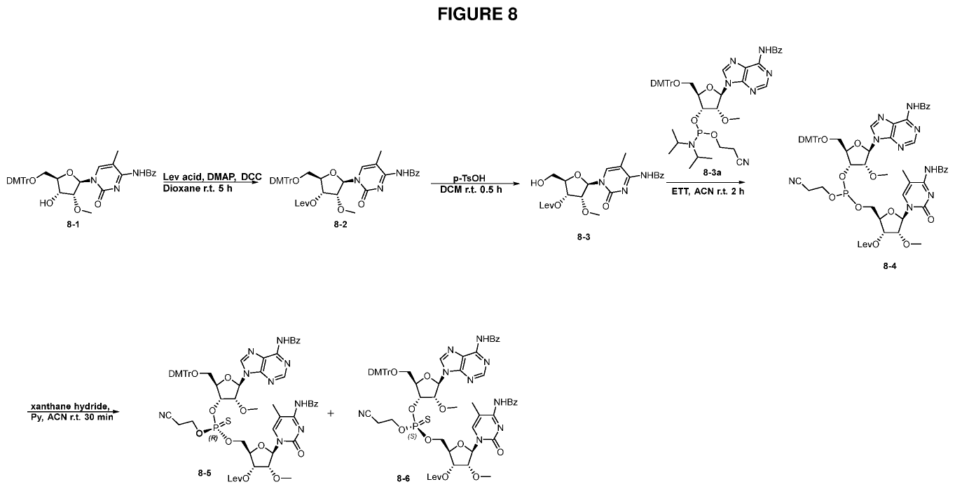

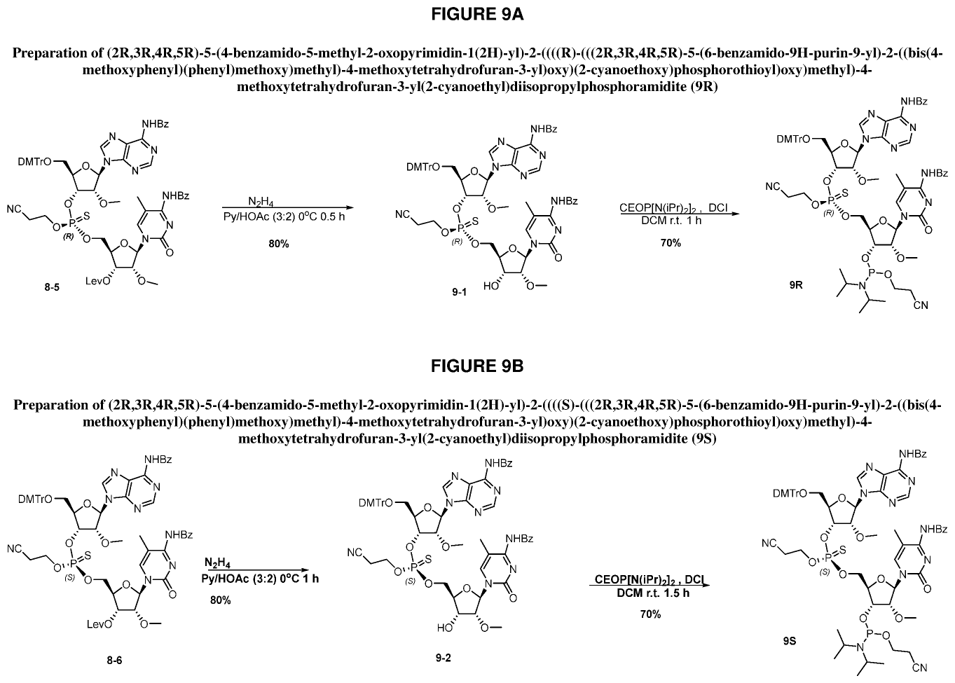

With reference to FIGS. 8, 9A and 9B, the dinucleotide building blocks 9R and 9S were prepared as follows:

Preparation of compound 8-2: To a solution of 8-1 (300.0 g, 445.1 mmol) in 3000 mL of dry dioxane with an inert atmosphere of nitrogen was added levulinic acid (309.3 g, 2.67 mol) dropwise at room temperature. Then the dicyclohexylcarbodiimide (274.6 g, 1.33 mol) and 4-dimethylaminopyridine (27.1 g, 222.0 mmol) were added in order at room temperature. The resulting solution was stirred at room temperature for 5 hours and diluted with 5000 mL of dichloromethane and filtered. The organic phase was washed with 2×3000 mL of 2% aqueous sodium bicarbonate and 1×3000 mL of water respectively. The organic phase was dried over anhydrous sodium sulfate, filtered and concentrated under reduced pressure. 345.0 g (crude) of 8-2 was obtained as a white solid and used for next step without further purification. ESI-LCMS: m/z 774 [M+H]⁺.

Preparation of compound 8-3: To a solution of 8-2 (345 g, 445.1 mmol) was dissolved in 3000 mL dichloromethane with an inert atmosphere of nitrogen was added p-toluenesulfonic acid (84.6 g, 445.1 mmol) dropwise at 0° C. The resulting solution was stirred at 0° C. for 0.5 hours and diluted with 3000 mL of dichloromethane and washed with 2×2000 mL of saturated aqueous sodium bicarbonate and 1×2000 mL of saturated aqueous sodium chloride respectively. The organic phase was dried over anhydrous sodium sulfate, and concentrated under reduced pressure and the residue was purified by silica gel column chromatography (SiO₂, dichloromethane:methanol=30:1) to give 8-3 (210.0 g, 90% over two steps) as a white solid. ¹H-NMR (400 MHz, DMSO-d₆) δ=12.88 (s, 1H), 8.17-8.10 (m, 3H), 7.62-7.60 (m, 1H), 7.58-7.48 (m, 2H), 5.97-5.91 (m, 1H), 5.42 (d, J=5.9 Hz, 1H), 5.25 (s, 1H), 4.21-4.08 (m, 2H), 3.78-3.59 (m, 2H), 2.75-2.74 (m, 2H), 2.57 (m, 2H), 2.13 (d, J=2.3 Hz, 3H), 2.02 (s, 3H), 1.81 (m, 1H), 1.77-1.56 (m, 1H), 1.33-0.98 (m, 1H). ESI-LCMS: m/z 474 [M+H]⁺.

Preparation of compound 8-4: To a solution of 8-3 (210.0 g, 444.9 mmol) in 2000 mL of acetonitrile with an inert atmosphere of nitrogen was added 8-3a (360.0 g, 405.4 mmol) and ETT (58.0 g, 445.9 mmol) in order at 0° C. The resulting solution was stirred for 2 hours at room temperature. Then the mixture was filtered and used for next step without further purification. ESI-LCMS: m/z 1258 [M+H]⁺.

Preparation of compounds 8-5 and 8-6: To a solution of 8-4 (509.9 g, 405.4 mmol) in 2000 mL of acetonitrile with an inert atmosphere of nitrogen was added pyridine (128.0 g, 1.62 mol) and 5-amino-3H-1,2,4-dithiazole-3-thione (121.8 g, 810.9 mmol) in order at room temperature. The reaction solution was stirred for 30 minutes at room temperature. The resulting solution was filtered and concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in a mixture of 8-5 and 8-6 (430.0 g, 90% over two steps) as a white solid. The fractions were diluted with 3000 mL of dichloromethane. The organic phase was dried over anhydrous sodium sulfate, filtered and concentrated under reduced pressure. The residue was purified by SFC with the following conditions: CHIRALPAK IB N-5(IB50CD-VD008)/SFC 0.46 cm I.D.×25 cm L 10.0 ul Mobile phase: (DCM/EtOAc=80/20(V/V)), Detector, UV 254 nm. The fractions were concentrated until no residual solvent left under reduced pressure. 105.0 g (35.0%) of 8-5 were obtained as a white solid and used to make 9R as described below. ¹H-NMR (400 MHz, DMSO-d₆) δ=12.88 (s, 1H), 11.26 (s, 1H), 8.62 (d, J=8.06 Hz, 2H), 8.18 (m, 2H), 8.05 (d, J=7.2 Hz, 2H), 7.79 (s, 1H), 7.67-7.48 (m, 6H), 7.40 (d, J=7.2 Hz, 2H), 7.28-7.18 (m, 7H), 6.86-6.83 (m, 4H), 6.21 (d, J=6.6 Hz, 1H), 5.91 (d, J=5.0 Hz, 1H), 5.44-5.41 (m, 1H), 5.28-5.26 (m, 1H), 5.06 (m, 1H), 4.45-4.24 (m, 7H), 3.71 (s, 6H), 3.39 (s, 4H), 3.31 (s, 3H), 2.98 (m, 2H), 2.75 (m, 2H), 2.56 (m, 2H), 2.01 (s, 3H). ³¹P-NMR (162 MHz, DMSO-d₆) δ=67.17. ESI-LCMS: m/z 1292 [M+H]⁺; 170.0 g (56.6%) of 8-6 were obtained as a white solid and used to make 9S as described below. ¹H-NMR (400 MHz, DMSO-d₆) δ=12.86 (s, 1H), 11.25 (s, 1H), 8.62 (d, J=16.6 Hz, 2H), 8.18 (d, J=7.2 Hz, 2H), 8.05 (m, 2H), 7.78 (s, 1H), 7.67-7.48 (m, 6H), 7.40 (d, J=7.2 Hz, 2H), 7.28-7.18 (m, 7H), 6.87-6.85 (m, 4H), 6.21 (d, J=6.8 Hz, 1H), 5.91 (d, J=5.2 Hz, 1H), 5.43-5.39 (m, 1H), 5.28-5.26 (m, 1H), 5.06 (m, 1H), 4.48-4.21 (m, 7H), 3.72 (s, 6H), 3.36 (s, 4H), 3.26 (s, 3H), 2.95 (m, 2H), 2.73 (m, 2H), 2.55 (m, 2H), 2.04 (s, 3H); ³¹P-NMR (162 MHz, DMSO-d₆) δ=66.84; ESI-LCMS: m/z 1292 [M+H]⁺.

Preparation of compound 9-1: To a solution of 8-5 (100.0 g, 77.4 mmol) in 700 mL acetonitrile with an inert atmosphere of nitrogen was added 0.5 M hydrazine hydrate (20.0 g, 0.4 mol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min. The solution was diluted with DCM (2000 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 9-1 (67.0 g, 80%) as a white solid. ¹H-NMR (400 MHz, DMSO-d₆) δ=12.97 (s, 1H), 11.26 (s, 1H), 8.62 (d, J=11.2 Hz, 2H), 8.19 (d, J=7.2 Hz, 2H), 8.05 (m, 2H), 7.74 (s, 1H), 7.67-7.48. (m, 6H), 7.40 (d, J=7.2 Hz, 2H), 7.28-7.18 (m, 7H), 6.85 (m, 4H), 6.21 (m, 1H), 5.90 (d, J=3.2 Hz, 1H), 5.49-5.43 (m, 2H), 5.05 (m, 1H), 4.45 (m, 1H), 4.40-4.30 (m, 4H), 4.18-4.11 (m, 2H), 3.93 (m, 1H), 3.71 (s, 6H), 3.40-3.32 (m, 8H), 2.98 (m, 2H), 2.04 (s, 3H). ³¹P-NMR (162 MHz, DMSO-d₆) δ=67.30. ESI-LCMS: m/z 1194 [M+H]⁺.

Preparation of compound 9R: To a solution of 9-1 (58.0 g, 48.6 mmol) in 600 mL of dichloromethane with an inert atmosphere of nitrogen was added CEP[N(iPr)₂]₂ (18.7 g, 62.1 mmol) and DCI (5.1 g, 43.7 mmol) in order at room temperature. The resulting solution was stirred for 1 hour at room temperature and diluted with 1000 mL dichloromethane and washed with 2×1000 mL of saturated aqueous sodium bicarbonate and 1×1000 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 9R (51.2 g, 70%) as a white solid. ¹H-NMR (400 MHz, DMSO-d₆) δ=12.94 (m, 1H), 11.26 (s, 1H), 8.62 (m, 2H), 8.19 (d, J=7.2 Hz, 2H), 8.05 (m, 2H), 7.77 (m, 1H), 7.69-7.46 (m, 6H), 7.39 (d, J=6.6 Hz, 2H), 7.26-7.20 (m, 7H), 6.84 (m, 4H), 6.20 (m, 1H), 5.90 (m, 1H), 5.43 (m, 1H), 5.06 (s, 1H), 4.46-4.17 (m, 7H), 4.12 (m, 1H), 3.82-3.80 (m, 2H), 3.73-3.66 (s, 6H), 3.64-3.58 (m, 2H), 3.48-3.29 (m, 8H), 2.98 (s, 2H), 2.82-2.77 (m, 2H), 2.03 (s, 3H), 1.24-1.15 (m, 12H). ³¹P-NMR (162 MHz, DMSO-d₆) δ=149.87, 149.80, 67.43, 67.33. ESI-LCMS: m/z 1394 [M+H]⁺.

Preparation of compound 9-2: To a solution of 8-6 (110.0 g, 85.1 mmol) in 700 mL acetonitrile with an inert atmosphere of nitrogen was added 0.5 M hydrazine hydrate (21.1 g, 423.6 mmol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min, The solution was diluted with DCM (2000 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 9-2 (72.0 g, 80%) as a white solid. ¹H-NMR (400 MHz, DMSO-d₆) δ=12.94 (s, 1H), 11.24 (s, 1H), 8.61-8.57 (m, 2H), 8.18 (d, J=7.6 Hz, 2H), 8.03 (d, J=7.6 Hz, 2H), 7.74 (s, 1H), 7.66-7.47 (m, 6H), 7.40 (d, J=7.1 Hz, 2H), 7.27-7.20 (m, 7H), 6.86 (m, 4H), 6.20 (d, J=6.6 Hz, 1H), 5.87 (d, J=4.0 Hz, 1H), 5.42 (m, 2H), 5.05 (m, 1H), 4.45 (m, 2H), 4.40-4.24 (m, 1H), 4.22-4.06 (m, 4H), 3.92 (m, 1H), 3.71 (s, 6H), 3.40-3.32 (m, 8H), 2.94 (m, 2H), 2.03 (m, 3H). ³¹P-NMR (162 MHz, DMSO-d₆) δ=66.87. ESI-LCMS: m/z 1194 [M+H]⁺.

Preparation of compound 9S: To a solution of 9-2 (62.0 g, 51.9 mmol) in 600 mL of dichloromethane with an inert atmosphere of nitrogen was added CEP[N(iPr)₂]₂ (19.0 g, 63.1 mmol) and DCI (5.55 g, 47.0 mmol) in order at room temperature. The resulting solution was stirred for 1 hour at room temperature and diluted with 1000 mL dichloromethane and washed with 2×1000 mL of saturated aqueous sodium bicarbonate and 1×1000 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 9S (51.5 g, 70%) as a white solid. ¹H-NMR (400 MHz, DMSO-d₆) δ=12.90 (s, 1H), 11.25 (s, 1H), 8.60 (m, 2H), 8.19 (d, J=6.6 Hz, 2H), 8.04 (m, 2H), 7.77 (s, 1H), 7.67-7.48 (m, 6H), 7.41 (d, J=8.0 Hz, 2H), 7.29-7.19 (m, 7H), 6.85 (m, 4H), 6.21 (d, J=6.8 Hz, 1H), 5.91-5.87 (m, 1H), 5.41 (m, 1H), 5.06 (m, 1H), 4.46-4.21 (m, 7H), 4.10 (m, 1H), 3.83-3.75 (m, 2H), 3.73-3.68 (s, 6H), 3.68-3.59 (m, 2H), 3.40-3.32 (m, 8H), 2.93 (m, 2H), 2.80 (m, 2H), 2.02 (s, 3H), 1.18-1.13 (m, 12H). ³¹P-NMR (162 MHz, DMSO-d₆) δ=149.96, 149.73, 66.99, 66.86. ESI-LCMS: m/z 1394 [M+H]⁺.

### Example A4

Embodiments of various oligonucleotides described herein were prepared by a modified method using a dinucleotide building block consisting of an A unit and a C unit connected by a stereochemically defined phosphorothioate linkage as follows:

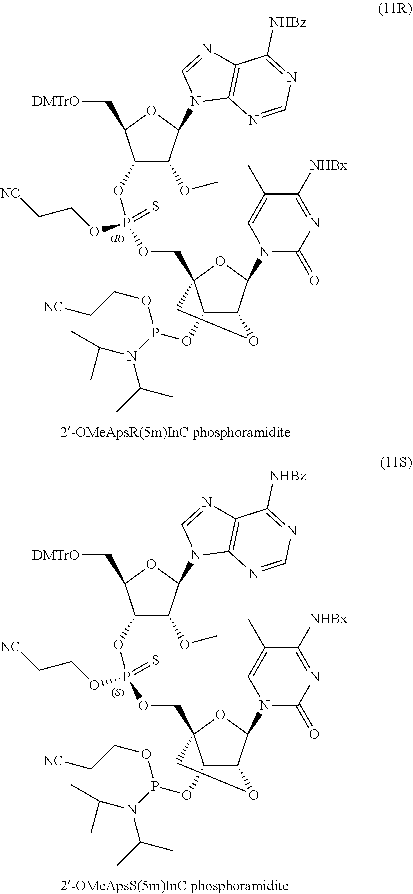

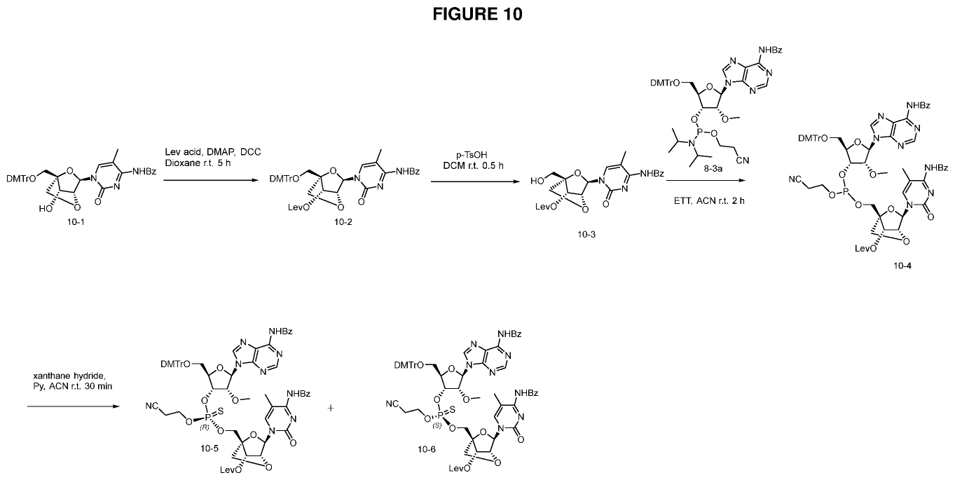

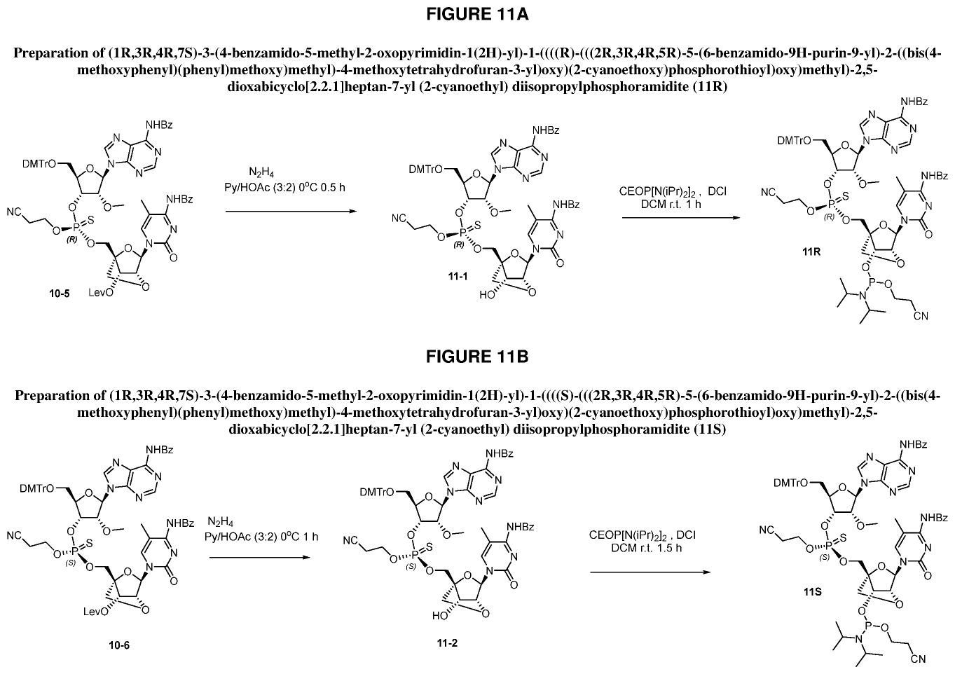

With reference to FIGS. 10, 11A and 11B, the dinucleotide building blocks 11R and 11S were prepared as follows:

Preparation of compound 10-2: To a solution of 10-1 (50.0 g, 74.0 mmol) in 500 mL of dry dioxane with an inert atmosphere of nitrogen was added levulinic acid (51.5 g, 44.4 mol) dropwise at room temperature. Then the dicyclohexylcarbodiimide (45.7 g, 0.2 mol) and 4-dimethylaminopyridine (4.6 g, 37.0 mmol) were added in order at room temperature. The resulting solution was stirred at room temperature for 5 hours and diluted with 3000 mL of dichloromethane and filtered. The organic phase was washed with 2×1000 mL of 2% aqueous sodium bicarbonate and 1×1000 mL of water respectively. The organic phase was dried over anhydrous sodium sulfate, filtered and concentrated under reduced pressure. 52.0 g (crude) of 10-2 was obtained as a white solid and used for next step without further purification. ESI-LCMS: m/z 774 [M+H]⁺.

Preparation of compound 10-3: To a solution of 10-2 (52.0 g, 67.0 mmol) was dissolved in 400 mL dichloromethane with an inert atmosphere of nitrogen was added p-toluenesulfonic acid (51.5 g, 0.4 mol) dropwise at 0° C. The resulting solution was stirred at 0° C. for 0.5 hours and diluted with 2000 mL of dichloromethane and washed with 2×1000 mL of saturated aqueous sodium bicarbonate and 1×1000 mL of saturated aqueous sodium chloride respectively. The organic phase was dried over anhydrous sodium sulfate and concentrated under reduced pressure and the residue was purified by silica gel column chromatography (SiO₂, dichloromethane:methanol=30:1) to give 10-3 (32.0 g, 80% over two steps) as a white solid. ¹H-NMR (400 MHz, DMSO-d₆) δ=13.05 (s, 1H), 8.20-7.91 (m, 4H), 7.60-7.49 (m, 4H), 5.57 (m, 2H), 5.32 (d, J=10.8 Hz, 1H), 4.88 (s, 1H), 4.49 (s, 1H), 4.18 (s, 1H), 3.91-3.78 (m, 5H), 2.74-2.69 (m, 4H), 2.59-2.49 (m, 7H), 2.10 (s, 5H), 2.06 (s, 4H), 1.74-1.49 (m, 3H), 1.26-1.02 (m, 3H). ESI-LCMS: m/z 472 [M+H]⁺.

Preparation of compound 10-4: To a solution of 10-3 (28.0 g, 59.4 mmol) in 300 mL of acetonitrile with an inert atmosphere of nitrogen was added 8-3a (50.0 g, 56.3 mmol) and ETT (7.9 g, 59.4 mmol) in order at 0° C. The resulting solution was stirred for 2 hours at room temperature. Then the mixture was filtered and used for next step without further purification. ESI-LCMS: m/z 1258 [M+H]⁺.

Preparation of compounds 10-5 and 10-6: To a solution of 10-4 (70.9 g, 56.3 mmol) in 300 mL of acetonitrile with an inert atmosphere of nitrogen was added pyridine (17.8 g, 225.2 mmol) and 5-amino-3H-1,2,4-dithiazole-3-thione (16.9 g, 112.6 mmol) in order at room temperature. The reaction solution was stirred for 30 minutes at room temperature. The resulting solution was filtered and concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in a mixture of 10-5 and 10-6. The fractions were diluted with 3000 mL of dichloromethane. The organic phase was dried over anhydrous sodium sulfate, filtered and concentrated under reduced pressure. The residue was purified by SFC with the following conditions: CHIRAL CEL OD-H/SFC 20 mm*250 mmL 5 um (Phase A: CO₂; Phase B: 50% ethanol-50% acetonitrile), Detector, UV 220 nm. The fractions were concentrated until no residual solvent left under reduced pressure. 9.0 g (25.7%) of 10-5 were obtained as a white solid and used to make 11R as described below. ¹H-NMR (400 MHz, DMSO-d₆) δ=13.06 (s, 1H), 11.28 (s, 1H), 8.63 (d, J=20 Hz, 2H), 8.20 (m, 2H), 8.05 (d, J=8 Hz, 2H), 7.84 (s, 1H), 7.67-7.39 (m, 8H), 7.28-7.19 (m, 7H), 6.86-6.83 (m, 4H), 6.24 (d, J=6.6 Hz, 1H), 5.66 (s, 2H), 5.45-5.43 (m, 1H), 5.10-5.03 (m, 2H), 4.82-4.76 (m, 1H), 4.60 (s, 1H), 4.50-4.33 (m, 4H), 4.03-3.96 (m, 2H), 3.72 (s, 6H), 3.41-3.35 (m, 7H), 3.03-3.00 (m, 2H), 2.75-2.72 (m, 2H), 2.56-2.53 (m, 2H), 2.08-2.05 (m, 6H). ³¹P-NMR (162 MHz, DMSO-d₆) δ=67.0; ESI-LCMS: m/z 1290 [M+H]⁺. 15.0 g (42.8%) of 10-6 were obtained as a white solid and used to make 11S as described below. ¹H-NMR (400 MHz, DMSO-d₆) δ=13.05 (s, 1H), 11.26 (s, 1H), 8.63 (d, J=24 Hz, 2H), 8.-7.96 (m, 4H), 7.76 (s, 1H), 7.67-7.39 (m, 8H), 7.28-7.19 (m, 7H), 6.86 (d, J=7.2 Hz, 4H), 6.24 (d, J=6.4 Hz, 1H), 5.76 (s, 1H), 5.63 (s, 1H), 5.43-5.41 (m, 1H), 5.12 (m, 1H), 4.97 (s, 1H), 4.82-4.79 (m, 1H), 4.57-4.49 (m, 3H), 4.27-4.25 (m, 2H), 4.07-4.03 (m, 2H), 3.72 (s, 6H), 3.44-3.36 (m, 6H), 2.96 (m, 2H), 2.74-2.71 (m, 2H), 2.55-2.53 (m, 2H), 2.08 (s, 3H), 1.94 (s, 3H). ³¹P-NMR (162 MHz, DMSO-d₆) δ=66.58. ESI-LCMS: m/z 1290 [M+H]⁺.

Preparation of compound 11-1: To a solution of 10-5 (10.0 g, 7.7 mmol) in 100 mL acetonitrile with an inert atmosphere of nitrogen was added 0.5 M hydrazine hydrate (1.8 g, 37.5 mmol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min, the solution was diluted with DCM (500 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 11-1 (6.0 g, 65%) as a white solid. ¹H-NMR (400 MHz, DMSO-d₆) δ=13.13 (s, 1H), 11.28 (s, 1H), 8.63 (d, J=20 Hz, 2H), 8.21 (d, J=8 Hz, 2H), 8.06-7.95 (m, 3H), 7.80 (s, 1H), 7.67-7.48. (m, 8H), 7.40 (d, J=7.6 Hz, 2H), 7.32-7.19 (m, 10H), 6.85 (m, 5H), 6.24 (d, J=8 Hz, 1H), 6.04 (d, J=4.0 Hz, 1H), 5.57 (s, 2H), 5.44-5.42 (m, 1H), 5.19-5.17 (m, 2H), 5.10-5.08 (m, 1H), 4.80-4.76 (m, 2H), 4.50 (d, J=5.6 Hz, 1H), 4.37-4.32 (m, 4H), 4.06-3.99 (m, 2H), 3.81 (m, 1H), 3.72 (s, 7H), 3.40-3.36 (m, 8H), 3.03-3.00 (m, 2H), 2.05 (m, 3H). ³¹P-NMR (162 MHz, DMSO-d₆) δ=67.21. ESI-LCMS: m/z 1192 [M+H]⁺.

Preparation of compound 11R: To a solution of 11-1 (6.0 g, 5.0 mmol) in 60 mL of dichloromethane with an inert atmosphere of nitrogen was added CEP[N(iPr)₂]₂ (1.9 g, 6.5 mmol) and DCI (0.6 g, 5.0 mmol,) in order at room temperature. The resulting solution was stirred for 1 hours at room temperature and diluted with 1000 mL dichloromethane and washed with 2×250 mL of saturated aqueous sodium bicarbonate and 1×250 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 11R (5.0 g, 70%) as a white solid. ¹H-NMR (400 MHz, DMSO-d₆) δ=13.10 (s, 1H), 11.28 (s, 1H), 8.20 (d, J=8.0 Hz, 2H), 8.04 (d, J=7.2 Hz, 2H), 7.79 (d, J=14 Hz, 2H), 7.67-7.48 (m, 6H), 7.39 (d, J=7.2 Hz, 2H), 7.27-7.18 (m, 7H), 6.85-6.82 (m, 4H), 6.23-6.20 (m, 1H), 5.64 (d, J=6.0 Hz, 1H), 5.44-5.41 (m, 1H), 5.08-5.07 (m, 1H), 4.82-4.77 (m, 1H), 4.56-4.46 (m, 3H), 4.36-4.30 (m, 2H), 4.22 (d, J=7.2 Hz, 1H), 3.98 (m, 1H), 3.89 (m, 1H), 3.71 (s, 7H), 3.59-3.55 (m, 2H), 3.40-3.34 (m, 10H), 3.02-2.98 (m, 2H), 2.77-2.72 (m, 2H), 2.08-2.05 (m, 3H), 1.13-1.08 (m, 12H). ³¹P-NMR (162 MHz, DMSO-d₆) δ=148.71, 148.11, 67.51, 67.44. ESI-LCMS: m/z 1392 [M+H]⁺.

Preparation of compound 11-2: To a solution of 10-6 (10.0 g, 7.7 mmol) in 100 mL acetonitrile with an inert atmosphere of nitrogen was added 0.5 M hydrazine hydrate (1.8 g, 37.5 mmol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min. The solution was diluted with DCM (500 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 11-2 (7.5 g, 80%) as a white solid. ¹H-NMR (400 MHz, DMSO-d₆) δ=13.11 (s, 1H), 11.26 (s, 1H), 8.63 (d, J=20 Hz, 2H), 8.20 (d, J=7.2 Hz, 2H), 8.15 (m, 3H), 7.73 (s, 1H), 7.66-7.47. (m, 8H), 7.41 (d, J=7.6 Hz, 2H), 7.32-7.19 (m, 10H), 6.85 (m, 5H), 6.24 (m, 1H), 5.99 (s, 1H), 5.54 (s, H), 5.41 (m, 1H), 5.10 (m, 1H), 4.79-4.75 (m, 1H), 4.57-4.49 (m, 3H), 4.30-4.24 (m, 4H), 4.02 (m, 2H), 3.85 (m, 1H), 3.72 (s, 7H), 3.38-3.35 (m, 7H), 2.95 (m, 2H), 1.98 (m, 3H). ³¹P-NMR (162 MHz, DMSO-d₆) δ=66.79. ESI-LCMS: m/z 1192 [M+H]⁺.

Preparation of compound 11S: To a solution of 11-2 (7.0 g, 5.0 mmol) in 70 mL of dichloromethane with an inert atmosphere of nitrogen was added CEP[N(iPr)₂]₂ (2.0 g, 6.5 mmol) and DCI (0.6 g, 5.0 mmol) in order at room temperature. The resulting solution was stirred for 1 hours at room temperature and diluted with 1000 mL dichloromethane and washed with 2×250 mL of saturated aqueous sodium bicarbonate and 1×250 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 11S (6.3 g, 70%) as a white solid. ¹H-NMR (400 MHz, DMSO-d₆) δ=13.10 (s, 1H), 11.27 (s, 1H), 8.65 (s, 1H), 8.61 (s, 1H), 8.19 (m, 2H), 8.02 (d, J=7.2 Hz, 2H), 7.76-7.73 (m, 1H), 7.66-7.47 (m, 6H), 7.40 (d, J=7.2 Hz, 2H), 7.28-7.19 (m, 7H), 6.86-6.85 (m, 4H), 6.24 (d, J=6.8 Hz, 1H), 5.62 (m, 1H), 5.43-5.41 (m, 1H), 5.10 (s, 1H), 4.84-4.78 (m, 1H), 4.66-4.49 (m, 3H), 4.30-4.18 (m, 3H), 4.04-3.95 (m, 2H), 3.83-3.77 (m, 1H), 3.72 (s, 7H), 3.62-3.54 (m, 2H), 3.44-3.32 (m, 6H), 2.96-2.92 (m, 2H), 2.77-2.72 (m, 2H), 1.98-1.97 (m, 3H), 1.12-1.11 (m, 12H). ³¹P-NMR (162 MHz, DMSO-d₆) δ=148.53, 148.09, 67.04. ESI-LCMS: m/z 1392 [M+H]⁺.

### Example A5

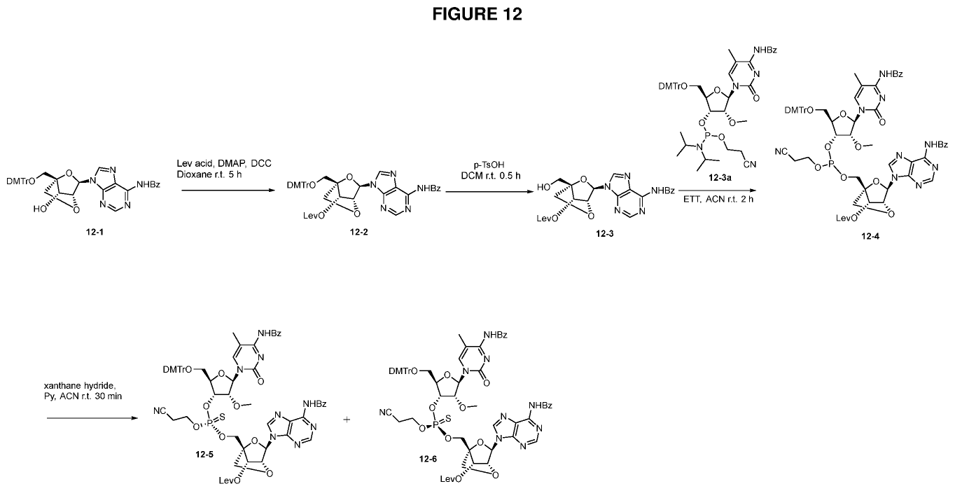

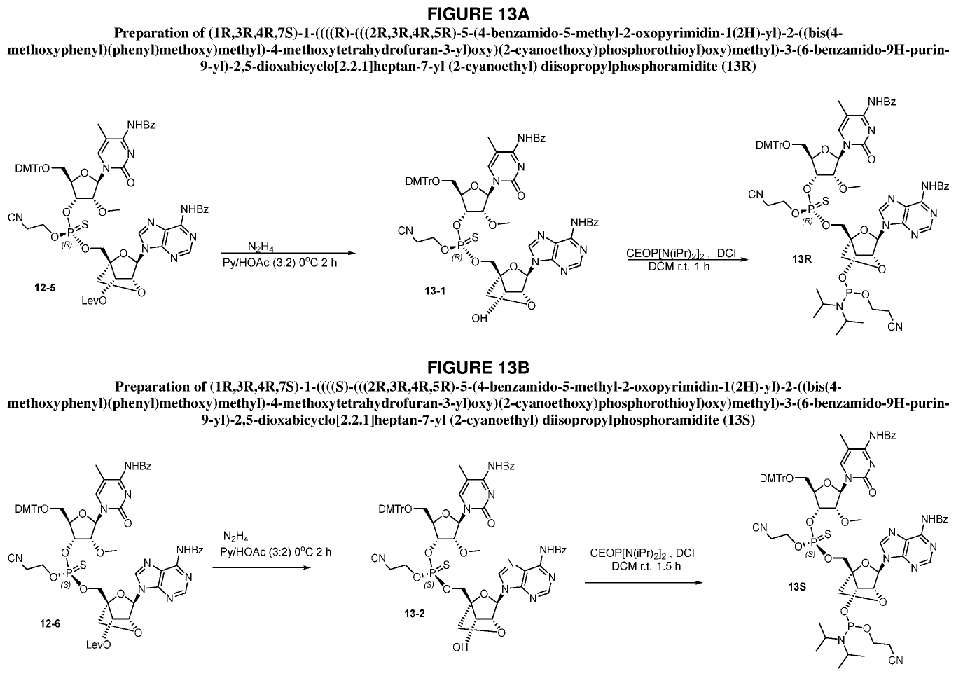

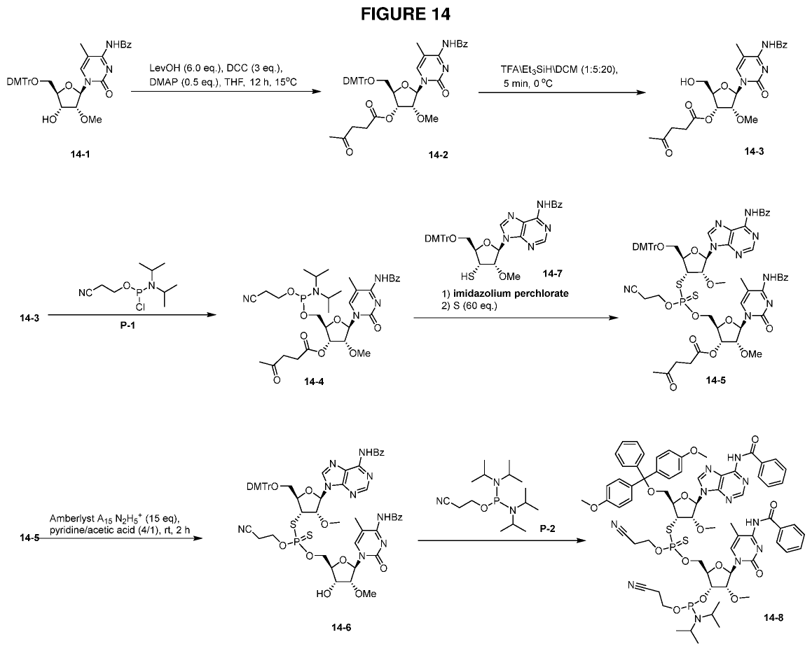

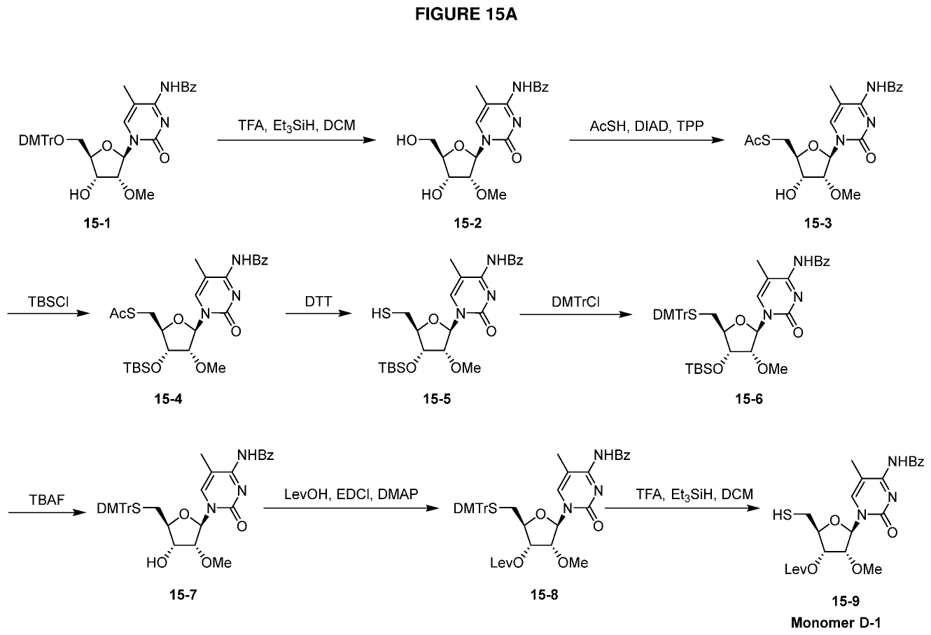

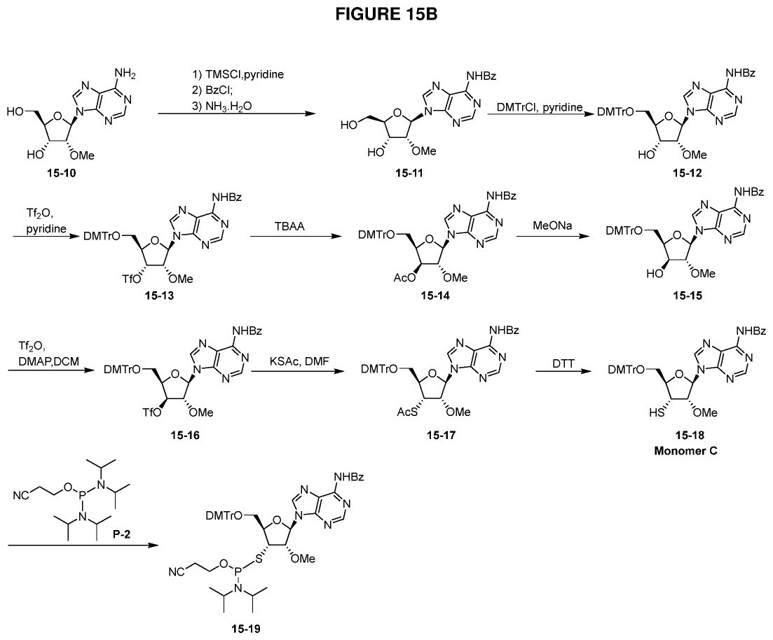

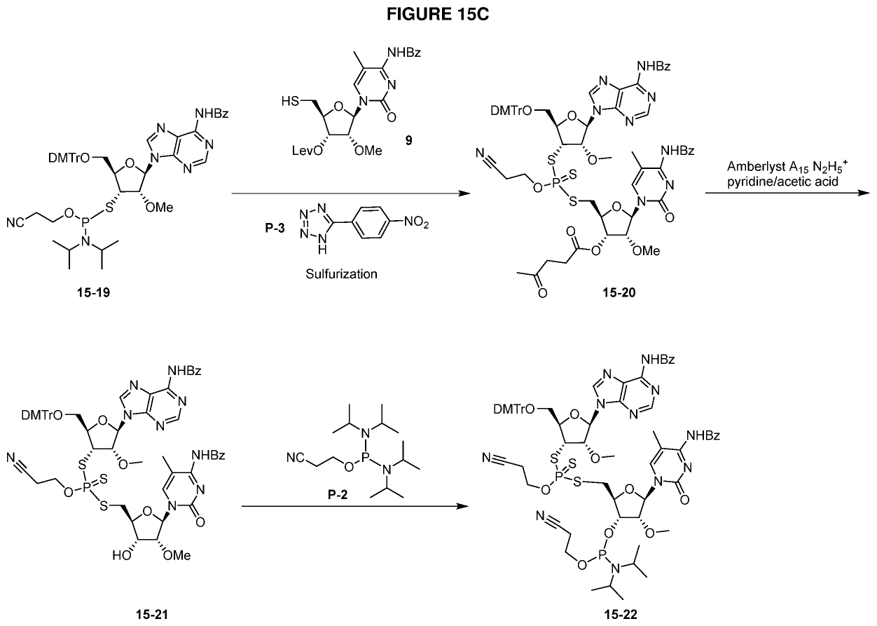

With reference to FIGS. 12-15, dinucleotide building blocks useful for making embodiments of modified phosphorothioated oligonucleotides were prepared as follows:

Preparation of compound 12-2: To a solution of 12-1 (33.0 g, 48.0 mmol) in 500 mL of dry dioxane with an inert atmosphere of nitrogen was added levulinic acid (33.4 g, 287.9 mmol) dropwise at room temperature. Then the dicyclohexylcarbodiimide (29.7 g, 144.0 mmol) and 4-dimethylaminopyridine (2.9 g, 24.0 mmol) were added in order at room temperature. The resulting solution was stirred at room temperature for 5 hours and diluted with 3000 mL of dichloromethane and filtered. The organic phase was washed with 2×1000 mL of 2% aqueous sodium bicarbonate and 1×1000 mL of water respectively. The organic phase was dried over anhydrous sodium sulfate, filtered and concentrated under reduced pressure. 40.0 g (crude) of 12-2 was obtained as a white solid and used for next step without further purification. ESI-LCMS: m/z 784 [M+H]⁺.

Preparation of compound 12-3: To a solution of 12-2 (39.0 g, 49.6 mmol) in 400 mL of dichloromethane with an inert atmosphere of nitrogen was added p-toluenesulfonic acid (9.4 g, 49.6 mmol) drop wise at 0° C. The resulting solution was stirred at 0° C. for 0.5 hours and diluted with 2000 mL of dichloromethane and washed with 2×1000 mL of saturated aqueous sodium bicarbonate and 1×1000 mL of saturated aqueous sodium chloride respectively. The organic phase was dried over anhydrous sodium sulfate, and concentrated under reduced pressure and the residue was purified by silica gel column chromatography (SiO₂, dichloromethane:methanol=30:1) to give 12-3 (20.0 g, 87.0% over two steps) as a white solid. ¹H-NMR (400 MHz, DMSO-d₆) δ=11.16 (s, 1H), 8.76 (s, 1H), 8.57 (s, 1H), 8.06-8.04 (m, 2H), 7.67-7.64 (m, 1H), 7.58-7.54 (m, 2H), 6.17 (m, 1H), 5.20 (s, 1H), 4.80 (s, 1H), 4.20 (s, 1H), 4.18-3.86 (m, 2H), 3.85-3.79 (m, 2H), 2.75-2.70 (m, 2H), 2.57-2.54 (m, 2H), 2.16 (s, 4H). ESI-LCMS: m/z 482 [M+H]⁺.

Preparation of compound 12-4: To a solution of 12-3 (18.0 g, 37.4 mmol) in 300 mL of 400 mL dry acetonitrile with an inert atmosphere of nitrogen was added 12-3a (35.0 g, 39.7 mmol) and ETT (5.3 g, 41.1 mmol) in order at 0° C. The resulting solution was stirred for 2 hours at room temperature. Then the mixture was filtered and used for next step without further purification. ESI-LCMS: m/z 1258 [M+H]⁺.

Preparation of compounds 12-5 and 12-6: To a solution of 12-4 (48.2 g, 37.4 mmol) in 400 mL of dry acetonitrile with an inert atmosphere of nitrogen was added pyridine (11.8 g, 149.6 mmol) and 5-amino-3H-1,2,4-dithiazole-3-thione (11.2 g, 74.8 mmol) in order at room temperature. The reaction solution was stirred for 30 minutes at room temperature. The resulting solution was filtered and concentrated at 25° C. under reduced pressure. The residue was purified by Flash-Prep-HPLC with the following conditions (IntelFlash-1): Column, C18 silica gel; mobile phase, CH₃CN/H₂O (0.5% NH₄HCO₃)=0/100 increasing to CH₃CN/H₂O (0.5% NH₄HCO₃)=100/0 within 35 min, the eluted product was collected at CH₃CN/H₂O (0.5% NH₄HCO₃)=95/5; Detector, UV 254 nm. The solution was concentrated under reduced pressure to get 33.0 g crude. The crude was purified by SFC with the following conditions: CHIRAL CEL OD-H/SFC 20 mm*250 mmL Sum (Phase A: CO₂; Phase B: 50% ethanol-50% acetonitrile), Detector, UV 220 nm. The fractions were concentrated until no residual solvent left under reduced pressure. 13.9 g (39.7%) of 12-5 were obtained as a white solid and used to make the corresponding dinucleotide as described below. ¹H-NMR (400 MHz, DMSO-d₆) δ=12.87 (s, 1H), 11.25 (s, 1H), 8.74 (s, 1H), 8.58 (s, 1H), 8.16 (m, 2H), 8.05 (d, J=7.2 Hz, 2H), 7.77 (s, 1H), 7.67-7.51 (m, 6H), 7.49 (d, J=7.2 Hz, 2H), 7.43-7.22 (m, 7H), 6.91 (d, J=8.8 Hz, 4H), 6.16 (s, 1H), 5.94 (d, J=4.0 Hz, 1H), 5.51 (s, 1H), 5.20 (m, 1H), 4.91 (s, 1H), 4.70 (m, 1H), 4.39-4.26 (m, 5H), 3.74 (s, 6H), 3.47 (s, 3H), 3.33 (s, 4H), 2.98 (m, 2H), 2.75 (m, 2H), 2.56 (m, 2H), 2.09 (s, 3H), 1.63 (s, 3H). ³¹P-NMR (162 MHz, DMSO-d₆) δ=67.00. ESI-LCMS: m/z 1290 [M+H]⁺. 18.0 g (51.4%) of 12-6 were obtained as a white solid and used to make the corresponding dinucleotide as described below. ¹H-NMR (400 MHz, DMSO-d₆) δ=12.84 (s, 1H), 11.25 (s, 1H), 8.74 (s, 1H), 8.57 (s, 1H), 8.15 (m, 2H), 8.05 (d, J=8 Hz, 2H), 7.77 (s, 1H), 7.67-7.51 (m, 6H), 7.41 (d, J=8 Hz, 2H), 7.40-7.23 (m, 7H), 6.91 (d, J=8.8 Hz, 4H), 6.16 (s, 1H), 5.94 (d, J=4.4 Hz, 1H), 5.48 (s, 1H), 5.20 (m, 1H), 4.93 (s, 1H), 4.70 (m, 1H), 4.49 (m, 1H), 4.36-4.32 (m, 2H), 4.18-4.13 (m, 3H), 3.74 (s, 6H), 3.44 (s, 3H), 3.38-3.33 (m, 4H), 2.87 (m, 2H), 2.76 (m, 2H), 2.58 (m, 2H), 2.10 (s, 3H), 1.61 (s, 3H). ³¹P-NMR (162 MHz, DMSO-d₆) δ=66.63. ESI-LCMS: m/z 1290 [M+H]⁺.

Preparation of compound 13-1: To a solution of 12-5 (10.0 g, 7.7 mmol) in 100 mL acetonitrile with an inert atmosphere of nitrogen was added 0.5 M Hydrazine hydrate (1.8 g, 37.5 mmol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min. The solution was diluted with DCM (500 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 13-1 (7.2 g, 78%) as a white solid. ¹H-NMR (400 MHz, DMSO-d₆) δ=12.88 (s, 1H), 11.22 (s, 1H), 8.75 (s, 1H), 8.55 (s, 1H), 8.18 (d, J=6.8 Hz, 2H), 8.05 (d, J=7.6 Hz, 2H), 7.77 (s, 1H), 7.65-7.48 (m, 6H), 7.49 (d, J=7.2 Hz, 2H), 7.35-7.22 (m, 7H), 6.92-6.89 (m, 4H), 6.06-6.04 (m, 2H), 5.93 (d, J=4.8 Hz, 1H), 5.20-5.18 (m, 1H), 4.71-4.66 (m, 1H), 4.61 (s, 1H), 4.60-4.23 (m, 6H), 4.01 (d, J=8.0 Hz, 1H), 3.84 (d, J=8.0 Hz, 1H), 3.73 (m, 6H), 3.47 (s, 3H), 3.34 (s, 4H), 2.99-2.96 (m, 2H), 2.08 (s, 5H), 1.65 (s, 3H). ³¹P-NMR (162 MHz, DMSO-d₆) δ=67.25. ESI-LCMS: m/z 1192 [M+H]⁺.

Preparation of compound 13R: To a solution of 13-1 (6.6 g, 5.5 mmol) in 60 mL of dichloromethane with an inert atmosphere of nitrogen was added CEP[N(iPr)₂]₂ (1.9 g, 6.5 mmol) and DCI (0.6 g, 5.0 mmol,) in order at room temperature. The resulting solution was stirred for 1 hours at room temperature and diluted with 1000 mL dichloromethane and washed with 2×250 mL of saturated aqueous sodium bicarbonate and 1×250 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 13R (5.6 g, 70%) as a white solid. ¹H-NMR (400 MHz, DMSO-d₆) δ=12.87 (s, 1H), 11.22 (s, 1H), 8.74 (m, 1H), 8.54-8.49 (m, 1H), 8.18 (d, J=6.8 Hz, 2H), 8.06-8.04 (m, 2H), 7.77 (s, 1H), 7.65-7.47 (m, 6H), 7.42-7.40 (m, 2H), 7.35-7.23 (m, 7H), 6.91 (d, J=8.8 Hz, 4H), 6.16-6.13 (m, 1H), 5.94 (m, 1H), 5.21-5.18 (m, 1H), 4.88-4.85 (m, 1H), 4.75-4.70 (m, 2H), 4.57-4.26 (m, 5H), 4.03-4.00 (m, 1H), 3.93-3.91 (m, 1H), 3.81-3.73 (m, 8H), 3.60-3.47 (m, 5H), 3.35-3.33 (m, 3H), 2.98-2.95 (m, 2H), 2.75-2.69 (m, 2H), 1.65 (s, 3H), 1.13-1.03 (m, 12H). ³¹P-NMR (162 MHz, DMSO-d₆) δ=148.68, 148.61, 67.47, 67.36. ESI-LCMS: m/z 1392 [M+H]⁺.

Preparation of compound 13-2: To a solution of 12-6 (10.0 g, 7.7 mmol) in 100 mL acetonitrile with an inert atmosphere of nitrogen was added 0.5 M hydrazine hydrate (1.8 g, 37.5 mmol) in pyridine/acetic acid (3:2) at 0° C. The resulting solution was stirred for 0.5 hours at 0° C. Then the reaction was added 2,4-pentanedione at once, the mixture was allowed to warm to room temperature and stirred for additional 15 min, the solution was diluted with DCM (500 mL) and washed with sat. aqueous NH₄Cl twice and washed with brine and dried over Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 13-2 (7.5 g, 80%) as a white solid. ¹H-NMR (400 MHz, DMSO-d₆) δ=12.84 (s, 1H), 11.22 (s, 1H), 8.74 (s, 1H), 8.56 (s, 1H), 8.17 (d, J=7.2 Hz, 2H), 8.05 (d, J=7.2 Hz, 2H), 7.77 (s, 1H), 7.67-7.47 (m, 6H), 7.40 (d, J=7.6 Hz, 2H), 7.35-7.23 (m, 7H), 6.92-6.89 (m, 4H), 6.06-6.04 (m, 2H), 5.94 (d, J=5.2 Hz, 1H), 5.21-5.19 (m, 1H), 4.69-4.62 (m, 1H), 4.55 (s, 1H), 4.50-4.44 (m, 2H), 4.36-4.19 (m, 2H), 4.14-4.08 (m, 3H), 3.93 (d, J=8.0 Hz, 1H), 3.73 (m, 6H), 3.46 (s, 3H), 3.34 (m, 3H), 2.88-2.85 (m, 2H), 2.08 (s, 5H), 1.61 (s, 3H). ³¹P-NMR (162 MHz, DMSO-d₆) δ=66.81. ESI-LCMS: m/z 1192 [M+H]⁺.

Preparation of compound 13S: To a solution of 13S (6.6 g, 5.5 mmol) in 60 mL of dichloromethane with an inert atmosphere of nitrogen was added CEP[N(iPr)₂]2 (1.9 g, 6.5 mmol) and DCI (0.6 g, 5.0 mmol) in order at room temperature. The resulting solution was stirred for 1 hours at room temperature and diluted with 1000 mL dichloromethane and washed with 2×250 mL of saturated aqueous sodium bicarbonate and 1×250 mL of saturated aqueous sodium chloride respectively. The organic phase was dried over anhydrous sodium sulfate, filtered and concentrated until no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 13S (5.5 g, 70%) as a white solid. ¹H-NMR (400 MHz, DMSO-d₆) δ=12.84 (s, 1H), 11.23 (s, 1H), 8.74 (s, 1H), 8.56-8.53 (m, 1H), 8.17 (d, J=7.2 Hz, 2H), 8.06 (d, J=7.2 Hz, 2H), 7.77 (s, 1H), 7.67-7.47 (m, 6H), 7.42-7.40 (m, 2H), 7.33-7.23 (m, 7H), 6.91 (d, J=8.4 Hz, 4H), 6.15 (m, 1H), 5.95 (m, 1H), 5.21 (m, 1H), 4.90 (d, J=10.8 Hz, 1H), 4.73-4.56 (m, 3H), 4.35-4.32 (m, 2H), 4.10-4.02 (m, 4H), 3.82-3.71 (m, 8H), 3.62-3.56 (m, 2H), 3.47-3.46 (m, 3H), 3.34-3.33 (m, 3H), 2.85-2.73 (m, 4H), 1.62 (s, 3H), 1.14-1.05 (m, 12H). ³¹P-NMR (162 MHz, DMSO-d₆) δ=148.68, 148.63, 67.22, 67.10; ESI-LCMS: m/z 1392 [M+H]⁺.

Preparation of compound 14-2: To a solution of 14-1 (15 g, 22.13 mmol, 1 eq) in THF (100 mL) were added DCC (13.70 g, 66.40 mmol, 13.43 mL, 3 eq), 4-oxopentanoic acid (15.42 g, 132.79 mmol, 6 eq), and DMAP (1.35 g, 11.07 mmol, 0.5 eq), and the mixture was stirred at 15° C. for 12 hours. The reaction mixture was quenched by addition of NaHCO₃ solution (100 mL) and then extracted with EA (200 mL*3). The combined organic layers were dried over Na₂SO₄, filtered and concentrated under reduced pressure to give a residue. The residue was purified by flash silica gel chromatography (ISCO®; 120 g SepaFlash® Silica Flash Column, Eluent of 0˜50% EtOAc/PE gradient at 55 mL/min, 40 minutes with total volume 2.0 L) to afford 14-2 (18.25 g, crude) as a white solid. LCMS (ESI): m/z calcd. for C₄₄H₄₆N₃O₁₀ 776.32 [M+H]⁺, found 776.4.

Preparation of compound 14-3: To a solution of 14-2 (10 g, 11.90 mmol, 1 eq) in DCM (100 mL) were added Et₃SiH (25 mL) and TFA (5 mL). The mixture was stirred at 0° C. for 5 min. The reaction mixture was quenched by addition of sat. NaHCO₃ (100 mL), and then extracted with DCM (100 mL*3). The combined organic layers were dried over Na₂SO₄, filtered and concentrated under reduced pressure to give a residue. The crude product was purified by flash silica gel chromatography (ISCO®; 80 g SepaFlash® Silica Flash Column, Eluent of 0˜90% Ethyl acetate/Petroleum ether gradient at 55 mL/min, 40 minutes with total volume 2.0 L) to give 14-3 (6.33 g, 11.38 mmol, 95.72% yield, 85.13% purity) as a white solid. LCMS (ESI): m/z calcd. for C₂₃H₂₈N₃O₈ 474.19 [M+H]⁺, found 474.2. ¹H NMR (400 MHz, CDCl₃) δ=8.36-8.29 (m, 2H), 7.66 (s, 1H), 7.57-7.52 (m, 1H), 7.49-7.42 (m, 2H), 5.72 (d, J=4.9 Hz, 1H), 5.35 (t, J=4.8 Hz, 1H), 4.32 (t, J=5.1 Hz, 1H), 4.28-4.23 (m, 1H), 4.00 (dd, J=1.8, 12.6 Hz, 1H), 3.85-3.79 (m, 1H), 3.47 (s, 3H), 2.81 (q, J=6.6 Hz, 2H), 2.71-2.64 (m, 2H), 2.22 (s, 3H), 2.12 (d, J=0.7 Hz, 3H).

Preparation of compound 14-4: To a mixture of 14-3 (1.0 g, 2.11 mmol, 1 eq) in DCM (8 mL) were added DIPEA (1.36 g, 10.56 mmol, 1.84 mL, 5 eq) and P-1 (749.81 mg, 3.17 mmol, 1.5 eq). The mixture was stirred at 20° C. for 2 hour. The reaction mixture was diluted with DCM (10 mL) and quenched by addition NaHCO₃ solution (30 mL), and the aqueous phase was extracted with DCM (10 mL*2). The combined organic layers were washed with NaCl solution (20 mL*2), dried over anhydrous Na₂SO₄, filtered, and concentrated to give a residue. The residue was purified by (12 g SepaFlash® Silica Flash Column, Eluent of 0˜50% Ethyl acetate/Petroleum ether gradient at 25 mL/min) to give compound 14-4 (1.1 g, 1.60 mmol, 75.76% yield) as a light yellow gum. LCMS (ESI): m/z calcd. for C₃₂H₄₅N₅O₉P 674.30 [M+H]⁺, found 674.3; ¹H NMR (400 MHz, CDCl₃) δ=13.25 (br s, 1H), 8.32 (br d, J=7.5 Hz, 2H), 7.85-7.67 (m, 1H), 7.56-7.50 (m, 1H), 7.47-7.41 (m, 2H), 6.12 (dd, J=5.4, 10.5 Hz, 1H), 5.33-5.23 (m, 1H), 4.30 (br d, J=2.2 Hz, 1H), 3.99 (br d, J=2.0 Hz, 1H), 3.96-3.79 (m, 4H), 3.71-3.60 (m, 2H), 3.41 (d, J=12.6 Hz, 3H), 2.87-2.74 (m, 2H), 2.67 (q, J=5.7 Hz, 4H), 2.20 (s, 3H), 2.15 (d, J=3.1 Hz, 3H), 1.26-1.20 (m, 12H). ³¹P NMR (162 MHz, CD₃CN) δ=149.71, 148.30.

Preparation of compound 14-5: To a solution of 14-7 (4.0 g, 5.68 mmol, 1 eq) in MeCN (5 mL) were added imidazolium perchlorate (6.23 g, 36.94 mmol, 6.5 eq) and 4A MS (7.2 g), followed by 14-4 (6.01 g, 8.92 mmol, 1.57 eq), and the mixture was stirred at 20° C. for 2 hours. Then S (10.93 g, 341.00 mmol, 60 eq) was added and the mixture was stirred for additional 2 hours. The reaction mixture was filtered and the filtrate was concentrated to give a residue, which was purified by flash C-18 column chromatography (Column, C18 silica gel; mobile phase, water and acetonitrile, 0%-70%) to give compound 14-5 (2.8 g, 2.14 mmol, 37.65% yield, 100% purity) as a light yellow solid. LCMS (ESI): m/z calcd. for C₆₅H₆₆N₉O₁₅PS₂ 1308.39 [M+H]⁺; found 1308.5 ¹H NMR (400 MHz, CD₃CN) δ=13.17 (br s, 1H), 9.30 (br s, 1H), 8.64 (br d, J=12.3 Hz, 1H), 8.33 (d, J=7.5 Hz, 1H), 8.28-8.20 (m, 2H), 7.94 (br dd, J=7.8, 12.7 Hz, 2H), 7.62-7.34 (m, 9H), 7.28-7.15 (m, 7H), 6.78 (dt, J=3.2, 9.0 Hz, 4H), 6.26 (s, 1H), 5.85 (dd, J=4.6, 14.8 Hz, 1H), 5.16 (q, J=5.3 Hz, 1H), 4.72-4.58 (m, 2H), 4.44-4.07 (m, 7H), 3.72 (dd, J=1.2, 6.7 Hz, 6H), 3.60 (d, J=1.1 Hz, 3H), 3.52 (br t, J=9.7 Hz, 1H), 3.35 (s, 4H), 2.79-2.68 (m, 4H), 2.58-2.47 (m, 2H), 2.10 (s, 3H), 2.00-1.98 (m, 1H), 1.99 (d, J=4.4 Hz, 2H). ³¹P NMR (162 MHz, CD₃CN) δ=95.97, 95.60.

Preparation of compound 14-6: To a mixture of 14-5 (1000.00 mg, 764.31 umol, 1 eq) in a mixed solvent of pyridine (8 mL) and acetic acid (2 mL) was added resin Amberlyst A₁₅N₂H₅⁺ (1.5 g). The mixture was stirred at 20° C. for 2 hours. The reaction mixture was quenched by addition of water (10 mL) and extracted with EtOAc (20 mL*3). The combined organic layers were washed with brine (20 mL*2), dried over Na₂SO₄, filtered, and concentrated to give a residue, which was purified by silica gel column chromatography (12 g SepaFlash® Silica Flash Column, Eluent of 0˜5% MeOH/DCM gradient at 25 mL/min) to give compound 14-6 (630 mg, 504.93 umol, 66.06% yield) as a light yellow solid. LCMS (ESI): m/z calcd. for (C₆₀H₆₂N₉O₁₃PS₂)/2, 605.68 [M+2H]²⁺, found 605.7; ¹H NMR (400 MHz, CDCl₃) δ=13.55-12.29 (m, 1H), 9.16 (br s, 1H), 8.82-8.69 (m, 1H), 8.34-8.25 (m, 3H), 8.00 (br d, J=6.6 Hz, 2H), 7.58 (br d, J=6.8 Hz, 1H), 7.49 (br d, J=6.8 Hz, 4H), 7.43 (br d, J=7.1 Hz, 4H), 7.30 (br t, J=7.6 Hz, 6H), 6.82 (br d, J=8.2 Hz, 4H), 6.31 (br s, 1H), 5.84 (br s, 1H), 4.54-4.38 (m, 3H), 4.37-3.99 (m, 6H), 3.96-3.88 (m, 1H), 3.81-3.73 (m, 7H), 3.71-3.63 (m, 4H), 3.61 (br s, 3H), 3.46 (br d, J=6.8 Hz, 1H), 2.64-2.45 (m, 3H), 2.06 (br s, 3H); ³¹P NMR (162 MHz, CD₃CN) δ=97.77, 96.99.

Preparation of compound 14-8: To a mixture of 14-6 (3.4 g, 2.81 mmol, 1 eq) in MeCN (20 mL) were added 1H-imidazole-4,5-dicarbonitrile (DCI) (497.65 mg, 4.21 mmol, 1.5 eq) and 4A MS (1.0 g), followed by P-2 (1.69 g, 5.62 mmol, 1.78 mL, 2 eq), and the mixture was stirred at 20° C. for 1 hour. The reaction mixture was poured into NaHCO₃ (sat. aqueous, 50 mL), and then diluted with H₂O (50 mL) and extracted with EtOAc (30 mL*3). The combined organic layers were washed with brine (50 mL*2), dried over anhydrous Na₂SO₄, filtered, and concentrated under reduced pressure to give a residue, which was purified by Flash C-18 column chromatography (Column, C18 silica gel; mobile phase, water and acetonitrile, 0%-90%) to give compound 14-8 (1380.1 mg, 961.07 umol, 34.21% yield) as a white solid. LCMS (ESI): m/z calcd. for C₆₉H₇₈N₁₁O₁₄P₂S₂ 1410.47 [M+H]⁺, found 1410.8; ¹H NMR (400 MHz, CDCl₃) δ=13.25 (br s, 1H), 9.00 (br s, 1H), 8.78 (d, J=1.3 Hz, 1H), 8.34-8.27 (m, 3H), 8.03-7.96 (m, 2H), 7.62-7.27 (m, 16H), 6.84-6.79 (m, 4H), 6.30 (d, J=10.0 Hz, 1H), 5.87-5.79 (m, 1H), 4.47-3.85 (m, 10H), 3.80-3.46 (m, 18H), 2.75-2.50 (m, 4H), 2.08-2.01 (m, 3H), 1.21-1.13 (m, 12H); ³¹P NMR (162 MHz, CD₃CN) δ=150.81, 150.54, 150.46, 150.05; 97.84, 97.47, 97.29, 96.68.

Preparation of compound 15-2: To a mixture of 15-1 (10 g, 14.75 mmol, 1 eq.) in DCM (100 mL) were added triethylsilane (20 mL) and TFA (2 mL) at 0° C. The reaction mixture was stirred at 20° C. for 2 hours. TLC (DCM/MeOH=10/1) indicated that the reaction was complete. The reaction mixture was neutralized by addition of NaHCO₃ (sat. aqueous 50 mL) and filtered. The collected solid was washed with DCM (50 mL×2) and then triturated using EtOAc (50 mL) at 20° C. to give compound 15-2 (5.3 g, 14.12 mmol, 95.69% yield) as a red solid. ¹H NMR (400 MHz, CDCl₃) δ=12.73 (br s, 1H), 8.19-8.08 (m, 3H), 7.58-7.52 (m, 1H), 7.50-7.44 (m, 2H), 5.88 (d, J=4.0 Hz, 1H), 5.44-5.04 (m, 2H), 4.15 (t, J=5.3 Hz, 1H), 3.91-3.86 (m, 1H), 3.85-3.81 (m, 1H), 3.77-3.71 (m, 1H), 3.61 (br dd, J=2.5, 12.0 Hz, 1H), 3.40 (s, 3H), 1.99 (s, 3H).

Preparation of compound 15-3: To a solution of PPh₃ (5.59 g, 21.31 mmol, 2 eq.) in THF (100 mL) was added dropwise DIAD (4.31 g, 21.31 mmol, 4.14 mL, 2 eq.) at 0° C. After stirring for 30 minutes at 15° C., 15-2 (4 g, 10.66 mmol, 1 eq.) was added and the reaction was stirred for additional 10 min. To the resulting yellow suspension at 0° C. was added dropwise a solution of ethanethioic S-acid (1.1 g, 14.45 mmol, 1.03 mL, 1.36 eq.) in THF (20 mL), and stirring was continued at 15° C. for additional 17 hours. The reaction mixture was concentrated under reduced pressure to give a residue, which was purified by flash silica gel chromatography (ISCO®; 80 g SepaFlash® Silica Flash Column, Eluent of 0˜60% Ethyl acetate/Petroleum ether gradient at 60 mL/min) to give 15-3 (2.5 g, 4.61 mmol, 43.30% yield) as a colorless oil. LC-MS (ESI): m/z calcd. for C₂₀H₂₃N₃O₆S 434.13 [M+H]⁺, found 434.1. ¹H NMR (400 MHz, CD₃OD) δ=8.26 (br d, J=7.6 Hz, 2H), 7.72 (s, 1H), 7.53-7.38 (m, 3H), 5.85 (d, J=3.2 Hz, 1H), 4.04-3.91 (m, 3H), 3.51 (s, 3H), 3.39-3.32 (m, 2H), 2.37 (s, 3H), 2.15 (s, 3H).

Preparation of compound 15-4: To the solution of 15-3 (5.5 g, 12.69 mmol, 1 eq.) in DCM (11 mL) were added imidazole (2.59 g, 38.06 mmol, 3 eq.) and TBSCl (3.82 g, 25.38 mmol, 3.11 mL, 2 eq.), and the mixture was stirred at 15° C. for 2 hour. The reaction mixture was filtered and concentrated under reduced pressure to give a residue, which was purified by flash silica gel chromatography (ISCO®; 20 g SepaFlash® Silica Flash Column, Eluent of 0˜20% Ethyl acetate/Petroleum ether gradient at 35 mL/min) to afford 15-4 (5.6 g, 10.22 mmol, 80.58% yield) as a colorless oil. LC-MS (ESI): m/z calcd. for C₂₆H₃₇N₃O₆SSi 548.22 [M+H]⁺, found 548.6. ¹H NMR (400 MHz, CD₃OD) δ=8.23 (br d, J=7.3 Hz, 2H), 7.67 (s, 1H), 7.55-7.34 (m, 3H), 5.84 (d, J=4.4 Hz, 1H), 4.22-4.12 (m, 1H), 4.00-3.89 (m, 2H), 3.40 (s, 3H), 3.34-3.27 (m, 2H), 2.33 (s, 3H), 2.11 (s, 3H), 0.89 (s, 9H), 0.10 (s, 6H).

Preparation of compound 15-5: To a solution of 15-4 (3.2 g, 5.84 mmol, 1 eq.) in MeCN (10 mL) were added DTT (2.70 g, 17.53 mmol, 2.60 mL, 3 eq.) and LiOH.H₂O (245.16 mg, 5.84 mmol, 1 eq.). The mixture was stirred at 15° C. for 2 hours. The reaction mixture was quenched by addition of water (30 mL) and then extracted with EtOAc (30 mL×3). The combined organic layers were washed with brine (30 mL×2), dried over Na₂SO₄, filtered and concentrated under reduced pressure to give a residue. The crude product was purified by flash silica gel chromatography (ISCO®; 20 g SepaFlash® Silica Flash Column, Eluent of 0˜20% Ethyl acetate/Petroleum ether gradient at 35 mL/min, 25 min) to give 15-5 (2.34 g, 4.53 mmol, 77.46% yield) as a white solid. LC-MS (ESI): m/z calcd. for C₂₄H₃₆N₃O₅SSi 506.20 [M+H]⁺, found 506.2. ¹H NMR (400 MHz, CDCl₃) δ=13.18 (br s, 1H), 8.19 (d, J=7.1 Hz, 2H), 7.57 (s, 1H), 7.45-7.38 (m, 1H), 7.35-7.29 (m, 2H), 7.13 (s, 1H), 5.73 (d, J=2.2 Hz, 1H), 4.09-3.96 (m, 2H), 3.65 (dd, J=2.3, 5.0 Hz, 1H), 3.40 (s, 3H), 2.94 (ddd, J=3.7, 8.5, 14.5 Hz, 1H), 2.70 (ddd, J=4.2, 8.7, 14.5 Hz, 1H), 2.03-1.98 (m, 3H), 0.79 (s, 9H), 0.00 (s, 6H).

Preparation of compound 15-6: To a solution of 15-5 (1.8 g, 3.56 mmol, 1 eq.) and TEA (720.35 mg, 7.12 mmol, 990.86 uL, 2 eq.) in DCM (20 mL) at 0° C. were added DMTrCl (1.45 g, 4.27 mmol, 1.2 eq.) and then DMAP (86.97 mg, 711.88 umol, 0.2 eq.), and the mixture was stirred at 20° C. for 16 hours. The reaction mixture was quenched by MeOH (10 mL) and evaporated under vacuum to give a residue, which was purified by silica gel column (PE:EtOAc=6:1) to give 15-6 (2.98 g, 3.47 mmol, 97.39% yield) as a yellow solid. LC-MS (ESI): RT=2.769 min, m/z calcd. for C₄₅H₅₄N₃O7SSi, 808.34 [M+H]⁺, found 808.4. ¹H NMR (400 MHz, CDCl₃) δ=13.30 (br s, 1H), 8.32 (d, J=7.3 Hz, 2H), 7.72 (s, 1H), 7.57-7.50 (m, 1H), 7.48-7.40 (m, 4H), 7.35-7.27 (m, 5H), 7.25-7.14 (m, 2H), 6.86-6.79 (m, 4H), 5.82 (d, J=2.9 Hz, 1H), 4.16-4.07 (m, 1H), 3.86 (t, J=5.8 Hz, 1H), 3.80 (s, 6H), 3.68 (dd, J=3.0, 5.0 Hz, 1H), 3.48 (s, 3H), 2.62 (dd, J=4.2, 12.8 Hz, 1H), 2.46 (dd, J=5.8, 12.7 Hz, 1H), 2.05 (s, 3H), 0.84 (s, 9H), 0.01 (d, J=19.4 Hz, 6H).

Preparation of compound 15-7: To a solution of 15-6 (2.98 g, 3.47 mmol, 1 eq.) in THF (30 mL) was added TBAF (1.0 M, 6.93 mL, 2 eq.) at 20° C. The reaction mixture was stirred at 20° C. for 16 hours. The reaction was diluted with EtOAc (100 mL), washed with water (50 mL×2), brine (50 mL), dried over Na₂SO₄, filtered, and concentrated under vacuum to give a residue. The residue was purified by silica gel column chromatography (DCM:MeOH=100:1) to give 15-7 (2.67 g, 3.46 mmol, 99.96% yield) as a white foam. LC-MS (ESI): RT=2.459 min, m/z calcd. for C₃₉H₄₀N₃O₇S, 694.25 [M+H]⁺, found 694.3. ¹H NMR (400 MHz, CDCl₃) δ=13.29 (br s, 1H), 8.31 (br d, J=8.2 Hz, 2H), 7.69 (s, 1H), 7.56-7.50 (m, 1H), 7.48-7.38 (m, 4H), 7.34-7.26 (m, 5H), 7.25-7.14 (m, 2H), 6.83 (br d, J=8.8 Hz, 5H), 5.87 (s, 1H), 3.93 (br s, 2H), 3.80 (s, 6H), 3.74-3.69 (m, 1H), 3.58 (s, 3H), 2.80 (br d, J=13.2 Hz, 1H), 2.60-2.44 (m, 1H), 2.03 (s, 3H).

Preparation of compound 15-8: To a solution of 15-7 (2.67 g, 3.46 mmol, 1 eq.) and 4-oxopentanoic acid (603.25 mg, 5.20 mmol, 1.5 eq.) in DCM (30 mL) was added EDCI (995.94 mg, 5.20 mmol, 1.5 eq.) at 0° C. The reaction was allowed to warm to 20° C., and DMAP (634.69 mg, 5.20 mmol, 1.5 eq.) was added. The reaction mixture was stirred at 20° C. for 1 hours and diluted with DCM (100 mL). The mixture was washed with water (50 mL×2), brine (50 mL), dried over Na₂SO₄, filtered, and concentrated under vacuum to give a residue. The residue was purified by silica gel column (DCM:MeOH=120:1) to give 15-8 (2.65 g, 3.28 mmol, 94.69% yield) as a white foam. LC-MS (ESI): m/z calcd. for C₄₄H₄₆N₃O₉S, 792.29 [M+H]⁺, found 792.3. ¹H NMR (400 MHz, CDCl₃) δ=13.22 (br s, 1H), 8.32 (br d, J=8.2 Hz, 2H), 7.63 (s, 1H), 7.57-7.50 (m, 1H), 7.48-7.37 (m, 4H), 7.30 (br d, J=8.6 Hz, 5H), 7.25-7.14 (m, 2H), 6.83 (br d, J=8.8 Hz, 4H), 5.92 (d, J=4.4 Hz, 1H), 4.82 (t, J=5.5 Hz, 1H), 4.16 (br d, J=5.1 Hz, 1H), 3.93 (t, J=5.0 Hz, 1H), 3.80 (s, 6H), 3.38 (s, 3H), 2.86-2.70 (m, 3H), 2.63-2.46 (m, 3H), 2.19 (s, 3H), 2.03 (s, 3H).

Preparation of compound 15-9 (Monomer D1): To a solution of 15-8 (2.55 g, 3.16 mmol, 1 eq.) in DCM (67 mL) was added triethylsilane (1.10 g, 9.47 mmol, 1.51 mL, 3 eq.) at 20° C. TFA (3.60 g, 31.56 mmol, 2.34 mL, 10 eq.) was added into the resulting mixture dropwise at 20° C. The reaction mixture was stirred at 20° C. for 0.5 hours The reaction mixture was quenched with sat.NaHCO₃ aqueous (100 mL) and extracted with DCM (100 mL×2). The combined organic phase was washed with brine (80 mL), dried over Na₂SO₄, filtered and concentrated under vacuum to give a residue. The residue was purified by column chromatography (silica gel, DCM:MeOH=100:1) to give 15-9 (1.36 g, 2.64 mmol, 83.63% yield) as a white foam. LC-MS: (ESI): m/z calcd. for C₂₃H₂₈N₃O7S, 490.16 [M+H]⁺, found 490.2. ¹H NMR (400 MHz, DMSO-d₆) δ=12.83 (br s, 1H), 9.71 (s, 1H), 8.92 (s, 1H), 8.19 (br d, J=7.3 Hz, 2H), 8.12-7.82 (m, 2H), 7.65-7.41 (m, 5H), 5.86 (br d, J=5.9 Hz, 1H), 5.34-5.23 (m, 1H), 4.32 (br t, J=5.7 Hz, 1H), 4.09 (br d, J=4.4 Hz, 1H), 3.28 (s, 4H), 2.13 (m, 4H), 2.04 (s, 3H).

Preparation of compound 15-11: To a solution of 15-10 (10 g, 35.55 mmol, 1.0 eq.) in pyridine (100 mL) was added TMSCl (23.18 g, 213.32 mmol, 27.07 mL, 6.0 eq.) at 0° C. under N₂ atmosphere, and the mixture was stirred at 0° C. for 5 hours under N₂ atmosphere. To the above mixture at 0° C. was added BzCl (10.00 g, 71.11 mmol, 8.26 mL, 2.0 eq.), and the mixture was allowed to warm to 15° C. and stirred for 2 hours. The mixture was then cooled to 0° C. H₂O (6.00 g, 333.05 mmol, 6 mL, 9.37 eq.) was added, and the mixture was stirred for 0.5 hours at 15° C. Then NH₃.H₂O (27.30 g, 233.69 mmol, 30 mL, 30% purity, 6.57 eq.) was added at 15° C. and the mixture was stirred at 15° C. for 2 hours. The reaction mixture was concentrated under reduced pressure to give a residue, which was triturated with DCM (100 mL) at 15° C. for 1 hr to give 15-11 (65 g, crude) as a white solid. The crude product was triturated again with H₂O (200 mL) at 15° C. for 1 hr to give compound 15-11 (26.5 g, 62.69 mmol, 88.17% yield) as a white solid. (2 batches in parallel, combined for purification) LC-MS (ESI): m/z calcd. for C₁₈H₂₀N₅O₅ 386.14 [M+H]⁺, found 386.1; ¹H NMR (400 MHz, DMSO-d₆) δ=11.25 (br s, 1H), 8.76 (d, J=5.1 Hz, 2H), 8.05 (d, J=7.6 Hz, 2H), 7.75-7.44 (m, 3H), 6.17 (d, J=5.9 Hz, 1H), 5.47-5.05 (m, 2H), 4.50-4.32 (m, 2H), 4.01 (q, J=3.7 Hz, 1H), 3.79-3.50 (m, 2H), 3.16 (s, 3H).

Preparation of compound 15-12: To a solution of 15-11 (25 g, 64.87 mmol, 1 eq.) in pyridine (400 mL) was added 1-[chloro-(4-methoxyphenyl)-phenyl-methyl]-4-methoxy-benzene (26.38 g, 77.85 mmol, 1.2 eq.) at 0° C. under N₂ atmosphere, and the mixture was degassed and purged with N₂ for 3 times. Then the mixture was warmed to 15° C. and stirred for 4 hours under N₂ atmosphere. The reaction was quenched by MeOH (20 mL) and concentrated under reduced pressure to give a residue. The residue was purified by column chromatography (SiO₂, PE:EtOAc=1:1-0:1) to give compound 15-12 (90.6 g, 131.47 mmol, 67.55% yield) as a yellow foam. (3 batches in parallel, combined for purification). LC-MS (ESI): m/z calcd. for C₃₉H₃₈N₅O₇ 688.27 [M+H]⁺, found 688.3.

Preparation of compounds 15-13 and 15-14: To a mixture of compound 15-12 (co-evaporated with pyridine twice) (26 g, 37.81 mmol, 1 eq.) in pyridine (260 mL) was added Tf₂O (13.87 g, 49.15 mmol, 8.11 mL, 1.3 eq.) dropwise at 0° C. under N₂, and the mixture was stirred at 0° C. for 2 hr under N₂ atmosphere. TBAA (68.39 g, 226.83 mmol, 69.08 mL, 6 eq.) was then added and the mixture (containing 15-13) was stirred at 15° C. for 12 hr under N₂ atmosphere. The reaction mixture was poured into H₂O (50 mL) and then filtered. The collected solid was dissolved in ethyl acetate (2000 mL), and the solution were washed with brine (300 mL×3), dried over anhydrous Na₂SO₄, filtered, and concentrated under reduced pressure to give crude compound 15-14 (84 g, 61.09 mmol, 53.86% yield) as a yellow foam, which was used without further purification. (3 batches in parallel, combined for purification). LC-MS (ESI): m/z calcd. for C₄₁H₃₉N₅O₈ 730.28 [M+H]⁺, found 730.3.

Preparation of compound 15-15: A mixture of 15-14 (36 g, 49.33 mmol, 1 eq.) and MeONa (10.66 g, 197.32 mmol, 4 eq.) in THF (500 mL) was degassed and purged with N₂ for 3 times, and the mixture was stirred at 15° C. for 16 hr under N₂ atmosphere. The reaction mixture was then diluted with ice water/NH₄Cl aqueous (1:1, 4000 mL) and then extracted with ethyl acetate (1200 mL×2). The combined organic layers were washed with brine (600 mL×3), dried over anhydrous Na₂SO₄, filtered, and concentrated under reduced pressure to give a residue. The residue was purified by column chromatography (SiO₂, DCM:MeOH/acetone (1:1)=1000:1˜100:1) to give compound 15-15 (40 g) (30 g, 57.13% purity), and (14 g, 70.57% purity) as yellow foam. (3 batches in parallel, combined for purification) LC-MS (ESI): m/z calcd. for C₃₉H₃₈N₅O₇ 688.27 [M+H]⁺, found 688.3.

Preparation of compound 15-16: To a solution of 15-15 (9 g, 13.09 mmol, 1 eq.) and DMAP (7.80 g, 63.85 mmol, 4.88 eq.) in DCM (300 mL) was added Tf₂O (11.08 g, 39.26 mmol, 6.48 mL, 3 eq.) at 0° C. The mixture was stirred at 0˜15° C. for 2 hours under N₂ atmosphere. The reaction mixture was diluted with DCM (500 mL), washed with ice water (500 mL×2), dried over Na₂SO₄, filtered, and concentrated under reduced pressure to give crude compound 15-16 (22 g, 23.09 mmol, 88.24% yield, 86.06% purity) as a yellow foam, which was used for next step without further purification. (2 batches in parallel, combined for purification). LC-MS (ESI): m/z calcd. for C₄₀H₃₇F₃N₅O₉S 820.22 [M+H]⁺, found 820.2.

Preparation of compound 15-17: To a solution of 15-16 (1.5 g, 1.34 mmol, 1 eq) in DMF (6 mL) was added KSAc (762.73 mg, 6.68 mmol, 5 eq), and the mixture was stirred at 20° C. for 2 hr to give a dark red solution. The reaction progress was monitored by LC-MS (m/z calcd. for C₄₁H₄₀N₅O₇S 746.26 [M+H]⁺, found 746.2). LC-MS conditions: 1.5 mL/4 L TFA in water (solvent A) and 0.75 mL/4LTFA in acetonitrile (solvent B), using the elution gradient 5%-95% (solvent B) over 0.7 minutes and holding at 95% for 0.4 minutes at a flow rate of 1.5 mL/min; Column: Agilent Pursuit 5 C18 20*2.0 mm. The reaction mixture was poured into ice-water (150 mL), and the resulting precipitate was filtered and washed with water (20 mL×3) to give a yellow solid. The solid was purified by silica gel column (DCM/Acetone=100:1) to give compound 15-17 (0.689 g, 775.98 umol, 58.10% yield) as a light yellow solid. LC-MS (ESI): m/z calcd. for C₄₁H₄₀N₅O₇S 746.26 [M+H]⁺, found 746.3. ¹H NMR (400 MHz, DMSO-d₆) δ=11.26 (s, 1H), 8.75 (s, 1H), 8.67 (s, 1H), 8.04 (d, J=7.3 Hz, 2H), 7.68-7.62 (m, 1H), 7.59-7.52 (m, 2H), 7.35-7.15 (m, 7H), 7.09-7.05 (m, 1H), 6.92-6.78 (m, 4H), 6.34 (s, 1H), 4.78-4.72 (m, 1H), 4.63-4.59 (m, 1H), 4.21-4.14 (m, 1H), 3.74-3.72 (m, 3H), 3.71 (s, 6H), 3.48 (s, 3H), 2.32 (s, 3H).

Preparation of compound 15-18 (Monomer C): To a solution of 15-17 (6 g, 7.15 mmol, 1 eq.) in DMA (60 mL) were added DTT (3.31 g, 21.44 mmol, 3.18 mL, 3 eq.) and NaHCO₃ (720.46 mg, 8.58 mmol, 333.54 uL, 1.2 eq.) under N₂. The mixture was stirred at 15° C. for 2 hours and then poured into ice-water (1200 mL). The resulting precipitate was filtered and washed with water (150 mL×3) to give a yellow solid, which was dissolved in ethyl acetate (1500 mL), dried over Na₂SO₄, filtered and concentrated under vacuum to give a yellow solid, which was purified by silica gel column (PE/EtOAc=1:1, 0.5% TEA) to give compound 15-18 (9 g, 11.18 mmol, 78.24% yield) as a yellow foam. (2 batches in parallel, combined for purification) LC-MS (ESI): m/z calcd. for C₃₉H₃₈N₅O₆S 704.25 [M+H]⁺, found 704.3; ¹H NMR (400 MHz, CDCl₃) δ=9.02 (s, 1H), 8.77 (s, 1H), 8.40 (s, 1H), 7.98 (br d, J=7.9 Hz, 2H), 7.61-7.52 (m, 1H), 7.51-7.44 (m, 2H), 7.37 (br d, J=7.7 Hz, 2H), 7.26 (s, 8H), 6.78 (br d, J=8.6 Hz, 4H), 6.20 (s, 1H), 4.17 (d, J=4.9 Hz, 1H), 4.09-4.02 (m, 2H), 3.73 (s, 6H), 3.68 (s, 3H), 3.61 (br d, J=11.0 Hz, 1H), 3.43 (dd, J=3.1, 11.2 Hz, 1H).

Preparation of compound 15-19: To a solution of 15-18 (1.6 g, 2.27 mmol, 1 eq., co-evaporated with toluene twice) and 1H-imidazole-4,5-dicarbonitrile (402.71 mg, 3.41 mmol, 1.5 eq.) in MeCN (50 mL) was added P-2 (1.37 g, 4.55 mmol, 1.44 mL, 2 eq.) in one portion at 20° C., and the mixture was stirred at 20° C. for 1 hour. The reaction was quenched with NaHCO₃ (sat. aqueous, 100 mL) and extracted with ethyl acetate (100 mL×2). The combined organic phase was washed with brine (150 mL), dried over Na₂SO₄, filtered, and concentrated under vacuum to give a residue. The residue was purified by prep-HPLC (ISCO®; 40 g, SepaFlash® C18 Column, Eluent of 20-90% ACN/water, gradient at 45 mL/min, 30 CV) to give 15-19 (1.88 g, 2.08 mmol, 91.48% yield) as a white foam. LC-MS: (ESI): m/z calcd. for C₄₈H₅₅N₇O₇PS 904.36 [M+H]⁺, found 904.4; ¹H NMR (400 MHz, CD₃CN) δ=8.71-8.61 (m, 1H), 8.37 (br d, J=5.1 Hz, 1H), 8.00 (br d, J=7.1 Hz, 2H), 7.69-7.60 (m, 1H), 7.58-7.50 (m, 2H), 7.38 (br t, J=6.8 Hz, 2H), 7.30-7.16 (m, 8H), 6.84-6.73 (m, 4H), 6.28-6.21 (m, 1H), 4.47 (br s, 1H), 4.34-4.23 (m, 1H), 4.12-3.76 (m, 3H), 3.73 (d, J=2.0 Hz, 6H), 3.71-3.62 (m, 2H), 3.62-3.53 (m, 4H), 3.37 (dt, J=4.3, 10.6 Hz, 1H), 2.71-2.58 (m, 1H), 2.49 (t, J=6.0 Hz, 1H), 1.30-1.13 (m, 6H), 1.11-0.98 (m, 6H); ³¹P NMR (162 MHz, CD₃CN) δ=164.020, 159.613.

Preparation of compound 15-20: To a suspension of 15-9 (850 mg, 1.74 mmol, 1 eq.), 4A-MS (2 g, 1.74 mmol, 1 eq.), and P-3 (1.33 g, 6.95 mmol, 4 eq.) in MeCN (6 mL) was added dropwise a solution of 15-19 (1.88 g, 2.08 mmol, 1.2 eq., co-evaporated with toluene twice) in MeCN (6 mL), and the mixture was stirred at 20° C. for 16 hours. Then a suspension of S (1.67 g, 52.09 mmol, 30 eq.) in DCM (10 mL) was added at 20° C. The mixture was stirred at 20° C. for 1 hr and diluted with DCM (250 mL). The mixture was filtered, and the filtrate was washed with NaHCO₃ (sat. aqueous, 200 mL), brine (150 mL), dried over Na₂SO₄, filtered, and concentrated under vacuum to give a yellow residue. The residue was purified by prep-HPLC (ISCO®; 40 g, SepaFlash® C18 Column, Eluent of 0˜85% ACN/water, gradient at 40 mL/min, 45 CV) to give 15-20 (1.58 g, 1.06 mmol, 61.15% yield) as a white solid. LC-MS (ESI): m/z calcd. for C₆₅H₆₇N₉O₁₄PS₃ 1324.37 [M+H]⁺, found 1324.2; ¹H NMR (400 MHz, CD₃CN) δ=13.12 (br s, 1H), 9.31-9.17 (m, 1H), 8.71-8.60 (m, 1H), 8.37-8.24 (m, 4H), 8.02-7.94 (m, 2H), 7.68-7.42 (m, 7H), 7.38-7.31 (m, 2H), 7.27-7.14 (m, 6H), 6.82-6.71 (m, 4H), 6.28-6.23 (s, 1H), 5.78-5.71 (m, 1H), 5.22-5.04 (m, 1H), 4.83-4.63 (m, 2H), 4.38-4.09 (m, 6H), 3.78-3.66 (m, 7H), 3.63-3.48 (m, 4H), 3.44-3.22 (m, 7H), 2.79-2.68 (m, 4H), 2.59-2.48 (m, 2H), 2.04 (s, 3H); ³¹P NMR (162 MHz, CD₃CN) δ=109.845, 109.661.

Preparation of compound 15-21: To a solution of 15-20 (1.58 g, 1.19 mmol, 1 eq.) in a mixed solvent of pyridine (36 mL) and HOAc (9 mL) was added resin Amberlyst A₁₅N₂H₅₊ (2.37 g) at 20° C. The mixture was stirred at 20° C. for 2 hr and then added into water (220 mL) to give a white precipitate, which was filtered and washed with water (20 mL×2) to give a white solid. The solid was purified by reverse-phase HPLC (ISCO®; 40 g, SepaFlash® C18 Column, Eluent of 0˜75% ACN/water, gradient at 50 mL/min, 50 CV) to give 15-21 (1.26 g, 996.63 umol, 83.54% yield) as a white solid. LC-MS (ESI): m/z calcd. for C₆₀H₆₁N₉O₁₂PS₃ 1226.33 [M+H]⁺, found 1226.3; ¹H NMR (400 MHz, CD₃CN) δ=13.20 (br s, 1H), 9.38-9.24 (m, 1H), 8.67-8.63 (m, 1H), 8.36-8.31 (m, 1H), 8.26 (br d, J=7.3 Hz, 2H), 7.97 (br d, J=7.3 Hz, 2H), 7.65-7.43 (m, 7H), 7.38-7.33 (m, 2H), 7.27-7.14 (m, 7H), 6.28 (s, 1H), 6.25 (s, 1H), 5.75 (d, J=2.9 Hz, 1H), 4.85-4.68 (m, 2H), 4.40-4.31 (m, 1H), 4.29-4.12 (m, 2H), 4.09-4.00 (m, 2H), 3.90 (br d, J=2.2 Hz, 1H), 3.76-3.68 (m, 6H), 3.61-3.57 (m, 3H), 3.57-3.53 (m, 1H), 3.45 (d, J=1.5 Hz, 4H), 3.44-3.19 (m, 3H), 2.78-2.69 (m, 2H), 2.06-2.00 (m, 3H); ³¹P NMR (162 MHz, CD₃CN) δ=109.891, 109.638.

Preparation of compound 15-22: To a mixture of 15-21 (1.26 g, 1.03 mmol, 1 eq., co-evaporated with toluene twice), 1H-imidazole-4,5-dicarbonitrile (182.01 mg, 1.54 mmol, 1.5 eq.), and 4A-MS (0.2 g) in MeCN (55 mL) at 20° C. was added P-2 (619.36 mg, 2.05 mmol, 652.64 uL, 2 eq.) dropwise, and the mixture was stirred at 20° C. for 1 hour. The reaction mixture was quenched with NaHCO₃ (sat. aqueous, 100 mL), extracted with ethyl acetate (2×100 mL). The combined organic phase was washed with brine (150 mL), dried over Na₂SO₄, filtered, and concentrated under vacuum to give a residue. The residue was purified by prep-HPLC (ISCO®; 40 g, SepaFlash® C18 Column, Eluent of 20-90% ACN/water, gradient at 45 mL/min, 30 CV) to give compound 15-22 (0.78 g, 541.30 umol, 52.68% yield) as a white solid. LC-MS: (ESI): m/z calcd. for C₆₉H₇₈N₁₁O₁₃P₂S₃, 1426.44 [M+H]⁺, found 1426.9; ¹H NMR (400 MHz, CD₃CN) δ=13.15 (br s, 1H), 9.35 (br s, 1H), 8.63 (d, J=6.5 Hz, 1H), 8.37-8.30 (m, 1H), 8.25 (br d, J=7.5 Hz, 2H), 8.02-7.91 (m, 2H), 7.64-7.42 (m, 7H), 7.38-7.31 (m, 2H), 7.27-7.13 (m, 7H), 6.80-6.71 (m, 4H), 6.29-6.23 (m, 1H), 5.78-5.71 (m, 1H), 4.86-4.66 (m, 2H), 4.39-4.09 (m, 5H), 4.08-4.00 (m, 1H), 3.91-3.73 (m, 2H), 3.72-3.69 (m, 6H), 3.69-3.62 (m, 2H), 3.61-3.58 (m, 3H), 3.58-3.47 (m, 2H), 3.47-3.40 (m, 3H), 3.40-3.19 (m, 2H), 2.81-2.63 (m, 4H), 2.05-2.00 (m, 3H), 1.23-1.13 (m, 12H); ³¹P NMR (162 MHz, CD₃CN) δ=149.863, 149.699, 149.663, 110.153, 109.808, 109.718.

### Example A6

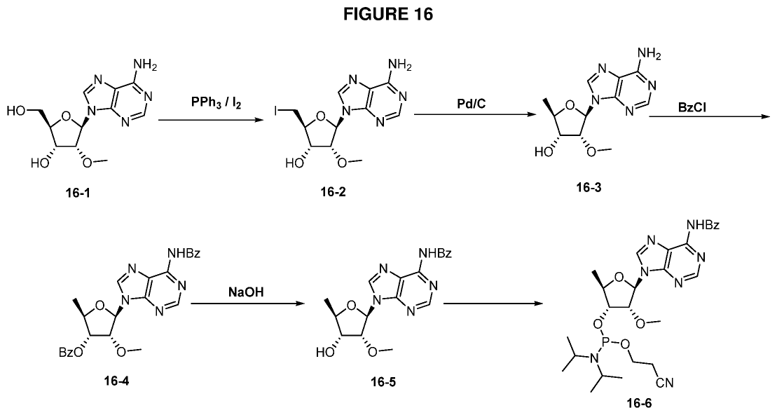

The building block compound 16-6 is useful for making embodiments of modified phosphorothioated oligonucleotides. With reference to FIG. 16, the compound 16-6 was prepared as follows:

Preparation of compound 16-2: To a stirred solution of 16-1 (10.0 g, 35.6 mmol) in pyridine (100 mL) were added PPh₃ (14.0 g, 53.3 mmol) at room temperature With cooling on an ice bath, to the reaction mixture was added I₂ (13.5 g, 53.3 mmol) and the reaction mixture was stirred at room temperature for 2 h, and saturated aqueous Na₂S₂O₃ was added and the resulting mixture was extracted with EA. The combined organic layer was washed with water and brine, dried over Na₂SO₄, and concentrated to give the crude 16-2 (13.9 g, 35.5 mmol) as a white solid which was used directly for next step. ESI-LCMS: m/z 392 [M+H]⁺.

Preparation of compound 16-3: To the solution of crude 16-2 (13.9 g, 35.5 mol) in MeOH (260 mL) was added TEA (18.0 g, 178.0 mmol) and Pd/C (10%) (3.5 g) at room temperature under H₂ atmosphere. After stirring at room temperature for 15 h, the reaction mixture was filtered, washed by DCM. The combined organic layer was washed with water and brine, dried over Na₂SO₄, and concentrated under reduced pressure and the residue was purified by silica gel column chromatography (SiO₂, DCM:MeOH=100:1-50:1) to give 16-3 (3.4 g, 12.8 mmol, 36.0% over two steps) as a white solid. ¹H-NMR (400 MHz, DMSO-d₆): δ=8.35 (s, 1H), 8.17 (s, 1H), 7.31 (s, 2H), 5.96 (d, J=4.0 Hz, 1H), 5.23 (d, J=6.0 Hz, 1H), 4.47-4.41 (m, 1H), 4.19-4.12 (m, 1H), 4.03-3.94 (m, 1H), 3.35 (s, 3H), 1.32 (d, J=6.2 Hz, 3H); ESI-LCMS: m/z 266 [M+H]⁺.

Preparation of compound 16-4: To the solution of 16-3 (3.4 g, 12.8 mmol) in pyridine (35 mL) was added BzCl (3.7 mL, 32.1 mmol) at 0° C. under N₂ atmosphere. After stirring at room temperature for 1 h, saturated aqueous NaHCO₃ was added and extracted with EA. The combined organic layer was washed with water and brine, dried over Na₂SO₄, and concentrated to give the crude 16-4 (6.0 g, 12.6 mmol) as a white solid which was used directly for next step. ESI-LCMS: m/z 474 [M+H]⁺.

Preparation of compound 16-5: To the solution of crude 16-4 (6.0 g, 12.6 mmol) in Pyridine (60 mL) was added 2N NaOH in MeOH/H₂O (4.5:1) at 0° C. under N₂ atmosphere. After stirring at 0° C. for 1 h, saturated aqueous NH₄Cl was added and the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/9 increasing to CH₃CN/H₂O (0.5% NH₄HCO₃)=1/1 within 20 min, the eluted product was collected at CH₃CN/H₂O (0.5% NH₄HCO₃)=1/1; Detector, UV 254 nm. This resulted in 16-5 (2.4 g, 6.8 mmol, 50.0% over two steps) as a white solid. ¹H-NMR (400 MHz, DMSO-d₆): δ=11.22 (s, 1H), 8.78 (s, 1H), 8.70 (s, 1H), 8.06 (d, J=7.2 Hz, 2H), 7.70-7.62 (m, 1H), 7.60-7.52 (m, 2H), 6.11 (d, J=4.8 Hz, 1H), 5.31 (d, J=5.6 Hz, 1H), 4.56-4.49 (m, 1H), 4.24-4.16 (m, 1H), 4.09-4.00 (m, 1H), 3.39 (s, 3H), 1.35 (d, J=6.4 Hz, 3H); ESI-LCMS: m/z 370 [M+H]⁺.

Preparation of compound 16-6: To a suspension of 16-5 (2.4 g, 6.5 mmol) in DCM (25 mL) was added DCI (613 mg, 5.2 mmol) and CEP[N(iPr)₂]2 (2.4 g, 7.8 mmol). The mixture was stirred at room temperature for 2 hours. LC-MS showed that the reaction worked well. The solution was washed with water twice and washed with brine and dried over Na₂SO₄. Then the solution was concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=4/1; Detector, UV 254 nm. This resulted in compound 16-6 (2.4 g, 4.2 mmol, 64.9%) as a white solid. ¹H-NMR (400 MHz, DMSO-d₆): δ=11.23 (s, 1H), 8.81-8.70 (m, 2H), 8.06 (d, J=7.2 Hz, 2H), 7.70-7.62 (m, 1H), 7.60-7.52 (m, 2H), 6.18-6.07 (m, 1H), 4.82-4.75 (m, 1H), 4.60-4.48 (m, 1H), 4.30-4.15 (m, 1H), 3.93-3.59 (m, 1H), 3.39 (d, J=16.0 Hz, 3H), 2.90-2.78 (m, 2H), 1.45-1.36 (m, 3H), 1.27-1.14 (m, 12H). ³¹P-NMR (162 MHz, DMSO-d₆): δ=149.30, 148.78. ESI-LCMS: m/z 570 [M+H]⁺.

### Example A7

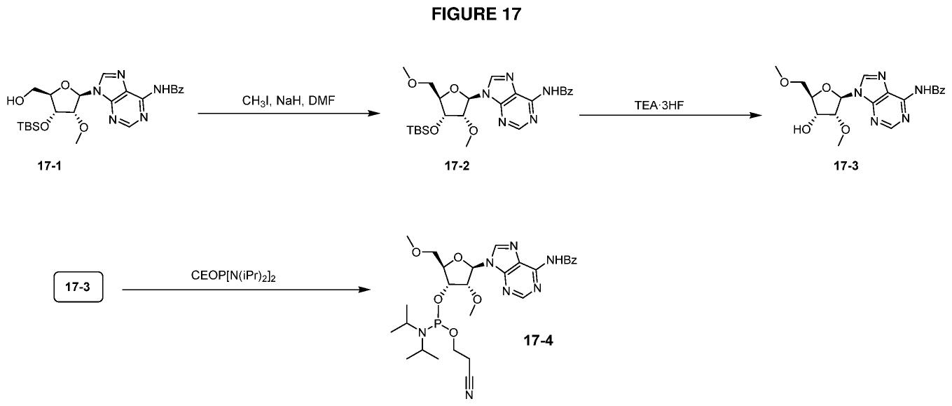

The building block compound 17-4 is useful for making embodiments of modified phosphorothioated oligonucleotides. With reference to FIG. 17, the compound 17-4 was prepared as follows:

Preparation of compound 17-2: To a solution of 17-1 (6 g, 12.01 mmol, 1 eq) in THF (100 mL) was added NaH (960.61 mg, 24.02 mmol, 60% purity, 2 eq) and the mixture was stirred at 0° C. for 30 min. Then CH₃I (1.70 g, 12.01 mmol, 747.59 uL, 1 eq) was added, and the mixture was stirred at 25° C. for 2 hour. TLC (PE:EA=1:1) and LCMS indicated that the reaction was complete. The reaction mixture was quenched by addition of sat. NaHCO₃ (100 mL) at 25° C. and then extracted with EA 150 mL (50 mL*3). The combined organic layers were dried over Na₂SO₄, filtered, and concentrated under reduced pressure to give a residue. The residue was purified by flash silica gel chromatography (ISCO®; 40 g SepaFlash® Silica Flash Column, Eluent of 0˜46% Ethyl acetate/Petroleum ether gradient at 40 mL/min) to give compound 17-2 (4 g, 7.48 mmol, 62.25% yield) as a colorless oil, which was confirmed by LCMS and ¹H NMR. LCMS (ESI): m/z calcd. for C₂₅H₃₆N₅O₅Si 514.24 [M+H]⁺, found 514.2; ¹H NMR (400 MHz, DMSO-d₆) δ=11.18 (br s, 1H), 8.71 (s, 1H), 8.62 (s, 1H), 7.99 (brd, J=7.6 Hz, 2H), 7.66-7.58 (m, 1H), 7.56-7.48 (m, 2H), 6.11 (d, J=5.1 Hz, 1H), 4.53 (t, J=4.2 Hz, 1H), 4.44-4.37 (m, 1H), 4.03 (q, J=3.9 Hz, 1H), 3.62-3.54 (m, 1H), 3.54-3.46 (m, 1H), 3.31 (s, 3H), 3.27 (s, 3H), 0.85 (s, 9H), 0.07 (s, 6H).

Preparation of compound 17-3: To a solution of N-[9-[(2R,3R,4R,5R)-4-[tert-butyl(dimethyl)silyl]oxy-3-methoxy-5-(methoxymethyl)tetrahydrofuran-2-yl]purin-6-yl]benzamide (17-2) (4 g, 7.79 mmol, 1 eq) in THF (10 mL) was added TEA.3HF (18.83 g, 116.81 mmol, 19.04 mL, 15 eq). The mixture was stirred at 25° C. for 12 hour. TLC (PE:EA=0:1) and LCMS indicated that the reaction was complete. The reaction mixture was concentrated under reduced pressure to give a residue, which was purified by flash C-18 column (40 g C-18 column: chromatography (10%˜60% H₂O (0.4 g NH₄HCO₃ in 1 L H₂O)/CH₃CN at 40 mL/min) to give N-[9-[(2R,3R,4R,5R)-4-hydroxy-3-methoxy-5-(methoxymethyl)tetrahydrofuran-2-yl]purin-6-yl]benzamide (compound 17-3) (2.2 g, 5.46 mmol, 70.14% yield, 99.16% purity) as a white solid, which was confirmed by LCMS: ¹H NMR: and ¹H NMR: (DMSO-d₆, D₂O exchange) LCMS (ESI): m/z calcd. for C₁₉H₂₂N₅O₅ 400.15 [M+H]⁺, found 400.1′¹H NMR (400 MHz, DMSO-d₆) δ=11.08 (s, 1H), 8.77 (s, 1H), 8.67 (s, 1H), 8.05 (d, J=7.6 Hz, 2H), 7.68-7.61 (m, 1H), 7.56 (t, J=7.1 Hz, 2H), 6.17 (d, J=5.2 Hz, 1H), 5.42 (br d, J=5.6 Hz, 1H), 4.47-4.35 (m, 2H), 4.09 (q, J=4.0 Hz, 1H), 3.69-3.48 (m, 2H), 3.40-3.37 (m, 3H), 3.33-3.30 (m, 3H); ¹H NMR (400 MHz, DMSO-d₆/D₂O) δ=8.73 (s, 1H), 8.61 (s, 1H), 8.03-7.93 (m, 2H), 7.68-7.59 (m, 1H), 7.58-7.48 (m, 2H), 6.13 (d, J=5.4 Hz, 1H), 4.36 (td, J=4.8, 14.6 Hz, 2H), 4.08 (q, J=4.0 Hz, 1H), 3.62-3.49 (m, 2H), 3.34 (s, 3H), 3.28 (s, 3H).

Preparation of compound 17-4: To a solution of 17-3 (2.4 g, 6.01 mmol, 1 eq) in DCM (20 mL) was added 1H-imidazole-4,5-dicarbonitrile (1.06 g, 9.01 mmol, 1.5 eq) followed by 3-bis(diisopropylamino)phosphanyloxypropanenitrile (3.62 g, 12.02 mmol, 3.82 mL, 2 eq). The reaction mixture was then stirred at 25° C. for 2 hours. TLC (PE:EA=1:1) and LCMS indicated that the reaction was complete. The reaction mixture was then quenched with sat. NaHCO₃ (40 mL), extracted with DCM 60 mL (20 mL*3), washed with brine (40 mL), dried over Na₂SO₄, filtered, and concentrated to give a residue (2.2 g), which was purified in 2 portions.

Portion 1: The residue (500 mg) was purified by flash C-18 column (40 g C-18 column: chromatography (10%˜60% water (0.4 g NH₄HCO₃ in 1 L H₂O)/CH₃CN at 40 mL/min) and then by flash silica gel chromatography (ISCO®; 12 g SepaFlash® Silica Flash Column, Eluent of 0˜75% ethyl acetate/petroleum ether gradient at 30 mL/min) to give N-[9-[(2R,3R,4R,5R)-4-[2-cyanoethoxy-(diisopropylamino)phosphanyl]oxy-3-methoxy-5-(methoxymethyl)tetrahydrofuran-2-yl]purin-6-yl]benzamide (compound 17-4) (330 mg, 545.84 umol, 9.08% yield, 99.18% purity), which was confirmed by ¹H NMR: ³¹P NMR:, LCMS:, and HPLC:; LCMS (ESI): m/z calcd. for C₂₈H₃₉N₇O₆P 600.27 [M+H]⁺, found 600.4, ¹H NMR (400 MHz, CD₃CN) δ=9.30 (br s, 1H), 8.67 (s, 1H), 8.47-8.40 (m, 1H), 8.01 (br d, J=7.3 Hz, 2H), 7.69-7.61 (m, 1H), 7.59-7.51 (m, 2H), 6.22-6.15 (m, 1H), 4.71-4.60 (m, 1H), 4.51-4.43 (m, 1H), 4.36-4.28 (m, 2H), 3.94-3.80 (m, 2H), 3.73-3.65 (m, 4H), 3.48 (s, 2H), 3.44-3.36 (m, 4H), 2.73-2.63 (m, 2H), 1.26-1.19 (m, 12H); ³¹P NMR (162 MHz, CD₃CN) δ=150.39, 149.54 (mixture of diastereomers).

Portion 2: The residue (1.7 g) was purified by flash C-18 column (80 g C-18 column: chromatography (10%˜60% water (0.4 g NH₄HCO₃ in 1 L H₂O)/CH₃CN at 40 mL/min) and then by flash silica gel chromatography (ISCO®; 12 g SepaFlash® Silica Flash Column, Eluent of 0˜75% Ethyl acetate/Petroleum ether gradient at 30 mL/min) to give N-[9-[(2R,3R,4R,5R)-4-[2-cyanoethoxy-(diisopropylamino)phosphanyl]oxy-3-methoxy-5-(methoxymethyl)tetrahydrofuran-2-yl]purin-6-yl]benzamide (compound 17-4) (900 mg, 1.49 mmol, 24.73% yield) as a white solid, which was confirmed by ¹H NMR:, ³¹P NMR:, LCMS:, and HPLC; LCMS (ESI): RT=2.528 min, m/z calcd. for C₂₈H₃₉N₇O₆P 600.27 [M+H]⁺, found 600.4; ¹H NMR (400 MHz, CD₃CN) δ=9.50 (brs, 1H), 8.65 (s, 1H), 8.47-8.40 (m, 1H), 8.00 (br d, J=7.5 Hz, 2H), 7.67-7.59 (m, 1H), 7.58-7.49 (m, 2H), 6.22-6.14 (m, 1H), 4.71-4.60 (m, 1H), 4.50-4.41 (m, 1H), 4.35-4.28 (m, 1H), 3.93-3.80 (m, 2H), 3.73-3.64 (m, 4H), 3.47 (s, 2H), 3.45-3.35 (m, 4H), 2.73-2.65 (m, 2H), 1.26-1.18 (m, 12H); ³¹P NMR (162 MHz, CD₃CN) δ=150.41, 149.54 (mixture of diastereomers).

### Example A8

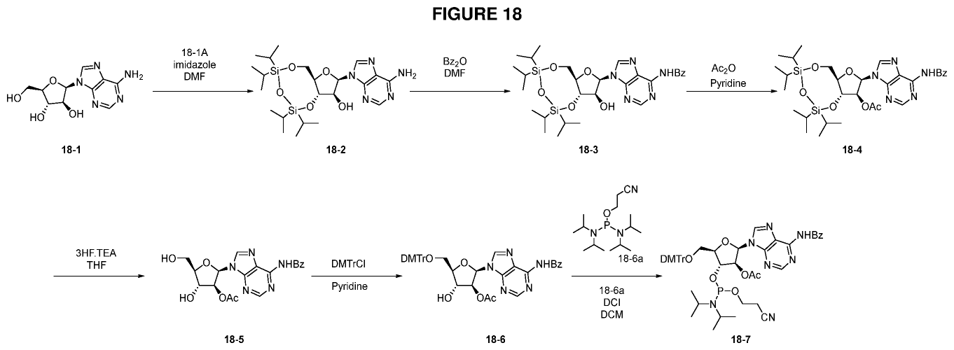

The building block compound 18-7 is useful for making embodiments of modified phosphorothioated oligonucleotides. With reference to FIG. 18, the compound 18-7 was prepared as follows:

Preparation of compound 18-2: Intermediate 18-2 was made in accordance with known procedures. See Bockman, Matthew R., et al Journal of Medicinal Chemistry, 2015, 58(18), 7349-7369. To a solution of 18-1 (16 g, 59.87 mmol) and imidazole (16.30 g, 239.48 mmol) in DMF (200 mL) was added 1,3-dichloro-1,1,3,3-tetraisopropyldisiloxane 18-1A (28.33 g, 89.81 mmol) at 0° C. under N₂, and stirred for 3 hours. The solution was diluted with EA (200 mL) and washed with cold water, saturated aqueous sodium bicarbonate and washed with brine and dry over by Na₂SO₄. Then the solution was concentrated under reduced pressure and the residue was purified by silica gel column (PE:EA, 0˜50%) to give 18-2 (27.2 g, 53.36 mmol, 89.12% yield) as a white solid. ¹H-NMR (400 MHz, DMSO-d₆): δ=8.11 (s, 1H), 8.04 (s, 1H), 7.28 (s, 2H), 6.21-6.19 (d, J=6.56 Hz, 1H), 5.78-5.77 (d, J=5.8 Hz, 1H), 4.59-4.55 (t, J=15.8 Hz, 1H), 4.52-4.49 (t, J=12.8 Hz, 1H), 4.12-4.08 (m, 1H), 3.93-3.90 (d, J=10.6 Hz, 1H), 3.80-3.78 (t, J=7.84 Hz, 1H), 1.13-1.01 (m, 28H). ESI-LCMS: m/z 510 [M+H]⁺.

Preparation of compound 18-3: To a solution of 18-2 (26 g, 51.01 mmol) in DMF (260 mL) was added benzoic anhydride (23.08 g, 102.01 mmol) at room temperature under N₂ and stirred at 80° C. for 24 hours. The mixture was cooled down to room temperature and the solution was diluted with EtOAc (500 mL) and washed with cold water, saturated aqueous sodium bicarbonate and washed with brine and dry over by Na₂SO₄. Then the solution was concentrated under reduced pressure and the residue was purified by silica gel column (PE:EA, 0˜50%) to give 18-3 (23 g, 37.47 mmol, 73.46% yield) as a white solid. ¹H-NMR (400 MHz, CDCl₃): δ=9.21 (s, 1H), 8.53 (s, 1H), 8.17 (s, 1H), 8.02-8.00 (d, J=7.72 Hz, 2H), 7.60-7.56 (t, J=14.6 Hz, 1H), 7.52-7.48 (t, 2H), 6.27-6.26 (d, J=5.64 Hz, 1H), 4.88 (s, 1H), 4.66-4.58 (m, 2H), 4.06-4.05 (d, J=3.48 Hz, 2H), 3.87-3.84 (m, 1H), 1.13-1.05 (m, 28H). ESI-LCMS: m/z 614 [M+H]⁺.

Preparation of 18-4: To a solution of 18-3 (22 g, 35.84 mmol) in pyridine (300 mL) was added Ac₂O (5.70 g, 53.76 mmol, 0.25 mL) at room temperature under N₂ and stirred for 3 hours. The solution was diluted with EA (300 mL) and washed with saturated aqueous sodium bicarbonate and washed with brine and dry over by Na₂SO₄. Then the solution was concentrated under reduced pressure and the residue was purified by silica gel column (PE:EA, 0˜30%) to give 4 (20 g, 30.49 mmol, 85.08% yield) as a white solid. ¹H-NMR (400 MHz, CDCl₃): δ=9.20 (s, 1H), 8.77 (s, 1H), 8.21 (s, 1H), 8.04-8.02 (d, J=7.76 Hz, 2H), 7.62-7.58 (t, J=14.76 Hz, 1H), 7.53-7.49 (t, 2H), 6.53-6.51 (d, J=6.48 Hz, 1H), 5.60-5.56 (t, J=14.68 Hz, 1H), 5.01-4.97 (t, J=16.52 Hz, 1H), 4.285-4.23 (m, 1H), 4.14-4.08 (m, 1H), 3.98-3.94 (m, 1H), 1.68 (s, 3H), 1.19 (s, 7H), 1.10-1.01 (m, 21H). ESI-LCMS: m/z 656 [M+H]⁺.

Preparation of 18-5: To a solution of 18-4 (19 g, 28.97 mmol) in THF (200 mL) was added 3HF.TEA (14.01 g, 86.91 mmol, 15 mL) at room temperature and stirred for 2 hours. The solution was diluted with EtOAc (200 mL) and washed with saturated aqueous sodium bicarbonate and washed with brine and dry over by Na₂SO₄. Then the solution was concentrated under reduced pressure and the residue was purified by silica gel column (MeOH-DCM, 0-10%) to give 18-5 (9 g, 21.77 mmol, 75.16% yield) as a white solid. ¹H-NMR (400 MHz, DMSO-d₆): δ=11.21 (s, 1H), 8.74 (s, 1H), 8.64 (s, 1H), 8.06-8.05 (d, J=7.8 Hz, 2H), 7.65-7.62 (t, J=14.6 Hz, 1H), 7.56-7.52 (t, 2H), 6.62-6.60 (d, J=5.8 Hz, 1H), 5.91-5.90 (d, J=4.76 Hz, 1H), 5.40-5.37 (t, J=11.72 Hz, 1H), 5.11 (s, 1H), 4.51-4.47 (m, 1H), 3.93-3.91 (m, 1H), 3.79-3.68 (m, 2H), 1.70 (s, 3H). ESI-LCMS: m/z 414 [M+H]⁺.

Preparation of 18-6: To a solution of 18-5 (8 g, 19.35 mmol) in pyridine (60 mL) was added DMTrCl (7.87 g, 23.22 mmol) at room temperature under N₂ atmosphere and stirred for 30 min. The solution was diluted with EtOAc (100 mL) and washed with saturated aqueous sodium bicarbonate, brine and dried over Na₂SO₄. Then the solution was concentrated under reduced pressure and the residue was purified by silica gel column (PE: EA, 0˜40%) to give 18-6 (8.7 g, 12.16 mmol, 62.81% yield) as a white solid. ¹H-NMR (400 MHz, DMSO-d₆): δ=11.22 (s, 1H), 8.69 (s, 1H), 8.38 (s, 1H), 8.06-8.05 (d, J=4.0 Hz, 2H), 7.66-7.62 (t, J=14.6 Hz, 1H), 7.57-7.53 (t, J=15 Hz, 2H), 7.41-7.39 (d, J=7.8 Hz, 2H), 7.28-7.24 (t, 6H), 7.21-7.18 (t, J=14.2 Hz, 1H), 6.87-6.82 (t, 4H), 6.68-6.66 (d, J=5.8 Hz, 1H), 5.96-5.95 (d, J=1.4 Hz, 1H), 5.34-5.32 (t, J=11.5 Hz, 1H), 4.57-4.52 (m, 1H), 4.15-4.11 (m, 1H), 3.71 (s, 6H), 3.46-3.42 (t, J=17.2 Hz, 1H), 3.32-3.30 (m, 1H), 1.65 (s, 3H). ESI-LCMS: m/z 716 [M+H]⁺.

Preparation of compound 18-7: To a solution of 18-6 (7.8 g, 10.90 mmol) and DCI (1.16 g, 9.81 mmol) in anhydrous DCM was added CEOP[N(iPr)₂]₂ 18-6a (3.61 g, 11.99 mmol) at room temperature under N₂ atmosphere. The resulting solution was stirred for 1 hours at room temperature, diluted with 50 mL dichloromethane and washed with saturated aqueous sodium bicarbonate (2×50 mL), brine (1×50 mL). The organic phase was dried over anhydrous sodium sulfate, filtered and concentrated under reduced pressure. The residue was purified by reverse phase preparative HPLC (Column: C18 spherical 20-35 μm 100A 40 g, mobile phase: 0.05% NH₄HCO₃ in water, ACN from 50% to 100%, flow rate: 20 ml/min) to give compound 18-7 (8.5 g, 9.28 mmol, 85.15% yield) as a white solid. ¹H-NMR (400 MHz, DMSO-d₆): δ=11.22 (s, 1H), 8.65 (s, 1H), 8.44-8.41 (d, J=9.44 Hz, 1H), 8.06-8.04 (d, J=7.6 Hz, 2H), 7.66-7.62 (t, J=14.6 Hz, 1H), 7.56-7.53 (t, 2H), 7.41-7.37 (t, 2H), 7.27˜7.21 (m, 6H), 6.86-6.79 (m, 4H), 6.72-6.69 (t, J=12.9 Hz, 1H), 5.58-5.48 (m, 1H), 4.96-4.85 (m, 1H), 4.28-4.26 (m, 1H), 3.741 (s, 6H), 3.60-3.48 (m, 4H), 3.45-3.41 (m, 1H), 3.33 (s, 2H), 2.74-2.71 (t, J=11.7 Hz, 1H), 2.65-2.62 (t, J=11.7 Hz, 1H), 1.65-1.61 (d, J=15.3 Hz, 3H), 1.13-1.07 (m, 9H), 0.98-0.96 (d, J=6.7 Hz, 3H). ³¹P NMR (162 MHz, DMSO-d₆): 149.60, 149.44. ESI-LCMS: m/z 916 [M+H]⁺.

### Example A9

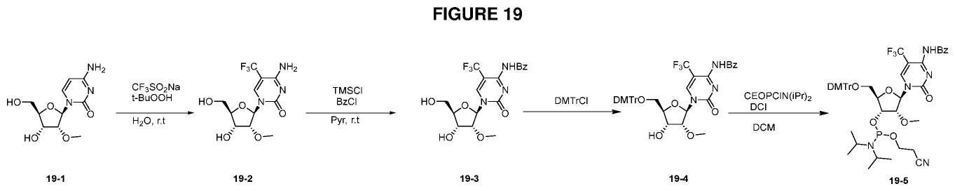

The building block compound 19-5 is useful for making embodiments of modified phosphorothioated oligonucleotides. With reference to FIG. 19, the compound 19-5 was prepared as follows:

Preparation of compound 19-2: Compound 19-2 was made in accordance with known procedures. See Musumeci, Domenica et al., Med Chem Comm, 2013, 4(10), 1405-1410. To a stirred solution of the compound 19-1 (20.0 g, 77.81 mmol) in H₂O (100 ml) was added CF₃SO₂Na (46.8 g, 233.4 mmol) and t-BuOOH (35.0 g, 385.0 mmol). The reaction was stirred for 16 hours. The reaction mixture was filtered off, washed with H₂O (used minimum). The mixture was evaporated to dryness and the resulting crude material was purified by flash column chromatography on silica gel (DCM:MeOH=20:1˜50:1) to obtain 19-2 (11.0 g, 25.64 mmol, 45.5% yield) as a white solid. ESI-MS: m/z 326.0 [M+H]⁺.

Preparation of compound 19-3: To a stirred solution of the compound 19-2 (11.0 g, 25.64 mmol) in pyridine (110 ml) was added TMSCl (102.02 mmol, 17.2 ml) dropwise at 5° C. The reaction was stirred for 30 min. To the reaction was added benzoyl chloride (18.04 g, 51.06 mmol) dropwise at 0° C. The reaction mixture was stirred at 0° C. for 30 min, ammonium hydroxide (1.02 g, 29.17 mmol) was added. The reaction was quenched with sat. NH₄Cl aqueous It was diluted with EtOAc, aqueous and organic layers were separated. Organic phase was washed with sat. aqueous NaHCO₃ (1×) and sat. aqueous NaCl (1×). The organic phases were evaporated to dryness and the resulting crude material was purified by flash column chromatography on silica gel (DCM:MeOH=20:1˜50:1) to get 19-3 (7.10 g, 15.49 mmol, 48.8% yield) as a yellow solid. ESI-MS: m/z 430 [M+H]⁺.

Preparation of compound 19-4: To a stirred solution of compound 19-3 (7.10 g, 15.49 mmol) in pyridine (70 mL) was added 4,4′-dimethoxytrityl chloride (5.77 g, 17.04 mmol) at 0° C. under N₂. The reaction mixture was stirred at room temperature for 1 h. The reaction was poured into sat. aqueous NaHCO₃ (1×). It was diluted with EtOAc, and layers were separated. Organic phase was washed with sat. aqueous NaCl (1×). The organic phases were evaporated to dryness, and the resulting crude material was purified by flash column chromatography on silica gel (DCM:MeOH=20:1˜50:1) to get 4 (6.50 g, 8.87 mmol, 65.5% yield) as a yellow solid. ¹H NMR (400 MHz, DMSO-d₆): δ=12.82 (s, 2H), 8.20-8.10 (m, 5H), 7.67-7.59 (m, 2H), 7.53 (t, J=7.5 Hz, 4H), 7.42 (d, J=7.8 Hz, 4H), 7.36-7.19 (m, 13H), 6.90 (dt, J=8.9, 1.8 Hz, 7H), 5.82-5.74 (m, 3H), 5.25 (d, J=7.0 Hz, 2H), 4.20-3.97 (m, 6H), 3.74 (d, J=1.7 Hz, 11H), 3.49 (s, 5H), 3.27 (s, 3H), 2.00 (s, 1H), 1.29-1.14 (m, 2H). ESI-MS: m/z 732.3 [M+H]⁺.

Preparation of compound 19-5: To a stirred solution of 19-4 (6.5 g, 8.87 mmol) in DCM (5 mL) was added DCI (484.21 mg, 4.10 mmol) at 0° C. under N₂. CEOPClN(iPr)₂ (4.94 g, 16.40 mmol) was added dropwise. The reaction mixture was stirred at room temperature for 2 hr. The reaction mixture was poured into water. It was diluted with EtOAc, and layers were separated. Organic phase was and washed with sat. aqueous NaCl (1×). The organic phases were evaporated to dryness, and the resulting crude material was by reverse phase preparative HPLC (Column: C18 spherical 20-35 μm 100A 40 g, mobile phase: 0.05% NH₄HCO₃ in water, m/m)-ACN from 50% to 100%, flow rate: 20 ml/min) to get 19-5 (5.5 g, 5.90 mmol, 72.0% yield) as a light-yellow solid. ¹H (400 MHz, DMSO-d₆): δ=12.78 (s, 1H), 8.28 (d, J=4.0 Hz, 1H), 8.12 (d, J=7.6 Hz, 2H), 7.63 (t, J=7.4 Hz, 1H), 7.53 (t, J=7.5 Hz, 2H), 7.41 (t, J=7.2 Hz, 2H), 7.36-7.19 (m, 7H), 6.89 (dt, J=9.3, 4.8 Hz, 4H), 5.86-5.78 (m, 1H), 4.23-4.14 (m, 2H), 3.67-3.41 (m, 7H), 3.41-3.30 (m, 2H), 3.23 (td, J=11.4, 5.3 Hz, 1H), 2.78 (t, J=5.8 Hz, 1H), 2.57 (q, J=5.7 Hz, 1H), 1.11 (dd, J=14.4, 6.7 Hz, 9H), 0.93 (d, J=6.7 Hz, 3H). ESI-MS: m/z 932.5 [M+H]⁺.

### Example A10

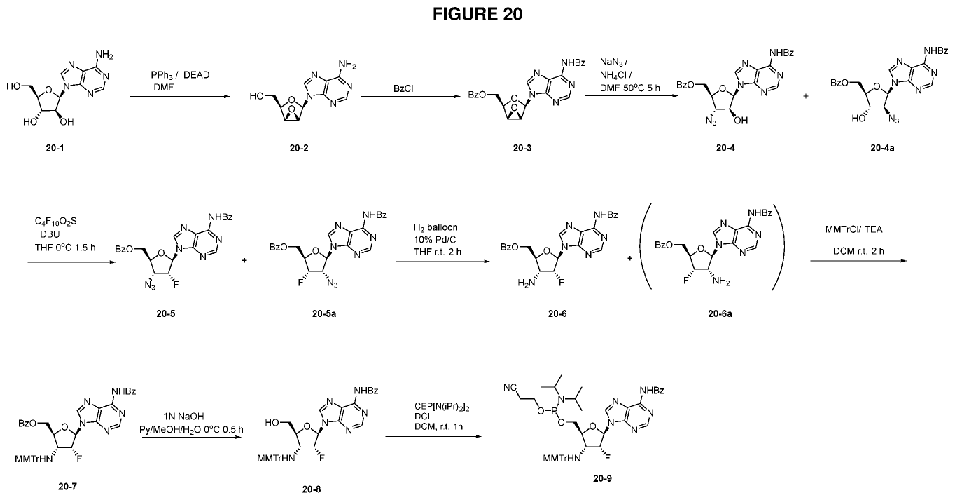

The building block compound 20-9 is useful for making embodiments of modified phosphorothioated oligonucleotides. With reference to FIG. 20, the compound 20-9 was prepared in accordance with known procedures. See Shultz, R. G. et al., Nucleic Acids Research, 1996, Vol. 24, No. 15 2966-2973.

Preparation of compound 20-2: To a solution of 20-1 (21.0 g, 78.7 mmol) in DMF (525 mL) with an inert atmosphere of nitrogen, was added PPh₃ (51.5 g, 196.6 mmol, 2.5 eq). The mixture was stirred for 15 minutes at 0° C. This was followed by the addition of a solution of DEAD (34.2 g, 196.6 mmol, 2.54 eq) in DMF (525 mL) dropwise with stirring at 0° C. in 1 hour. The resulting solution was stirring for 2 hours at 25° C. The resulting mixture was concentrated under reduced pressure. The product was precipitated by the addition of ether. The solids were collected by filtration. The crude product was purified by re-crystallization from methanol. The solid was dried in an oven under reduced pressure. This resulted in 13.0 g (66% yield) of 20-2 as a white solid. ESI-LCMS: m/z 250 [M+H]⁺.

Preparation of compound 20-3: To a solution of 20-2 (13.0 g, 52.2 mmol) in pyridine (130 mL) with an inert atmosphere of nitrogen, was added BzCl (22.8 g, 161.8 mmol, 3.1 eq) dropwise with stirring at 0° C. in 30 min. The resulting solution was stirred for 3 hours at room temperature The mixture was diluted with EA (200 mL). The resulting mixture was washed with 3×150 mL of water and 2×100 mL of saturated sodium bicarbonate solution respectively. The resulting mixture was washed with 1×150 mL of brine. The mixture was dried over anhydrous sodium sulfate, filtered, and concentrated under reduced pressure. The residue was applied onto a silica gel column with EA:PE=2:1. This resulted in 19.0 g (80% yield) of 20-3 as a white solid. ESI-LCMS: m/z 458 [M+H]⁺.

Preparation of compound 20-4: To a solution of 20-3 (19.0 g, 41.6 mmol) in DMF (180 mL) was added NaN₃ (27 g, 415.7 mmol, 10.0 eq), NH₄Cl (4.5 g, 83.2 mmol, 2.0 eq). The resulting solution was stirred for 2 hours at 80° C. The reaction mixture was cooled to room temperature. The resulting solution was diluted with EA (400 mL). The resulting mixture was washed with 2×400 mL of water and 2×400 mL of brine respectively. The mixture was dried over anhydrous sodium sulfate. The solids were filtered out. The resulting mixture was concentrated under vacuum. This resulted in 15.0 g (90% yield) of 20-4:20-4a=5:1 as a white solid. ESI-LCMS: m/z 501 [M+H]⁺.

Preparation of compound 20-5: To a solution of 20-4:20-4a=5:1 (15.0 g, 30.0 mmol) in THF (150 mL) with an inert atmosphere of nitrogen, was added DBU (16.0 g, 105.0 mmol, 3.5 eq). This was followed by the addition of C₄F₁₀O₂S (19.0 g, 63.0 mmol, 2.1 eq) dropwise with stirring at 0° C. in 10 min. The resulting solution was stirred for 1.5 hours at 0° C. The resulting solution was diluted with DCM (200 mL). The resulting mixture was washed with 3×200 mL of water, 1×200 mL of saturated sodium bicarbonate solution and 1×200 mL of brine respectively. The mixture was dried over anhydrous sodium sulfate, filtered, and concentrated under reduced pressure. The crude product was re-crystallized from EA:PE=1:1. This resulted in 9.2 g (60% yield) of 20-5:20-5a=5:1 as a white solid. ESI-LCMS: m/z 503 [M+H]⁺.

Preparation of compound 20-6: To a solution of 20-5:20-5a=5:1 (9.2 g, 18.3 mmol) in THF (230 mL) was added 10% palladium carbon (0.9 g, 10% M). The flask was evacuated and flushed three times with nitrogen, followed by flushing with hydrogen. The resulting solution was stirred for 4 hours at 20° C. The solids were filtered out. The resulting mixture was concentrated under vacuum. The crude product was purified by column chromatography (SiO₂, DCM:MeOH=80:1) to give 6.0 g of 20-6 as a white solid. ESI-LCMS: m/z 47 [M+H]⁺.

Preparation of compound 20-7: To a solution of 20-6 (6.0 g, 12.6 mmol) in DCM (60 mL) was added TEA (2.5 g, 25.2 mmol, 2.0 eq) and MMTrCl (4.3 g, 13.9 mmol, 1.1 eq). The mixture was stirred at room temperature for 1 hours. TLC showed 20-6 was consumed completely. Filtered and the organic layer was washed by water and dried over Na₂SO₄ and purified by silica gel column by (SiO₂, PE:EA=10:1˜5:1˜1:1) to give 20-7 (8.5 g, 11.3 mmol, 90% yield) as a white solid. ESI-LCMS: m/z 749 [M+H]⁺.

Preparation of compound 20-8: Compound 20-7 (8.5 g, 11.3 mmol) was added to 100 mL of 1 N NaOH solution in pyridine:MeOH:H₂O=65:30:5 at 0° C. The suspension was stirred at 0° C. for 15 min. TLC showed starting material was consumed completely. The reaction was quenched by addition of sat. NH₄Cl solution (200 mL). The solution was extracted with EA (200 mL*2) and the combined organic layers were washed with sat. NaHCO₃ solution (200 mL), brine (200 mL), dried over Na₂SO₄, filtered and concentrated. The residue was purified by silica gel column (SiO₂, PE:EA=5:1˜1:2) to give 20-8 (6.2 g, 9.6 mmol, 85% yield) as white solid. ¹H-NMR (DMSO-d₆, 400 MHz): δ ppm 12.96 (s, 1H), 8.10-7.92 (m, 3H), 7.63-7.59 (m, 1H), 7.49 (m, J=8.2 Hz, 6H), 7.37-7.18 (m, 9H), 6.85 (d, J=8.92 Hz, 2H), 5.60 (d, J=16.75 Hz, 1H), 4.34-5.32 (m, 1H), 4.08-3.96 (m, 2H), 3.72 (s, 3H), 3.33-3.26 (m, 1H), 3.12-2.96 (m, 1H), 2.79 (d, J=10.92 Hz, 1H), 1.92 (s, 3H). ESI-LCMS: m/z 635 [M+H]⁺.

Preparation of compound 20-9: To a solution of 20-8 (6.2 g, 9.6 mmol) in DCM (60 mL) was added DCI (0.9 g, 7.4 mmol, 0.8 eq). Then CEP[N(iPr)₂]₂ (3.5 g, 11.5 mmol, 1.2 eq) was added. The reaction mixture was stirred at room temperature for 1 hour TLC showed 20-8 was consumed. The reaction mixture was diluted with dichloromethane and washed with of saturated aqueous sodium bicarbonate and of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 20-9 (5.5 g, 6.5 mmol, 82% yield) as a white solid. H-NMR (400 MHz, DMSO-d₆): δ ppm 13.20 (s, 1H), 8.33-8.28 (m, 2H), 7.73 (s, 0.5H), 7.55-7.41 (m, 9H), 7.29-7.17 (m, 6.5H), 6.80 (dd, J=8.9 Hz, 2H), 5.73 (d, J=17.1 Hz, 0.5H), 5.43 (d, J=20.8 Hz, 0.5H), 4.35-4.28 (m, 1H), 4.13 (d, J=10 Hz, 0.5H), 4.07-4.03 (m, 1H), 3.93-3.83 (m, 0.5H), 3.84-3.79 (m, 1H), 3.77 (d, J=1.52 Hz, 3H), 3.69-3.47 (m, 3H), 3.39-3.25 (m, 1H), 3.15 (d, J=4.1 Hz, 0.25H), 3.02 (d, J=4.1 Hz, 0.25H), 2.61-2.47 (m, 2.5H), 2.39 (t, J=6.4 Hz, 1H), 2.07 (d, J=18.7 Hz, 3H), 1.27-1.22 (m, 12H). ³¹PNMR (400 MHz, CDCl₃): 147.98, 146.35. ESI-LCMS: m/z 835 [M+H]⁺.

### Example A11

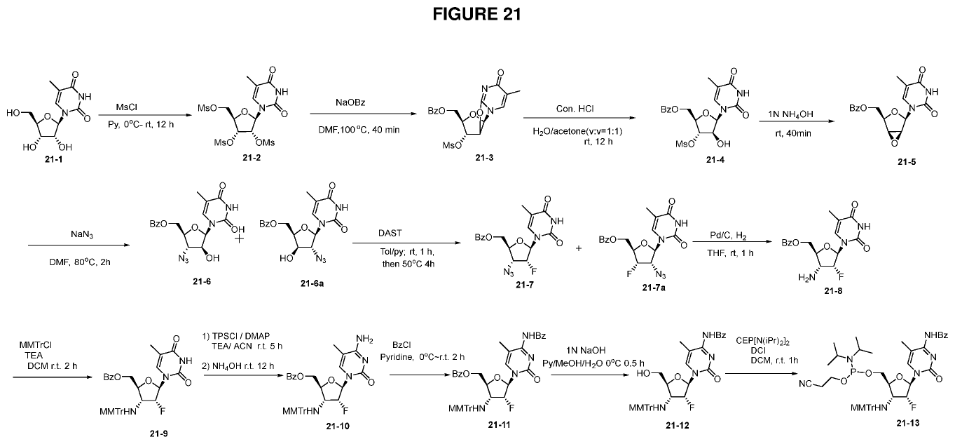

The building block compound 21-13 is useful for making embodiments of modified phosphorothioated oligonucleotides. With reference to FIG. 21, the compound 21-13 was prepared as follows:

Preparation of compound 21-2: To a solution of 21-1 (50 g, 0.2 mol) in pyridine (500 mL) was added MsCl (77.5 g, 0.6 mol) dropwise with stirring at 0° C. for 40 min. The resulting solution was stirred for 16 hours at 20° C. The reaction was then quenched by the addition of 5 L of water/ice. The solids were collected by filtration. This resulted in 85.1 g (85% yield) of 21-2 as a yellow solid. ESI-LCMS: m/z 493 [M+H]⁺.

Preparation of compound 21-3: To a solution of sodium benzoate (78.1 g, 920.0 mmol) in DMF (2.5 L) was followed by the addition of 21-2 (85.1 g, 160.0 mmol) in DMF (800 mL) in portions at 100° C. The resulting solution was stirred for 50 minutes at 100° C. The reaction mixture was cooled to room temperature. The reaction mixture was poured into 100 L of water. The solid was collected by filtration. The solid was dried under infrared light. This resulted in 45 g (72% yield) of 21-3 as a light yellow solid. ESI-LCMS: m/z 423 [1\4+H]⁺.

Preparation of compound 21-4: To a solution of 21-3 (45 g, 100.1 mmol) in acetone/water (v/v=1:1) (4 L) was added hydrochloric acid (220 mL). The resulting solution was stirred for 24 hours at 20° C. The acetone was removed under reduced pressure. The isolated solid was collected and washed with 2×10 L of water. The solid was dried. This resulted in 45 g (96% yield) of 21-4 as a white solid. ESI-LCMS: m/z 441 [M+H]⁺.

Preparation of compound 21-5: Into a 5000-mL round-bottom flask purged and maintained with an inert atmosphere of nitrogen, was placed 21-4 (45 g, 102.27 mmol), ammonia (1N, 3.5 L). The resulting solution was stirred for 1 hours at 20° C. The pH value of the solution was adjusted to 7-8 with acetic acid. The solids were collected by filtration. The solid was dried. This resulted in 26.2 g (72% yield) of 21-5 as a light yellow solid. ESI-LCMS: m/z 345 [M+H]⁺.

Preparation of compound 21-6: To a solution of 21-5 (13.1 g*2, 38.08 mmol) in DMF (180 mL) was added sodium azide (9.0 g*2,), NH₄Cl (3.1 g, *2). The resulting solution was stirred for 2 hours at 80° C. The reaction mixture was cooled to room temperature. The resulting solution was diluted with 400 mL of ethyl acetate. The resulting mixture was washed with 2×800 mL of water and 2×800 mL of sodium chloride (aq.) respectively. The mixture was dried over anhydrous sodium sulfate. The solids were filtered out. The resulting mixture was concentrated under vacuum. This resulted in 28.5 g (92% yield) of 21-6/21-6a (4/1) as a white solid. ESI-LCMS: m/z 388 [M+H]⁺.

Preparation of compound 21-7: To a solution of 21-6/21-6a (4/1) (14 g*2, 36.18 mmol) in toluene/pyridine (150/15 mL) was added DAST (15.1 g*2,). The resulting solution was stirred for 2 hours at 20° C. The resulting solution was allowed to react, with stirring, for an additional 2 hours at 50° C. The reaction mixture was cooled to room temperature. The resulting solution was diluted with 500 mL of ethyl acetate. The resulting mixture was washed with 2×500 mL of sodium bicarbonate (aq.) and 1×500 mL of sodium chloride (aq.) respectively. The mixture was dried over anhydrous sodium sulfate. The solids were filtered out. The resulting mixture was concentrated under vacuum. The crude product was purified by c.c. by (PE:EA=3:1). This resulted in 24.2 g (92% yield) of 21-7/21-7a (5/1) as a light yellow solid. ESI-LCMS: m/z 390 [M+H]⁺.

Preparation of compound 21-8: To a solution of 21-7/21-7a (5/1) (24.2 g, 62.2 mmol) in THF (600 mL) was added 10% palladium carbon (3 g). The flask was evacuated and flushed three times with nitrogen, followed by flushing with hydrogen. The resulting solution was stirred for 4 hours at 20° C. The solids were filtered out. The resulting mixture was concentrated under vacuum. The crude product was purified by column chromatography (SiO₂, DCM:MeOH=80:1) to give 14.0 g of 21-8 as a white solid. ESI-LCMS: m/z 364 [M+H]⁺.

Preparation of compound 21-9: To a solution of 21-8 (14.0 g, 38.5 mmol) in DCM (150 mL) was added TEA (7.8 g, 77.0 mmol) and MMTrCl (17.8 g, 57.8 mmol), the mixture was stirred at room temperature for 1 hours. TLC showed 21-8 was consumed completely. Filtered and the organic layer was washed by water and dried over Na₂SO₄ and purified by silica gel column by (SiO₂, PE:EA=10:1˜5:1˜1:1) to give 21-9 (22 g, 34.6 mmol, 94% yield) as a white solid. ESI-LCMS: m/z 636 [M+H]⁺.

Preparation of compound 21-10: To a solution of 21-9 (22 g, 34.6 mmol) in ACN (250 mL) was added TPSCl (15.7 g, 51.9 mmol) and DMAP (8.4 g, 69.2 mmol). Then TEA (7.0 g, 69.2 mmol) was added, the reaction mixture was stirred at room temperature for 5 hours under N₂. TLC showed 21-9 was consumed. Then NH₄OH (30 mL) was added, the mixture was stirred at room temperature overnight. Water was added and the product was extracted with EA. The organic layer was washed with brine and dried over Na₂SO₄. The organic layer was concentrated to give crude 21-10 (23.0 g) as a white solid. ESI-LCMS: m/z 635 [M+H]⁺.

Preparation of compound 21-11: To a solution of crude 21-10 (23.0 g) in Pyridine (200 mL) was added BzCl (7.5 g) at 0° C., the mixture was stirred at room temperature for 1 hours. TLC showed 21-10 was consumed completely. Water was added, concentrated and purified by silica gel column (SiO₂, PE:EA=3:1˜1:1) to give 21-11 (16.0 g, 64% over 2 steps) as a white solid. ESI-LCMS: m/z 739 [M+H]⁺.

Preparation of compound 21-12: Compound 21-11 (16.0 g, 21.68 mmol) was added to 200 mL of 1 N NaOH solution in pyridine/MeOH/H₂O (65/30/5) at 0° C. The suspension was stirred at 0° C. for 15 min. TLC showed starting material was consumed completely. The reaction was quenched by addition of sat. NH₄Cl solution (500 mL). The solution was extracted with EA (400 mL*2) and the combined organic layers were washed with sat. NaHCO₃ solution (200 mL), brine (200 mL), dried over Na₂SO₄, filtered and concentrated. The residue was purified by silica gel column (SiO₂, PE:EA=5:1˜1:2) to give 21-12 (6.5 g, 10.5 mmol) as white solid. ¹H-NMR (DMSO-d₆, 400 MHz): δ ppm 12.96 (s, 1H), 8.10-7.92 (m, 3H), 7.63-7.59 (m, 1H), 7.49 (m, J=8.2 Hz, 6H), 7.37-7.18 (m, 9H), 6.85 (d, J=8.92 Hz, 2H), 5.60 (d, J=16.75 Hz, 1H), 4.34-5.32 (m, 1H), 4.08-3.96 (m, 2H), 3.72 (s, 3H), 3.33-3.26 (m, 1H), 3.12-2.96 (m, 1H), 2.79 (d, J=10.92 Hz, 1H), 1.92 (s, 3H). ESI-LCMS: m/z 635 [M+H]⁺.

Preparation of compound 21-13: To a solution of 21-12 (6.5 g, 10.5 mmol) in DCM (60 mL) was added DCI (1.0 g, 8.9 mmol). Then CEP[N(iPr)₂]2 (4.1 g, 13.5 mmol) was added. The reaction mixture was stirred at room temperature for 1 hour TLC showed 21-12 was consumed. The reaction mixture was diluted with dichloromethane and washed with of saturated aqueous sodium bicarbonate and of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 21-13 (6.5 g, 82%) as a white solid. ¹H-NMR (400 MHz, DMSO-d₆): δ ppm 13.20 (s, 1H), 8.33-8.28 (m, 2H), 7.73 (s, 0.5H), 7.55-7.41 (m, 9H), 7.29-7.17 (m, 6.5H), 6.80 (dd, J=8.9 Hz, 2H), 5.73 (d, J=17.1 Hz, 0.5H), 5.43 (d, J=20.8 Hz, 0.5H), 4.35-4.28 (m, 1H), 4.13 (d, J=10 Hz, 0.5H), 4.07-4.03 (m, 1H), 3.93-3.83 (m, 0.5H), 3.84-3.79 (m, 1H), 3.77 (d, J=1.52 Hz, 3H), 3.69-3.47 (m, 3H), 3.39-3.25 (m, 1H), 3.15 (d, J=4.1 Hz, 0.25H), 3.02 (d, J=4.1 Hz, 0.25H), 2.61-2.47 (m, 2.5H), 2.39 (t, J=6.4 Hz, 1H), 2.07 (d, J=18.7 Hz, 3H), 1.27-1.22 (m, 12H). ³¹PNMR (400 MHz, CDCl₃): 147.98, 146.35. ESI-LCMS: m/z 835 [M+H]⁺.

### Example A12

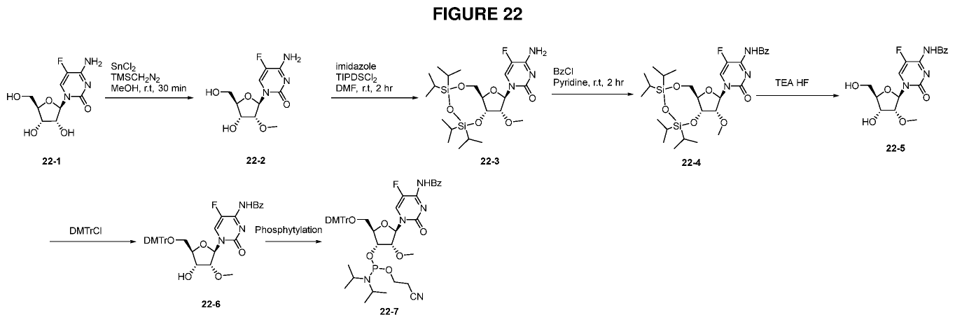

The building block compound 22-7 is useful for making embodiments of modified phosphorothioated oligonucleotides. With reference to FIG. 22, the compound 22-7 was prepared as follows:

Preparation of compound 22-2: Intermediate 22-2 was prepared by modifying a procedure disclosed in Cramer, Hagen et al., Helvetica Chimica Acta, 1996, 79(8), 2114-2136. To a solution of 22-1 (10.0 g, 38.3 mmol) and SnCl₂ (260 mg, 1.1 mmol) in MeOH (100 mL) was added 1M TMSCH₂N₂ (50 mL, 50 mmol) in n-hexane drop wise at room temperature. The resulting solution was stirred at room temperature for 10 min. The resulting solution was concentrated under reduced pressure. 10 g (crude) of 22-2 was obtained as a white solid and used for next step without further purification. ESI-LCMS: m/z 276 [M+H]⁺.

Preparation of compound 22-3: To a solution of 22-2 (27.0 g, 103.4 mmol) in DMF (250 mL), imidazole (24.6 g, 362.1 mmol) and TIPDSCl₂ (48.9 g, 155 mmol) was added at 0° C. The resulting solution was stirred at room temperature for 2 hours. The mixture was added water and extracted with EA. The organic layer was washed with water and brine, dried over Na₂SO₄, and concentrated under reduced pressure and the residue was purified by silica gel column chromatography (SiO₂, PE:EA=2:1) to give 22-3 (65 g, 11.0 mmol, 70.0% yield) as a white solid. ¹H-NMR (400 MHz, DMSO-d₆): δ=7.84 (s, 1H), 7.77 (d, J=4.8 Hz, 1H), 7.30-7.12 (m, 1H), 5.57 (s, 1H), 4.22-4.14 (m, 2H), 4.00 (d, J=4.1 Hz, 1H), 3.91 (d, J=8.2 Hz, 1H), 3.76 (d, J=3.8 Hz, 1H), 3.53 (s, 3H), 1.02-0.96 (m, 28H). ESI-LCMS: m/z 518 [M+H]⁺.

Preparation of compound 22-4: To a solution of 22-3 (15.0 g, 29.0 mmol) in pyridine (100 mL), BzCl (5.3 g, 37.6 mmol) was added at room temperature The resulting solution was stirred at room temperature for 2 hours. The mixture was added water and extracted with EA. The organic layer was washed with water and brine, dried over Na₂SO₄, and concentrated under reduced pressure and the residue was purified by silica gel column chromatography (SiO₂, PE:EA=3:1) to give 22-4 (12.4 g, 20.0 mmol, 68.0%) as a white solid. ESI-LCMS: m/z 622 [M+H]⁺.

Preparation of compound 22-5: To a solution of 22-4 (20.0 g, 32.2 mmol) in THF (200 mL), triethylamine trihydrofluoride (36.6 g, 96.6 mmol) was added at room temperature. The resulting solution was stirred at room temperature for 3 hours. The resulting solution was filtered and concentrated under reduced pressure. 6.0 g (crude) of 22-5 was obtained as a white solid and used for next step without further purification. ESI-LCMS: m/z 380 [M+H]⁺.

Preparation of compound 22-6: To the solution of 22-5 (6.0 g, 15.8 mmol) in dry pyridine (60 mL) was added DMTrCl (6.1 g, 18.2 mmol) at room temperature, and the reaction mixture was stirred at room temperature for 3 hours under N₂ atmosphere. After addition of water, the resulting mixture was extracted with EA. The organic layer was washed with water and brine, dried over Na₂SO₄, and concentrated under reduced pressure and the residue was purified by silica gel column chromatography (SiO₂, PE:EA=2:1-EA) to give 22-6 (7.5 g, 11.0 mmol, 70.0%) as a white solid. 1H-NMR (400 MHz, DMSO-d₆) δ=8.01-7.99 (d, J=8 Hz, 2H), 7.65 (m, 1H), 7.55-7.51 (m, 2H), 7.43-7.41 (d, J=8 Hz, 2H), 7.33-7.28 (m, 6H), 7.24-7.20 (m, 1H), 6.91-6.89 (d, J=8 Hz, 4H), 5.81 (s, 1H), 5.24-5.22 (d, J=8 Hz, 1H), 4.28 (m, 1H), 3.88 (s, 1H), 3.73 (s, 6H), 3.50 (s, 3H), 3.39 (m, 1H), 3.27 (m, 1H). ESI-LCMS: m/z 682 [M+H]⁺.

Preparation of compound 22-7: To a suspension of 22-6 (7.5 g, 11.0 mmol) in DCM (70 mL) was added DCI (1.16 g, 9.9 mmol) and CEP[N(iPr)₂]2 (3.9 g, 13.2 mmol). The mixture was stirred at room temperature for 0.5 hours. LC-MS showed work well. The solution was washed with water twice and washed with brine and dried over Na₂SO₄. Then concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; detector, UV 254 nm. This resulted in 22-7 (7.5 g, 8.5 mmol, 77.3% yield) as a white solid. 1H-NMR (400 MHz, DMSO-d₆): δ=8.00-7.98 (d, J=8 Hz, 2H), 7.66-7.62 (m, 1H), 7.55-7.51 (m, 2H), 7.44-7.40 (m, 2H), 7.33-7.20 (m, 7H), 6.91-6.87 (m, 4H), 5.83-5.80 (d, J=12 Hz, 1H), 4.52-4.37 (m, 1H), 4.19-4.03 (m, 2H), 3.73-3.72 (m, 7H), 3.50 (m, 5H), 3.38-3.37 (m, 2H), 2.80-2.77 (m, 1H), 2.63-2.58 (m, 1H), 1.16-1.10 (m, 9H), 0.98-0.97 (d, J=4 Hz, 3H). ³¹P-NMR (162 MHz, DMSO-d₆): δ=149.52, 148.84. ESI-LCMS: m/z 882 [1\4+H]⁺.

### Example A13

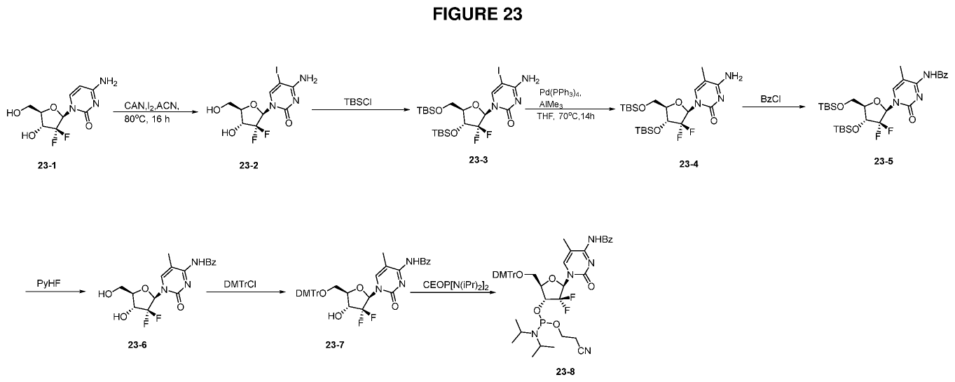

The building block compound 23-8 is useful for making embodiments of modified phosphorothioated oligonucleotides. With reference to FIG. 23, the compound 23-8 was prepared in accordance with known methods. See Schroeder, Arne S. et al, Organic Letters, 2016, 18(17), 4368-4371.

Preparation of compound 23-2: A mixture of ACN (60 mL), 23-1 (20 g, 76.0 mmol), 12 (12.1 g, 47.9 mmol) and CAN (15.4 g, 38.0 mmol) stirred at room temperature for 10 min. Then reaction mixture warmed to 80° C. and stirred for 14 hours. TLC showed 23-1 was consumed completely. Reaction mixture was cooled and filtered to give 23-2 (24 g, 29.30 mmol, 83.8% yield) as a slightly yellow solid. ESI-LCMS: 390 [M+H]⁺.

Preparation of compound 23-3: To a suspension of crude 23-2 in pyridine (250 mL) with was added imidazole (12.6 g, 184.4 mmol) and TBSCl (27.8 g, 184.4 mmol) at 0° C. under an inert atmosphere of nitrogen. The reaction solution was stirred for 14 hours at room temperature. The solution was diluted with EA (500 mL) and washed with H₂O, saturated aqueous sodium bicarbonate and washed with brine and dry over by Na₂SO₄. Then the solution was concentrated under reduced pressure and the residue was purified by column chromatography (SiO₂, 0-10% MeOH-DCM). This resulted in 23-3 (24.0 g, 71.6% yield) as a white solid. ESI-LCMS: m/z 619 [M+H]⁺.

Preparation of compound 23-4: To a solution of 23-3 (17.3 g, 28.0 mmol) in THF (280 mL) was added Pd(PPh₃)₄ (3.2 g, 2.8 mmol), then added AlMe₃ (77 mL, 72.8 mmol) at 0° C. The mixture was stirred at 70° C. for 14 hours. TLC showed 23-3 was consumed completely. NH₄Cl aqueous was added to the reaction. The product was extracted with EA, the organic layer was washed with NaHCO₃ and brine. Then the solution was concentrated under reduced pressure and purified by column chromatography (SiO₂, 0-10% MeOH-DCM). This resulted in 23-4 (8.0 g, 56% yield) as a white solid. ESI-LCMS: m/z 507 [M+H]⁺.

Preparation of compound 23-5: To a solution of 23-4 (8.0 g, 15.8 mmol) in 80 mL of dichloromethane, pyridine (12.5 g, 158.1 mmol) and BzCl (2.6 g, 18.9 mmol) were added at 0° C. with an inert atmosphere of nitrogen. The reaction solution was stirred for 30 minutes at room temperature. The solution was diluted with EA (100 mL) and washed with H₂O, saturated aqueous sodium bicarbonate and washed with brine and dry over by Na₂SO₄. Then the solution was concentrated under reduced pressure and the residue was purified by column chromatography (SiO₂, 0-5% EA-PE). This resulted in 23-5 (7.3 g, 75% yield) as a white solid. ESI-LCMS: m/z 610 [M+H]⁺.

Preparation of compound 23-6: To a solution of 23-5 (7.3 g, 11.9 mmol) in 100 mL EA with an inert atmosphere of nitrogen was added HF.pyridine (17.7 g, 179.5 mmol) in order at room temperature. The resulting solution was stirred for 3 hours. The solution was diluted with EA (100 mL) and washed with H₂O, saturated aqueous sodium bicarbonate and washed with brine and dry over by Na₂SO₄. Then the solution was concentrated under reduced pressure and the residue was purified by column chromatography (SiO₂, 0-5% MeOH-DCM). This resulted in 23-6 (4.1 g, 90% yield) as a white solid. ESI-LCMS: m/z 382 [M+H]⁺.

Preparation of compound 23-7: To a solution of 23-6 (4.1 g, 10.7 mmol) in 45 mL of pyridine with an inert atmosphere of nitrogen was added DMTrCl (4.36 g, 12.9 mmol) in order at room temperature. The resulting solution was stirred for 2 hours at room temperature and diluted with 100 mL ether and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=4/1; detector, UV 254 nm. This resulted in 23-7 (5.8 g, 80% yield) as a white solid. ¹H-NMR (400 MHz, DMSO-d₆) δ=12.80 (s, 1H), 8.18-8.16 (m, 2H), 7.68-7.60 (m, 2H), 7.53-7.49 (m, 2H), 7.43-7.41 (m, 2H), 7.32-7.16 (m, 10H), 6.94-6.91 (m, 4H), 6.42-6.40 (m, 1H), 6.17 (m, 1H), 4.44 (m, 1H), 4.08-4.05 (m, 1H), 3.73 (s, 6H), 3.45 (m, 1H), 2.35 (s, 2H), 1.75 (s, 3H). ¹⁹F-NMR (162 MHz, DMSO-d₆) δ=−115.38, −115.80. ESI-LCMS: m/z 683 [M+H]⁺.

Preparation of compound 23-8: To a solution of 23-7 (5.8 g, 8.4 mmol) in 60 mL of dichloromethane with an inert atmosphere of nitrogen was added CEOP[N(iPr)₂]2 (3.0 g, 10.1 mmol) and DCI (800.8 mg, 6.7 mmol) in order at room temperature. The resulting solution was stirred for 1 hours at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; detector, UV 254 nm. This resulted in 23-8 (6.6 g, 82% yield) as a white solid. ¹H-NMR (400 MHz, CD₃CN-d₆) δ=13.24 (s, 1H), 8.30 (m, 2H), 7.68-7.57 (m, 2H), 7.52-7.48 (m, 4H), 7.40-7.28 (m, 7H), 6.93-6.89 (m, 4H), 6.30-6.24 (m, 1H), 4.79-4.75 (m, 1H), 4.20-4.18 (m, 1H), 3.87-3.54 (m, 10H), 3.47-3.41 (m, 1H), 2.67-2.48 (m, 2H), 1.81-1.76 (m, 10H), 1.01-1.00 (m, 2H). ³¹P-NMR (162 MHz, CD₃CN-d₆) δ=152.80, 152.77. ESI-LCMS: m/z 884 [M+H]⁺.

### Example A14

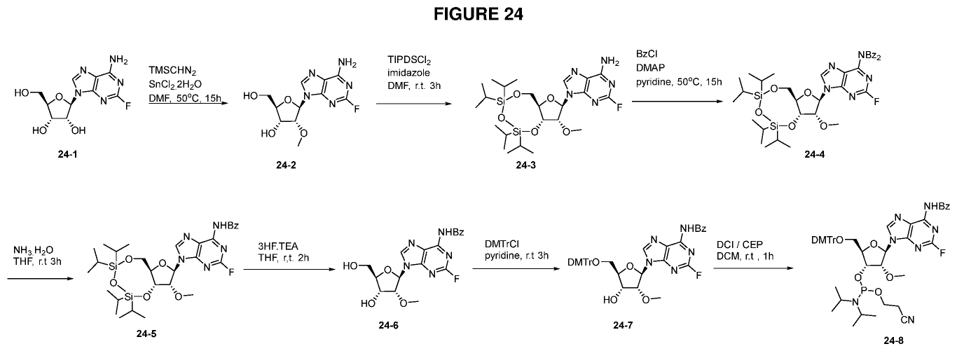

The building block compound 24-8 is useful for making embodiments of modified phosphorothioated oligonucleotides. With reference to FIG. 24, the compound 24-8 was prepared as follows:

Preparation of compound 24-2: Compound 24-2 was prepared by modifying the procedure disclosed in Cramer, Hagen et. al., Helvetica Chimica Acta, 1996, 79(8), 2114-2136. To a solution of 24-1 (20.0 g, 70.1 mmol) in DMF (1.4 L), SnCl_{2.2}H₂O (789 mg, 3.5 mmol) was added, mixture was stirred at room temperature for 10 min, after stirred at 50° C. for 1 min, TMSCHN₂ (105.0 ml, 210.5 mmol, 2.0 M) was added all at once. Reaction was stirred at 50° C. for 15 h, LCMS showed 1 was consumed completely. Solvent was removed by reduced pressure to give crude 24-2 (30.0 g) which was used in next step. ESI-LCMS: m/z 300.1 [M+H]⁺.

Preparation of compound 24-3: To a solution of 24-2 (28.0 g, 93.6 mmol) in DMF (250.0 ml), CF₃COOH (2.0 ml) was added stirred for 10 min, followed by imidazole (25.0 g, 374.1 mmol), TIPDSCl₂ (44.1 g, 140.0 mmol) were added at 0° C. Reaction was stirred at room temperature for 3 h, LCMS showed 24-2 was consumed completely. H₂O (200.0 ml) was added, aqueous phase was extracted with EA (200.0 mL*3), organic phase was concentrated to give crude which was purified by column chromatography (SiO₂, DCM/MeOH=200:1 to 150:1) to give 24-3 (12.0 g, 85% purity, 38% yield over 2 steps) as a white solid. ESI-LCMS: m/z 542.3 [M+H]⁺.

Preparation of compound 24-4: To a solution of 24-3 (12.0 g, 22.1 mmol) in DCM (120.0 mL), DIPEA (11.4 g, 88.7 mmol), DMAP (1.34 g, 11.3 mmol), BzCl (9.2 g, 66.3 mmol) were added at 0° C. Reaction mixture was stirred at room temperature for 3 h, TLC showed 24-3 was consumed completely. H₂O (200.0 mL) was added, aqueous phase was extracted with DCM (200.0 mL*3), organic phase was concentrated to give crude which was purified by column chromatography (SiO₂, PE/EA=20:1 to 3:1) to give 24-4 (13.7 g, 90% purity, 82% yield) as a white solid. ESI-LCMS: m/z 750.4 [M+H]⁺.

Preparation of compound 24-5: To a solution of 24-4 (12.0 g, 18.2 mmol) in THF (240.0 mL), con. NH₄OH (5.0 mL) was added at 0° C. Reaction was stirred at room temperature for 4 h, TLC showed 24-4 was consumed completely. H₂O (200.0 mL) was added, aqueous phase was extracted with EA (200.0 mL*3), organic phase was washed with citric acid aqueous (100.0 mL*2) and concentrated to give crude 24-5 (10.3 g) used next step directly. ESI-LCMS: m/z 646.4 [M+H]⁺.

Preparation of compound 24-6: To a solution of 24-5 (10.3 g, 15.9 mmol) in THF (100.0 mL), 3HF.TEA (7.7 g, 47.9 mmol) was added. Reaction was stirred at room temperature for 2 h, TLC showed 24-5 was consumed completely. Solvent was removed under reduced pressure, the residue was purified by Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1.5/1 within 20 min, the eluted product was collected at CH₃CN/H₂O (0.5% NH₄HCO₃)=1/1; Detector, UV 254 nm. This resulted in 24-6 (5.1 g, 95% purity, 68% yield over 2 steps) as a white solid. ¹H-NMR (400 MHz, DMSO-d₆) δ=11.52 (s, 1H), 8.74 (s, 1H), 8.05-8.02 (m, 2H), 7.69-7.54 (m, 3H), 6.07-6.06 (d, J=4.0 Hz, 1H), 5.35-5.33 (d, J=8.0 Hz, 1H), 5.13-5.10 (m, 1H), 4.39-4.35 (m, 2H), 4.02-3.99 (m, 1H), 3.73-3.57 (m, 2H), 3.39 (m, 3H). ESI-LCMS: m/z 404.2 [M+H]⁺.

Preparation of compound 24-7: To a solution of 24-6 (5.1 g, 12.6 mmol) in pyridine (50.0 mL), DMTrCl (5.13 g, 15.1 mmol) was added at 0° C. Reaction was stirred at room temperature for 2 h, TLC showed 24-6 was consumed completely. H₂O (100.0 mL) was added, aqueous layers was extracted with EA (100.0 mL*2). The organic phase was concentrated, the residue was purified by column chromatography (SiO₂, PE/EA=5:1 to 1:1) to give 24-7 (7.6 g, 85% yield) as a white solid. ¹H-NMR (400 MHz, DMSO-d₆) δ=11.54 (s, 1H), 8.60 (s, 1H), 8.05-8.02 (m, 2H), 7.69-7.65 (m, 1H), 7.58-7.54 (m, 2H), 7.37-7.35 (m, 2H), 7.26-7.18 (m, 7H), 6.86-6.81 (m, 4H), 6.11-6.10 (d, J=4.0 Hz, 1H), 5.37-5.36 (d, J=4.0 Hz, 1H), 4.48-4.42 (m, 2H), 4.39-4.35 (m, 2H), 4.13-4.10 (m, 1H), 3.72 (s, 6H), 3.41 (s, 3H), 3.32-3.22 (m, 2H). ESI-LCMS: m/z 706.4 [M+H]⁺.

Preparation of compound 24-8: To a solution of 24-7 (7.6 g, 10.7 mmol) in dichloromethane (70.0 mL) with an inert atmosphere of nitrogen was added CEOP[N(iPr)₂]2 (3.8 g, 12.8 mmol) and DCI (1.1 g, 9.7 mmol) in order at room temperature. The resulting solution was stirred for 1 hours at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 24-8 (7.6 g, 75% yield) as a white solid. ¹H-NMR (400 MHz, DMSO-d₆) δ=11.52 (s, 1H), 8.63-8.62 (m, 1H), 8.03-8.01 (m, 2H), 7.68-7.64 (m, 1H), 7.57-7.53 (m, 2H), 7.36-7.33 (m, 2H), 7.25-7.17 (m, 7H), 6.84-6.78 (m, 4H), 6.14-6.09 (m, 1H), 4.72-4.64 (m, 2H), 4.28-4.19 (m, 1H), 3.83-3.79 (m, 1H), 3.71 (s, 6H), 3.68-3.53 (m, 3H). 3.41-3.38 (d, J=12.0 Hz, 3H), 3.32-3.31 (m, 2H), 2.81-2.78 (m, 1H), 2.63-2.60 (m, 1H), 1.15-1.12 (m, 9H), 1.03-1.01 (m, 3H). ³¹P-NMR (162 MHz, DMSO-d₆) δ=149.53, 149.37. ESI-LCMS: m/z 906.6 [M+H]⁺.

### Example A15

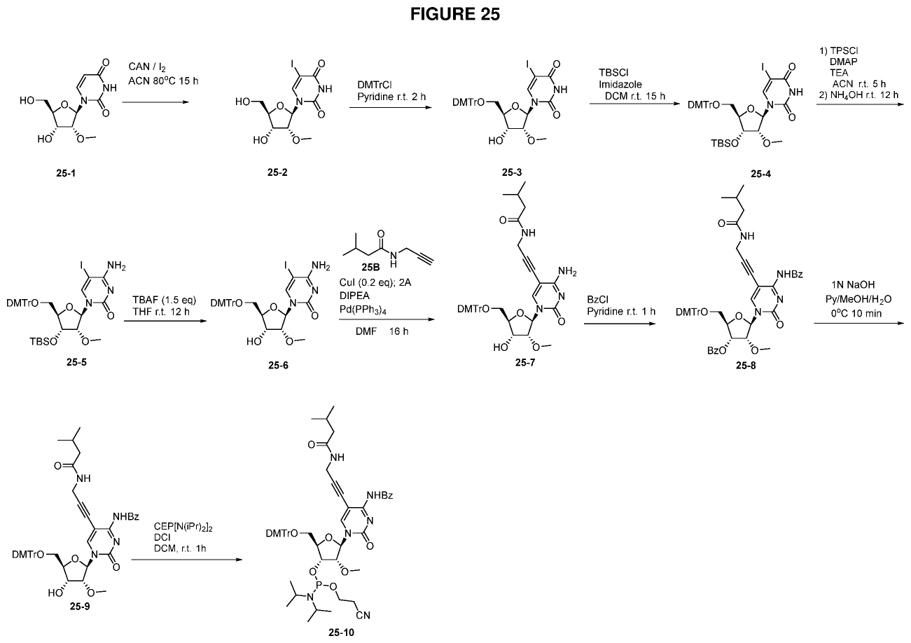

The building block compound 25-10 is useful for making embodiments of modified phosphorothioated oligonucleotides. With reference to FIG. 25, the compound 25-10 was prepared as follows:

Preparation of compound 25-2: To a solution of 25-1 (35.0 g, 135.5 mmol) in MeCN (150 mL) was added I₂ (20.6 g, 81.3 mmol) and CAN (37.1 g, 67.7 mmol). Then the solution was stirred at 80° C. and stirred for 2.5 hours. After the reaction, the solution was cooled down to −10° C. and filtrated at −10° C. to get a yellow solid. The yellow solid was washed with ice MeCN and ice water to give 25-2 (48.0 g, 124.9 mmol, 92.4% yield) as a white solid ¹H NMR (400 MHz, DMSO-d₆) δ 11.71 (s, 1H), 8.54 (s, 1H), 5.80 (d, J=3.9 Hz, 1H), 5.31 (d, J=4.7 Hz, 1H), 5.14 (d, J=6.3 Hz, 1H), 4.12 (q, J=5.7 Hz, 1H), 3.92-3.84 (m, 1H), 3.79 (d, J=4.6 Hz, 1H), 3.71 (d, J=12.4 Hz, 1H), 3.58 (d, J=12.5 Hz, 1H), 3.39 (s, 3H). ESI-LCMS: m/z 385 [M+H]⁺.

Preparation of compound 25-3: To the solution of 25-2 (45.0 g, 117.1 mmol) in dry pyridine was added DMTrCl (47.5 g, 140.6 mmol) slowly under ice bath. Then the solution was stirred at room temperature overnight. Compound 25-2 was consumed as indicated by TLC and LCMS. The solvent was concentrated to get a residue. The residue was purified by silica gel column (SiO₂, PE:EA=5:1˜3:1˜1:1) to give 25-3 (68.0 g, 99.1 mmol, 84.5% yield) as pale yellow solid ¹H NMR (400 MHz, DMSO-d₆) δ 11.80 (s, 1H), 8.01 (s, 1H), 7.46-7.37 (m, 2H), 7.38-7.26 (m, 6H), 7.28-7.19 (m, 1H), 6.95-6.86 (m, 4H), 5.80 (d, J=4.3 Hz, 1H), 5.21 (d, J=6.7 Hz, 1H), 4.17 (q, J=5.9 Hz, 1H), 3.96 (ddd, J=17.6, 5.2, 3.5 Hz, 2H), 3.75 (s, 6H), 3.41 (s, 3H), 3.22 (qd, J=10.8, 3.8 Hz, 2H); ESI-LCMS: m/z 709 [M+Na]⁺.

Preparation of compound 25-4: To the solution of 25-3 (65.0 g, 94.7 mmol) in dry DCM (600 mL) was added imidazole (19.3 g, 284.0 mmol). Then TBSCl (21.3 g, 142.0 mmol) was slowly added to the reaction mixture under ice bath. Then reaction mixture was stirred at room temperature overnight. Water was added to the solution. The product was extracted with EA. The combined organic layer was washed with brine and dried over Na₂SO₄ and concentrated to give the crude. The crude was purified by silica gel column (SiO₂, PE:EA=5:1˜3:1˜1:1) to give 25-4 (70.0 g, 87.4 mmol, 92.3% yield) as a white solid. ¹H NMR (400 MHz, DMSO-d₆) δ 8.07 (s, 1H), 7.43-7.35 (m, 2H), 7.29 (dd, J=11.9, 8.0 Hz, 6H), 7.24-7.16 (m, 1H), 6.91-6.82 (m, 4H), 5.74 (d, J=3.4 Hz, 1H), 4.31-4.23 (m, 1H), 3.97-3.86 (m, 2H), 3.70 (s, 6H), 3.35 (s, 3H), 3.30 (dd, J=11.1, 2.7 Hz, 1H), 3.10 (dd, J=11.0, 4.6 Hz, 1H), 0.73 (s, 9H), −0.01 (s, 3H), −0.09 (s, 3H). ESI-LCMS: m/z 823 [M+Na]⁺.

Preparation of compound 25-5: To the solution of 25-4 (60.0 g, 74.9 mmol) in dry MeCN (600 mL) was added TEA (15.1 g, 149.8 mmol), DMAP (18.3 g, 149.8 mmol) and TPSCl (45.4 g, 149.9 mmol) slowly under N₂. Then the solution was stirred at room temperature for 5 hours. TLC showed 25-4 was consumed completely. NH₄OH (6.5 g, 382.3 mmol) was added to the mixture and the combined reaction mixture was stirred at room temperature overnight. Then the solvent was concentrated to give a crude product 25-5 (46.0 g, 57.5 mmol, 90.2% yield) which was used directly for the next step. ESI-LCMS: m/z 800 [M+H]⁺.

Preparation of compound 25-6: To a solution of 25-5 (40.0 g, 50.0 mmol) in THF (400 mL) was added TBAF (19.6 g, 75.0 mmol). After stirring at room temperature for 12 h, the solvent was concentrated to get a residue. The residue was purified by silica gel column (PE:EA=5:1 to 3:1 to 1:1 to EA) to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in the 25-6 (24.0 g, 35.7 mmol, 70.5% yield) as yellow solid. ¹H NMR (400 MHz, DMSO-d₆) δ 7.94 (s, 2H), 7.43 (d, J=7.8 Hz, 2H), 7.37-7.27 (m, 6H), 7.23 (td, J=11.5, 10.7, 6.5 Hz, 1H), 6.94-6.82 (m, 4H), 6.68 (s, 1H), 5.83 (d, J=3.6 Hz, 1H), 5.15 (d, J=6.7 Hz, 1H), 4.15 (q, J=6.2 Hz, 1H), 3.97 (dt, J=6.7, 3.7 Hz, 1H), 3.81 (t, J=4.5 Hz, 1H), 3.75 (s, 6H), 3.43 (s, 3H), 3.22 (d, J=3.7 Hz, 2H). ESI-LCMS: m/z 686 [M+H]⁺.

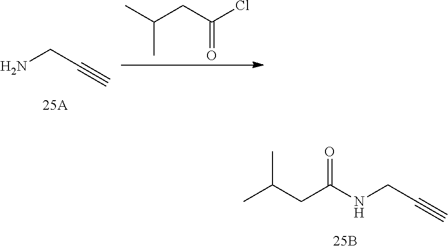

Preparation of compound 25B: This prepared was prepared in accordance with known methods. See Tetrahedron Letters, 2019, vol. 60, #11, p. 777-779. To a solution of 25A (5.0 g, 90.7 mmol) in DCM (100 mL) was added TEA (27.5 g, 272.3 mmol) then isobutyryl chloride (13.1 g, 108.9 mmol) was dropwise into the mixture under ice bath. Then the solution was stirred at room temperature for 3 hours. After the reaction, water was added into the mixture. The product was extracted with EA. The combined organic layer was washed with brine and dried over Na₂SO₄ and concentrated to give the crude. The crude was purified by silica gel column (SiO₂, PE:EA=20:1 to 10:1 to 5:1 to 3:1) to give 25B (11.0 g, 79.0 mmol, 87.0% yield) as a yellow oil. ¹H NMR (400 MHz, DMSO-d₆) δ 8.22 (t, J=5.6 Hz, 1H), 3.84 (dd, J=5.8, 2.5 Hz, 2H), 3.06 (t, J=2.6 Hz, 1H), 1.96 (d, J=3.1 Hz, 3H), 0.87 (d, J=5.5 Hz, 6H). ESI-LCMS: m/z 140 [M+H]⁺.

Preparation of compound 25-7: To a solution of 25-6 (10.0 g, 14.8 mmol) in dry DMF (100.0 mL) was added 25B (4.1 g, 29.7 mmol), CuI (567.2 mg, 2.9 mmol), Pd(P(Ph)₃)₄ (1.7 g, 1.4 mmol), DIPEA (4.8 g, 37.2 mmol). Then the solution was stirred at room temperature overnight under N₂. Then water was added into the mixture, and the obtained mixture was extracted with EA. The combined organic layers was washed with brine, filtered and concentrated to give the residue. The residue was purified by silica gel column (SiO₂, PE:EA=20:1 to 10:1 to EA) to give 25-7 (8.0 g, 11.4 mmol, 77.1% yield) as yellow solid. ¹H NMR (400 MHz, DMSO-d₆): δ 8.13 (t, J=4.8 Hz, 1H), 7.88 (s, 2H), 7.44-7.40 (m, 2H), 7.34-7.27 (m, 6H), 7.25-7.20 (m, 1H), 6.92-6.88 (m, 4H), 6.81 (s, 1H), 5.81 (d, J=3.1 Hz, 1H), 5.17 (d, J=7.0 Hz, 1H), 4.19 (td, J=6.9, 5.1 Hz, 1H), 3.98 (ddd, J=7.1, 5.1, 2.3 Hz, 1H), 3.86 (dd, J=4.8, 3.2 Hz, 2H), 3.75 (s, 8H), 3.44 (s, 3H), 3.14 (dd, J=10.8, 2.3 Hz, 1H), 1.99-1.96 (m, 3H), 0.87 (q, J=2.7 Hz, 6H). ESI-LCMS: m/z 697 [M+H]⁺.

Preparation of compound 25-8: To a solution of 25-7 (7.0 g, 10.0 mmol) in pyridine (60 mL) was added BzCl (3.6 g, 25.0 mmol) at 0° C. Then warmed up and the mixture was stirred at room temperature for 1 hours. LCMS showed 25-7 was consumed completely. Water was added to the mixture. The product was extracted with EA. The combined organic layer was washed with brine and dried over Na₂SO₄ and concentrated to give the crude. The crude was purified by silica gel column by (SiO₂, PE:EA=10:1˜5:1˜1:1) to give 25-8 (7.0 g, 7.7 mmol, 77% yield) as a yellow solid. ESI-LCMS: m/z 905 [M+H]⁺.

Preparation of compound 25-9: Compound 25-8 (7.0 g, 7.7 mmol) was added to 60 mL of 1 N NaOH solution in pyridine/MeOH/H₂O (65/30/5) at 0° C. The suspension was stirred at 0° C. for 30 min. TLC showed starting material was consumed completely. The reaction was quenched by addition of sat. NH₄Cl solution (300 mL). The solution was extracted with EA (200 mL*2) and the combined organic layers were washed with sat. NaHCO₃ solution (200 mL), brine (200 mL), dried over Na₂SO₄, filtered and concentrated. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in 25-9 (5.3 g, 6.6 mmol, 86% yield) as a white solid. ¹H-NMR (DMSO-d₆, 400 MHz): δ ppm 12.76 (s, 1H), 8.10-7.95 (m, 4H), 7.62-7.43 (m, 5H), 7.35-7.22 (m, 7H), 6.93-6.90 (m, 4H), 5.80 (s, 1H), 5.26 (d, J=5.68 Hz, 1H), 4.28-4.25 (m, 1H), 4.06-3.97 (m, 2H), 3.81-3.69 (m, 8H), 3.57-3.37 (m, 4H), 3.18 (d, J=10.08 Hz, 1H), 1.99-1.81 (m, 3H), 0.87-0.85 (m, 6H). ESI-LCMS: m/z 801 [M+H]⁺.

Preparation of compound 25-10: To a solution of 25-9 (5.3 g, 6.6 mmol) in DCM (50 mL) was added DCI (660.1 mg, 5.6 mmol). Then CEP[N(iPr)₂]2 (2.6 g, 8.6 mmol) was added. The reaction mixture was stirred at room temperature for 1 hour LCMS showed 25-9 was consumed. The reaction mixture was diluted with DCM and washed with H₂O (40 mL*2) and brine (50 mL*2). The combined organic layer was dried over Na₂SO₄ and concentrated to give the residue. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 25-10 (4.6 g, 4.6 mmol, 70% yield) as a white solid. ¹H-NMR (400 MHz, DMSO-d₆): δ ppm 12.7 (s, 1H), 8.35-7.94 (m, 4H), 7.61-7.41 (m, 5H), 7.36-7.21 (m, 7H), 6.92-6.88 (m, 4H), 5.81-5.74 (m, 1H), 4.53-4.36 (m, 1H), 4.18-4.15 (m, 2H), 3.79-3.67 (m, 9H), 3.66-3.51 (m, 3H), 3.47-3.32 (m, 4H), 2.79-2.58 (m, 2H), 1.94-1.82 (m, 3H), 1.19-1.10 (m, 10H), 0.98 (d, J=6.68 Hz, 3H), 0.87-0.79 (m, 6H). ³¹PNMR (162 MHz, CDCl₃): 149.41, 148.94. ESI-LCMS: m/z 1001 [M+H]⁺.

### Example A16

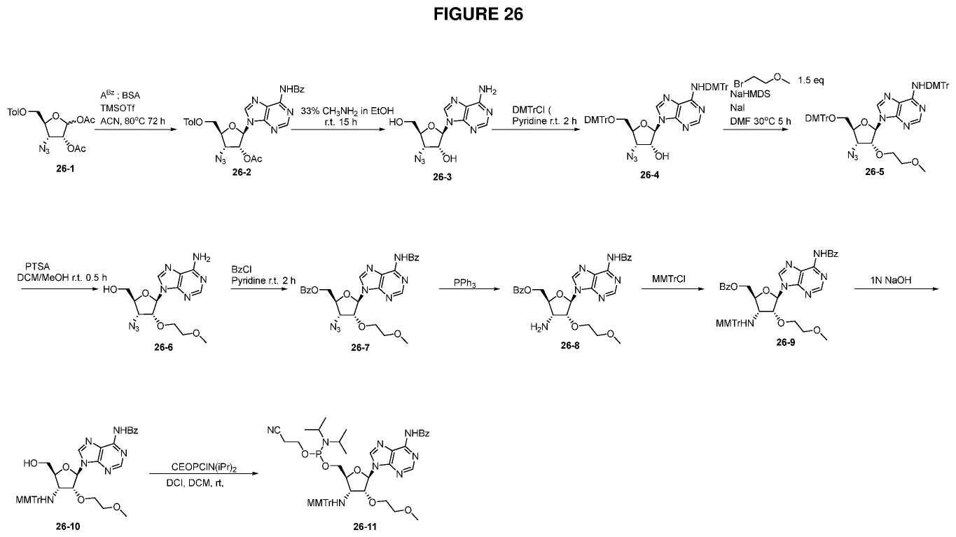

The building block compound 26-11 is useful for making embodiments of modified phosphorothioated oligonucleotides. With reference to FIG. 26, the compound 26-11 was prepared in accordance with known methods. See WO 2019053659 A1.

Preparation of compound 26-2: To a solution of compound 26-1 (12 g, 31.83 mmol) in dry ACN (170 mL) was added N-(5H-purin-6-yl)benzamide (11.42 g, 47.73 mmol) and BSA (180.07 g, 884.86 mmol). The resulting suspension was stirred at 50° C. until clear. Then the mixture was cooled at −20° C. and TMSOTf (10.61 g, 47.73 mmol) was added by syringe. Then the mixture was stirred at 70° C. for 72 hours under N₂, LC-MS showed 26-1 was consumed. Quenched with sat NaHCO₃ and extracted with DCM. The organic layer was dried over Na₂SO₄, then solvent was evaporated, and the residue was purified on silica gel with (PE:EA=1:1) to afford compound 26-2 (16.28 g, 29.27 mmol, 91.9% yield) as a yellow solid. ¹H-NMR (400 MHz, DMSO): δ=11.28 (s, 1H), 8.64 (d, J=6.4 Hz, 2H), 8.05 (d, J=8.0 Hz, 2H), 7.84 (d, J=8.0 Hz, 2H), 7.66 (t, J=7.6 Hz, 1H), 7.56 (t, J=8.0 Hz, 2H), 7.33 (d, J=8.0 Hz, 2H), 6.37 (d, J=3.6 Hz, 1H), 6.17 (dd, J=6.0 Hz, 1H), 5.09 (t, J=6.8 Hz, 1H), 4.69-4.56 (m, 2H), 4.40-4.38 (m, 1H), 2.39 (s, 3H), 2.17 (s, 3H). ESI-LCMS: m/z 557.2 [M+H]⁺.

Preparation of compound 26-3: To a solution of compound 26-2 (16.28 g, 29.27 mmol) dissolved in 33 wt. % methylamine in ethanol (170.00 mL), then the mixture were stirred at 20° C. for 16 h, TLC showed 26-2 was consumed. Then solvent was evaporated, washed with 50% EtOAc in petroleum ether (200 mL), filtered to afford compound 26-3 (8.04 g, 27.52 mmol, 94% yield) as a slightly yellow solid. ESI-LCMS: m/z 293.1 [M+H]⁺.

Preparation of compound 26-4: To a solution of 26-4 (8.04 g, 27.52 mmol) and 4,4′-dimethoxytrityl chloride (27.97 g, 82.55 mmol) in pyridine (80 mL) was stirred for 2 hours at room temperature The LC-MS showed 26-4 completely disappeared. The mixture was quenched with water and extracted by EA. The organic layer was dried over Na₂SO₄, concentrated to give the residue which was purified on silica gel with 16-50% EA in PE to afford 26-4 (17.27 g, 19.26 mmol, 69.9% yield) as a white solid. ESI-LCMS: m/z 897.4 [M+H]⁺.

Preparation of compound 26-5: The crude 26-4 (20.0 g, 22.3 mmol) was dried with toluene for three times. To a solution of 26-4 (20.0 g, 22.3 mmol) in anhydrous DMF (250 mL) was added NaHMDS (26.7 mL, 1M) slowly at −10° C. Then 2-Bromoethyl methyl ether (3.7 g, 26.8 mmol) was added to the reaction mixture. Then NaI (334 mg, 2.2 mmol) was added. Warmed up. The mixture was stirred at 30° C. for 2 hours. LC-MS showed 26-4 was consumed. The reaction mixture was cooled to 0° C. Saturated NH₄Cl solution was slowly added to the mixture. Then water (100 mL) was added. The product was extracted with EA (100 mL*3). The organic layer was washed with brine and dried over Na₂SO₄. The organic solution was concentrated to give the crude 26-5 (23.0 g) as a yellow oil. ESI-LCMS: m/z 955.5 [M+H]³⁰.

Preparation of compound 26-6: To a solution of 26-5 (23.0 g, 23.1 mmol) in DCM (200.0 mL) was added PTSA (9.0 g, 48.2 mmol) in Methanol (10.0 mL). The mixture was stirred at room temperature for 1 hours. TLC showed 26-5 was consumed completely. The reaction mixture was slowly added to cold con. NH₄OH to give the pH=8. Water (100.0 mL) was added. The mixture was extracted with DCM (100.0 mL*3). The organic layer was concentrated and purified by c.c. (PE:EA=5:1˜1:2) to give 26-6 (6.5 g, 18.18 mol, 81.5% yield over two steps) as a yellow solid. ESI-LCMS: m/z 351 [M+H]⁺.

Preparation of compound 26-7: To a stirred solution of compound 26-6 (6.5 g, 18.18 mmol) in pyridine (65 mL) was added benzoyl chloride (7.67 g, 54.55 mmol) at 5° C. The reaction mixture was stirred at room temperature for 1 hour To the reaction mixture was added ammonium hydroxide (637.30 mg, 18.18 mmol, 2 mL) at 5° C. The reaction mixture was poured into water and extracted with EtOAc, layers were separated. Organic phase was washed with sat. aqueous NaCl (1×). The organic phases were evaporated to dryness and the resulting crude material was purified by flash column chromatography on silica gel (PE:EA=3:1˜10:1) to get 26-7 (9.2 g, 15.54 mmol, 85.44% yield) as a yellow solid. ¹H NMR (400 MHz, DMSO-d₆): δ=8.66 (d, J=17.0 Hz, 4H), 8.10-8.01 (m, 4H), 8.01-7.94 (m, 4H), 7.72-7.61 (m, 4H), 7.55 (ddd, J=9.9, 8.3, 7.0 Hz, 8H), 6.25 (d, J=4.7 Hz, 2H), 5.22 (t, J=5.1 Hz, 2H), 4.86 (t, J=5.6 Hz, 2H), 4.71-4.53 (m, 4H), 4.44 (td, J=5.3, 3.8 Hz, 2H), 3.86-3.75 (m, 4H), 3.48-3.40 (m, 4H), 3.35 (s, 1H), 3.13 (s, 6H), 1.25-1.13 (m, 1H), ESI-MS: m/z 559 [M+H]⁺.

Preparation of compound 26-8: To a stirred solution of 26-7 (9.2 g, 15.54 mmol) in EtOAc (100 mL) was added 10% Pd/C (1.92 g, 10%). The reaction mixture was stirred at room temperature for 5 hours under H₂ atmosphere. The reaction was filtered and the filtrate was concentrated under reduced pressure to dryness to give 26-8 (8.41 g, 15.48 mmol, 92.01% yield) as a light-yellow solid. ESI-MS: m/z 533.3 [M+H]⁺.

Preparation of compound 26-9: To a stirred solution of 26-8 (8.41 g, 15.48 mmol) in DCM (80 mL) was added triethylamine (8.38 g, 82.81 mmol, 11.55 mL). 4-Methoxytrityl Chloride (6.14 g, 19.87 mmol) was added at 5° C. The reaction mixture was stirred at room temperature for 2 h under N₂. The reaction was poured into water and extracted with EtOAc, and layers were separated. Organic phase was washed with sat. aqueous NaCl (1×). The organic phases were evaporated to dryness, and the resulting crude material was purified by flash column chromatography on silica gel (PE:EA=1:1˜10:1) to get 26-9 (6.50 g, 8.87 mmol, 65.5% yield) as a light-yellow solid. ¹H NMR (400 MHz, DMSO-d₆): δ=11.17 (s, 1H), 8.56 (s, 1H), 8.36 (s, 1H), 8.08-8.00 (m, 2H), 7.70-7.55 (m, 6H), 7.55-7.46 (m, 5H), 7.46-7.36 (m, 4H), 7.17 (td, J=7.7, 3.5 Hz, 4H), 7.10-7.00 (m, 2H), 6.72-6.65 (m, 2H), 6.06 (s, 1H), 4.82 (dd, J=12.8, 2.0 Hz, 1H), 4.72 (dd, J=12.8, 3.4 Hz, 1H), 4.30 (dt, J=10.1, 2.6 Hz, 1H), 4.09-3.93 (m, 2H), 3.53 (s, 4H), 3.44-3.33 (m, 2H), 3.19 (s, 3H), 3.03-2.88 (m, 2H), 2.48 (d, J=4.8 Hz, 5H), 2.00 (s, 2H), 1.18 (t, J=7.1 Hz, 2H). ESI-MS: m/z 805.4 [M+H]⁺.

Preparation of compound 26-10: To a stirred solution of 26-9 (6.50 g, 8.87 mmol) in pyridine (60 mL) was added 2N NaOH (MeOH:H₂O=4:1) at 5° C. to adjust pH=12-13. The reaction mixture was stirred at 5° C. for 1 hour To the reaction was added sat. aqueous NH₄Cl to adjust pH=7-8. The mixture was extracted with EtOAc, and layers were separated. Organic phase was washed with saturated aqueous NaHCO₃ (1×) and saturated aqueous NaCl (1×). The organic phases were evaporated to dryness and the resulting crude material was purified by flash column chromatography on silica gel (DCM:MeOH=20:1˜50:1) to get 26-10 (6.0 g, 7.41 mmol, 81% yield,) as a yellow solid. ESI-MS: m/z 701.3 [M+H]⁺.

Preparation of compound 26-11: To a stirred solution of 26-10 (6.0 g, 7.41 mmol) in DCM (5 mL) was added DCI (584.21 mg, 4.35 mmol) at 0° C. under N₂. CEOP[N(iPr)₂]₂ (5.64 g, 12.50 mmol) was added dropwise. The reaction mixture was stirred at room temperature for 2 hours. The reaction mixture was poured into water. It was diluted with EtOAc, and layers were separated. Organic phase was and washed with sat. aqueous NaCl (1×). The organic phases were evaporated to dryness, and the resulting crude material was purified by reverse phase preparative HPLC (Column: C18 spherical 20-35 μm 100A 40 g, mobile phase: 0.05% NH₄HCO₃ in water, m/m)-ACN from 50% to 100%, flow rate: 20 ml/min) to get 26-11 (5.7 g, 6.30 mmol, 85.0% yield) as a white solid. ¹H NMR (400 MHz, DMSO-d₆): δ 11.18 (s, 1H), 8.66 (d, J=10.3 Hz, 1H), 8.41 (d, J=11.4 Hz, 1H), 8.10-8.03 (m, 2H), 7.69-7.60 (m, 1H), 7.60-7.44 (m, 6H), 7.42-7.30 (m, 2H), 7.28-7.05 (m, 6H), 6.82-6.70 (m, 2H), 6.10 (d, J=11.4 Hz, 1H), 4.23-4.06 (m, 3H), 3.75-3.48 (m, 8H), 3.48-3.36 (m, 4H), 3.17 (d, J=7.9 Hz, 3H), 3.14-2.97 (m, 2H), 2.87-2.66 (m, 2H), 1.28-0.99 (m, 13H). ³¹P NMR (162 MHz, D₂O): 147.75, 146.54. ESI-MS: m/z 901.0 [M+H]⁺.

### Example A17

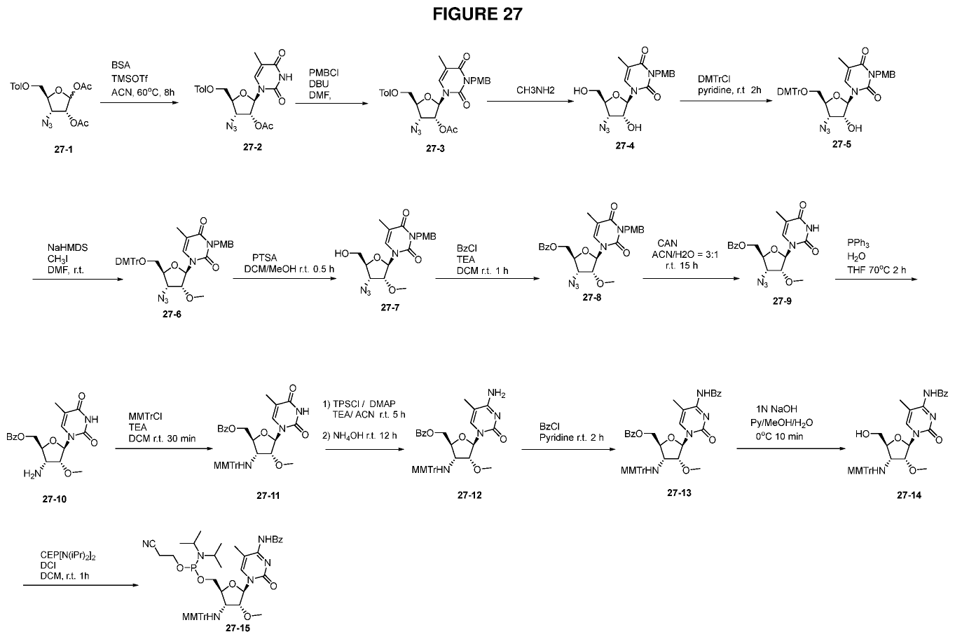

The building block compound 27-15 is useful for making embodiments of modified phosphorothioated oligonucleotides. With reference to FIG. 27, the compound 27-15 was prepared in accordance with known methods. See U.S. Pat. No. 6,608,036.

Preparation of compound 27-2: To a solution of 27-1 (prepared according to Ozols, A. et. al, Synthesis, 1980 (7), 557-9) (34.0 g, 90.1 mmol) in dry ACN (780 mL) was added thymine (34.1 g, 269.8 mmol) and BSA (76.8 g, 386.4 mmol). The resulting suspension was stirred at 50° C. until clear. Then the mixture was cooled at −20° C. and TMSOTf (30.0 g, 135.2 mmol) was added dropwise. Then the mixture was stirred at 70° C. for 12 h, LC-MS showed 27-1 was consumed. Quenched with sat NaHCO₃ (100 mL) and extracted with DCM (200 mL*2). The organic layer was dried over Na₂SO₄ and concentrated, then purified by a silica gel column by (PE:EA=3:1) to afford 27-2 (36.2 g, 80.3 mmol, 89.2% yield) as a white solid. ESI-LCMS: m/z 444 [M+H]⁺.

Preparation of compound 27-3: To a solution of 27-2 (36.2 g, 80.3 mmol) in DMF (280 mL) was added PMBCl (19.1 g, 120.1 mmol) and DBU (24.4 g, 160.5 mol). The mixture was stirred at room temperature for 2 hours. TLC showed 2 was consumed completely. Water (500 mL) was added. The mixture was extracted with EA (200 mL*2). The organic layer was concentrated to give the crude 27-3 (50.0 g) as a yellow oil which was used directly for the next step. ESI-LCMS: m/z 564 [M+H]⁺.

Preparation of compound 27-4: To a solution of 27-3 (50.0 g) in THF (300 mL) was added NaOH (18.6 g) in H₂O (100 mL). The mixture was stirred at room temperature overnight. LC-MS showed 27-3 was consumed completely. Water (200 mL) was added to the mixture. The THF layer was dried over Na₂SO₄. The aqueous layer was extracted with DCM (100 mL) for 3 times. The organic layer was dried over Na₂SO₄. The organic solution was concentrated to give the crude residue. The residue was washed with (PE:EA=3:1) to give 27-4 (23.0 g, 57 mmol, 70.2% yield over two steps) as a white solid. ESI-LCMS: m/z 404.1 [M+H⁺].

Preparation of compound 27-5: To a solution of 27-4 (23.0 g 57.0 mmol) in DCM (250 mL) was added Pyridine (30 mL). Then DMTrCl (20.3 g, 59.9 mmol) dissolved in DCM (80 mL) was slowly added to the mixture. The reaction mixture was stirred at room temperature for 1 hours. LC-MS showed 27-4 was consumed completely. MeOH (10 mL) was added to the mixture. Water (200 mL) was added to the mixture. The organic layer was washed with brine and dried over Na₂SO₄. The organic solution was concentrated and purified by silica gel column (SiO₂, PE:EA=5:1 to 3:1) to give the 27-5 (33.0 g, 42.5 mmol, 82.0% yield), ESI-LCMS: m/z 706.2 [M+H]⁺.

Preparation of compound 27-6: The crude 27-5 was dried with toluene for three times. To a solution of 5 (30.0 g, 42.5 mmol) in anhydrous DMF (300 mL) was added NaHMDS (80 mL) slowly at −10° C. Then CH₃I (9.1 g, 63.8 mmol) was added to the reaction mixture, stirred at 30° C. for 2 hours. LC-MS showed 27-5 was consumed. The reaction mixture was cooled to 0° C. Saturated NH₄Cl solution was slowly added to the mixture. Then water (200 mL) was added. The product was extracted with EA (100 mL*3). The organic layer was washed with brine and dried over Na₂SO₄. The organic solution was concentrated to give the crude 27-6 (52.6 g) as a yellow oil. ESI-LCMS: m/z 720.1 [M+H]⁺.

Preparation of compound 27-7: To a solution of 27-6 (56.2 g) in DCM (400 mL) was added PTSA (7.3 g, 42.5 mmol) in Methanol (15 mL). The mixture was stirred at room temperature for 1 hours. TLC showed 27-6 was consumed completely. The reaction mixture was slowly added to cold Con. NH₄OH to give the pH=8. Water (200 mL) was added. The mixture was extracted with DCM (100 mL*3). The organic layer was concentrated and purified by silica gel column (SiO₂, PE:EA=5:1˜1:2) to give 27-7 (13.0 g, 31.2 mmol, 73.2% yield over three steps) as a yellow oil. ESI-LCMS: m/z 418.2 [M+H]⁺.

Preparation of compound 27-8: To a solution of 27-7 (13.0 g, 31.2 mmol) in DCM (150 mL) was added TEA (8.7 mL, 62.5 mmol). Then BzCl (5.3 g, 37.4 mmol) was added to the mixture. The reaction mixture was stirred at room temperature for 30 min. TLC showed 27-7 was consumed completely. The reaction was added water. The organic layer was washed with brine and dried over Na₂SO₄. The organic layer was concentrated to give the crude 27-8 (14.3 g) as a yellow oil which was used directly for the next. ESI-LCMS: m/z 522.2 [114+H]+.

Preparation of compound 27-9: To a solution of 27-8 (14.3 g) in acetonitrile (150 mL) and water (50 mL) was added Ceric ammonium nitrate (34.2 g, 62.4 mmol). The reaction mixture was stirred at room temperature for 12 hours. LC-MS showed 27-8 was consumed completely. The reaction mixture was concentrated and extracted with EA (50 mL*3). The organic layer was concentrated and purified by silica gel column (SiO₂, PE:EA=4:1˜1:3) to give 27-9 (8.5 g, 21.2 mmol, 78.0% yield over two steps) as a yellow solid. ESI-LCMS: m/z 402.3 [M+H]⁺.

Preparation of compound 27-10: To a solution of 27-9 (8.5 g, 21.2 mmol) in THF (100 mL) was added PPh₃ (7.2 g, 27.6 mmol) and water (1 mL). The reaction mixture was stirred at 70° C. for 3 hours. LC-MS showed 27-9 was consumed completely. Concentrated, the residue was purified by silica gel column (SiO₂, PE:EA=3:1) to give 27-10 (6.8 g, 18.1 mmol, 86.0 yield) as a yellow solid. ESI-LCMS: m/z 376.2 [M+H]⁺.

Preparation of compound 27-11: To a solution of 27-10 (6.8 g, 18.1 mmol) in DCM (100 mL) was added TEA (5.1 mL, 36.3 mmol) and MMTrCl (6.7 g, 21.7 mmol). The reaction mixture was stirred at room temperature for 30 min. TLC showed 27-10 was consumed completely. Water (100 mL) was added to the mixture. The organic layer was washed with brine and dried over Na₂SO₄. The organic solution was concentrated and purified by c.c. (PE:EA=3:1˜1:1.5) to give 27-11 (10.2 g, 15.8 mmol, 86.0% yield) as a yellow solid. ESI-LCMS: m/z 648.2 [M+H]⁺.

Preparation of compound 27-12: To a solution of 27-11 (10.2 g, 15.8 mol) in ACN (100 mL) was added TEA (3.6 mL, 2.5 mol) and DMAP (3.0 g, 2.5 mmol), then TPSCl (5.4 g, 1.8 mmol) was added to the solution. The reaction mixture was stirred at room temperature for 5 hours under N₂. TLC showed 27-11 was consumed completely. Con. NH₄OH (30 mL) was added to the reaction mixture. The mixture was stirred at room temperature for 12 hours. The solution was concentrated and extracted with EA (100 mL*3). The organic layer was washed by brine and dried over Na₂SO₄. The organic layer was concentrated give crude 27-12 (11.0 g) as a yellow oil. ESI-LCMS: m/z 647.2[M+H]⁺.

Preparation of compound 27-13: The crude 27-12 was dried with toluene for three times. To a solution of 27-12 (11.0 g) in Pyridine (140 mL) was added BzCl (4.9 g, 34.7 mmol) at 0° C. The mixture was stirred at room temperature for 1 hours. TLC showed 27-12 was consumed completely. The solution was added water and concentrated to give the residue. The residue was dissolved in EA (500 mL) and water (300 mL). The organic layer was washed with brine and dried over Na₂SO₄. The organic solution was concentrated and purified by silica gel column (SiO₂, PE:EA=5:1˜1:2) to give 27-13 (8.1 g, 10.8 mmol, 70.0% yield over two steps) as a yellow oil. ESI-LCMS: m/z 751.0 [M+H]⁺.

Preparation of compound 27-14: Compound 27-13 (8.1 g, 10.8 mmol) was added to 100 mL of 1 N NaOH solution in pyridine/MeOH/H₂O (65/30/5) at 0° C. The suspension was stirred at 0° C. for 30 min. LC-MS showed 27-13 was consumed completely. The reaction mixture was quenched by addition of sat. NH₄Cl solution (100.0 mL). The solution was added to water (50.0 mL) to give the solid. The solid was filtered and washed with (PE:EA=3:1) to give 27-14 (5.6 g, 8.6 mmol, 80.0% yield) as white solid. ESI-LCMS: m/z 647.2 [M+H]⁺. ¹H-NMR (DMSO-d₆, 400 MHz): δ ppm 13.06 (s, 1H), 8.27-8.19 (m, 3H), 7.59-7.17 (m, 15H), 6.83 (d, J=8.7 Hz, 2H), 5.53 (s, 1H), 5.31 (t, J=3.6 Hz, 1H), 4.08 (s, 1H), 3.95 (d, J=9.5 Hz, 1H), 3.71 (s, 3H), 3.19-3.14 (m, 1H), 3.02 (s, 3H), 2.64 (d, J=10.8 Hz, 1H), 1.93 (s, 3H), 1.57 (s, 1H).

Preparation of compound 27-15: To a solution of 27-14 (5.6 g, 8.6 mmol) in DCM (60 mL) was added DCI (920 mg, 7.8 mmol), then CEOP[N(iPr)₂]₂ (3.4 g, 11.3 mmol) was added. The mixture was stirred at room temperature for 40 min. LC-MS showed 27-14 was consumed completely. The reaction was quenched with saturated NaHCO₃. The organic layer was washed with water and brine, dried over Na₂SO₄, concentrated to give the crude product. The crude was purified by Flash-Prep-HPLC with the following conditions (IntelFlash-1): Column, C18 silica gel; mobile phase, CH₃CN/H₂O (0.5% NH₄HCO₃)=5/5, increasing to CH₃CN/H₂O (0.5% NH₄HCO₃)=1/0 within 20 minutes and hold CH₃CN/H₂O (0.5% NH₄HCO₃)=1/0 for 10 min. The eluted product was collected at CH₃CN/H₂O (0.5% NH₄HCO₃)=1/0; Detector, UV 254 nm. The product was drying in vacuum at 40° C. overnight to give 27-15 (5.2 g, 6.1 mmol, 71.2% yield) as a white solid. ESI-LCMS: m/z 847.1 [M+H]⁺. ¹H-NMR (DMSO-d₆, 400 MHz): δ ppm 13.27 (s, 1H), 8.28 (d, J=7.2 Hz, 2H), 7.99 (s, 1H), 7.57-7.41 (m, 9H), 7.29-7.25 (m, 4H), 7.22-7.15 (m, 2H), 6.82 (d, J=8.8 Hz, 2H), 5.60 (s, 1H), 4.30-4.26 (m, 1H), 4.10 (d, J=10.0 Hz, 1H), 4.02-3.98 (m, 1H), 3.73 (s, 3.5H), 3.66-3.60 (m, 2H), 3.47-3.43 (m, 1H), 3.28-3.16 (m, 1.5H), 3.08 (s, 2.5H), 3.03 (s, 0.7H), 2.88-2.80 (m, 1H), 2.59 (t, J=6.0 Hz, 0.4H), 2.39 (t, J=6.1 Hz, 0.4H), 2.13 (s, 1H), 2.08 (s, 2.4H), 2.04 (s, 0.6H), 1.38 (d, J=4.5 Hz 0.8H), 1.23-1.20 (m, 12.2H). ³¹PNMR (DMSO-d₆, 162 MHz): 148.23, 145.92.

### Example A18

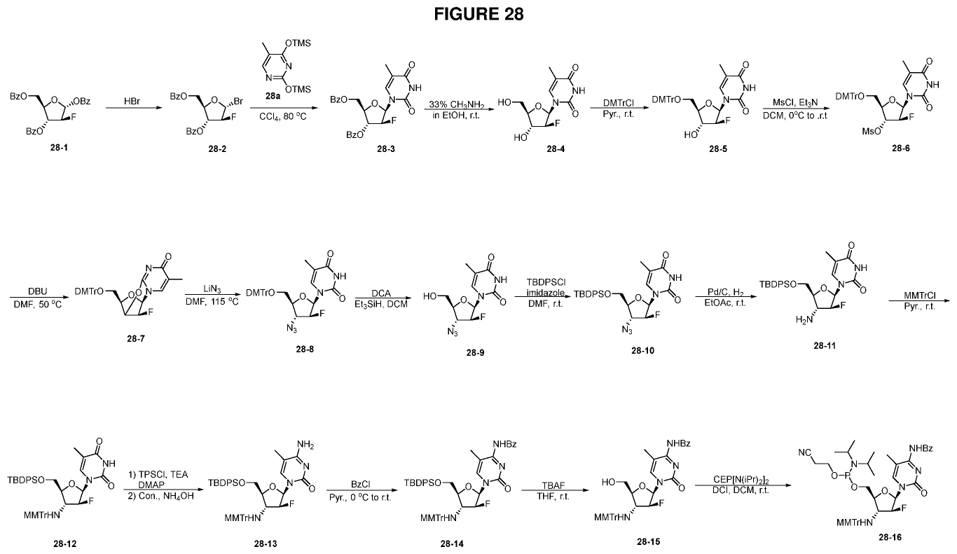

The building block compound 28-16 is useful for making embodiments of modified phosphorothioated oligonucleotides. With reference to FIG. 28, the compound 28-16 was prepared in accordance with known methods. See Schultz, R. G. et al., Tetrahedron Letters, 2000, 41, 1895-1899.

Preparation of compound 28-2: To a solution of 28-1 (85 g, 0.183 mmol) in DCM (900 mL) was added a solution of HBr in acetic acid (30%, 150 mL) at room temperature. The resulting mixture was stirred at room temperature overnight. Excess acid was quenched by the careful addition of saturated aqueous NaHCO₃ solution at 0° C. The mixture was then diluted with EA (500 mL) and the phases were separated. The aqueous phase was twice extracted into ethyl acetate. The extracts were combined, washed with 20% aqueous sodium thiosulfate (300 mL), water (500 mL) and brine (500 mL) then dried over anhydrous Na₂SO₄, evaporated in the vacuo to give 28-2 (73 g, 0.173 mol, 94.5% yield) as an oil which was used without further treatment. ESI-MS: m/z 423.1 [M+H]⁺.

Preparation of compound 28a: To a suspension of thymine (40 g, 0.317 mmol) in CH₃CN (500 mL) was added hexamethyldisilazane (153.5 g, 0.951 mol) and ammonium sulfate (12.56 g, 0.0951 mol) at room temperature. Then the mixture was heated to 85° C. and stirred at this temperature for 5 h, until a clear solution was obtained. The solution was evaporated in vacuo to give an oil which was used without further treatment.

Preparation of compound 28-3: To a solution of 28-2 (73 g, 0.173 mol) in CCl₄ (500 mL) was added 28a (70 g, 0.259 mol) at room temperature and the mixture was heated to 80° C. and stirred at this temperature for 48 hours. The 28-2 was consumed and major desired product were detected by TLC and LC-MS. The reaction was poured to water (1000 mL) and extracted with DCM (300 mL*3). The combined organic layers were washed with (500 mL*2), brine (1000 mL), dried over anhydrous Na₂SO₄ and evaporated in the vacuo to give crude product, which was purified by column chromatography with a gradient of 10 to 35% EtOAc in PE to give 28-3 (70 g, 149.44 mmol, 86.6% yield) as white solid. ¹H NMR (400 MHz, CDCl₃) δ 9.49 (s, 1H), 8.10 (dd, J=16.7, 7.9 Hz, 4H), 7.72-7.57 (m, 2H), 7.49 (dt, J=11.6, 7.7 Hz, 4H), 7.38 (s, 1H), 6.39 (dd, J=22.1, 2.6 Hz, 1H), 5.66 (dd, J=17.9, 2.6 Hz, 1H), 5.36 (dd, J=50.2, 2.6 Hz, 1H), 4.83 (qd, J=12.2, 4.0 Hz, 2H), 4.51 (d, J=3.4 Hz, 1H), 1.77 (s, 3H). ESI-MS: m/z 469.0 [M+H]⁺.

Preparation of compound 28-4: To a solution of 28-3 (see Tann, Chou hours. et al., Journal of Organic Chemistry, 1985, 50(19), 3644-7) (70 g, 149.44 mmol) in CH₃NH₂ in EtOH (500 mL) and the solution was stirred at room temperature for 3 hours until 28-3 was consumed and major 28-4 was detected by TLC and LC-MS. The solvent was removed in the vacuo to give crude product, which was purified by recrystallization with (EA/PE/DCM=1/0.5/1) to give 28-4 (35 g, 134.50 mmol, 90.0% yield) as white solid. ¹H NMR (400 MHz, DMSO-d₆) δ 11.38 (s, 1H), 7.59 (s, 1H), 6.11 (dd, J=15.6, 4.2 Hz, 1H), 5.82 (d, J=50.8 Hz, 1H), 5.36-4.80 (m, 2H), 4.38-4.13 (m, 1H), 3.87-3.74 (m, 1H), 3.63 (dt, J=12.1, 8.4 Hz, 2H), 3.36 (s, 1H), 1.79 (s, 3H). ESI-MS: m/z 261.0 [M+H]⁺.

Preparation of compound 28-5: To a solution of 28-4 (35 g, 134.50 mmol) in dry pyridine (300 mL) was added DMTrCl (54.7 g, 161.4 mmol) at room temperature and the mixture was stirred at this temperature for 2 hours until 28-4 was consumed and major 28-5 was detected by TLC and LC-MS. The reaction was quenched with CH₃OH/H₂O (20 mL/500 mL) and the mixture was extracted with EA (300 mL*3), combined organic layers were washed water (500 mL*2), brine (1000 mL), dried over anhydrous Na₂SO₄ and evaporated in the vacuo to give crude product, which was purified by column chromatography with a gradient of 10 to 60% EtOAc in PE to give 28-5 (69.7 g, 123.78 mmol, 92.0% yield) as white solid. ESI-MS: m/z 585.1 [M+Na]⁺.

Preparation of compound 28-6: To a solution of 28-5 (69.7 g, 123.78 mmol) in dry DCM (500 mL) was added Et₃N (62.6 g, 618.9 mmol) and DMAP (1.51 g, 12.4 mmol) at room temperature and then the mixture was ice-cooled to 0° C. and stirred at this temperature for 30 min. Then MsCl (21.2 g, 185.67 mmol) was dropwise slowly to the mixture and the mixture was stirred at this temperature for 2 hours until 28-5 was consumed and major 28-6 was detected by TLC and LC-MS. The reaction was warmed to room temperature and quenched with water (300 mL), the mixture was extracted with DCM (300 mL*2), the combined organic layers were washed with water (500 mL*2), brine (1000 mL), dried over anhydrous Na₂SO₄ and evaporated in the vacuo to give crude product. The crude purified by column chromatography with a gradient of 10 to 30% EtOAc in PE to give 28-6 (72 g, 112.64 mmol, 91.0% yield) as light yellow solid. ESI-MS: m/z 663.1 [M+Na]+.

Preparation of compound 28-7: To a solution of 28-6 (72 g, 112.64 mmol) in dry DMF (600 mL) was added DBU (34.3 g, 225.28 mmol) at room temperature, and the mixture was heated to 50° C. and stirred at this temperature for 12 hours until 28-6 was consumed and major 28-7 was detected by TLC and LC-MS. The reaction was cooled to room temperature and poured into 1 L water and extracted with EA (400 mL*4), combined organic layers were washed water (500 mL*5), brine (1000 mL), dried over anhydrous Na₂SO₄ and evaporated in the vacuo to give crude product. The crude purified by column chromatography with a gradient of 10 to 40% EtOAc in PE to give 28-7 (42.8 g, 78.85 mmol, 70% yield) as white solid. ¹H NMR (400 MHz, CDCl₃) δ 7.43-7.36 (m, 2H), 7.34-7.24 (m, 6H), 7.21 (dt, J=9.5, 4.2 Hz, 1H), 7.00 (d, J=1.2 Hz, 1H), 6.88-6.73 (m, 4H), 5.52-5.48 (m, 0.5H), 5.45 (dd, J=3.8, 2.4 Hz, 1H), 5.39-5.34 (m, 0.5H), 5.07 (t, J=2.7 Hz, 1H), 4.41-4.29 (m, 1H), 3.79 (d, J=1.0 Hz, 6H), 3.37 (d, J=6.6 Hz, 2H), 1.97 (s, 3H). ESI-MS: m/z 545.2 [M+H]⁺.

Preparation of compound 28-8: LiN₃ (3.70 g, 142.0 mmol) was suspended in DMF (80 mL) and heated at 105° C. with stirring. To the stirred suspension was added TMEDA (67 mL) followed by azidotrimethylsilane (18.7 mL, 142.0 mmol). After stirring for 2.5 h, 28-7 (42.8 g, 78.85 mmol) dissolved in DMF (30 mL) was added to the solution, and the reaction was allowed to proceed for 20 hours at 115° C. The mixture was cooled, poured into EA (300 mL) and filtered through celite. The solvent was extracted with EA (200 mL*3), combined organic layers were washed with H₂O (500 mL*4), brine (500 mL), dried over anhydrous Na₂SO₄, evaporated in the vacuo to give crude product. The crude product was purified by column chromatography with a gradient of 20 to 60% EtOAc in PE to give 28-8 (16 g, 27.25 mmol, 35% yield) as light yellow solid. ¹H NMR (400 MHz, DMSO-d₆) δ 11.52 (s, 1H), 7.50-7.39 (m, 3H), 7.38-7.22 (m, 7H), 6.91 (d, J=8.7 Hz, 4H), 6.22 (dd, J=11.8, 5.4 Hz, 1H), 5.41 (dt, J=53.2, 5.1 Hz, 1H), 4.66 (ddd, J=22.4, 7.6, 4.9 Hz, 1H), 3.98 (dt, J=7.7, 4.2 Hz, 1H), 3.75 (s, 6H), 3.34 (d, J=6.8 Hz, 2H), 1.63 (s, 3H). ESI-MS: m/z 610.2 [M+Na]⁺.

Preparation of compound 28-9: To a solution of 28-8 (15.3 g, 26.05 mmol) in DCM (60 mL) was added 3% DCA (150 mL) at room temperature and then Et₃SiH (80 mL) was added to the mixture and stirred at this temperature for 4 h, until 28-8 was consumed and major 28-9 was detected by TLC and LC-MS. The reaction was quenched with pyridine and the solvent was removed in the vacuo to give crude product, which was purified by column chromatography with a gradient of 0 to 15% CH₃OH in DCM to give 28-9 (6.12 g, 21.47 mmol, 82.6% yield) as white solid. ESI-MS: m/z 610.2 [M+Na]⁺.

Preparation of compound 28-10: To a solution of 28-9 (6.12 g, 21.47 mmol) in DMF (60 mL) was added imidazole (5.84 g, 85.88 mmol) at room temperature and then ice-cooled to 0° C. and stirred for 30 min. TBDPSCl (8.85 g, 32.21 mmol) was dropwise slowly to the mixture and warmed to room temperature, and stirred at room temperature for 2 hours until 28-9 was consumed and major 28-10 was detected by TLC and LC-MS. The reaction was poured into water (200 mL), and extracted with EA (200 mL*3), combined organic layers was washed water (200 mL*4), brine (300 mL*2), dried over anhydrous Na₂SO₄, evaporated in the vacuo to give crude product. The crude product was purified by column chromatography with a gradient of 10 to 60% EtOAc in PE to give 28-10 (10.2 g, 19.50 mmol, 90.8% yield) as white solid. ESI-MS: m/z 524.2 [M+H]⁺.

Preparation of compound 28-11: 28-10 (10.2 g, 19.50 mmol) was dissolved in EtOAc (200 mL), then Pd/C (2.0 g of 10 percent Pd) was added and the mixture was hydrogenated under hydrogen balloon for 2 hours. The catalyst was removed by filtration through celite and the solvent was removed in the vacuo to give crude product 28-11 (9.2 g, 18.50 mmol, 95% yield) as yellow solid, which was used directly for next step without any purification. ESI-MS: m/z 498.3 [M+H]⁺.

Preparation of compound 28-12: To a solution of 28-11 (9.2 g, 18.50 mmol) in dry pyridine (90 mL) was added MMTrCl (8.57 g, 27.75 mmol) at room temperature and the mixture was stirred at this temperature for 2 hours until 28-11 was consumed and major 28-12 was detected by TLC and LC-MS. The reaction was quenched with water (200 mL) and CH₃OH (30 mL), the solution was extracted with EA (200 mL*2), combined organic layers were washed water (200 mL*3), brine (300 mL), dried over anhydrous Na₂SO₄, evaporated in the vacuo to give crude product. The crude product was purified by column chromatography with a gradient of 10 to 50% EtOAc in PE to give 28-12 (12.4 g, 16.12 mmol, 87.2% yield) as white solid. ¹H NMR (400 MHz, DMSO-d₆) δ 11.41 (s, 1H), 7.62 (ddd, J=13.2, 7.9, 1.4 Hz, 4H), 7.50-7.34 (m, 12H), 7.29-7.22 (m, 5H), 7.18 (td, J=7.1, 1.1 Hz, 2H), 6.82 (d, J=9.0 Hz, 2H), 6.13 (dd, J=22.8, 3.0 Hz, 1H), 4.05 (dd, J=12.4, 5.3 Hz, 2H), 3.93 (d, J=10.4 Hz, 1H), 3.83 (dd, J=11.6, 3.5 Hz, 1H), 3.75 (d, J=2.8 Hz, 0.5H), 3.69 (s, 3H), 3.62 (d, J=3.0 Hz, 0.5H), 3.52 (ddd, J=26.2, 10.3, 5.6 Hz, 1H), 1.45 (d, J=0.7 Hz, 3H), 0.96-0.84 (m, 9H). ESI-MS: m/z 770.4[M+H]⁺.

Preparation of compound 28-13: To a solution of 28-12 (12.4 g, 16.12 mmol) in dry CH₃CN (170 mL) was added TEA (4.50 mL, 32.24 mmol) and DMAP (3.94 g, 32.24 mmol) at room temperature, then TPSCl (9.76 g, 32.24 mmol) was added to the mixture and stirred at this temperature for 6 hours until 28-12 was consumed, detected by TLC and LC-MS. Then conc. NH₄OH (33 mL) was added to the solution and stirred at this temperature for 12 hours until major 28-13 was detected by TLC and LC-MS. The reaction was poured into water (300 mL) and extracted with EA (200 mL*3), combined organic layers was washed water (300 mL*2), brine (300 mL), dried over anhydrous Na₂SO₄, evaporated in the vacuo to give crude product 28-13 (14.1 g, crude). The crude product was used directly for next step without any purification. ESI-MS: m/z 769.6 [M+H]⁺.

Preparation of compound 28-14: To a solution of 28-13 (14.1 g, crude) in dry pyridine (100 mL) was ice-cooled to 0° C. and BzCl (3.3 mL, 2.0 eq.) was dropwise slowly to the mixture and stirred at this temperature for 3 h, until 28-13 was consumed and major 28-14 was detected by TLC and LC-MS. The reaction was quenched with water (200 mL), and extracted with EA (200 Ml*2), combined organic layers were washed with water (300 mL*2), brine (300 mL), dried over anhydrous Na₂SO₄, evaporated in the vacuo to give crude product. The crude product was purified by column chromatography with a gradient of 0 to 15% EtOAc in PE to give 28-14 (12.3 g, 14.1 mmol, 87.0% yield for two steps) as white solid. ¹⁹F NMR (376 MHz, DMSO-d₆) δ −186.14 (s). ESI-MS: m/z 873.4 [M+H]⁺.

Preparation of compound 28-15: To a solution of 28-14 (12.3 g, 14.1 mmol) in THF (100 mL) was added TBAF (21.2 mL, 21.15 mmol, 1 M in THF) at room temperature and stirred for 1 hours until 28-14 was consumed and major 28-15 was detected by TLC and LC-MS. The solution was poured into EA (300 mL), and the mixture was washed with water (200 mL*10), brine (300 mL*3), dried over anhydrous Na₂SO₄, and evaporated in the vacuo to give crude product. The crude product was purified by column chromatography with a gradient of 10 to 50% EtOAc in PE to give 28-15 (8.1 g, 12.77 mmol, 90.6% yield) as white solid. ESI-MS: m/z 635.4 [M+H]⁺.

Preparation of compound 28-16: To a solution of 28-15 (6.0 g, 9.46 mmol) in 100 mL of dichloromethane with an inert atmosphere of nitrogen was added CEOP[N(iPr)₂]₂ (3.65 g, 12.30 mmol) and DCI (1.02 g, 8.51 mmol) in order at room temperature. The resulting solution was stirred for 1 hours at room temperature and diluted with 100 mL dichloromethane and washed with saturated aqueous sodium bicarbonate (100 mL), water (200 mL*2), brine (200 mL), dried over anhydrous Na₂SO₄, evaporated in the vacuo to give crude product. The crude product was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 28-16 (5.9 g, 7.07 mmol, 74.7% yield) as white solid. ¹H NMR (400 MHz, DMSO-d₆) δ 12.90 (s, 1H), 8.10 (t, J=45.0 Hz, 2H), 7.58 (d, J=6.8 Hz, 1H), 7.56-7.42 (m, 7H), 7.33 (ddd, J=13.5, 9.8, 5.5 Hz, 6H), 7.21 (t, J=7.2 Hz, 2H), 6.88 (dd, J=9.0, 2.1 Hz, 2H), 6.12 (ddd, J=21.6, 5.8, 2.9 Hz, 1H), 4.04 (t, J=10.0 Hz, 2H), 3.93-3.62 (m, 8H), 3.62-3.39 (m, 3H), 2.75 (td, J=5.9, 3.5 Hz, 2H), 1.93 (d, J=2.4 Hz, 3H), 1.13 (d, J=6.3 Hz, 6H), 1.04 (dd, J=11.9, 6.7 Hz, 6H). ¹⁹F NMR (376 MHz, DMSO-d₆) δ −185.43, −186.25. ³¹P NMR (162 MHz, DMSO-d₆) δ 148.19, 147.30. ESI-MS: m/z 835.6 [M+H]⁺.

### Example A19

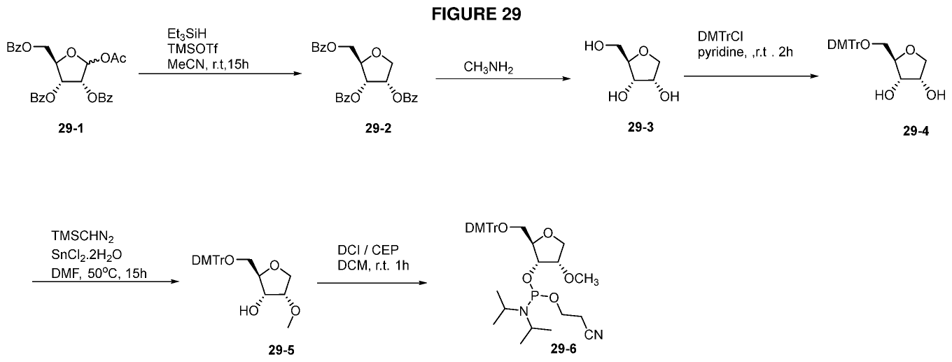

The building block compound 29-6 is useful for making embodiments of modified phosphorothioated oligonucleotides. With reference to FIG. 29, the compound 29-6 was prepared as follows:

Preparation of compound 29-2: To a solution of 29-1 (see Taniho, K. et al., Bioorganic and Medicinal Chemistry Letters, 2012, 22(7), 2518-2521) (50.4 g, 100.1 mmol) and Et₃SiH (34.8 g, 300.3 mmol) in ACN (110.0 mL), TMSOTf (44.4 g, 200.2 mmol) was added at room temperature, mixture was stirred at room temperature for 15 h, TLC showed 29-1 was consumed completely. Mixture was cooled down to 0° C., NaHCO₃ aqueous (500.0 mL) was added to change pH to 7-8, aqueous was extracted with EA (200.0 mL*2), organic phase was dried by Na₂SO₄, concentrated by reduced pressure to give crude which was purified by column chromatography (SiO₂, PE/EA=20:1 to 5:1) to give 29-2 (42.0 g, 94% yield) as an oil. ESI-LCMS: m/z 447.1 [M+H]⁺. ¹H-NMR (400 MHz, CDCl₃) δ=8.06-8.04 (m, 2H), 7.97-7.93 (m, 4H), 7.55-7.50 (m, 3H), 7.42-7.32 (m, 6H), 5.79-5.77 (m, 1H), 5.66-5.63 (m, 1H), 4.71-4.68 (m, 1H), 4.57-4.47 (m, 3H), 4.16-4.13 (m, 1H).

Preparation of compound 29-3: To a solution of 29-2 (37.0 g, 82.9 mmol) was added CH₃NH₂ (400.0 mL). Reaction was stirred at room temperature for 15 h, TLC showed 29-2 was consumed completely. Solvent was removed under reduced pressure, crude was washed with 200.0 mL of PE/EA=1:1 to give 29-3 (9.5 g, 86% yield) as a white solid. ¹H-NMR (400 MHz, MeOD) δ=4.17-4.14 (m, 1H), 4.05-3.97 (m, 2H), 3.80-3.71 (m, 3H), 3.60-3.56 (m, 1H).

Preparation of compound 29-4: To a solution of 29-3 (see Parsch, Joerg et. al., Helvetica Chimica Acta, 2000, 83(8), 1791-1808) (7.0 g, 51.4 mmol) in pyridine (250.0 ml), DMTrCl (19.0 g, 56.6 mmol) was added at 0° C. Reaction was stirred at room temperature for 4.0 h, TLC showed 29-3 was consumed completely. H₂O (200.0 mL) was added, aqueous phase was extracted with EA (200.0 mL*3), organic phase was concentrated to give crude which was purified by column chromatography (SiO₂, PE/EA=5:1 to 1:1) to give 29-4 (19.0 g, 95% purity, 83.7% yield) as a yellow oil. ESI-LCMS: m/z 459.1 [M+Na]⁺. ¹H-NMR (400 MHz, DMSO-d6) δ=7.42-7.40 (m, 2H), 7.33-7.20 (m, 7H), 6.91-6.87 (m, 4H), 4.81-4.75 (m, 2H), 4.04-3.94 (m, 2H), 3.78-3.76 (m, 2H), 3.74 (s, 6H), 3.62-3.59 (m, 1H), 3.13-3.10 (m, 1H), 2.98-2.94 (m, 1H).

Preparation of compound 29-5: To a solution of 29-4 (19.0 g, 43.5 mmol) in DMF (900.0 mL), SnCl₂.H₂O (490 mg, 2.1 mmol) was added, mixture was stirred at room temperature for 10 min, after stirred at 50° C. for 1 min, TMSCHN₂ (65.0 mL, 130.7 mmol, 2.0 M) was added all at once. Reaction was stirred at 50° C. for 15 h, LCMS showed 29-4 was consumed completely. 1.0 L water was added, aqueous was extracted with EA (500.0 mL*3), organic phase was concentrated to give crude which was purified by purified by Flash-Prep-HPLC with the following conditions (IntelFlash-1): Column, SiO₂ silica gel; mobile phase, EA/PE=1/3 increasing to EA/PE=1/0 within 25 min, the eluted product was collected at EA/PE=3/2; Detector, UV 254 nm. This resulted in 29-5 (6.7 g, 34% yield) as an oil. ESI-LCMS: m/z 451.1 [M+H]⁺. ¹H-NMR (400 MHz, DMSO-d₆) δ=7.41-7.39 (m, 2H), 7.33-7.20 (m, 7H), 6.91-6.87 (m, 4H), 4.82-4.80 (d, J=8.0 Hz, 1H), 3.94-3.87 (m, 2H), 3.80-3.69 (m, 3H), 3.74 (s, 6H), 3.34 (s, 3H), 3.11-3.08 (m, 1H), 2.97-2.93 (m, 1H).

Preparation of compound 29-6: To a solution of 29-5 (6.1 g, 13.5 mmol) in dichloromethane (61.0 mL) with an inert atmosphere of nitrogen was added CEOP[N(iPr)₂]₂ (5.3 g, 17.6 mmol) and DCI (1.44 g, 12.19 mmol) in order at room temperature. The resulting solution was stirred for 1.0 hours at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 29-6 (7.5 g, 98% purity, 80% yield) as an oil. ESI-LCMS: m/z 651.5 [M+H]⁺. ¹H-NMR (400 MHz, DMSO-d₆) δ=7.41-7.39 (m, 2H), 7.33-7.21 (m, 7H), 6.91-6.87 (m, 4H), 4.25-4.11 (m, 1H), 4.01-3.85 (m, 3H), 3.79-3.72 (m, 8H), 3.57-3.44 (m, 3H), 3.37-3.33 (m, 3H), 3.25-3.17 (m, 1H), 2.99-2.93 (m, 1H), 2.78-2.75 (m, 1H), 2.57-2.54 (m, 1H), 1.12-1.08 (m, 9H), 0.93-0.91 (m, 3H). ³¹P-NMR (162 MHz, DMSO-d₆) δ=148.61, 148.48.

### Example A20

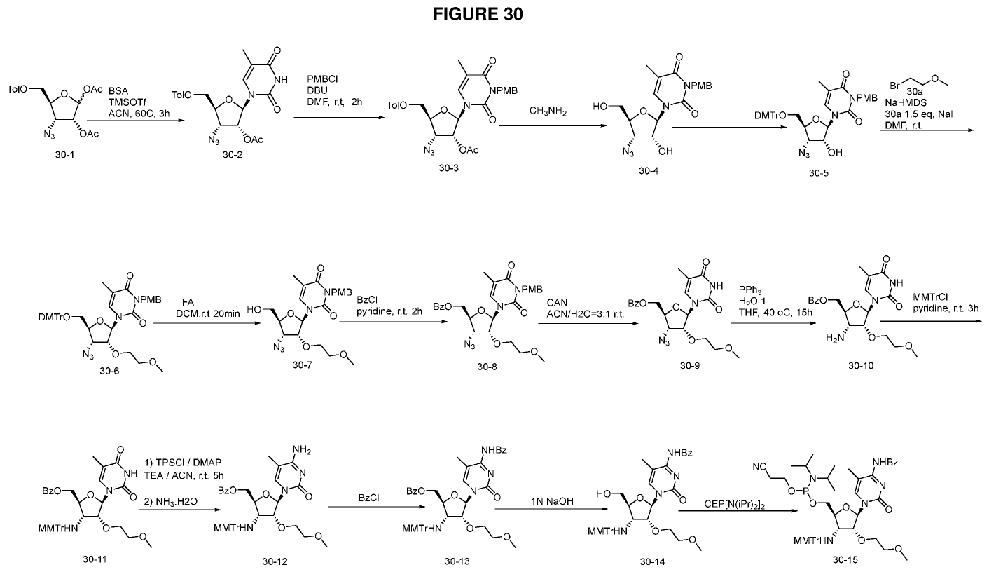

The building block compound 30-15 is useful for making embodiments of modified phosphorothioated oligonucleotides. With reference to FIG. 30, the compound 30-15 was prepared as follows:

Preparation of compound 30-2: To a solution of 30-1 (2.3 g, 18.5 mmol) in ACN (30.0 mL) was added 5-methyl-1H-pyrimidine-2,4-dione (3.5 g, 9.3 mmol), the suspension was purged with N₂ several times. Then BSA (7.9 g, 38.9 mmol) was added, the reaction was stirred at 50° C. After clear, the TMSOTf (4.1 g, 18.5 mmol) was added dropwise at 0° C. and stirred 3 hr at 60° C. Checking the reaction by LCMS showed the completion of the conversion. The mixture was put in NaHCO₃ aqueous (200.0 mL) and extracted with EA (20.0 mL*3), washed with brine aqueous (20.0 mL). The organic layer was dried, separated. The product was obtained as white solid, which was purified by column chromatography (SiO₂, PE/EA=10:1 to 1:1) to give 30-2 (7.8 g, 98.0% purity, 99.1% yield) as white solid. ¹H NMR (400 MHz, DMSO-d₆) δ 11.45 (s, 1H), 7.91 (d, J=8.2 Hz, 2H), 7.44 (d, J=1.2 Hz, 1H), 7.34 (d, J=8.1 Hz, 2H), 5.90 (d, J=4 Hz, 1H), 5.57-5.54 (m, 1H), 4.71-4.75 (m, 1H), 4.62-4.66 (m, 1H), 4.18-4.22 (m, 1H), 2.4 (s, 3H), 2.14 (s, 3H, 1.61 (d, J=1 Hz, 3H). ESI-LCMS: m/z 444.1 [M+H]⁺.

Preparation of compound 30-3: To a solution of 30-2 (41.0 g, 92.5 mmol) in DMF (230.0 mL) was added DBU (46.6 g, 184.9 mmol), the suspension was purged with N₂ several times. Then PMBCl (21.7 g, 138.7 mmol) was added, the reaction was stirred at 25° C. for 2 hr. Checking the reaction by LCMS showed the completion of the conversion. The mixture was put in aqueous NaCl aqueous (1000.0 mL) and extracted with EA (300.0 mL*3), washed with brine (200.0 mL). The organic layer was dried, separated to give crude 30-3 (50.0 g) as yellow oil. ESI-LCMS: m/z 564.0 [M+H]⁺.

Preparation of compound 30-4: To a solution of 30-3 (61.0 g, 108.2 mmol) in MeOH (500.0 mL) was added CH₃NH₂ (432.9 mmol), the reaction was stirred at 25° C. for 17 h. Checking the reaction by LCMS showed the completion of the conversion. The mixture was concentrated to dryness and purified by column chromatography (SiO₂, PE/EA=10:1 to 1:1) to give 30-4 (53.0 g, 98% purified, 98.1% yield) as yellow oil. ¹H NMR (400 MHz, DMSO-d₆) δ 8.57-8.59 (m, 2H), 8.33 (d, J=4 Hz, 2H), 7.83 (d, J=1.1 Hz, 1H), 7.76-7.81 (m, 1H), 7.37-7.40 (m, 2H), 7.24-7.27 (m, 4H), 6.85-6.87 (m, 2H), 6.16-6.17 (d, J=5.0 Hz 1H), 5.82 (d, J=4.0 Hz, 1H), 5.31-5.33 (m, 1H), 4.92 (d, J=1 Hz, 1H), 4.420-4.45 (m, 1H), 4.05-4.08 (m, 1H), 3.89-3.92 (m, 1H), 3.72 (s, 3H), 3.56-3.69 (m, 2H) 2.76 (d, J=4.6 Hz, 3H), 2.34 (s, 2H), 1.84 (d, J=0.8 Hz, 3H). ESI-LCMS: m/z 404.2 [M+H]⁺.

Preparation of compound 30-5: To a solution of 30-4 (55.0 g, 136.4 mmol) in pyridine (450.0 mL) was added DMTrCl (48.5 g, 143.2 mmol) at 0° C. under N₂ atmosphere. The reaction was stirred at 20° C. for 17 h. Checking the reaction by LCMS showed the completion of the conversion. The mixture of H₂O/MeOH=1:1 (3.0 mL) was added and the reaction was stirred 3 min. extracted with EA (20.0 mL*3), washed with brine aqueous (10.0 mL). The organic layer was dried, separated. The residue was purified by column chromatography (SiO₂, PE/EA=10:1 to 5:1) to give 30-5 (59.0 g, 90.0% yield) as white solid. ¹H NMR (400 MHz, DMSO-d₆) δ 7.56 (s, 1H), 7.38-7.40 (d, J=4.0 Hz, 2H), 7.29-7.33 (m, 2H), 7.24-7.27 (m, 7H), 6.89-6.91 (m, 4H), 6.84-6.86 (m, 2H), 6.25-6.26 (d, J=5.4 Hz, 1H), 5.83-5.84 (d, J=4 Hz, 1H), 4.92 (s, 1H), 4.56-4.60 (m, 1H), 4.28-4.31 (m, 1H), 4.01-4.04 (m, 1H), 3.74 (s, 6H), 3.71 (s, 3H), 3.2-3.3 (m, 2H), 1.4 (s, 3H). ESI-LCMS: m/z 728.1 [M+Na]⁺.

Preparation of compound 30-6: To a solution of 30-5 (53.0 g, 75.1 mmol) in DMF (212.0 mL) was added sodium bis(trimethylsilyl)azide (150.2 mg, 150.2 mmol, 150.0 mL) at 0° C. under N₂ atmosphere, the reaction was stirred at 0° C. for 30 min. Then 1-bromo-2-methoxy-ethane (15.6 g, 112.6 mmol) and NaI (1.1 g, 7.5 mmol) was added, the mixture was stirred for 4 h at 25° C. Checking the reaction by LCMS showed half of the conversion was reacted. The mixture was put in EA (500.0 mL), and extracted with H₂O (300.0 mL*3) concentrated and purified by column chromatography (SiO₂, EA/DCM=1:100) to give 30-6 (45.0 g, 80.1% yield) as yellow oil. ESI-LCMS: m/z 462.2 [M+Na]⁺.

Preparation of compound 30-7: To a solution of 30-6 (50 g, 65.46 mmol) in DCM (200.0 mL) was added TFA (150.0 mL) dropwise, the reaction was stirred at 25° C. for 0.5 h. Checking the reaction by LCMS showed the completion of the conversion. The mixture was put in NH₄OH aqueous (200.0 mL) and extracted with DCM (200.0 mL*3), washed with brine aqueous (100.0 mL). Concentrated and dryness to give crude 30-7 (46.0 g) as white oil. ESI-LCMS: m/z 462.2 [M+H]⁺.

Preparation of compound 30-8: To a solution of 30-7 (46.0 g, 99.7 mmol) in pyridine (200.0 mL) was added BzCl (18.2 g, 129.6 mmol, 150.0 mL) dropwise, the reaction was stirred at 25° C. for 17 h. Checking the reaction by LCMS showed the completion of the conversion. Then MeOH (50.0 mL) was added extracted with EA (200.0 mL*3), washed with citric acid aqueous (50.0 mL*3) and then washed with NaHCO₃ aqueous (50.0 mL). The organic layer was dried, separated. The residue was purified by column chromatography (SiO₂, PE/EA=10:1) to give 30-8 (22.0 g, 40.1% yield) as yellow oil. ESI-LCMS: m/z 566.0 [M+H]⁺.

Preparation of compound 30-9: To a solution of 30-8 (20.0 g, 35.3 mmol) in to the mixture of ACN (160.0 mL) and H₂O (56.0 mL) was added CAN (58.1 g, 106.1 mmol), the reaction was stirred at 25° C. for 17 h, LCMS showed the completion of the conversion. Reaction mixture was extracted with EA (200.0 mL*3), washed with NaCl aqueous (100.0 mL). The organic layer was dried, separated. The residue was purified by column chromatography (SiO₂, PE/EA=2:1) to give 30-9 (8.0 g, 50.5% yield) as yellow oil. ESI-LCMS: m/z 446.3 [M+H]⁺.

Preparation of compound 30-10: To a solution of 30-9 (8.0 g, 18.5 mmol) in THF (80.0 mL) was added PPh₃ (9.7 g, 37.1 mmol), the suspension was purged with N₂ several times and stirred at 40° C. for 17 h. Checking the reaction by LCMS showed the completion of the conversion. Extracted with EA (100.0 mL*3), washed with 0.5N/HCl (20 mL*3), and NaHCO₃ aqueous (30.0 mL). The organic layer was dried, separated. The residue was purified by column chromatography (SiO₂, PE/EA=5:1) to give 30-10 (6.4 g, 85.6% yield) as brown solid. ¹H-NMR (400 MHz, DMSO-d₆): δ ppm 7.99-8.01 (m, 2H), 7.66-7.70 (m, 1H), 7.52-7.56 (m, 2H), 7.376 (d, J=0.4 Hz, 1H), 5.78 (d, J=0.4 Hz, 1H), 4.65-4.68 (m, 1H), 4.45-4.49 (m, 1H), 3.88-3.91 (m, 2H), 3.81-3.85 (m, 1H), 3.67-3.72 (m, 3H), 3.48-3.51 (m, 3H), 3.24 (s, 3H), 1.57 (d, J=0.4 Hz 3H). ESI-LCMS: m/z 420.5 [M+H]⁺.

Preparation of compound 30-11: To a solution of 30-10 (6.0 g, 14.3 mmol) in pyridine (71.0 mL) was added TEA (2.9 g, 28.6 mmol, 3.9 mL), MMTrCl (5.7 g, 18.6 mmol), the suspension was purged with N₂ several times and stirred at 25° C. 0.5 h. Checking the reaction by LCMS showed the completion of the conversion. Extracted with EA (10.0 mL*3), washed with NaCl aqueous (20.0 mL*3). The organic layer was dried, separated. The residue was purified by Flash-Prep-HPLC with the following conditions (IntelFlash-1): Column, C18 silica gel; mobile phase, CH₃CN/H₂O (0.5% NH₄HCO₃)=1/5 increasing to CH₃CN/H₂O (0.5% NH₄HCO₃)=1/0 within 20 min, the eluted product was collected at CH₃CN/H₂O (0.5% NH₄HCO₃)=7/1; Detector, UV 254 nm to give 30-11 (9.4 g, 95.2% yield) as brown solid. ¹H-NMR (400 MHz, DMSO-d₆): δ ppm 11.32 (s, 2H), 7.60-7.64 (m, 9H), 7.48-7.53 (m, 12H), 7.39-7.44 (m, 12H), 7.18-7.25 (m, 12H), 7.05-7.13 (m, 9H), 6.72 (d, J=8.5 Hz, 6H), 5.45 (s, 3H), 4.85 (d, J=11.6 Hz, 3H), 4.65-4.69 (m, 3H), 4.21-4.24 (m, 3H), 3.49-3.55 (m, 12H), 3.16 (s, 3H) 2.85-2.84 (m, 6H), 1.99 (s, 3H), 1.73 (d, J=4 Hz, 3H), 1.25 (s, 9H). ESI-LCMS: m/z 692.2 [M+H]⁺.

Preparation of compound 30-12: To a solution of 11 (0.6 g, 867.3 umol) in ACN (8.0 mL) were added TEA (175.5 mg, 1.7 mmol, 241.9 uL), TPSCl (523.9 mg, 1.7 mmol) and N,N-dimethylpyridin-4-amine (211.9 mg, 1.7 mmol), the suspension was purged with N₂ several times and stirred at 25° C. 5 h. Checking the reaction by LCMS showed the completion of the conversion. NH₄OH aqueous (16.0 mL) was added and extracted with EA (10.0 mL*3), washed with NaCl aqueous (20.0 mL*3). The organic layer was dried, separated to give crude 30-12 (0.5 g) brown solid. ESI-LCMS: m/z 691.3 [M+H]⁺.

Preparation of compound 30-13: To a solution of 30-12 (16.00 g, 23.64 mmol) in pyridine (100.00 mL) was added BzCl (4.96 g, 35.46 mmol) at 0° C. The mixture was stirred at room temperature for 1 hours. TLC showed 13 was consumed completely. The solution was concentrated and purified by silica gel column by (PE:EA=3:1˜1:1˜1:2) to give 30-13 (17.40 g, 22.28 mmol) as a white solid. ESI-LCMS: m/z 795.4 [M+H]+

Preparation of compound 30-14: Compound 30-13 (17.40 g, 22.28 mmol) was added to 180 mL of 1 N NaOH solution in pyridine/MeOH/H₂O (65/30/5) at 0° C. The suspension was stirred at 0° C. for 15 min. TLC showed 30-13 was consumed completely. The reaction mixture was quenched by addition of sat. NH₄Cl solution (200 mL). The solution was extracted with EA (400 mL*2) and the combined organic layers were washed with sat. NaHCO₃ solution (200 mL), brine (200 mL), dried over Na₂SO₄, filtered and concentrated. The residue was purified by c.c. (DCM:MeOH=100:1˜50:1) to give 30-14 (12.50 g, 18.47 mmol) as white solid. ESI-LCMS: m/z 691.2 [M+H]⁺.

Preparation of compound 30-15: To a solution of 30-14 (6.3 g, 9.2 mmol) in DCM (63.0 mL) was added DCI (965.7 mg, 8.2 mmol, 241.9 uL) the suspension was purged with N₂ several times. Then CEOP[N(iPr)₂]₂ (3.6 g, 11.8 mmol) was added and stirred at 25° C. 0.5 h. Reaction was monitored by LCMS, and showed the completion of the conversion. H₂O (10.0 mL) was added and extracted with DCM (10.0 mL*3), washed with NaCl aqueous (20.0 mL*3). The organic layer was dried, separated, The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm to give 30-15 (7.3 g, 90.2% yield) as white solid. ¹H-NMR (400 MHz, DMSO-d₆): δ ppm 8.27-8.29 (d, J=8 Hz, 2H), 8.0 (d, 1H), 7.4-7.58 (m, 10H), 7.16-7.28 (m, 6H), 6.8 (d, J=8 Hz, 2H), 5.58-5.62 (m, 10H), 3.89-4.28 (m, 3H), 3.55-3.76 (m, 7H), 3.34-3.46 (m, 3H), 3.16-3.26 (m, 4H), 2.93-3.08 (m, 3H), 2.56 (q, J=6 Hz, 1H), 2.37 (m, 1H) 2.03-2.09 (m, 3H), 1.53 (s, 1H), 1.16-1.23 (m, 12H). ESI-LCMS: m/z 891.2 [M+H]⁺.

### Example A21

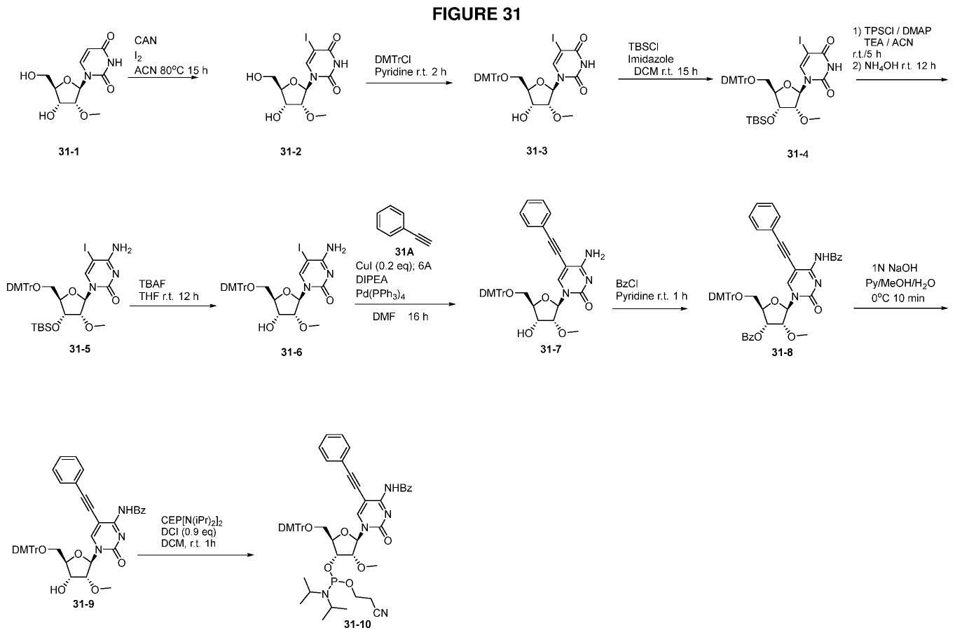

The building block compound 31-10 is useful for making embodiments of modified phosphorothioated oligonucleotides. With reference to FIG. 31, the compound 31-10 was prepared as follows:

Preparation of compound 31-2: To a solution of 31-1 (35.0 g, 135.5 mmol) in MeCN (150 mL) was added I₂ (20.6 g, 81.3 mmol) and CAN (37.1 g, 67.7 mmol). Then the solution was stirred at 80° C. and stirred for 2.5 hours. After the reaction, the solution was cooled down to −10° C. and filtrated at −10° C. to get a yellow solid. The yellow solid was washed with ice MeCN and ice water to give 31-2 (48.0 g, 124.9 mmol, 92.4% yield) as a white solid. ¹H NMR (400 MHz, DMSO-d₆) δ 11.71 (s, 1H), 8.54 (s, 1H), 5.80 (d, J=3.9 Hz, 1H), 5.31 (d, J=4.7 Hz, 1H), 5.14 (d, J=6.3 Hz, 1H), 4.12 (q, J=5.7 Hz, 1H), 3.92-3.84 (m, 1H), 3.79 (d, J=4.6 Hz, 1H), 3.71 (d, J=12.4 Hz, 1H), 3.58 (d, J=12.5 Hz, 1H), 3.39 (s, 3H). ESI-LCMS: m/z 385 [M+H]⁺.

Preparation of compound 31-3: To the solution of 31-2 (45.0 g, 117.1 mmol) in dry pyridine was added DMTrCl (47.5 g, 140.6 mmol) slowly under ice bath. Then the solution was stirred at room temperature overnight. The 31-2 was consumed as indicated by TLC and LCMS. The solvent was concentrated to get a residue. The residue was purified by silica gel column (SiO₂, PE:EA=5:1˜3:1˜1:1) to give 31-3 (68.0 g, 99.1 mmol, 84.5% yield) as pale yellow solid ¹H NMR (400 MHz, DMSO-d₆) δ 11.80 (s, 1H), 8.01 (s, 1H), 7.46-7.37 (m, 2H), 7.38-7.26 (m, 6H), 7.28-7.19 (m, 1H), 6.95-6.86 (m, 4H), 5.80 (d, J=4.3 Hz, 1H), 5.21 (d, J=6.7 Hz, 1H), 4.17 (q, J=5.9 Hz, 1H), 3.96 (ddd, J=17.6, 5.2, 3.5 Hz, 2H), 3.75 (s, 6H), 3.41 (s, 3H), 3.22 (qd, J=10.8, 3.8 Hz, 2H). ESI-LCMS: m/z 709 [M+Na]⁺.

Preparation of compound 31-4: To the solution of 31-3 (65.0 g, 94.7 mmol) in dry DCM (600 mL) was added imidazole (19.3 g, 284.0 mmol). Then TBSCl (21.3 g, 142.0 mmol) was slowly added to the reaction mixture under ice bath. Then reaction mixture was stirred at room temperature overnight. Water was added to the solution. The product was extracted with EA. The combined organic layer was washed with brine and dried over Na₂SO₄ and concentrated to give the crude. The crude was purified by silica gel column (SiO₂, PE:EA=5:1˜3:1˜1:1) to give 31-4 (70.0 g, 87.4 mmol, 92.3% yield) as a white solid. ¹H NMR (400 MHz, DMSO-d₆) δ 8.07 (s, 1H), 7.43-7.35 (m, 2H), 7.29 (dd, J=11.9, 8.0 Hz, 6H), 7.24-7.16 (m, 1H), 6.91-6.82 (m, 4H), 5.74 (d, J=3.4 Hz, 1H), 4.31-4.23 (m, 1H), 3.97-3.86 (m, 2H), 3.70 (s, 6H), 3.35 (s, 3H), 3.30 (dd, J=11.1, 2.7 Hz, 1H), 3.10 (dd, J=11.0, 4.6 Hz, 1H), 0.73 (s, 9H), −0.01 (s, 3H), −0.09 (s, 3H). ESI-LCMS: m/z 823 [M+Na]⁺.

Preparation of compound 31-5: To the solution of 31-4 (60.0 g, 74.9 mmol) in dry MeCN (600 mL) was added TEA (15.1 g, 149.8 mmol), DMAP (18.3 g, 149.8 mmol) and TPSCl (45.4 g, 149.9 mmol) slowly under N₂. Then the solution was stirred at room temperature for 5 hours. TLC showed 31-4 was consumed completely. NH₄OH (6.5 g, 382.3 mmol) was added to the mixture and the combined reaction mixture was stirred at room temperature overnight. Then the solvent was concentrated to give a crude product 31-5 (46.0 g, 57.5 mmol, 90.2% yield) which was used directly for the next step. ESI-LCMS: m/z 800 [M+H]⁺.

Preparation of compound 31-6: To a solution of 31-5 (40.0 g, 50.0 mmol) in THF (400 mL) was added TBAF (19.6 g, 75.0 mmol). After stirring at room temperature for 12 h, the solvent was concentrated to get a residue. The residue was purified by silica gel column (PE:EA=5:1 to 3:1 to 1:1 to EA) to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in the 31-6 (24.0 g, 35.7 mmol, 70.5% yield) as yellow solid. ¹H NMR (400 MHz, DMSO-d₆) δ 7.94 (s, 2H), 7.43 (d, J=7.8 Hz, 2H), 7.37-7.27 (m, 6H), 7.23 (td, J=11.5, 10.7, 6.5 Hz, 1H), 6.94-6.82 (m, 4H), 6.68 (s, 1H), 5.83 (d, J=3.6 Hz, 1H), 5.15 (d, J=6.7 Hz, 1H), 4.15 (q, J=6.2 Hz, 1H), 3.97 (dt, J=6.7, 3.7 Hz, 1H), 3.81 (t, J=4.5 Hz, 1H), 3.75 (s, 6H), 3.43 (s, 3H), 3.22 (d, J=3.7 Hz, 2H). ESI-LCMS: m/z 686 [M+H]⁺.

Preparation of compound 31-7: To a solution of 31-6 (10.0 g, 14.8 mmol) in dry DMF (100.0 mL) was added 31A (4.1 g, 29.7 mmol), CuI (567.2 mg, 2.9 mmol), Pd(P(Ph)₃)₄ (1.7 g, 1.4 mmol), DIPEA (4.8 g, 37.2 mmol). Then the solution was stirred at room temperature overnight under N₂. Then water was added into the mixture, and the obtained mixture was extracted with EA. The combined organic layers were washed with brine, filtered and concentrated to give the residue. The residue was purified by silica gel column (SiO₂, PE:EA=20:1 to 10:1 to EA) to give 31-7 (8.0 g, 12.1 mmol, 81.7% yield) as yellow solid. ESI-LCMS: m/z 660 [M+H]⁺, 1319 [2M+H]⁺.

Preparation of compound 31-8: To a solution of 31-7 (8.0 g, 12.1 mmol) in Pyridine (60 mL) was added BzCl (3.6 g, 25.0 mmol) at 0° C. Then warmed up and the mixture was stirred at room temperature for 1 hours. LCMS showed 31-7 was consumed completely. Water was added to the mixture. The product was extracted with EA. The combined organic layer was washed with brine and dried over Na₂SO₄ and concentrated to give the crude. The crude was purified by silica gel column by (SiO₂, PE:EA=10:1˜5:1˜1:1) to give 31-8 (7.5 g, 8.7 mmol, 72.0% yield) as a yellow solid. ESI-LCMS: m/z 868 [M+H]⁺.

Preparation of compound 31-9: Compound 31-8 (7.5 g, 8.7 mmol) was added to 60 mL of 1 N NaOH solution in pyridine/MeOH/H₂O (65/30/5) at 0° C. The suspension was stirred at 0° C. for 30 min. TLC showed starting material was consumed completely. The reaction was quenched by addition of sat. NH₄Cl solution (300 mL). The solution was extracted with EA (200 mL*2) and the combined organic layers were washed with sat. NaHCO₃ solution (200 mL), brine (200 mL), dried over Na₂SO₄, filtered and concentrated. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in 31-9 (5.7 g, 7.5 mmol, 86.2% yield) as a white solid. 1H-NMR (DMSO-d₆, 400 MHz): δ ppm 12.76 (s, 1H), 8.46-8.09 (m, 3H), 7.60-7.35 (m, 5H), 7.33-7.13 (m, 10H), 6.89-6.78 (m, 6H), 5.86 (s, 1H), 5.26 (d, J=6.08 Hz, 1H), 4.73-3.99 (m, 4H), 3.65 (s, 6H), 3.51-3.27 (m, 4H). ESI-LCMS: m/z 764 [M+H]⁺.

Preparation of compound 31-10: To a solution of 31-9 (5.7 g, 7.5 mmol) in DCM (50 mL) was added DCI (660.1 mg, 5.6 mmol). Then CEP[N(iPr)₂]₂ (2.6 g, 8.6 mmol) was added. The reaction mixture was stirred at room temperature for 1 hour LCMS showed 31-9 was consumed. The reaction mixture was diluted with DCM and washed with H₂O (40 mL*2) and brine (50 mL*2). The combined organic layer was dried over Na₂SO₄ and concentrated to give the residue. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 31-10 (5.6 g, 5.8 mmol, 77% yield) as a white solid. ¹H-NMR (400 MHz, CD₃CN): δ ppm 12.24 (s, 1H), 8.47-8.29 (m, 3H), 7.64-7.51 (m, 5H), 7.48-7.40 (m, 4H), 7.34-7.17 (m, 6H), 6.98-6.82 (m, 6H), 5.92-5.88 (m, 1H), 4.71-4.58 (m, 1H), 4.28-4.14 (m, 2H), 3.88-3.61 (m, 13H), 3.54-3.38 (m, 2H), 2.71-2.68 (m, 1H), 2.52-2.49 (m, 1H), 1.22-1.08 (m, 9H), 1.10 (d, J=6.76 Hz, 3H). ³¹PNMR (162 MHz, DMSO-d₆): 149.54, 148.82. ESI-LCMS: m/z 964 [M+H]⁺.

### Example A22

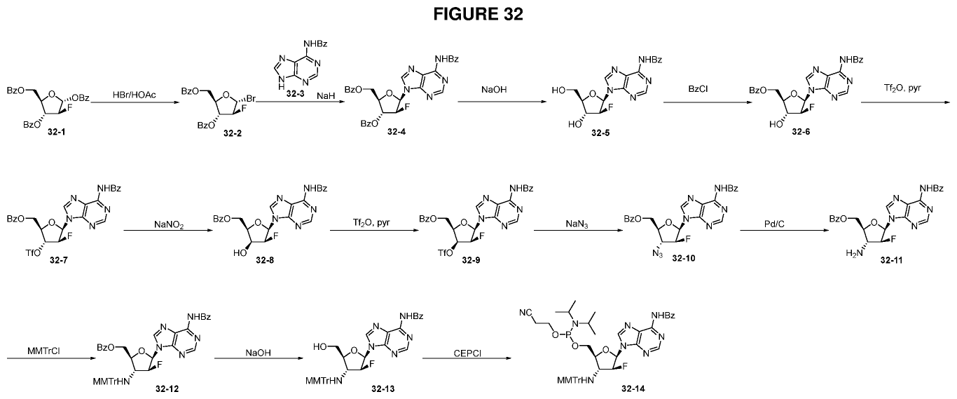

The building block compound 32-14 is useful for making embodiments of modified phosphorothioated oligonucleotides. With reference to FIG. 32, the compound 32-14 was prepared as follows:

Preparation of compound 32-2: To a stirred 32-1 (450.0 g, 969.8 mmol, 1.00 eq.) in dichloromethane (3 L) at 20° C., HBr/HOAc (33%, 472 g, 2 eq.) was added at room temperature The resulting suspension was stirred at room temperature for 16 hours. The reaction mixture was extracted by DCM and washed with sat. NaHCO₃. The Acid was neutralized with sat. NaHCO₃ (2000 mL*5), the pH of the aqueous layer was 7-8. The organic phase was separated, dried over Na₂SO₄ and NaHCO₃, TLC showed no benzoic acid remaining. Filtered and the filtrate was concentrated in vacuo to give 32-2 (414.0 g, 978.7 mmol, 97.0% yield) as yellow oil. ESI-LCMS: m/z 423 [M+H]⁺. ¹H NMR (400 MHz, Chloroform-d) δ 8.24-8.04 (m, 4H), 7.72-7.40 (m, 6H), 6.66 (d, J=12.1 Hz, 1H), 5.73-5.51 (m, 2H), 4.93-4.70 (m, 3H).

Preparation of compound 32-4: To a solution of 32-3 (225.87 g, 943.2 mmol) in THF (4.5 L) was added NaH 60% (38.0 g, 988.1 mmol) at 25° C., the mixture solution was stirred at reflux for 3 h, then a solution of compound 32-2 (380.0 g, 898.3 mmol) in THF (1.2 L) was added to the mixture at 25° C., the resulting solution was stirred at 66° C. for 2 hours. TLC (DCM:Methanol=30:1) showed that compound 32-2 was consumed. The reaction was quenched with ice-water, extracted with ethyl acetate (10 L*3), the organic layer was washed with brine (30 L), concentrated. The product was purified by column chromatography (PE:EA=5:1-1:2 to give 32-4 (257.0 g, 442.3 mmol, 49.0% yield) as a white solid. ¹H NMR (400 MHz, DMSO-d₆) δ 11.29 (s, 1H), 8.79 (d, J=1.2 Hz, 1H), 8.54 (t, J=1.8 Hz, 1H), 8.14-8.10 (m, 2H), 8.08-8.04 (m, 2H), 8.03-7.99 (m, 2H), 7.75 (t, J=7.4 Hz, 1H), 7.59 (ddt, J=34.5, 18.5, 7.6 Hz, 8H), 6.81 (dd, J=17.9, 4.2 Hz, 1H), 6.03 (d, J=19.1 Hz, 1H), 5.86 (dt, J=50.8, 3.6 Hz, 1H), 4.90-4.66 (m, 3H). ESI-LCMS: m/z 582 [M+H]⁺.

Preparation of compound 32-5: To a solution of 32-4 (see WO 2012159047) (252.0 g, 433.30 mmol) was dissolved in pyridine (2.5 L), the mixture solution was cooled to 0° C. Then a solution of 2N NaOH (MeOH:H₂O=4:1) (800 mL, 1.6 mol) was added to the mixture at 0° C., the resulting solution was stirred at 0° C. to −5° C. for 30 min. LCMS showed that 32-4 was consumed. The mixture was adjusted to pH=7 with sat.NH₄C1. Extract with EA (2.5 L*3), washed by brine. The mixture was concentrated under vacuum. The product was purified by silica gel column (PE:EA(2:1)) to give 32-5 (140.0 g, 374.3 mmol, 86.7% yield) as a white solid. ¹H NMR (400 MHz, DMSO-d₆) δ 11.24 (s, 1H), 8.78 (s, 1H), 8.61 (d, J=2.0 Hz, 1H), 8.09-8.03 (m, 2H), 7.69-7.63 (m, 1H), 7.59-7.54 (m, 2H), 6.59 (dd, J=13.5, 4.7 Hz, 1H), 6.02 (d, J=5.0 Hz, 1H), 5.32 (dt, J=52.6, 4.3 Hz, 1H), 5.14 (t, J=5.7 Hz, 1H), 4.51 (dq, J=19.0, 5.0 Hz, 1H), 3.91 (q, J=4.9 Hz, 1H), 3.77-3.64 (m, 2H). ESI-LCMS: m/z 374 [M+H]⁺.

Preparation of compound 32-6: To a solution of 32-5 (130.0 g, 347.6 mmol) in pyridine (1.3 L) was added a solution of benzoyl chloride (49 g, 348.5 mmol) in DCM (400.0 mL). The mixture was stirred at −20° C. for 2 hours. LCMS showed compound 32-5 was consumed. Then the mixture was poured into ice-water and extracted by DCM and washed with cat. NaHCO₃. The mixture was concentrated in vacuum. The product was purified by silica gel column (PE:EA=3:1˜1:1) to give 32-6 (110.0 g, 230.1 mmol, 66.2% yield) as a white solid. ¹H NMR (400 MHz, DMSO-d₆) δ 11.25 (s, 1H), 8.77 (s, 1H), 8.52 (d, J=2.3 Hz, 1H), 8.11-7.97 (m, 4H), 7.70-7.63 (m, 2H), 7.59-7.52 (m, 4H), 6.65 (dd, J=15.2, 4.6 Hz, 1H), 6.24 (d, J=5.0 Hz, 1H), 5.37 (ddd, J=52.4, 4.6, 3.6 Hz, 1H), 4.79-4.59 (m, 3H), 4.27 (td, J=5.6, 3.3 Hz, 1H). ESI-LCMS: m/z 478 [M+H]⁺.

Preparation of compound 32-7: To a solution of 32-6 (104.0 g, 217.6 mmol) in dichloromethane (840 mL) was added pyridine (154.7 g, 1.96 mol, 156 mL). This was followed by the addition of Tf₂O (92.2 g, 326.7 mmol,) drop wise with stirring at 0° C. The resulting solution was stirred for 3 hours at room temperature. TLC (PE:EA=1:1) showed compound 32-6 was consumed. The reaction was then quenched by the addition of sodium bicarbonate (aq.). The resulting solution was extracted with 2 L of dichloromethane and the organic layers combined. The resulting mixture was washed with aqueous sodium chloride. The mixture was dried over anhydrous sodium sulfate and concentrated under vacuum. The mixture was concentrated in vacuo to give 32-7 (120.0 g, 196.7 mmol) as a yellow oil. ESI-LCMS: m/z 610 [M+H]⁺.

Preparation of compound 32-8: To a solution of 32-7 (120.0 g, 196.7 mmol) in N, N-dimethylformamide (2.5 L) was added sodium nitrite (43.0 g, 590.1 mmol). The resulting mixture was stirred overnight at room temperature. The reaction was then quenched by the addition of ice-water. The solid was collected by filtration, extracted by DCM and washed with sat. NaHCO₃. The mixture was concentrated in vacuum, and the product was purified by silica gel column (PE:EA=3:1˜1:3, 1:1 for major impurity, and 1:3 for product) to give 32-8 (18 g, 37.6 mmol, 20.0% over two steps) as white solid. ¹H NMR (400 MHz, DMSO-d₆) δ 11.24 (s, 1H), 8.78 (s, 1H), 8.66 (d, J=1.9 Hz, 1H), 8.09-8.04 (m, 2H), 8.01-7.97 (m, 2H), 7.70-7.63 (m, 2H), 7.55 (dt, J=10.2, 7.7 Hz, 4H), 6.67 (dd, J=8.8, 5.7 Hz, 1H), 6.33 (d, J=5.1 Hz, 1H), 5.57 (ddd, J=51.6, 5.8, 4.5 Hz, 1H), 4.77-4.61 (m, 3H), 4.50 (dt, J=8.3, 4.3 Hz, 1H). ESI-LCMS: m/z 478 [M+H]⁺.

Preparation of compound 32-9: To a solution of 32-8 (16.0 g, 33.5 mmol) in dichloromethane (130 mL) was added pyridine (23.8 g, 301.2 mmol, 24.0 mL). This was followed by the addition of Tf₂O (14.2 g, 50.3 mmol) drop wise with stirring at −10° C., the reaction mixture was stirred at 0° C.˜10° C. for 1.5 hours. The reaction was then quenched by the addition of sodium bicarbonate (aq.). The resulting solution was extracted with 2*1 L of dichloromethane and the organic layers combined. The resulting mixture was washed with 2*1 L of sodium chloride (aq.). The mixture was dried over anhydrous sodium sulfate and concentrated under vacuum. The mixture was concentrated in vacuo to give 32-9 (18.3 g, 30.0 mmol) as a yellow oil. ESI-LCMS: m/z 610 [M+H]⁺.

Preparation of compound 32-10: To a solution of 32-9 (18.3 g, 30.0 mmol) in N, N-dimethylformamide (80 mL) was added sodium azide (6.0 g, 90.0 mmol). The resulting solution was stirred overnight at room temperature. The resulting mixture was concentrated under vacuum. The resulting solution was diluted with 300 mL of dichloromethane. The resulting mixture was washed with 2*200 mL of water and 2*200 mL of sodium chloride (aq.) respectively. The mixture was dried over anhydrous sodium sulfate and concentrated under vacuum. The product was purified by silica gel column (PE:EA=3:1˜1:1) to give 32-10 (12.0 g, 23.8 mmol, 79.3% over two steps) as white solid. ¹H NMR (400 MHz, DMSO-d₆) δ 11.26 (s, 1H), 8.71 (s, 1H), 8.57 (d, J=1.9 Hz, 1H), 8.08-8.00 (m, 4H), 7.71-7.63 (m, 2H), 7.56 (td, J=7.7, 3.4 Hz, 4H), 6.66 (dd, J=10.9, 5.4 Hz, 1H), 5.73 (dt, J=52.6, 5.4 Hz, 1H), 5.25 (ddd, J=20.2, 7.4, 5.3 Hz, 1H), 4.76-4.58 (m, 2H), 4.35 (td, J=6.1, 3.7 Hz, 1H). ESI-LCMS: m/z 503 [M+H]⁺.

Preparation of compound 32-11: To a solution of 32-10 (80% purity)(12.0 g, 23.8 mmol) in THF (120 mL) was added palladium 10% on carbon (1.2 g), the mixture was stirred at room temperature for 5 hours at H₂. Filtered and the filtrate was concentrated in vacuo. The product was purified by silica gel column (PE:EA=1:2 to MeOH:DCM=1:10) to give 32-11 (8.1 g, 17.0 mmol, 92.5% yield) as brown solid. ESI-LCMS: m/z 477 [M+H]⁺. ¹H NMR (400 MHz, DMSO-d₆) δ 11.25 (s, 1H), 8.77 (s, 1H), 8.49 (d, J=2.4 Hz, 1H), 8.09-7.97 (m, 4H), 7.72-7.63 (m, 2H), 7.55 (td, J=7.7, 6.0 Hz, 4H), 6.70 (dd, J=15.7, 4.3 Hz, 1H), 5.19 (dt, J=53.0, 4.0 Hz, 1H), 4.65 (qd, J=12.0, 4.9 Hz, 2H), 4.14 (td, J=6.2, 3.3 Hz, 1H), 3.86 (ddd, J=21.3, 6.0, 3.7 Hz, 1H).

Preparation of compound 32-12: To a solution of 32-11 (8.1 g, 17.0 mmol) in pyridine (80 mL) was added MMTrCl (7.87 g, 25.5 mmol) at 0° C., The mixture was stirred at room temperature for 2 hours under N₂. TLC showed 32-11 was consumed. Filtered and the organic layer was washed by water and dried over Na₂SO₄, concentrated to give the crude product which was purified by silica gel column (PE:EA=3:1˜1:1) to give 32-12 (10.2 g, 13.6 mmol, 81.5% yield) as a white solid. ESI-LCMS: m/z 749 [M+H]⁺. ¹H NMR (400 MHz, DMSO-d₆) δ 11.24 (s, 1H), 8.77 (s, 1H), 8.21 (d, J=3.3 Hz, 1H), 8.08-8.01 (m, 2H), 7.94-7.88 (m, 2H), 7.65 (dddd, J=11.1, 5.6, 3.0, 1.7 Hz, 2H), 7.53 (qd, J=7.9, 1.8 Hz, 8H), 7.45-7.40 (m, 2H), 7.32 (t, J=7.6 Hz, 4H), 7.20 (t, J=7.3 Hz, 2H), 6.91-6.86 (m, 2H), 6.69 (dd, J=23.4, 2.8 Hz, 1H), 4.39-4.14 (m, 5H), 3.69 (s, 3H), 3.52 (ddd, J=23.3, 9.8, 4.6 Hz, 1H).

Preparation of compound 32-13: To a solution of 32-12 (10.2 g, 13.6 mmol) in pyridine (100 mL) was added 2 N NaOH (30 mL) dropwise at 0° C., the mixture was stirred at 0° C. for 30 min. Then the reaction was neutralized with saturated NH₄Cl (aq.) to pH=7-8, and 150 mL H₂O and 400 mL DCM were added in to separate the solution, the aqueous was extracted by DCM, the combined organic layer was washed with brine, dried over anhydrous Na₂SO₄, the solvent was removed and the residue was purified on silica gel (DCM:Methanol=100:1) to give 32-13 (7.9 g, 12.24 mmol, 90.0% yield) as a white solid. ¹H-NMR (400 MHz, DMSO): δ ppm 11.26 (br s, 1H), 8.76 (s, 1H), 8.48-8.38 (m, 1H), 8.10-8.00 (m, 2H), 7.69-7.61 (m, 1H), 7.59-7.48 (m, 6H), 7.46-7.39 (m, 2H), 7.37-7.29 (m, 4H), 7.29-7.13 (m, 6H), 6.95-6.87 (m, 2H), 6.65-6.52 (m, 1H), 4.93-4.93 (m, 1H), 4.13-4.02 (m, 2H), 3.92-3.74 (m, 1H), 3.72 (s, 3H), 3.65-3.57 (m, 1H), 3.53-3.38 (m, 2H). ¹⁹F NMR (376.5 MHz, DMSO): −184.73. ESI-LCMS: m/z 645 [M+H]⁺.

Preparation of compound 32-14: To a solution of 32-13 (7.9 g, 12.24 mmol) in DCM (60 mL) was added DMAP (417 mg, 3.4 mmol) and DIPEA (6.3 g, 48.8 mmol, 8.5 mL). Then CEPCl (4.9 g, 20.7 mmol) was added. The reaction mixture was stirred at room temperature for 1 hours. TLC showed 32-13 was consumed, the mixture was washed with saturated NaHCO₃ and brine, dried over Na₂SO₄, purified by Flash-Prep-HPLC with the following conditions (IntelFlash-1): Column, C18 silica gel; mobile phase, CH₃CN/H₂O (0.5% NH₄HCO₃)=5/5 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 32-14 (9.7 g, 11.47 mmol, 93.7% yield) as a white solid. ¹H-NMR (400 MHz, CDCl₃): δ ppm 9.06 (br s, 1H), 8.78 (s, 1H), 8.21 (t, J=3.4 Hz, 1H), 8.00 (d, J=7.4 Hz, 2H), 7.59 (t, J=7.48 Hz, 1H), 7.54-7.47 (m, 6H), 7.43-7.38 (m, 2H), 7.35-7.29 (m, 4H), 7.25-7.25 (m, 3H), 6.88-6.81 (m, 2H), 6.48-6.37 (m, 1H), 4.21-4.05 (m, 1H), 4.03-3.97 (m, 1H), 3.84-3.47 (m, 10H), 2.65-2.49 (m, 2H), 2.21-2.13 (m, 1H), 1.22-1.22 (m, 12H). ³¹P NMR (162 MHz. CDCl₃): 149.17, 149.02. ¹⁹F NMR (376.5 MHz, CDCl₃): 486.76, −187.09. ESI-LCMS: m/z 845 [M+H]⁺.

### Example A23

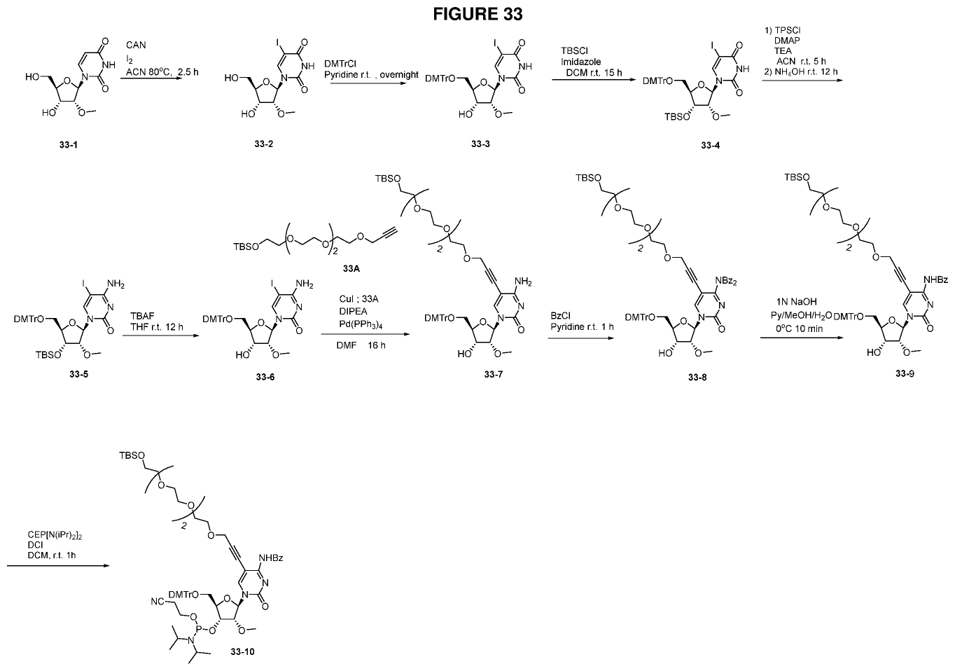

The building block compound 33-10 is useful for making embodiments of modified phosphorothioated oligonucleotides. With reference to FIG. 33, the compound 33-10 was prepared as follows:

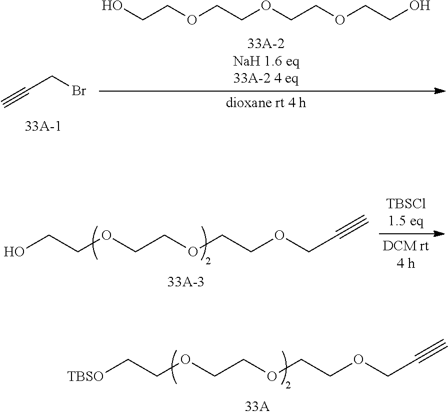

Preparation of intermediate compound 33A-3: NaH (4.7 g, 118.4 mmol, 60% purity) was added in portions to the solution of 33A-2 (46.0 g, 236.8 mmol) in dry dioxane (220.0 mL) under N₂ and kept stirring for 20 minutes on an ice bath. A solution of 33A-1 (7.7 g, 59.2 mmol) in dry dioxane (30.0 mL) was added to this mixture dropwise over 1 hour. The resulting mixture was stirred for another 3 hours at room temperature and 20.0 ml of water was added slowly and stirred for another 20 minutes. The solvent was removed under vacuum and leftover was dissolved in DCM and washed with brine. Finally, purification using column chromatography was done eluting EA which gave the desired liquid colorless product 33A-3 (12 g, 54.98 mmol, 92.86% yield). ¹H NMR (400 MHz, CDCl₃) δ 4.02 (d, J=2.4 Hz, 2H), 3.54-3.45 (m, 14H), 3.41 (dd, J=5.5, 3.9 Hz, 2H), 3.20 (s, 1H), 2.38 (t, J=2.4 Hz, 1H).

Preparation of intermediate compound 33A: To a solution of 33A-3 (12.0 g, 51.6 mmol) in DCM (200.0 mL) was added imidazole (10.5 g, 154.9 mmol) and TBSCl (11.6 g, 77.5 mmol) on an ice bath, then the mixture was stirred at room temperature overnight. After the reaction, water was added into the mixture, extracted, filtered, concentrated, then the product 33A (see Fujiwara, Koichi et. al, Tetrahedron, 1998, 54(10), 2049-2058) (16.0 g, 46.1 mmol, 89.3% yield) was purified by silica gel column (PE:EA=20:1 to 10:1 to 5:1). ¹H NMR (400 MHz, DMSO-d₆) δ 4.14 (d, J=2.4 Hz, 2H), 3.69 (t, J=5.2 Hz, 2H), 3.61-3.50 (m, 12H), 3.46 (t, J=5.2 Hz, 2H), 3.36 (t, J=2.4 Hz, 1H), 0.87 (s, 9H), 0.05 (s, 6H).

Preparation of compound 33-2: To a solution of 33-1 (35.0 g, 135.5 mmol) in MeCN (150 mL) was added I₂ (20.6 g, 81.3 mmol) and CAN (37.1 g, 67.7 mmol). Then the solution was stirred at 80° C. and stirred for 2.5 hours. After the reaction, the solution was cooled down to −10° C. and filtrated at −10° C. to get a yellow solid. The yellow solid was washed with ice MeCN and ice water to give 33-2 (48.0 g, 124.9 mmol, 92.4% yield) as a white solid. ¹H NMR (400 MHz, DMSO-d₆) δ 11.71 (s, 1H), 8.54 (s, 1H), 5.80 (d, J=3.9 Hz, 1H), 5.31 (d, J=4.7 Hz, 1H), 5.14 (d, J=6.3 Hz, 1H), 4.12 (q, J=5.7 Hz, 1H), 3.92-3.84 (m, 1H), 3.79 (d, J=4.6 Hz, 1H), 3.71 (d, J=12.4 Hz, 1H), 3.58 (d, J=12.5 Hz, 1H), 3.39 (s, 3H). ESI-LCMS: m/z 385 [M+H]⁺.

Preparation of compound 33-3: To the solution of 33-2 (45.0 g, 117.1 mmol) in dry pyridine was added DMTrCl (47.5 g, 140.6 mmol) slowly under ice bath. Then the solution was stirred at room temperature overnight. 33-2 was consumed as indicated by TLC and LCMS. The solvent was concentrated to get a residue. The residue was purified by silica gel column (SiO₂, PE:EA=5:1˜3:1˜1:1) to give 33-3 (68.0 g, 99.1 mmol, 84.5% yield) as pale yellow solid. ¹H NMR (400 MHz, DMSO-d₆) δ 11.80 (s, 1H), 8.01 (s, 1H), 7.46-7.37 (m, 2H), 7.38-7.26 (m, 6H), 7.28-7.19 (m, 1H), 6.95-6.86 (m, 4H), 5.80 (d, J=4.3 Hz, 1H), 5.21 (d, J=6.7 Hz, 1H), 4.17 (q, J=5.9 Hz, 1H), 3.96 (ddd, J=17.6, 5.2, 3.5 Hz, 2H), 3.75 (s, 6H), 3.41 (s, 3H), 3.22 (qd, J=10.8, 3.8 Hz, 2H). ESI-LCMS: m/z 709 [M+Na]⁺.

Preparation of compound 33-4: To the solution of 33-3 (65.0 g, 94.7 mmol) in dry DCM (600 mL) was added imidazole (19.3 g, 284.0 mmol). Then TBSCl (21.3 g, 142.0 mmol) was slowly added to the reaction mixture under ice bath. Then reaction mixture was stirred at room temperature overnight. Water was added to the solution. The product was extracted with EA. The combined organic layer was washed with brine and dried over Na₂SO₄ and concentrated to give the crude. The crude was purified by silica gel column (SiO₂, PE:EA=5:1˜3:1˜1:1) to give 33-4 (70.0 g, 87.4 mmol, 92.3% yield) as a white solid. ¹H NMR (400 MHz, DMSO-d₆) δ 8.07 (s, 1H), 7.43-7.35 (m, 2H), 7.29 (dd, J=11.9, 8.0 Hz, 6H), 7.24-7.16 (m, 1H), 6.91-6.82 (m, 4H), 5.74 (d, J=3.4 Hz, 1H), 4.31-4.23 (m, 1H), 3.97-3.86 (m, 2H), 3.70 (s, 6H), 3.35 (s, 3H), 3.30 (dd, J=11.1, 2.7 Hz, 1H), 3.10 (dd, J=11.0, 4.6 Hz, 1H), 0.73 (s, 9H), −0.01 (s, 3H), −0.09 (s, 3H). ESI-LCMS: m/z 823 [M+Na]⁺.

Preparation of compound 33-5: To the solution of 33-4 (60.0 g, 74.9 mmol) in dry MeCN (600 mL) was added TEA (15.1 g, 149.8 mmol), DMAP (18.3 g, 149.8 mmol) and TPSCl (45.4 g, 149.9 mmol) slowly under N₂. Then the solution was stirred at room temperature for 5 hours. TLC showed 33-4 was consumed completely. NH₄OH (6.5 g, 382.3 mmol) was added to the mixture and the combined reaction mixture was stirred at room temperature overnight. Then the solvent was concentrated to give a crude product 33-5 (46.0 g, 57.5 mmol, 90.2% yield) which was used directly for the next step. ESI-LCMS: m/z 800 [M+H]⁺.

Preparation of compound 33-6: To a solution of 33-5 (40.0 g, 50.0 mmol) in THF (400 mL) was added TBAF (19.6 g, 75.0 mmol). After stirring at room temperature for 12 h, the solvent was concentrated to get a residue. The residue was purified by silica gel column (PE:EA=5:1 to 3:1 to 1:1 to EA) to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in the 33-6 (24.0 g, 35.7 mmol, 70.5% yield) as yellow solid. ¹H NMR (400 MHz, DMSO-d₆) δ 7.94 (s, 2H), 7.43 (d, J=7.8 Hz, 2H), 7.37-7.27 (m, 6H), 7.23 (td, J=11.5, 10.7, 6.5 Hz, 1H), 6.94-6.82 (m, 4H), 6.68 (s, 1H), 5.83 (d, J=3.6 Hz, 1H), 5.15 (d, J=6.7 Hz, 1H), 4.15 (q, J=6.2 Hz, 1H), 3.97 (dt, J=6.7, 3.7 Hz, 1H), 3.81 (t, J=4.5 Hz, 1H), 3.75 (s, 6H), 3.43 (s, 3H), 3.22 (d, J=3.7 Hz, 2H). ESI-LCMS: m/z 686 [M+H]⁺.

Preparation of compound 33-7: To a solution of 33-6 (10.0 g, 14.8 mmol) in dry DMF (100.0 mL) was added 33A (4.1 g, 29.7 mmol), CuI (567.2 mg, 2.9 mmol), Pd(P(Ph)₃)₄ (1.7 g, 1.4 mmol), DIPEA (4.8 g, 37.2 mmol). Then the solution was stirred at room temperature overnight under N₂. Then water was added into the mixture, and the obtained mixture was extracted with EA. The combined organic layers were washed with brine, filtered and concentrated to give the residue. The residue was purified by silica gel column (SiO₂, PE:EA=20:1 to 10:1 to EA) to give 33-7 (8.0 g, 11.4 mmol, 77.1% yield) as yellow solid. ¹H NMR (400 MHz, DMSO-d₆) δ 7.99 (s, 1H), 7.85 (s, 1H), 7.43 (d, J=7.2 Hz, 2H), 7.32 (dd, J=8.2, 6.2 Hz, 6H), 7.23 (t, J=7.2 Hz, 1H), 6.97-6.86 (m, 4H), 5.83 (d, J=3.1 Hz, 1H), 5.15 (d, J=7.0 Hz, 1H), 4.27-4.13 (m, 2H), 4.07-3.95 (m, 2H), 3.84-3.72 (m, 6H), 3.68 (t, J=5.1 Hz, 2H), 3.52-3.45 (m, 10H), 3.45-3.39 (m, 4H), 3.39-3.26 (m, 6H), 3.20 (dd, J=10.9, 2.2 Hz, 1H), 0.85 (s, 9H), 0.03 (s, 6H). ESI-LCMS: m/z 904 [M+H]⁺.

Preparation of compound 33-8: To a solution of 33-7 (7.0 g, 10.0 mmol) in pyridine (60 mL) was added BzCl (3.6 g, 25.0 mmol) at 0° C. Then warmed up and the mixture was stirred at room temperature for 1 hours. LCMS showed 33-7 was consumed completely. Water was added to the mixture. The product was extracted with EA. The combined organic layer was washed with brine and dried over Na₂SO₄ and concentrated to give the crude. The crude was purified by silica gel column by (SiO₂, PE:EA=10:1˜5:1˜1:1) to give 33-8 (7.0 g, 7.7 mmol, 77% yield) as a yellow solid. ESI-LCMS: m/z 1112 [M+H]⁺.

Preparation of compound 33-9: Compound 33-8 (7.0 g, 7.7 mmol) was added to 60 mL of 1 N NaOH solution in pyridine/MeOH/H₂O (65/30/5) at 0° C. The suspension was stirred at 0° C. for 30 min. TLC showed starting material was consumed completely. The reaction was quenched by addition of sat. NH₄Cl solution (300 mL). The solution was extracted with EA (200 mL*2) and the combined organic layers were washed with sat. NaHCO₃ solution (200 mL), brine (200 mL), dried over Na₂SO₄, filtered and concentrated. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in 33-9 (5.3 g, 6.6 mmol, 86% yield) as a white solid. ¹H NMR (400 MHz, DMSO-d₆) δ 8.17 (s, 1H), 8.05 (d, J=7.6 Hz, 2H), 7.65-7.57 (m, 1H), 7.51 (t, J=7.6 Hz, 2H), 7.47-7.41 (m, 2H), 7.33 (dq, J=8.0, 2.4 Hz, 6H), 7.28-7.20 (m, 1H), 6.99-6.81 (m, 4H), 5.83 (d, J=2.7 Hz, 1H), 5.25 (d, J=7.1 Hz, 1H), 4.30 (td, J=7.1, 5.1 Hz, 1H), 4.12-3.99 (m, 1H), 3.99-3.89 (m, 3H), 3.75 (s, 6H), 3.67 (t, J=5.1 Hz, 2H), 3.58-3.39 (m, 14H), 3.35 (dd, J=12.0, 5.5 Hz, 5H), 3.23 (dd, J=11.0, 2.2 Hz, 1H), 0.85 (s, 9H), 0.03 (s, 6H); ESI-LCMS: m/z 1008 [M+H]⁺.

Preparation of compound 33-10: To a solution of 33-9 (5.3 g, 6.6 mmol) in DCM (50 mL) was added DCI (660.1 mg, 5.6 mmol). Then CEP[N(iPr)₂]₂ (2.6 g, 8.6 mmol) was added. The reaction mixture was stirred at room temperature for 1 hour LCMS showed 33-9 was consumed. The reaction mixture was diluted with DCM and washed with H₂O (40 mL*2) and brine (50 mL*2). The combined organic layer was dried over Na₂SO₄ and concentrated to give the residue. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 33-10 (4.6 g, 4.6 mmol, 70% yield) as a white solid. ¹H NMR (400 MHz, Acetonitrile-d₃) δ 8.45-8.13 (m, 3H), 7.62 (t, J=7.4 Hz, 1H), 7.59-7.49 (m, 4H), 7.49-7.39 (m, 4H), 7.39-7.32 (m, 2H), 7.28 (td, J=7.2, 1.6 Hz, 1H), 6.92 (dd, J=8.9, 3.8 Hz, 4H), 5.88 (dd, J=14.0, 2.4 Hz, 1H), 4.73-4.54 (m, 1H), 4.30-4.16 (m, 2H), 4.14-3.95 (m, 3H), 3.95-3.75 (m, 8H), 3.75-3.58 (m, 8H), 3.58-3.40 (m, 16H), 2.80-2.60 (m, 1H), 2.54 (t, J=6.0 Hz, 1H), 1.25-1.15 (m, 9H), 1.11 (d, J=6.8 Hz, 3H), 0.90 (s, 9H), 0.07 (s, 6H). ESI-LCMS: m/z 1208 [M+H]⁺.

### Example A24

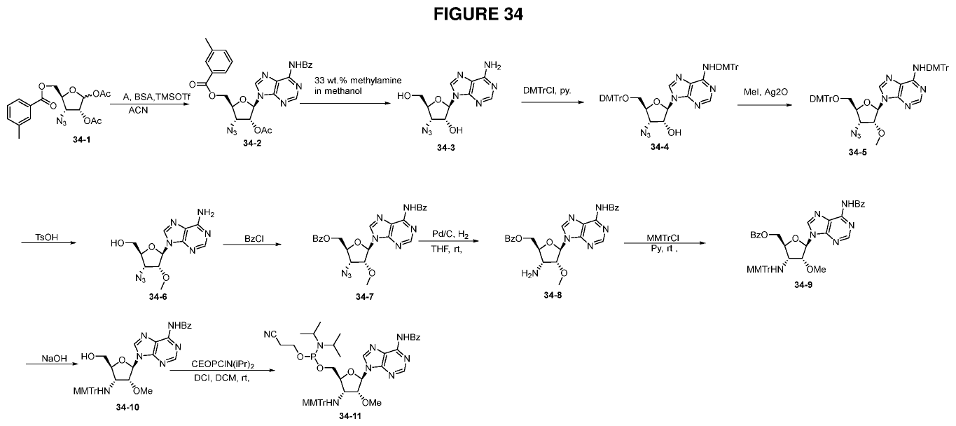

The building block compound 34-11 is useful for making embodiments of modified phosphorothioated oligonucleotides. With reference to FIG. 34, the compound 34-11 (see U.S. Pat. No. 6,608,036) was prepared as follows:

Preparation of compound 34-2: To a solution of compound 34-1 (10.00 g, 26.60 mmol) in dry ACN (100.00 ML) was added N-(5H-purin-6-yl)benzamide (12.73 g, 53.20 mmol) and BSA (22.80 g, 111.80 mmol). The resulting suspension was stirred at 50° C. until clear. Then the mixture was cooled at −20° C. and TMSOTf (11.81 g, 53.20 mmol) was added by syringe. Then the mixture was stirred at 70° C. for 72 hours under N₂, LC-MS showed 34-1 was consumed. The mixture was concentrated, the residue was quenched with sat NaHCO₃ and extracted with DCM. The organic layer was dried over Na₂SO₄, concentrated to give the residue which was purified on silica gel with 1-2% MeOH in DCM to afford compound 34-2 (13.27 g, 23.87 mmol, 90% yield) as a yellow solid. ¹H-NMR (400 MHz, DMSO-d₆): δ=11.28 (s, 1H), 8.64 (d, J=6.4 Hz, 2H), 8.05 (d, J=8.0 Hz, 2H), 7.84 (d, J=8.0 Hz, 2H), 7.66 (t, J=7.6 Hz, 1H), 7.56 (t, J=8.0 Hz, 2H), 7.33 (d, J=8.0 Hz, 2H), 6.37 (d, J=3.6 Hz, 1H), 6.17 (dd, J=6.0 Hz, 1H), 5.09 (t, J=6.8 Hz, 1H), 4.69-4.56 (m, 2H), 4.40-4.38 (m, 1H), 2.39 (s, 3H), 2.17 (s, 3H). ESI-LCMS: m/z 557.2 [M+H]⁺.

Preparation of compound 34-3: A solution of compound 34-2 (13.87 g, 23.87 mmol) in 33 wt. % methylamine in methanol (150.00 ML) was stirred at 20° C. for 16 h, TLC showed 34-2 was consumed. Then solvent was evaporated, washed with 50% EtOAc in petroleum ether (7.00 L), filtered to afford compound 34-3 (6.30 g, 21.58 mmol, 90.10% yield) as a slightly yellow solid. ESI-LCMS: m/z 293.1 [M+H]⁺.

Preparation of compound 34-4: A solution of compound 34-3 (6.30 g, 21.58 mmol) in pyridine (100.00 ML) was added DMTr-Cl (18.26 g, 53.94 mmol) was added, the mixture were stirred at 20° C. for 6 hours under N₂. LC-MS showed 34-3 was consumed, quenched with sat. NaHCO₃ and extracted with DCM. The organic layer was dried over Na₂SO₄ and concentrated to give the crude product which was purified on silica gel with 20-50% EtOAc in petroleum ether to afford compound 34-4 (17.40 g, 19.42 mmol, 90.00% yield) as a slightly yellow solid. ¹H-NMR (400 MHz, DMSO-d₆): δ=8.38 (s, 1H), 7.84 (s, 1H), 7.60-7.55 (m, 4H), 7.46-7.20 (m, 19H), 6.84 (d, J=8.8 Hz, 2H), 6.35 (d, J=5.6 Hz, 1H), 5.95 (d, J=4.4 Hz, 1H), 5.14-5.12 (m, 1H), 4.46 (t, J=5.6 Hz, 2H), 4.11-4.05 (m, 1H), 3.90-3.82 (m, 2H), 3.72 (s, 3H), 0.94 (s, 8H). ESI-LCMS: m/z 897.01 [M+H]⁺.

Preparation of compound 34-5: To a solution of compound 34-4 (17.40 g, 19.42 mmol) in CH₃I (100.00 ML), Ag₂O (4.48 g, 19.42 mmol) was added. The mixture was refluxed at 50° C. for 1 h, TLC showed 34-4 was consumed. Then filtered to get the compound 34-5 (17.67 g) as a yellow solid. ESI-LCMS: m/z 911.4 [M+H]⁺.

Preparation of compound 34-6: A solution of compound 34-5 (17.67 g, 19.42 mmol) was dissolved in DCM (200.00 ML) was added TsOH (5.01 g, 29.13 mmol) in MeOH (40.00 ML), the mixture was stirred at 20° C. for 4 h, LC-MS showed 34-5 was consumed, then washed with saturated NaHCO₃, concentrated to give the crude product which was purified on silica gel with 1-3% MeOH in DCM to afford compound 34-6 (4.70 g, 15.35 mmol, 80.00% yield) as a white solid. ESI-LCMS: m/z 307.3 [M+H]⁺.

Preparation of compound 34-7: To a solution of compound 34-6 (4.70 g, 15.35 mmol) in pyridine (50.00 ML) at 0° C., BzCl (6.52 g, 46.59 mmol) was added by syringe over 15 minutes, then the mixture was allowed to warm up to 20° C. Then stirred at room temperature under N₂ for 1 hours. The solution was cooled to 0° C., and ammonium hydroxide (10 mL, 30%) was added and the mixture was allowed to warm to room temperature and stirred at room temperature for 2 hours. The mixture was diluted with EA and Water, extracted with EA, the combined organic layer was washed with brine, dried over anhydrous Na₂SO₄, concentrated to give the crude product which was purified on silica gel (PE:EA=1:1) to afford compound 34-7 (7.58 g, 14.75 mmol, 95.31% yield) as a white solid. ESI-LCMS: m/z 515.1 [M+H]⁺.

Preparation of compound 34-8: To a solution of compound 34-7 (7.58 g, 14.75 mmol) in THF (100.00 ML), Pd/C (0.75 g) were added, the mixture was stirred at 20° C. for 15 hours under H₂. TLC showed 34-7 was consumed, then filtered and the filtrate concentrated to afford compound 34-8 (7.00 g, 14.45 mmol, 98.01% yield) as a white solid. ESI-LCMS: m/z 489.1 [M+H]⁺.

Preparation of compound 34-9: To a solution of compound 34-8 (7.00 g, 14.45 mmol) in anhydrous pyridine (100.00 ML), MMTr-Cl (5.78 g, 18.78 mmol) were added, the mixture was stirred at 20° C. for 1 hours under N₂. TLC showed 34-8 was consumed, then filtered, washed with H₂O and dried over Na₂SO₄, concentrated to give the residue which was purified on silica gel with 20-50% EA in petroleum ether to afford compound 34-9 (8.78 g, 11.56 mmol, 80% yield) as a white solid. ESI-LCMS: m/z 761.32 [M+H]⁺.

Preparation of compound 34-10: To a solution of compound 34-9 ((8.78 g, 11.56 mmol) in THF (2.00 L), aqueous NaOH (2M) (20.00 mL) was added, the mixture was stirred at 0° C. for 1 hour TLC showed 34-9 was consumed, then washed with saturated NH₄C1, The mixture was diluted with EA and Water, extracted with EA, the combined organic layer was washed with brine, dried over anhydrous Na₂SO₄, concentrated to give the residue which was purified on silica gel with 1-2% MeOH in DCM to afford compound 34-10 (6.07 g, 9.25 mmol, 80% yield) as a white solid.

Preparation of compound 34-11: To a solution of compound 34-10 (6.07 g, 9.25 mmol) in DCM (1.50 L) was added CDI (0.98 g, 8.33 mmol) and CEPCl (3.34 g, 11.10 mmol) was added. The reaction mixture was stirred at room temperature for 1 hours. TLC showed 34-10 was consumed, washed with saturated NaHCO₃ and brine, dried over Na₂SO₄, concentrated to give the crude product which was purified by silica gel column by (PE:EA=4:1˜1:1) and Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 34-11 (6.0 g, 7.12 mmol, 78.53% yield) as a white solid. ¹H-NMR (400 MHz, CDCl₃): δ=8.72 (d, J=11.6 Hz, 1H), 8.51-8.23 (m, 1H), 8.06 (t, J=7.2 Hz, 2H), 7.59 (t, J=7.2 Hz, 1H), 7.54-7.48 (m, 6H), 7.39 (dd, J=8.8 Hz, 2H), 7.19-7.07 (m, 6H), 6.72-6.68 (m, 2H), 6.00 (d, J=7.6 Hz, 1H), 4.75 (s, 1H), 4.36-4.30 (m, 1H), 4.23-4.18 (m, 1H), 4.13-3.98 (m, 2H), 3.84-3.78 (m, 1H), 3.68-3.48 (m, 6H), 3.39-2.30 (m, 1H), 3.17 (d, J=16.4 Hz, 3H), 2.79 (dd, J=18.8 Hz, 1H), 2.66-2.48 (m, 2H), 2.26 (s, 3H), 2.07-1.59 (m, 1H), 1.23-1.14 (m, 12H). ³¹P NMR (CDCl₃): 149.05, 148.25. ESI-LCMS: m/z 855.2 [M−H]⁻.

### Example A25

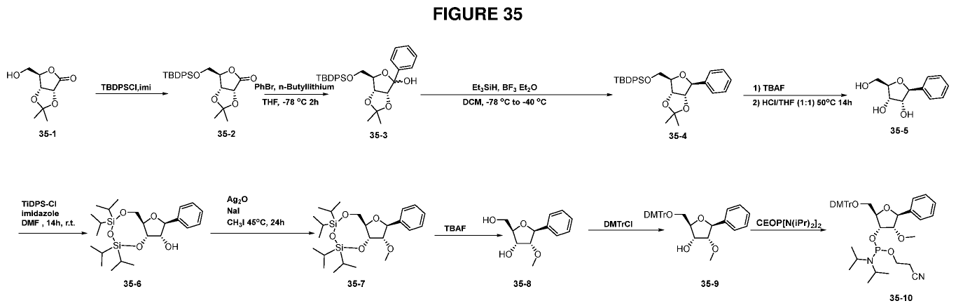

The building block compound 35-10 is useful for making embodiments of modified phosphorothioated oligonucleotides. With reference to FIG. 35, the compound 35-10 (see Taniguchi, Yosuke et al., Tetrahedron, 2013, 69(2), 14, 600-606) was prepared as follows:

Preparation of compound 35-2: To a suspension of 35-1 (50.0 g, 265 mmol) in DCM (265 mL) with an inert atmosphere of nitrogen was added imidazole (45.0 g, 660 mmol) and TBDPSCl (94.7 g, 344 mmol) in order at 0° C. The reaction solution was stirred for 14 hours at room temperature. The solution was diluted with DCM and washed with H₂O, saturated aqueous sodium bicarbonate and washed with brine and dry over by Na₂SO₄. Then the solution was concentrated under reduced pressure and the residue was purified by column chromatography (SiO₂, 0-10% EA-PE). This resulted in 35-2 (100 g, 90% yield) as a white solid; ESI-LCMS: m/z 486 [M+H]⁺.

Preparation of compound 35-3: To a suspension of PhBr (23.5 g, 150 mmol) in THF (200 mL) with an inert atmosphere of nitrogen at −78° C. was added n-buLi (8.32 g, 130 mmol). After stirring for 30 min, solution of 35-2 (42.6 g, 100 mmol) was added dropwise via cannula. The mixture was stirred at −78° C. for 1 hours. LCMS showed 35-2 was consumed completely. The solution was poured into NH₄Cl solution. Then was diluted with EA and washed with sat. aqueous NH₄Cl twice and washed with brine and dry over by Na₂SO₄. Then the solution was concentrated under reduced pressure and the residue was purified by column chromatography (SiO₂, 0-10% EA-PE) to give 35-3 (42.0 g, 715.2 umol, 71.5% yield) as oil. ¹H-NMR (400 MHz, CDCl₃) δ=7.76-7.63 (m, 9H), 7.47-7.33 (m, 14H), 4.96-4.94 (m, 1H), 4.71 (d, J=5.6 Hz, 1H), 4.47 (m, 2H), 4.0-3.97 (m, 2H), 3.82-3.79 (m, 1H), 1.70 (s, 1H), 1.42 (s, 1H), 1.40 (s, 3H), 1.27 (s, 1H), 1.15 (s, 9H), 1.11 (s, 4H). ESI-LCMS: m/z 504 [M+H]⁺.

Preparation of compound 35-4: To a suspension of 35-3 (35.0 g, 69.3 mmol) in DCM (400 mL) and Et₃SiH (9.5 g, 83.2 mmol) was added BF₃.Et₂O (14.7 g, 104.0 mmol) with an inert atmosphere of nitrogen at −78° C. After stirring for 40 minutes at −40° C., The solution was poured into NH₄Cl solution. Then was diluted with EA and washed with sat. aqueous NH₄Cl twice and washed with brine and dry over by Na₂SO₄. Then the solution was concentrated under reduced pressure and the residue was purified by purified by column chromatography (SiO₂, 0-10% EA-PE) to give 35-4 (30.0 g, 61.3 mmol, 70.0% yield) as oil. ¹H-NMR (400 MHz, CDCl₃) δ=7.76-7.71 (m, 4H), 7.47-7.28 (m, 11H), 4.94 (d, J=5.2 Hz, 1H), 4.85-4.82 (dd, 1H), 4.57-4.54 (m, 1H), 4.23 (m, 1H), 3.99-3.88 (m, 2H), 1.65 (s, 3H), 1.38 (s, 3H), 1.09 (s, 9H); ESI-LCMS: m/z 487[M+H]⁺.

Preparation of compound 35-5: To a suspension of 35-4 (30.0 g, 61.3 mmol) in THF (100 mL) and HCl (100 mL, 61.3 mmol) was added. After stirring for 14 hours at 50° C., then added con. NH₄OH to give the pH=7.5 and diluted with EA. Then the solution was concentrated under reduced pressure and the residue was purified by column chromatography (SiO₂, 0-10% MeOH in DCM) to give 35-5 (9.5 g, 42.9 mmol, 69.9% yield) as a white solid. ¹H-NMR (400 MHz, MeOD-d₆) δ=7.47-7.26 (m, 5H), 4.73 (d, 1H), 4.06 (m, 1H), 3.99 (m, 1H), 3.88 (m, 1H), 3.83-3.72 (m, 2H); ESI-LCMS: m/z 211 [M+H]⁺.

Preparation of compound 35-6: To a suspension of 35-5 (9 g, 42.81 mmol) in DMF (90 mL) and imidazole (12.8 g, 188.3 mmol), TIDPS-Cl (14.8 g, 47.0 mmol) was added. Mixture was stirred for 14 hours at room temperature, then diluted with EA and washed with H₂O. Then the solution was concentrated under reduced pressure and the residue was purified by column chromatography (SiO₂, 0-10% EA-PE) to give 35-6 (13.5 g, 28.6 mmol, 66.8% yield) as oil. ¹H-NMR (400 MHz, CDCl₃) δ=7.45-7.27 (m, 5H), 4.86 (d, 1H), 4.40 (m, 1H), 4.16-4.02 (m, 3H), 3.97 (m, 1H), 1.15-1.00 (m, 28H); ESI-LCMS: m/z 455 [M+H]³⁰.

Preparation of compound 35-7: To a suspension of 35-6 (13.5 g, 29.8 mmol) in MeI (12 mL) and Ag₂O (20.73 g, 89.4 mmol), NaI (2.2 g, 14.9 mmol) was added. The mixture was stirred for 24 hours at 45° C., then filtered and the solution was concentrated under reduced pressure and the residue was purified by column chromatography (SiO₂, 0-10% EA in PE) to give 35-7 (6.4 g, 13.4 mmol, 45.0% yield) as oil. ¹H-NMR (400 MHz, CDCl₃) δ=7.45-7.27 (m, 5H), 4.99 (s, 1H), 4.40 (m, 1H), 4.25-4.21 (m, 1H), 4.07-4.03 (m, 2H), 3.61-3.58 (m, 4H), 1.15-1.00 (m, 28H); ESI-LCMS: m/z 467 [M+H]⁺.

Preparation of compound 35-8: To a suspension of 35-7 (6.4 g, 13.7 mmol) in THF (70 mL) and TBAF (3.5 g, 13.7 mmol) was added. The mixture was stirred for 1 hours at 20° C., LCMS showed 35-7 was consumed completely. The solution was diluted with EA (200 mL) and washed with H₂O, then washed with brine and dry over by Na₂SO₄. Then filtered and the solution was concentrated under reduced pressure and the residue was purified by column chromatography (SiO₂, 0-10% MeOH in DCM) to 35-8 (2.9 g, 12.6 mmol, 92.4% yield) as oil. ¹H-NMR (400 MHz, CDCl₃) δ=7.40-7.31 (m, 5H), 4.88 (d, 1H), 4.23 (t, 1H), 4.04-3.95 (m, 2H), 3.84-3.80 (m, 1H), 3.66 (m, 1H), 3.45 (s, 1H); ESI-LCMS: m/z 225 [M+H]⁺.

Preparation of compound 35-9: To a suspension of 35-8 (2.9 g, 12.9 mmol) in pyridine (30 mL) and added DMTr-Cl (4.3 g, 12.9 mmol) in order. The mixture was stirred at room temperature for 3 hours. LCMS showed 35-8 was consumed completely. The solution was diluted with EA (200 mL) and washed with H₂O and sat. aqueous NaHCO₃ twice, then washed with brine and dry over by Na₂SO₄. Then the solution was concentrated under reduced pressure and the residue was purified by column chromatography (DCM:MeOH=20:1). This resulted in 35-9 (5.6 g, 10.4 mmol, 80.6% yield) as a yellow solid. ¹H-NMR (400 MHz, DMSO-d₆) δ=7.45-7.21 (m, 14H), 6.90-6.88 (m, 4H), 4.98 (m, 1H), 4.80 (m, 1H), 4.05-3.99 (m, 2H), 3.74 (s, 6H), 3.51 (m, 1H), 3.24-3.15 (m, 2H); ESI-LCMS: m/z 527 [M+H]⁺.

Preparation of compound 35-10: To a suspension of 35-9 (5.4 g, 10.5 mmol) in DCM (55 mL) and added CEOP[N(iPr)₂]₂ (3.0 g, 10.2 mmol), DCI (1.4 g, 10.2 mmol). The mixture was stirred at room temperature for 1 hours. LCMS showed 35-9 was consumed completely. The solution was diluted with DCM (50 mL) and washed with H₂O and sat. NaHCO₃ twice, then washed with brine and dry over by Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 35-10 (7 g, 9.4 mmol, 92.0% yield) as a white solid. ¹H-NMR (400 MHz, DMSO-d₆) δ=7.45-7.21 (m, 14H), 6.90-6.88 (m, 4H), 4.81 (m, 1H), 4.30-4.14 (m, 2H), 3.82-3.77 (m, 1H), 3.74 (s, 6H), 3.69-3.51 (m, 4H), 3.36-3.27 (m, 4H), 3.22-3.17 (m, 1H) 2.78 (m, 1H), 2.58 (m, 1H), 1.13-0.95 (m, 12H): ³¹P-NMR (DMSO-d₆) δ=149.00, 148.89. ESI-LCMS: m/z 727 [M+H]⁺.

### Example A26

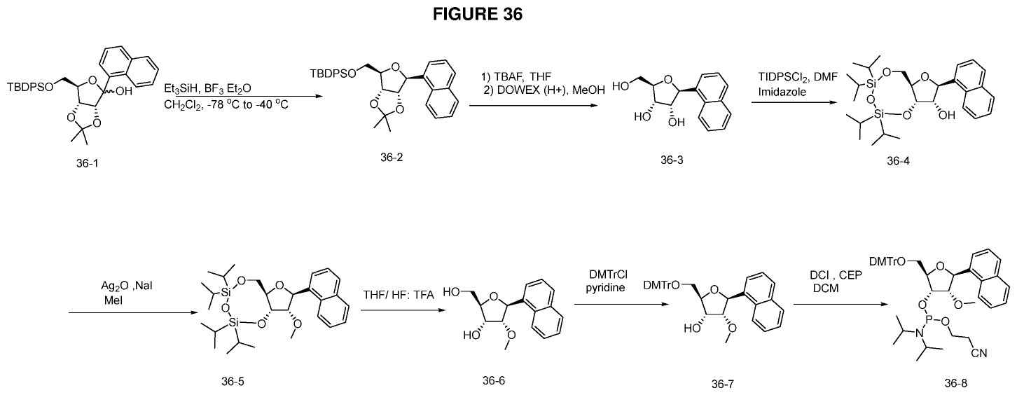

The building block compound 36-8 is useful for making embodiments of modified phosphorothioated oligonucleotides. With reference to FIG. 36, the compound 36-8 was prepared as follows:

Preparation of compound 36-2: To a solution of 36-1 (41.6 g, 75.0 mmol) in DCM (750.0 mL) was added Et₃SiH (10.3 g, 90.0 mmol) at −78° C. under N₂ atmosphere, then trifluoroborane (7.6 g, 112.5 mmol) was added next, the mixture was stirred 1 hr at −40° C. Checking the reaction by LCMS showed the completion of the conversion. The mixture was put in NaHCO₃ aq (1000.0 mL and diluted with DCM (200.0 ml*3), washed with brine (100.0 mL). The organic layer was dried, separated purified by column chromatography (SiO₂, PE/EA=150:1 to 10:1) to give 36-2 (34.5 g, 84.5% yield) as yellow oil. ESI-LCMS: m/z 561.3 [M+Na]⁺.

Preparation of compound 36-3: To a solution of 36-2 (5.0 g, 9.3 mmol) in TFA (10.3 g, 2.5 mL) was added H₂O (0.1 mL) the mixture was stirred at 25° C. for 1 hour Checking the reaction by LCMS showed the completion of the conversion. The mixture was separated to give crude 36-3 (1.0 g) as brownish oil. ESI-LCMS: m/z 283.3 [M+Na]⁺.

Preparation of compound 36-4: To a solution of 36-3 (11.2 g, 43.0 mmol) in TIDPSCl (14.9 g, 47.3 mmol) was added imidazole (10.2 g, 150.6 mmol) at 0° C. under N₂ atmosphere and the mixture was stirred at 0° C. for 1 hour Checking the reaction by LCMS showed the completion of the conversion. MeOH (10.0 mL) and H₂O (200.0 mL) was added. Then diluted with EA (200.0 mL*3), washed with brine (100.0 mL). The organic layer was dried separated. The residue was purified by column chromatography (SiO₂, DCM/PE=1:10 to 10:1) to give 36-4 (21.0 g, 94.0% yield) as colorless oil. ESI-LCMS: m/z 503.2 [M+H]⁺.

Preparation of compound 36-5: To a solution of 36-4 (17.0 g, 33.8 mmol) in MeI (33.8 mmol, 50.0 mL) were added Ag₂O (23.5 g, 101.4 mmol) and NaI (2.5 g, 16.9 mmol). The mixture was stirred at 45° C. for night. Checking the reaction by LCMS showed the completion of the conversion. Filtered the mixture and washed with DCM (100.0 mL*3). The filtrate was concentrated. The residue was purified by column chromatography (SiO₂, PE/EA=50:1 to 10:1) to give 36-5 (17.4 g, 97.1% purity, 70.0% yield) as white solid. ¹H NMR (400 MHz, DMSO-d₆) δ 7.99 (s, 0.5H), 7.97 (s, 0.5H), 7.29-7.33 (m, 2H), 7.85-7.91 (m, 2H), 7.80-7.82 (d, J=6.8 Hz 1H), 7.61-7.65 (m, 1H), 7.55-7.59 (m, 1H), 4.24-4.31 (m, 2H), 3.97-4.05 (m, 2H), 3.66 (d, J=4.2 Hz 1H), 3.62 (s, 3H), 0.84-1.09 (m, 42H). ESI-LCMS: m/z 517.1 [M+H]⁺.

Preparation of compound 36-6: To a solution of 36-5 (15.3 g, 29.6 mmol) in THF (150.0 mL) was added HF/THF (23.5 g, 88.8 mmol, 14.3 mL) and the mixture was stirred 2 hr at 25° C. Checking the reaction by LCMS showed the completion of the conversion. The mixture was extract with EA (100.0 mL*3) washed with NaCl aqueous (50.0 mL). The residue purified by column chromatography (SiO₂, MeOH/DCM=1:100) to give 36-6 (5.8 g, 71.2% yield) as colorless oil. ¹H NMR (400 MHz, DMSO-d₆) δ 8.1 (d, J=8.0 Hz 1H), 7.693-7.96 (m, 1H), 7.85 (d, J=8.0 Hz 1H), 7.76 (d, J=8.0 Hz 1H), 7.48-7.58 (m, 3H), 5.46 (d, J=4.8 Hz 1H), 5.03 (d, J=6.0 Hz 1H), 4.89 (s, 1H), 4.10-4.14 (m, 1H), 3.91-3.14 (m, 1H), 3.60-3.71 (m, 3H), 3.36 (s, 3H). ESI-LCMS: m/z 297.3 [M+Na]⁺.

Preparation of compound 36-7: To a solution of 36-6 (5.5 g, 20.0 mmol) in pyridine (55.0 mL) was added DMTrCl (7.1 g, 21.1 mmol, 14.3 mL) at 0° C. under N₂ atmosphere and then the mixture was stirred for 1 h at 25° C. Checking the reaction by LCMS showed the completion of the conversion. MeOH (5.0 mL) was added and extract with EA (100.0 mL) washed with NaCl aqueous (50.0 mL*3). The residue purified by column chromatography (SiO₂, MeOH/DCM=1:100) to give 36-7 (10.0 g, 86.2% yield) as white solid. ¹H NMR (400 MHz, DMSO-d₆) δ 8.14-8.16 (m, 1H), 7.96-7.98 (m, 1H), 7.87 (d, J=8.0 Hz 1H), 7.74 (d, J=8.0 Hz 1H), 7.53-7.57 (m, 3H), 7.40-7.47 (m, 3H), 7.22-7.25 (m, 1H), 6.86-6.89 (m, 4H), 5.53 (d, J=4.0 Hz 1H), 5.10 (d, J=4.0 Hz 1H), 4.06-4.17 (m, 2H), 3.71-3.74 (m, 7H) 3.42 (s, 3H), 3.23-3.30 (m, 2H). ESI-LCMS: m/z 577.6 [M+H]⁺.

Preparation of compound 36-8: To a solution of 7 (9.5 g, 16.5 mmol) in DCM (95.0 mL) was added DCI (1.7 g, 14.8 mmol) at 25° C. under N₂ atmosphere and then CEOP[N(iPr)₂]₂ (5.4 g, 18.1 mmol, 14.3 mL) was added the mixture was stirred for 1 h at 25° C. Checking the reaction by LCMS showed the completion of the conversion. H₂O (20.0 mL) was added and extract with DCM (100.0 mL*3) washed with NaCl aqueous (50.0 mL). Concentrate the organic, the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm to give 36-8 (11.0 g, 84.4% yield) as white solid. ¹H-NMR (400 MHz, DMSO-d₆): δ ppm 8.17-8.21 (m, 1H), 7.95-7.97 (m, 1H), 7.88 (d, J=4.0 Hz, 1H), 7.77-7.83 (m, 1H), 7.51-7.55 (m, 2H), 7.43-7.49 (m, 3H), 7.26-7.36 (m, 6H), 7.20-7.25 (m, 1H), 6.84-6.89 (m, 4H), 5.60-5.63 (m, 1H), 4.37-4.45 (m, 1H), 4.22-4.29 (m, 1H), 3.90-3.94 (m, 1H), 3.69-3.82 (m, 7H), 3.49-3.62 (m, 3H), 3.39-3.43 (m, 3H), 3.24-3.30 (m, 1H), 2.73-2.76 (m, 1H), 2.55-2.58 (m, 1H), 1.08-1.12 (m, 8H), 0.96 (d, J=6.0 Hz, 1H). ³¹P NMR (DMSO-d₆): 149.14, 148.77. ESI-LCMS: m/z 777.6 [M+H]⁺.

### Example A27

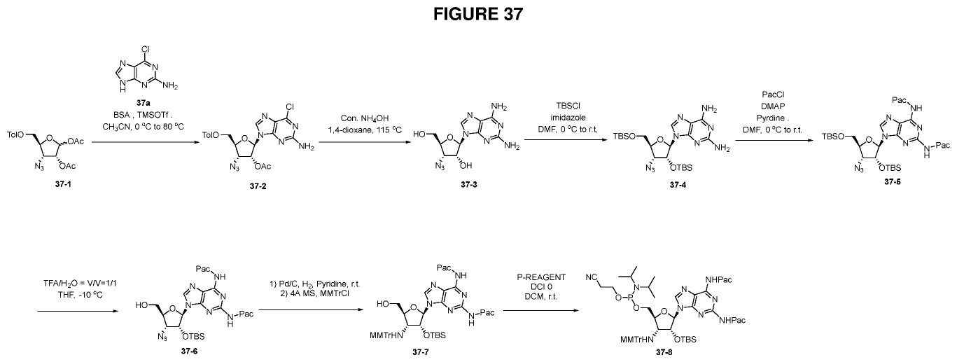

The building block compound 37-8 is useful for making embodiments of modified phosphorothioated oligonucleotides. With reference to FIG. 37, the compound 37-8 (see Matray, T. et. al, Nucleosides, Nucleotides and Nucleic Acids, 2000, 19(10-12), 1553-1567) was prepared as follows:

Preparation of compound 37-2: To a suspension of 37-1 (20 g, 53.03 mmol) and 37a (13.49 g, 79.54 mmol) in CH₃CN (250 mL) was added BSA (34.53 g, 169.61 mmol) and heated to 50° C. and stirred at this temperature for 1.5 hours until a clear solution obtained. The mixture was cooled to room temperature and ice-cooled to 0° C., then TMSOTf (14.13 g, 63.60 mmol) was added dropwise slowly to the mixture within 15 min. The reaction was heated to 78° C. and stirred at this temperature for 12 hours until major desired product was detected by TLC and LC-MS. The reaction was cooled to room temperature and quenched with sat. aqueous NaHCO₃ (300 mL) and filtered through celite cake and the filtrate was extracted with EA (300 mL*3). The combined organic layers were washed with water (300 mL*2), brine (500 mL), dried over anhydrous Na₂SO₄ and evaporated in the vacuo to give crude product. The crude was purified by column chromatography with a gradient of 20 to 50% EtOAc in PE to give 37-2 (23.2 g, 47.62 mmol, 89.8% yield) as a white solid. ¹H NMR (400 MHz, DMSO-d₆) δ 8.27 (s, 1H), 7.81 (d, J=8.0 Hz, 2H), 7.29 (d, J=8.0 Hz, 2H), 7.16 (s, 2H), 6.14 (d, J=2.5 Hz, 1H), 5.95 (dd, J=5.6, 2.7 Hz, 1H), 5.16 (dd, J=7.5, 6.3 Hz, 1H), 4.68 (dd, J=12.3, 3.0 Hz, 1H), 4.49 (dd, J=12.3, 4.9 Hz, 1H), 4.32 (dd, J=7.4, 3.7 Hz, 1H), 2.37 (s, 3H), 2.17 (s, 3H). ESI-MS: m/z 487.2 [M+H]⁺.

Preparation of compound 37-3: To a solution of 37-2 (23.2 g, 47.62 mmol) in 1,4-dioxane (200 mL) was added Con. NH₄OH (300 mL) at room temperature in a autoclave and the mixture was heated to 115° C. and stirred at this temperature for 16 hours until 37-2 was consumed and major desired product was detected by TLC and LC-MS. The reaction was cooled to room temperature and the solvent was removed in the vacuo to give crude product, the crude product was purified by recrystallization (DCM/PE/EtOAc) to give 37-3 (12.4 g, 40.38 mmol, 84.8% yield) as a white solid. ¹H NMR (400 MHz, DMSO-d₆) δ 7.94 (s, 1H), 6.82 (s, 2H), 6.17 (d, J=5.6 Hz, 1H), 5.88-5.70 (m, 3H), 5.61 (dd, J=7.1, 4.6 Hz, 1H), 4.92 (dd, J=11.6, 5.7 Hz, 1H), 4.28 (dd, J=5.5, 3.4 Hz, 1H), 3.92 (q, J=3.5 Hz, 1H), 3.66 (dt, J=12.1, 4.1 Hz, 1H), 3.61-3.50 (m, 1H). ESI-MS: m/z 308.1 [M+H]⁺.

Preparation of compound 37-4: To a solution of 37-3 (12.4 g, 40.38 mmol) in DMF (150 mL) was ice-cooled to 0° C. and added imidazole (22.0 g, 323.04 mmol) and stirred at this temperature for 20 min. Then TBSCl (24.3 g, 161.52 mmol) was added slowly to the mixture and warmed to room temperature and stirred at room temperature overnight until 37-3 was consumed and major desired product was detected by TLC and LC-MS. The reaction was quenched with water (300 mL) and extracted with EA (200 mL*3), combined organic layers was washed with water (300 mL*2), brine (500 mL), dried over anhydrous Na₂SO₄ and evaporated in the vacuo to give crude product. The crude product was purified by column chromatography with a gradient of 10 to 60% EtOAc in PE to give 37-4 (20.2 g, 37.73 mmol, 93.4% yield) as a white solid. ¹H NMR (400 MHz, DMSO-d₆) δ 7.85 (s, 1H), 6.77 (s, 2H), 5.90-5.70 (m, 3H), 4.90 (t, J=5.6 Hz, 1H), 4.33 (dd, J=5.3, 3.6 Hz, 1H), 4.01 (q, J=3.5 Hz, 1H), 3.84 (t, J=3.5 Hz, 2H), 0.97-0.87 (m, 9H), 0.79 (s, 9H), 0.10 (d, J=1.8 Hz, 6H), −0.02 (s, 3H), −0.17 (s, 3H). ESI-MS: m/z 536.4 [M+H]⁺.

Preparation of compound 37-5: To a solution of 37-4 (14.0 g, 26.15 mmol) in DMF (140 mL) was added DMAP (12.78 mmol, 104.6 mmol) and pyridine (8.3 g, 104.6 mmol), then the mixture was ice-cooled to 0° C. and stirred at this temperature for 30 min. Then phenoxyacetyl chloride (17.8 g, 104.6 mmol) was dropwise slowly to the mixture within 10 min, the mixture was warmed to room temperature and stirred overnight until 37-4 was consumed and major desired product was detected by TLC and LC-MS. The reaction was poured into water (200 mL) and extracted with EA (150 Ml*3), combined organic layers were washed with water (300 mL*2), brine (500 mL), dried over anhydrous Na₂SO₄ and evaporated in the vacuo to give crude product. The crude product was purified by column chromatography with a gradient of 10 to 50% EtOAc in PE to give 37-5 (12.0 g, 14.93 mmol, 57.1% yield) as a light yellow solid. ESI-MS: m/z 804.5 [M+H]⁺.

Preparation of compound 37-6: To a solution of 37-5 (12.0 g, 14.93 mmol) in THF (120 mL) was cooled to −10° C. in an ice salt bath and stirred at this temperature for 20 min, then a mixture of TFA (120 mL) and water (120 mL) was dropwise slowly to the mixture within 1 hours. The mixture was stirred at this temperature for 3 hours until 37-5 was consumed and major desired product was detected by TLC and LC-MS. The reaction was quenched with NH₄OH in an ice salt bath until pH was 7-8, the mixture was extracted with EA (200 mL*3), combined organic layers was washed with water (200 mL*2), brine (500 mL), dried over anhydrous Na₂SO₄ and evaporated in the vacuo to give crude product. The crude product was purified by column chromatography with a gradient of 20 to 60% EtOAc in PE to give 37-6 (5.2 g, 7.54 mmol, 50.5% yield) as a white solid. ¹H NMR (400 MHz, DMSO-d₆) δ 11.02 (s, 1H), 10.73 (s, 1H), 8.63 (s, 1H), 7.41-7.20 (m, 5H), 6.95 (dt, J=7.3, 5.7 Hz, 7H), 5.98 (d, J=5.6 Hz, 1H), 5.28-5.15 (m, 4H), 5.08 (s, 2H), 4.59-4.38 (m, 1H), 4.03 (dd, J=8.3, 4.3 Hz, 1H), 3.75 (dt, J=9.7, 4.8 Hz, 1H), 3.70-3.59 (m, 1H), 0.77 (d, J=8.4 Hz, 9H), 0.00 (s, 3H), −0.21 (s, 3H). ESI-MS: m/z 690.3 [M+H]⁺.

Preparation of compound 37-7: To a solution of 37-6 (5.2 g, 7.54 mmol) in pyridine (320 mL), then Pd/C (1.04 g of 10 percent Pd) was added and the mixture was hydrogenated under hydrogen balloon for 4 hours. The catalyst was removed by filtration through celite, washed with pyridine (80 mL) and the filtrate was combined, then 4 A MS (30 g, dried at 600° C. for 4 hours before used) was added to the solution. The mixture was stirred at room temperature for 1 hours and MMTrCl (2.91 g, 9.43 mmol) was added to the solution and stirred at room temperature overnight. The reaction was quenched with MeOH (10 mL) and water (200 mL), the mixture was extracted with EA (300 mL*2), combined organic layers was washed with water (200 mL*2), brine (500 mL), dried over anhydrous Na₂SO₄ and evaporated in the vacuo to give crude product. The crude product was purified by column chromatography with a gradient of 20 to 60% EtOAc in PE to give 37-7 (4.9 g, 5.24 mmol, 69.5% yield) as a white solid. ESI-MS: m/z 936.5 [M+H]⁺.

Preparation of compound 37-8: To a solution of 37-7 (4.7 g, 5.24 mmol) in 100 mL of dichloromethane with an inert atmosphere of nitrogen was added CEOP[N(iPr)₂]₂ (2.05 g, 6.81 mmol) and DCI (0.557 g, 4.72 mmol) in order at room temperature. The resulting solution was stirred for 1.5 hours at room temperature and diluted with 100 mL dichloromethane and washed with saturated aqueous sodium bicarbonate (100 mL), water (200 mL*2), brine (200 mL), dried over anhydrous Na₂SO₄, evaporated in the vacuo to give crude product. The crude product was purified by repeated Flash-Prep-HPLC with the following conditions (IntelFlash-1): Column, C18 silica gel; mobile phase, CH₃CN/H₂O (0.5% NH₄HCO₃)=10/7 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 37-8 (3.6 g, 3.17 mmol, 60.5% yield) as white solid. ¹H NMR (400 MHz, DMSO-d₆) δ 10.99 (s, 1H), 10.68 (s, 0.5H), 10.40 (s, 0.5H), 8.51 (s, 0.5H), 8.40 (s, 0.5H), 7.52-7.44 (m, 4H), 7.36-7.19 (m, 12H), 7.15-7.10 (m, 1H), 7.00-6.92 (m, 6H), 6.86 (d, J=9.0 Hz, 1H), 6.73 (d, J=8.9 Hz, 1H), 6.31 (d, J=5.9 Hz, 0.5H), 6.01 (d, J=2.4 Hz, 0.5H), 5.21 (d, J=3.1 Hz, 2H), 5.11 (d, J=2.4 Hz, 1H), 5.06 (d, J=2.7 Hz, 1H), 4.09-4.00 (m, 1H), 3.78-3.88 (m, 1H), 3.75-3.56 (m, 4H), 3.53-3.36 (m, 3H), 3.22-3.14 (m, 1H), 2.98-2.95 (m, 1H), 2.80-2.67 (m, 2H), 1.11-1.04 (m, 10H), 0.95 (d, J=6.8 Hz, 3H), 0.77 (d, J=22.4 Hz, 9H), −0.08 (J=8.2 Hz, 3H), −0.15 (s, 3H). ³¹P NMR (DMSO-d₆) δ 148.20, 148.00; ESI-MS: m/z 1136.6 [M+H]⁺.

### Example A28

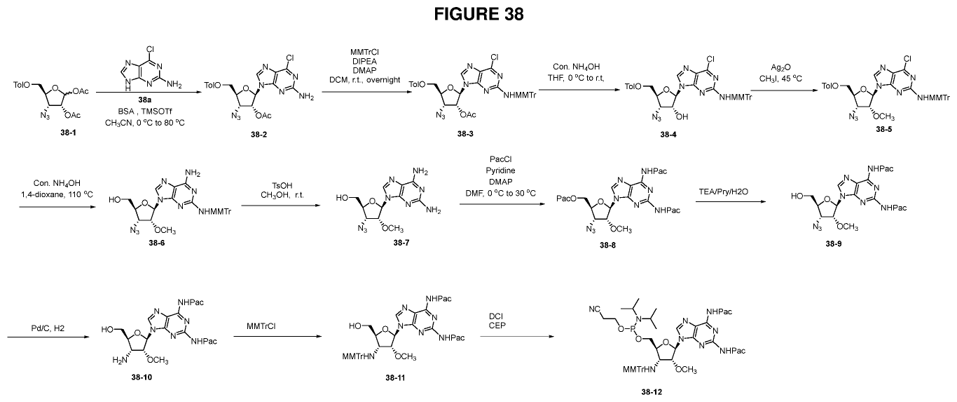

The building block compound 38-12 is useful for making embodiments of modified phosphorothioated oligonucleotides. With reference to FIG. 38, the compound 38-12 was prepared as follows:

Preparation of compound 38-2: To a suspension of 38-1 (25.0 g, 66.29 mmol) and 38a (16.86 g, 99.43 mmol) in CH₃CN (300 mL) was added BSA (43.16 g, 212.01 mmol) and heated to 50° C. and stirred at this temperature for 1.5 hours until a clear solution obtained. The mixture was cooled to room temperature and ice-cooled to 0° C., then TMSOTf (17.66 g, 79.50 mmol) was dropwise slowly to the mixture within 15 min. The reaction was heated to 78° C. and stirred at this temperature for 12 hours until major desired product was detected by TLC and LC-MS. The reaction was cooled to room temperature and quenched with sat. aqueous NaHCO₃ (300 mL) and filtered through celite cake and the filtrate was extracted with EA (300 mL*3). The combined organic layers were washed with water (300 mL*2), brine (500 mL), dried over anhydrous Na₂SO₄ and evaporated in the vacuo to give crude product. The crude was purified by column chromatography with a gradient of 20 to 50% EtOAc in PE to give 38-2 (29.4 g, 60.39 mmol, 91.2% yield) as a white solid. ¹H NMR (400 MHz, DMSO-d₆) δ 8.27 (s, 1H), 7.81 (d, J=8.0 Hz, 2H), 7.29 (d, J=8.0 Hz, 2H), 7.16 (s, 2H), 6.14 (d, J=2.5 Hz, 1H), 5.95 (dd, J=5.6, 2.7 Hz, 1H), 5.16 (dd, J=7.5, 6.3 Hz, 1H), 4.68 (dd, J=12.3, 3.0 Hz, 1H), 4.49 (dd, J=12.3, 4.9 Hz, 1H), 4.32 (dd, J=7.4, 3.7 Hz, 1H), 2.37 (s, 3H), 2.17 (s, 3H); ESI-MS: m/z 487.2 [M+H]⁺.

Preparation of compound 38-3: To a solution of 38-2 (29.4 g, 60.39 mmol) in DCM (300 mL) was added DIPEA (31.2 g, 241.56 mmol), DMAP (781.9 mg, 6.4 mmol) and stirred at this temperature for 15 min, then MMTrCl (37.3 g, 120.78 mmol) was added to the mixture and stirred for overnight until starting material 38-2 was consumed and major desired product was detected by TLC and LC-MS. The reaction was quenched with water (200 mL), extracted with DCM (200 mL*2), combined organic layers was washed with water, brine, dried over anhydrous and evaporated in the vacuo to give crude product. The crude product was purified by column chromatography with a gradient of 10 to 35% EtOAc in PE to give 38-3 (41.7 g, 54.98 mmol, 91.0% yield) as a light yellow solid. ¹H NMR (400 MHz, DMSO-d₆) δ 8.22 (d, J=15.9 Hz, 2H), 7.77 (d, J=8.2 Hz, 2H), 7.40-7.21 (m, 12H), 7.21-7.07 (m, 2H), 6.82 (d, J=8.9 Hz, 2H), 5.76 (s, 1H), 5.32 (s, 1H), 4.42 (s, 1H), 4.25 (d, J=31.1 Hz, 2H), 3.69 (s, 3H), 2.39 (s, 3H), 2.11 (s, 3H); ESI-MS: m/z 759.4 [M+H]⁺.

Preparation of compound 38-4: To a solution of 38-3 (41.7 g, 54.98 mmol) in THF (500 mL) was ice-cooled to 0° C., then Con. NH₄OH (130 mL) was dropwise slowly to the mixture and warmed to room temperature and stirred at this temperature for 48 hours until 38-3 was consumed and major desired product 38-4 was detected by TLC and LC-MS. The reaction was quenched with sat.aq. NH₄Cl (500 mL) and extracted with EA (300 mL*2), combined organic layers was washed with (300 mL*2), brine (500 mL), dried over anhydrous Na₂SO₄ and evaporated in the vacuo to give crude product. The crude product was purified by column chromatography with a gradient of 10 to 40% EtOAc in PE to give 38-4 (31.1 g, 43.35 mmol, 78.8% yield) as a light yellow solid. ¹H NMR (400 MHz, DMSO-d₆) δ 8.26 (s, 1H), 8.17 (s, 1H), 7.78 (d, J=8.2 Hz, 2H), 7.29 (ddd, J=13.2, 10.2, 5.9 Hz, 12H), 7.21-7.05 (m, 2H), 6.82 (d, J=8.9 Hz, 2H), 6.14 (s, 1H), 5.62 (s, 1H), 4.46-4.11 (m, 3H), 3.69 (s, 3H), 2.38 (s, 3H); ESI-MS: m/z 717.4 [M+H]⁺.

Preparation of compound 38-5: To a solution of 38-4 (31.1 g, 43.35 mmol) in CH₃I (250 mL) was added Ag₂O (20.09 g, 86.7 mmol) at room temperature and the mixture was heated to 45° C. and stirred at this temperature for 4 hours until 38-4 was consumed and major desired product was detected by TLC and LC-MS. The reaction was recovered to room temperature and filtered through celite cake and washed with EA (100 mL), combined filtrate was evaporated in the vacuo to give crude product, which was purified by column chromatography with a gradient of 10 to 35% EtOAc in PE to give 38-5 (26.5 g, 36.28 mmol, 84% yield) as a white solid. ¹H NMR (400 MHz, DMSO-d₆) δ 8.22 (s, 2H), 7.72 (d, J=8.1 Hz, 2H), 7.38-7.20 (m, 13H), 6.83 (d, J=8.9 Hz, 2H), 5.73 (s, 1H), 4.37 (s, 1H), 4.21 (s, 3H), 3.70 (d, J=10.9 Hz, 3H), 3.14 (s, 3H), 2.38 (s, 3H); ESI-MS: m/z 731.4 [M+H]⁺.

Preparation of compound 38-6: To a solution of 38-5 (26.5 g, 36.28 mmol) in 1,4-dioxane (200 mL) was added Con. NH₄OH (300 mL) at room temperature in a autoclave and the mixture was heated to 110° C. and stirred at this temperature for 16 hours until 38-5 was consumed and major desired product was detected by TLC and LC-MS. The reaction was cooled to room temperature and the solvent was removed in the vacuo to give crude product, which was purified by column chromatography with a gradient of 0 to 5% CH₃OH in DCM to give 38-6 (18.6 g, 31.34 mmol, 86.4% yield) as a light yellow solid. ESI-MS: m/z 594.4 [M+H]⁺.

Preparation of compound 38-7: To a solution of 38-6 (18.6 g, 31.34 mmol) in CH₃OH (200 mL) was ice-cooled to 0° C. and stirred at this temperature for 30 min, then a solution TsOH (6.48 g, 37.61 mmol) in CH₃OH (50 mL) was dropwise slowly to the mixture within 15 min. Then the reaction was warmed to room temperature and stirred at this temperature for 3 hours until 38-6 was consumed and major desired product was detected by TLC and LC-MS. The reaction was quenched with pyridine (20 mL) and the solvent was removed in the vacuo to give crude product, which was purified by column chromatography with a gradient of 0 to 12.5% CH₃OH in DCM to give 38-7 (8.4 g, 26.17 mmol, 83.5% yield) as a light yellow solid. ¹H NMR (400 MHz, DMSO-d₆) δ 8.03 (s, 1H), 6.85 (s, 2H), 6.06-5.79 (m, 3H), 5.59 (s, 1H), 4.63 (dt, J=8.6, 5.2 Hz, 2H), 3.99 (d, J=3.4 Hz, 1H), 3.62 (ddd, J=33.9, 12.1, 3.4 Hz, 2H), 3.39 (s, 3H). ESI-MS: m/z 322.1 [M+H]⁺.

Preparation of compound 38-8: To a solution of 38-7 (7.3 g, 22.7 mmol) in DMF (200 mL), pyridine (18.0 g, 227.4 mmol) and DMAP (11.1 g, 90.96 mmol) was added, followed by phenoxyacetyl chloride (19.33 g, 113.7 mmol) was added dropwise at 0° C. Then the reaction was warmed to 30° C. for 3 h, LCMS showed 38-7 was consumed completely, 100.0 mL H₂O was added and stirred for 1 h, extracted with EA (100.0 mL*2), organic phase was washed with citric acid and concentrated to give crude which was purified by column chromatography (SiO₂, PE/EA=5:1 to 1:2) to give 38-8 (12.0 g, 97% purity) as a white solid. ESI-LCMS: m/z 724.3 [M+H]⁺; ¹H NMR (400 MHz, DMSO-d₆) δ 11.04 (s, 1H), 10.79 (s, 1H), 8.58 (s, 1H), 7.31-7.28 (m, 7H), 6.98-6.86 (m, 9H), 6.17-6.16 (d, J=4.0 Hz, 1H), 5.16 (s, 2H), 5.07 (s, 2H), 5.03-4.99 (m, 1H), 4.82-4.67 (m, 4H), 4.50-4.38 (m, 2H), 4.29-4.25 (m, 1H), 3.33 (s, 3H).

Preparation of compound 38-9: To a solution of 38-8 (10.0 g, 13.8 mmol) was added in mixture of TEA/pyridine/H₂O=1:1:2 (600 mL). Then the reaction was stirred at room temperature for 40 min, 100.0 mL citric acid was added to change pH to 7-8, extracted with EA (100.0 mL*2), organic phase was washed with brine and concentrated to give crude which was purified by column chromatography (SiO₂, DCM/MeOH=25:1 to 10:1) to give 38-9 (3.5 g, 97% purity) as a white solid. ESI-LCMS: m/z 590.3 [M+H]⁺. ¹H NMR (400 MHz, DMSO-d₆) δ 11.01 (s, 1H), 10.75 (s, 1H), 8.61 (s, 1H), 7.30-7.25 (m, 4H), 6.98-6.92 (m, 6H), 6.10-6.09 (d, J=4.0 Hz, 1H), 5.22-5.09 (m, 5H), 4.75-4.67 (m, 2H), 4.07-4.02 (m, 1H), 3.74-3.59 (m, 2H), 3.47 (s, 3H).

Preparation of compound 38-10: To a solution of 38-9 (2.8 g, 4.7 mmol) in Pyridine (170.0 mL), 10% Pd/C (840 mg) was added under H₂, the reaction was stirred at room temperature for 2 h, LCMS showed 38-9 was consumed completely, filtered and filter cake was washed with pyridine (110.0 mL), the filtrate was used next step directly.

Preparation of compound 38-11: 4A molecular sieves (28 g) was added to the 38-10 in solution and stirred at room temperature for 10 min, MMTrCl (1.7 g, 5.704 mmol) was added, mixture was stirred at room temperature for 15 h, LCMS showed 38-10 was consumed completely, filtered and filter cake was washed with DCM (110.0 mL), organic phase was concentrated to give crude which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=4/1; Detector, UV 254 nm. This resulted in 38-11 (3.5 g, 98% purity) as an oil. ESI-LCMS: m/z 836.2 [M+H]⁺; ¹H NMR (400 MHz, DMSO-d₆) δ 10.89 (s, 1H), 10.55 (s, 1H), 8.49 (s, 1H), 7.44-7.41 (m, 4H), 7.30-7.09 (m, 12H), 6.99-6.92 (m, 4H), 6.72-6.70 (d, J=8.0 Hz, 2H), 5.93 (s, 1H), 5.19-5.03 (m, 5H), 4.07-3.98 (m, 2H), 3.63 (s, 3H), 3.27-3.21 (m, 1H), 3.13 (s, 3H), 2.64-2.61 (m, 1H), 1.61-1.60 (m, 1H).

Preparation of compound 38-12: To a solution of 38-11 (5.0 g, 5.9 mmol) in dichloromethane (50.0 mL) with an inert atmosphere of nitrogen was added CEOP[N(iPr)₂]₂ (2.1 g, 7.1 mmol) and DCI (636 mg, 5.3 mmol) in order at room temperature. The resulting solution was stirred for 1.0 hours at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in 38-12 (12.8 g, 93% yield) as an oil. ESI-LCMS: m/z 1036.2 [M+H]⁺; ¹H-NMR (400 MHz, DMSO-d₆) δ=10.91 (s, 1H), 10.56-10.53 (d, J=12.0 Hz, 1H), 8.25-8.21 (d, J=16.0 Hz, 1H), 7.49-7.44 (m, 4H), 7.34-7.09 (m, 12H), 6.99-6.92 (m, 6H), 6.76-6.69 (m, 2H), 5.96-5.93 (d, J=12.0 Hz, 1H), 5.19-5.03 (m, 4H), 4.25-3.98 (m, 3H), 3.70-3.50 (m, 6H), 3.49-3.38 (m, 1H), 3.30-3.20 (m, 1H), 3.17-3.10 (m, 3H), 2.86-2.72 (m, 3H), 1.87-1.36 (m, 1H), 1.17-1.05 (m, 12H), ³¹P-NMR (DMSO-d₆) δ=148.02, 146.65.

### Example A29

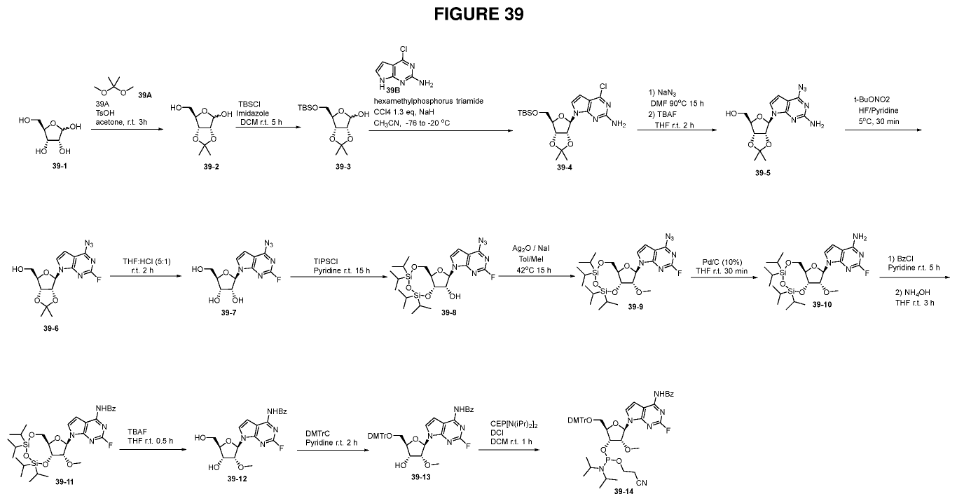

The building block compound 39-14 is useful for making embodiments of modified phosphorothioated oligonucleotides. With reference to FIG. 39, the compound 39-14 was prepared as follows:

Preparation of compound 39-2: To a solution of 39-1 (200.0 g, 1.3 mol) in acetone (1.5 L) was added TsOH (20.6 g, 81.3 mmol) and 39A (166.3 g, 1.6 mmol). Then the reaction mixture was stirred at room temperature for 3 hours. TLC showed 39-1 was consumed. The reaction was quenched with 50 g of NaHCO₃. Then the suspension was filtered and the combined filtrate was concentrated to give the crude 39-2 (240 g) which was used directly for the next step. ESI-LCMS: m/z 213 [M+Na]⁺.

Preparation of compound 39-3: To a solution of crude 39-2 (240 g, 1.3 mol) in DCM (1.8 L) was added imidazole (300.3 g, 4.4 mol) and TBSCl (292.0 g, 2.0 mol) at room temperature TLC showed 39-2 was consumed. Water (3 L) was added and the product was extracted with DCM. The organic phase was washed with brine (500 mL), dried over Na₂SO₄ and concentrated to give the crude. The crude was purified by silica gel column (SiO₂, PE:EA=100:1˜50:1) to give 39-3 (160.0 g, 525.5 mmol, 40.1% yield over 2 steps). ¹H NMR (400 MHz, DMSO-d₆) δ=6.44 (d, J=4.5, 1H), 5.18 (d, J=4.5, 1H), 4.63 (d, J=6.0, 1H), 4.46 (d, J=6.0 Hz, 1H), 3.96-3.93 (m, 1H), 3.59 (d, J=6.8, 2H), 1.37 (s, 3H), 1.25 (s, 3H), 0.88 (s, 9H), 0.06 (s, 6H). ESI-LCMS: m/z 305 [M+H]⁺.

Preparation of compound 39-4: To a solution of 39-3 (55.0 g, 180.6 mmol) in dry THF (550 mL) and carbon tetrachloride (36.6 g, 238.4 mmol) was added P(NMe₂)₃ (36.8 g, 225.8 mmol) slowly at −78° C. over 30 min. Then the mixture was stirred at −20° C. for 1 hours. To resultant, a freshly prepared suspension of 39B (60.9 g, 361.3 mmol) in anhydrous ACN (4 L)/NaH (9.9 g, 415.5 mmol) was slowly added at room temperature over 30 min. The final reaction mixture was stirred at room temperature for 15 hours. LCMS showed 39-3 was consumed. Water was added and the product was extracted with EA. The organic layer was washed with brine and dried over Na₂SO₄ and concentrated to give the crude. The crude was purified by silica gel column (SiO₂, PE:EA=7:1˜3:1) to give 39-4 (50.0 g, 109.8 mmol, 60.8% yield) as a yellow oil. ¹H NMR (400 MHz, DMSO-d₆) δ=7.30 (d, J=4.5, 1H), 6.79 (s, 2H, exchanged with D₂O), 6.37 (d, J=4.5, 1H), 6.12 (d, J=2.6, 1H), 5.18-5.15 (m, 1H), 4.97-4.95 (m, 1H), 4.10-4.04 (m, 1H), 3.71-3.69 (m, 2H), 1.52 (s, 3H), 1.32 (s, 3H), 0.82 (s, 9H), −0.03 (s, 6H). ESI-LCMS: m/z 305 [M+H]⁺. ESI-LCMS: m/z 455 [M+H]⁺.

Preparation of compound 39-5: To a solution of 39-4 (see Ramasamy, Kandasamy et al., Journal of Heterocyclic Chemistry, 1988, 25(6), 1893-1898) (40.0 g, 87.9 mmol) in DMF (300 mL) was added NaN₃ (17.1 g, 263.7 mmol). The reaction mixture was stirred at 90° C. for 15 hours. LCMS showed 39-4 was consumed completely. Water was added and the product was extracted with EA. The organic layer was washed with brine and dried over Na₂SO₄ and concentrated to give the crude. The crude was dissolved in THF (300 mL) and 1M TBAF (90 mL, 90.0 mmol) was added. The combined mixture was stirred at room temperature for 2 hours. Water was added and the product was extracted with EA. The organic layer was washed with brine and dried over Na₂SO₄ and concentrated to give the crude. The crude was purified by silica gel column (SiO₂, PE:EA=5:1˜3:1˜1:1) to give 39-5 (20.0 g, 57.5 mmol, 65.5% yield) as a white solid. ¹H NMR (400 MHz, DMSO-d₆) δ=8.22 (s, 2H, exchanged with D₂O), 7.44 (d, J=3.6, 1H), 6.83 (d, J=3.6, 1H), 6.24 (d, J=2.6, 1H), 5.18-5.15 (m, 1H), 5.04-4.98 (m, 2H), 4.12-4.09 (m, 1H), 3.58-3.54 (m, 2H), 1.55 (s, 3H), 1.33 (s, 3H). ESI-LCMS: m/z 348 [M+H]⁺.

Preparation of compound 39-6: To a solution of 39-5 (10.0 g, 28.8 mmol) in Pyridine (80 mL) was added HF.pyridine (120 mL) slowly at −20° C. over 20 min. Then tert-butyl nitrite (5.9 g, 57.5 mmol) was added to the reaction mixture. The mixture was stirred at 5° C. for 30 min, the mixture was diluted with EA and quenched with sat. NaHCO₃. Then the organic layer was dried over Na₂SO₄ and concentrated to give the residue. The crude was purified by silica gel column (SiO₂, PE:EA=5:1˜2:1) to give 39-6 (7.0 g, 19.9 mmol, 69.4% yield) as a yellow solid. ¹H NMR (400 MHz, DMSO-d₆) δ=7.78 (d, J=4.6, 1H), 6.63 (d, J=4.6, 1H), 6.18 (d, J=2.6, 1H), 5.17-5.14 (m, 1H), 4.93-4.91 (m, 1H), 4.19-4.17 (m, 1H), 3.55 (d, J=4.8, 1H), 1.55 (s, 3H), 1.33 (s, 3H). ¹⁹F NMR (DMSO-d₆) δ=−53.22. ESI-LCMS: m/z 351 [M+H]⁺.

Preparation of compound 39-7: To a solution of 39-6 (24 g, 68.5 mmol) in THF (150 mL) was added HCl (30 mL). The mixture was stirred at room temperature for 2 hours. LCMS showed 39-6 was consumed. Water was added and the product was extracted with EA. The organic layer was washed with saturated NaHCO₃ and brine. Then the organic layer was dried over Na₂SO₄ and concentrated to give the crude. The crude was washed with DCM:PE=1:1 to give compound 39-7 (20.0 g, 64.4 mmol, 94.1% yield) as a yellow solid. ¹H NMR (400 MHz, DMSO-d₆) δ=7.79 (d, J=4.6, 1H), 6.63 (d, J=4.6, 1H), 6.03 (d, J=6.1, 1H), 5.40 (d, J=6.6, 1H, exchanged with D₂O), 5.21 (d, J=4.8, 1H, exchanged with D₂O), 5.03 (t, J=5.3, 1H, exchanged with D₂O), 4.39-4.35 (m, 1H), 4.11-4.08 (m, 1H), 3.94-3.91 (m, 1H), 3.66-3.53 (m, 2H). ¹⁹F NMR (DMSO-d₆) δ=−53.62. ESI-LCMS: m/z 311 [M+H]⁺.

Preparation of compound 39-8: To a solution of 39-7 (21 g, 67.7 mmol) in pyridine (200 mL) was added TIPSCl (25.6 g, 81.2 mmol). The mixture was stirred at room temperature for 15 hours. LCMS showed 39-7 was consumed. Water was added and the product was extracted with EA. The organic layer was washed with brine and dried over Na₂SO₄ and concentrated to give the crude. The crude was purified by silica gel column (SiO₂, PE:EA=50:1 to 30:1 to 15:1) to give 39-8 (31 g, 56.08 mmol, 82.85% yield) as a yellow oil. ¹H NMR (400 MHz, DMSO-d₆) δ=7.63 (d, J=3.8, 1H), 6.58 (d, J=3.8, 1H), 5.93 (d, J=1.6, 1H), 5.64 (d, J=5.0, 1H, exchanged with D₂O), 4.57-4.53 (m, 1H), 4.41-4.38 (m, 1H), 4.08-3.91 (m, 3H), 1.05-1.01 (m, 28H). ¹⁹F NMR (DMSO-d₆) δ=−53.46. ESI-LCMS: m/z 553 [M+H]⁺.

Preparation of compound 39-9: To a solution of 39-8 (6.5 g, 11.7 mmol) in iodomethane (70 mL) and Toluene (45 mL) was added Ag₂O (5.4 g, 23.5 mmol) and NaI (1.7 g, 11.7 mmol). The mixture was stirred at 42° C. for 15 hours. The mixture was filtered and the filtrate was concentrated and purified by silica gel column (SiO₂, PE:EA=45:1˜20:1) to give 39-9 (1.4 g, 2.5 mmol, 21.0% yield) as a white solid; ¹H NMR (400 MHz, DMSO-d₆) δ=7.63 (d, J=3.8, 1H), 6.60 (d, J=3.8, 1H), 6.00 (s, 1H), 4.74-4.70 (m, 1H), 4.25-4.23 (m, 1H), 4.08-3.91 (m, 3H), 3.53 (s, 3H), 1.07-1.00 (m, 28H). ¹⁹F NMR (DMSO-d₆) δ=−53.22. ESI-LCMS: m/z 567 [M+H]⁺.

Preparation of compound 39-10: To a solution of 39-9 (4.0 g, 7.0 mmol) in THF (50 mL) was added 10% Pd/C (400 mg). The reaction mixture was stirred at room temperature for 30 min. LCMS showed 39-9 was consumed. The mixture was filtered and the filtrate was concentrated to give the crude 39-10 (3.8 g, 7.0 mmol) as a white solid which was used directly for the next step. ¹H NMR (400 MHz, DMSO-d₆) δ=7.60 (s, 2H, exchanged with D₂O), 7.20 (d, J=3.6, 1H), 6.61 (d, J=3.6, 1H), 5.87 (s, 1H), 4.73-4.40 (m, 1H), 4.11-3.91 (m, 4H), 3.52 (s, 3H), 1.08-1.03 (m, 28H). ESI-LCMS: m/z 541 [M+H]⁺.

Preparation of compound 39-11: To a solution of 39-10 (3.8 g, 7.0 mmol) in pyridine (45 mL) was added BzCl (4.9 g, 35.1 mmol) at room temperature. The mixture was stirred at room temperature for 5 hours. LCMS showed 39-10 was consumed. The reaction was quenched with ice water at 0° C. and the product was extracted with EA. The organic layer was washed with brine and dried over Na₂SO₄ and concentrated to give the crude. The crude was purified by prep-HPLC with the following conditions (IntelFlash-1): Column, C18 silica gel; mobile phase, CH₃CN/H₂O (0.5% NH₄HCO₃)=40/60 increasing to CH₃CN/H₂O (0.5% NH₄HCO₃)=100/0 within 20 min, the eluted product was collected at CH₃CN/H₂O (0.5% NH₄HCO₃)=100/0; Detector, UV 254 nm. This resulted in bisBz protected product 150 mg. The double Bz protected product was dissolved in THF (45 mL) and NH₄OH (2 mL) was added. The mixture was stirred at room temperature for 3 hours. TLC (PE:EA=5:1, Rf, d-bZ=0.5, s-Bz=0.6) showed double Bz protected product was all transformed to desired product. The reaction was quenched with sat. NH₄Cl and the product was extracted with EA. The organic layer was washed with brine and dried over Na₂SO₄ and concentrated to give the crude. The crude was purified by prep-HPLC with the following conditions (IntelFlash-1): Column, C18 silica gel; mobile phase, CH₃CN/H₂O (0.5% NH₄HCO₃)=40/60 increasing to CH₃CN/H₂O (0.5% NH₄HCO₃)=100/0 within 20 min, the eluted product was collected at CH₃CN/H₂O (0.5% NH₄HCO₃)=100/0; Detector, UV 254 nm. This resulted in 39-11 (4.5 g, 6.9 mmol, 99.3% yield) as a white solid. ¹H NMR (400 MHz, DMSO-d₆) δ=11.45 (s, 1H, exchanged with D₂O), 8.07-8.04 (m, 2H), 7.68-7.64 (m, 1H), 7.57-7.73 (m, 3H), 6.78 (d, J=3.8, 1H), 6.05 (d, J=1.1, 1H), 4.74-4.70 (m, 1H), 4.22-4.20 (m, 1H), 4.12-4.07 (m, 1H), 4.00-3.93 (m, 2H), 3.56 (s, 3H), 1.06-1.01 (m, 28H). ¹⁹F NMR (DMSO-d₆) δ=−54.52. ESI-LCMS: m/z 645 [M+H]⁺.

Preparation of compound 39-12: To a solution of 39-11 (5.1 g, 7.9 mmol) in THF (50 mL) was added TBAF (7.9 mmol, 8 mL) at room temperature. The reaction mixture was stirred at room temperature for 0.5 hours. LCMS showed 39-11 was consumed. The mixture was concentrated and purified by silica gel column (SiO₂, PE:EA=5:1˜3:1˜1:1 to EA) to give 39-12 (2.8 g, 6.9 mmol, 87.9% yield) as a white solid. ¹H NMR (400 MHz, DMSO-d₆) δ=11.46 (s, 1H, exchanged with D₂O), 8.07-8.05 (m, 2H), 7.74-7.71 (m, 1H), 7.68-7.64 (m, 1H), 7.58-7.54 (m, 2H), 6.79 (d, J=3.8, 1H), 6.20 (d, J=6.5, 1H), 5.40-5.11 (br, 2H), 4.31-4.28 (m, 1H), 4.19-4.16 (m, 1H), 3.97-3.94 (m, 1H), 3.66-3.56 (m, 2H), 3.30 (s, 3H). ¹⁹F NMR (DMSO-d₆) δ=−54.79. ESI-LCMS: m/z 403 [M+H]⁺.

Preparation of compound 39-13: To a solution of 39-12 (2.8 g, 6.9 mmol) in pyridine (30 mL) was added DMTrCl (2.8 g, 8.3 mmol). The mixture was stirred at room temperature for 2 hours. LCMS showed 39-12 was consumed completely. Water was added and the product was extracted with EA. The organic layer was washed with brine and dried over Na₂SO₄ and concentrated to give the crude. The crude was purified by Prep-HPLC with the following conditions (IntelFlash-1): Column, C18 silica gel; mobile phase, CH₃CN/H₂O (0.5% NH₄HCO₃)=30/70 increasing to CH₃CN/H₂O (0.5% NH₄HCO₃)=100/0 within 25 min, the eluted product was collected at CH₃CN/H₂O (0.5% NH₄HCO₃)=80/20; Detector, UV 254 nm. This resulted in 39-13 (4.8 g, 6.8 mmol, 97.8% yield) as a white solid. ¹H NMR (400 MHz, DMSO-d₆) δ=11.44 (s, 1H, exchanged with D₂O), 8.07-8.05 (m, 2H), 7.68-7.64 (m, 1H), 7.58-7.55 (m, 3H), 7.40-7.36 (m, 2H), 7.31-7.20 (m, 7H), 6.89-6.85 (m, 4H), 6.77 (d, J=3.8, 1H), 6.20 (d, J=6.5, 1H), 5.32 (d, J=6.0, 1H, exchanged with D₂O), 4.34-4.30 (m, 1H), 4.22-4.19 (m, 1H), 4.08-4.06 (m, 1H), 3.73 (s, 6H), 3.36 (s, 3H), 3.30-3.21 (m, 2H). ¹⁹F NMR (DMSO-d₆) δ=−54.54. ESI-LCMS: m/z 705 [M+H]⁺.

Preparation of compound 39-14: To a solution of 39-13 (5 g, 7.1 mmol) in DCM (50 mL) was added DCI (711 mg, 6.0 mmol). Then CEP[N(iPr)₂]₂ (2.5 g, 8.5 mmol) was added to the mixture in one port. The reaction mixture was stirred at room temperature for 1 hour. LCMS showed 39-13 was consumed completely. The solution was washed with water twice and then washed with brine and dried over Na₂SO₄. The organic solution was concentrated to give the residue and the combined residue was purified by Flash-Prep-HPLC with the following conditions (IntelFlash-1): Column, C18 silica gel; mobile phase, CH₃CN/H₂O (0.5% NH₄HCO₃)=50/50 increasing to CH₃CN/H₂O (0.5% NH₄HCO₃)=100/0 within 20 min, the eluted product was collected at CH₃CN/H₂O (0.5% NH₄HCO₃)=100/0; Detector, UV 254 nm. This resulted in 39-14 (6 g, 6.6 mmol, 93.4% yield) as a white solid. ¹H NMR (400 MHz, DMSO-d₆) δ=11.47 (s, 1H, exchanged with D₂O), 8.07-8.05 (m, 2H), 7.68-7.64 (m, 1H), 7.58-7.54 (m, 3H), 7.41-7.37 (m, 2H), 7.31-7.20 (m, 7H), 6.88-6.79 (m, 5H), 6.20-6.18 (m, 1H), 4.58-4.52 (m, 1H), 4.44-4.39 (m, 1H), 4.26-4.17 (m, 1H), 3.86-3.52 (m, 10H), 3.38-3.30 (m, 4H), 2.82-2.79 (m, 1H), 2.63-2.60 (m, 1H), 1.16-1.02 (m, 12H). ¹⁹F NMR (DMSO-d₆) δ=−54.34, −54.39. ³¹P NMR (DMSO-d₆) δ=149.54, 149.34; ESI-LCMS: m/z 705 [M+H]⁺.

### Example A30

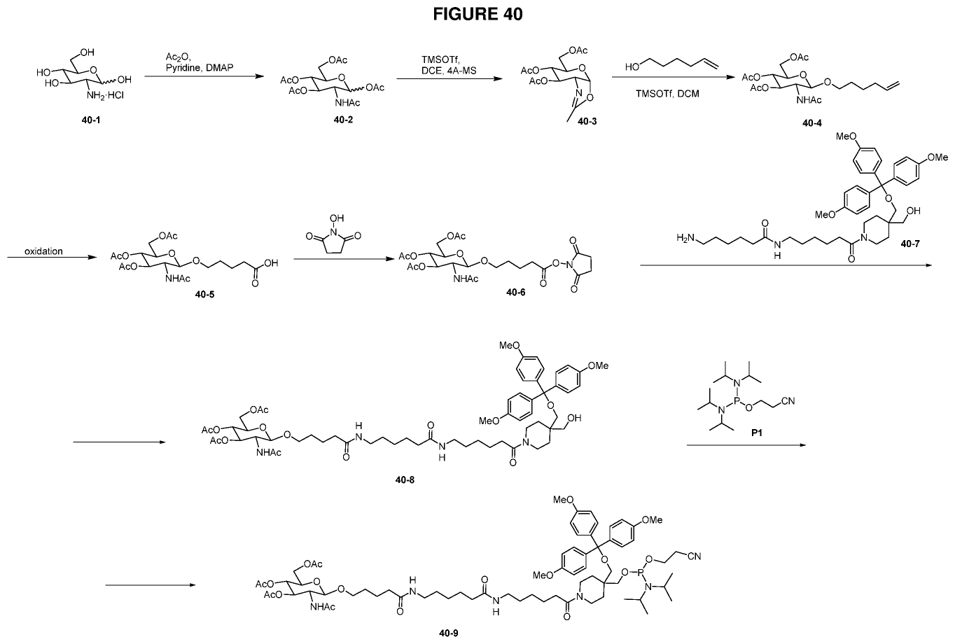

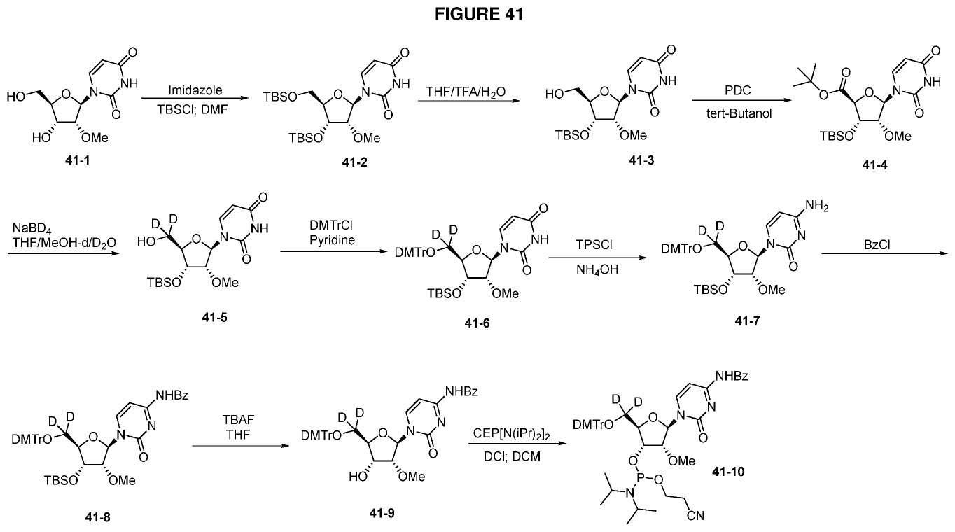

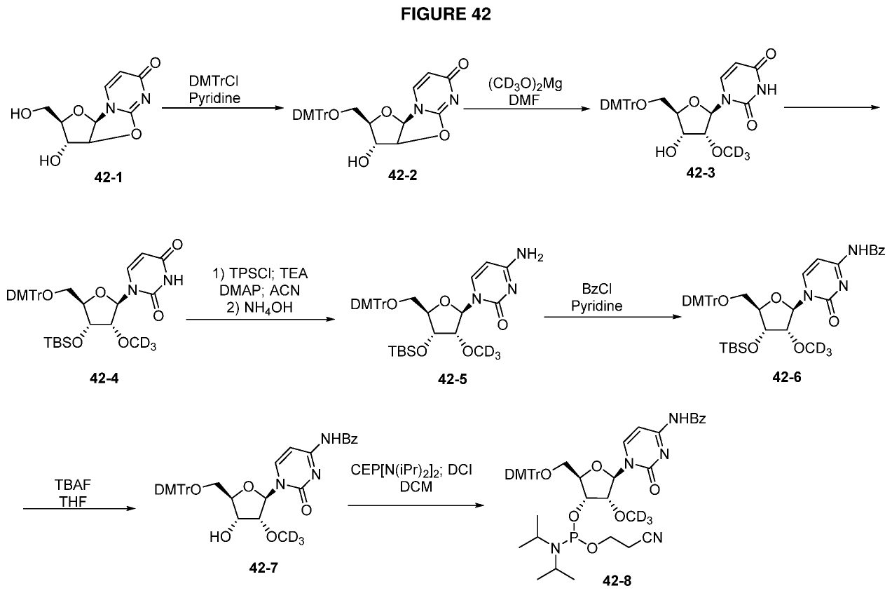

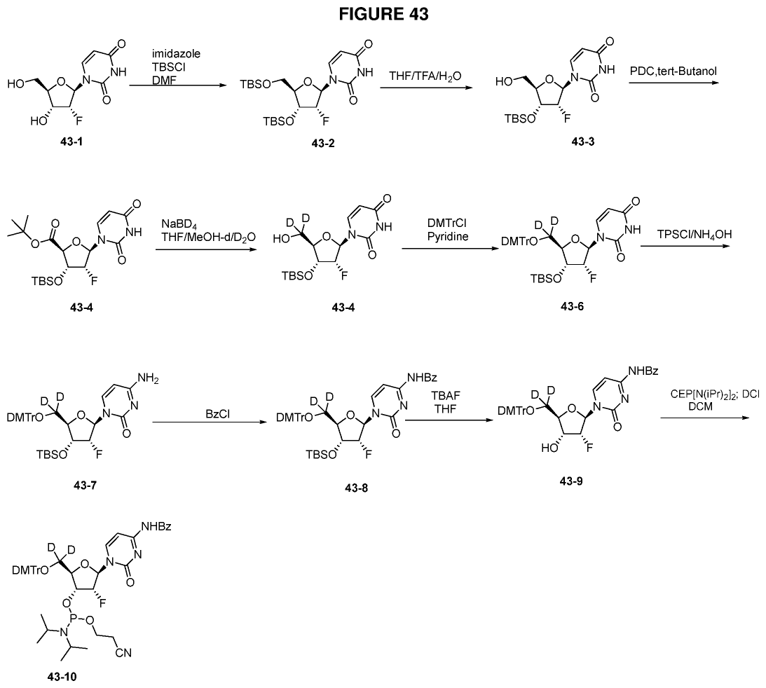

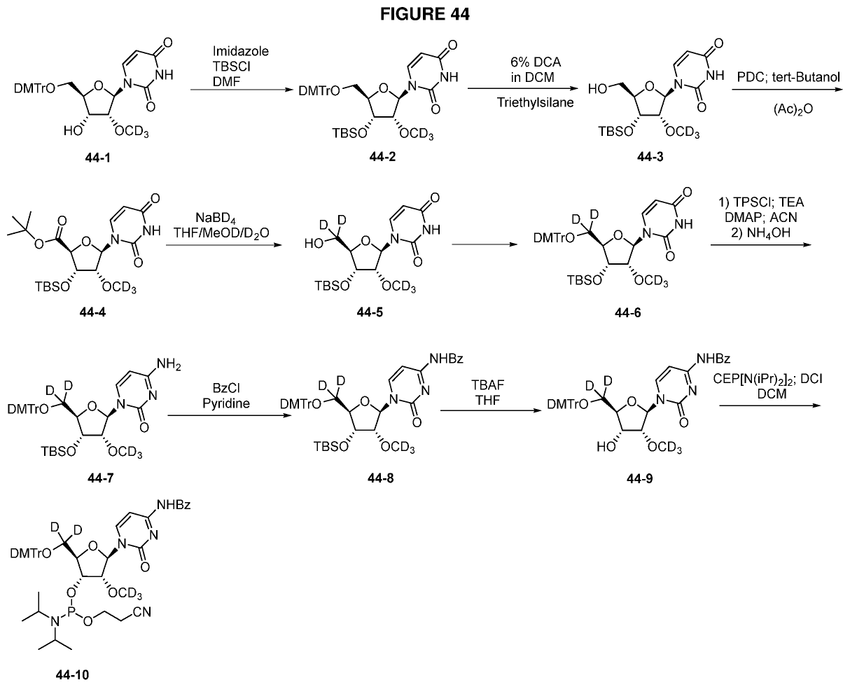

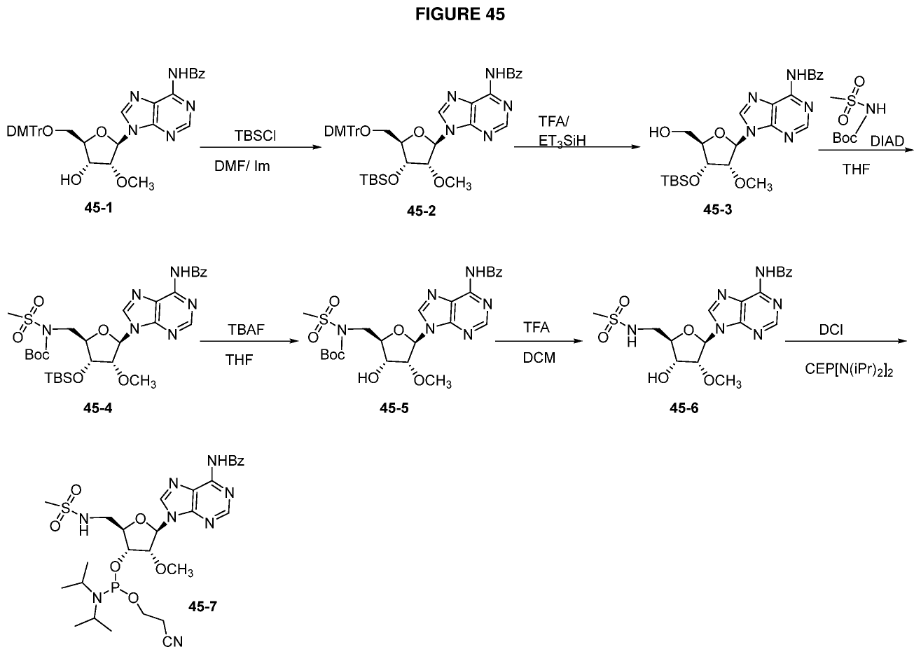

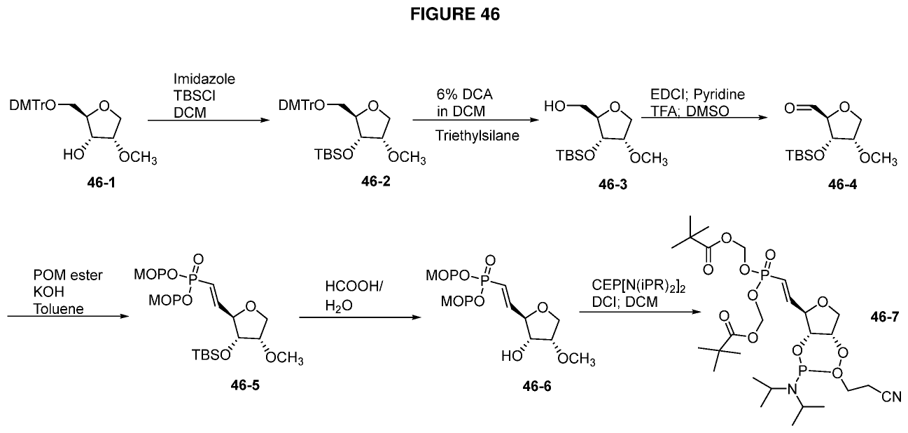

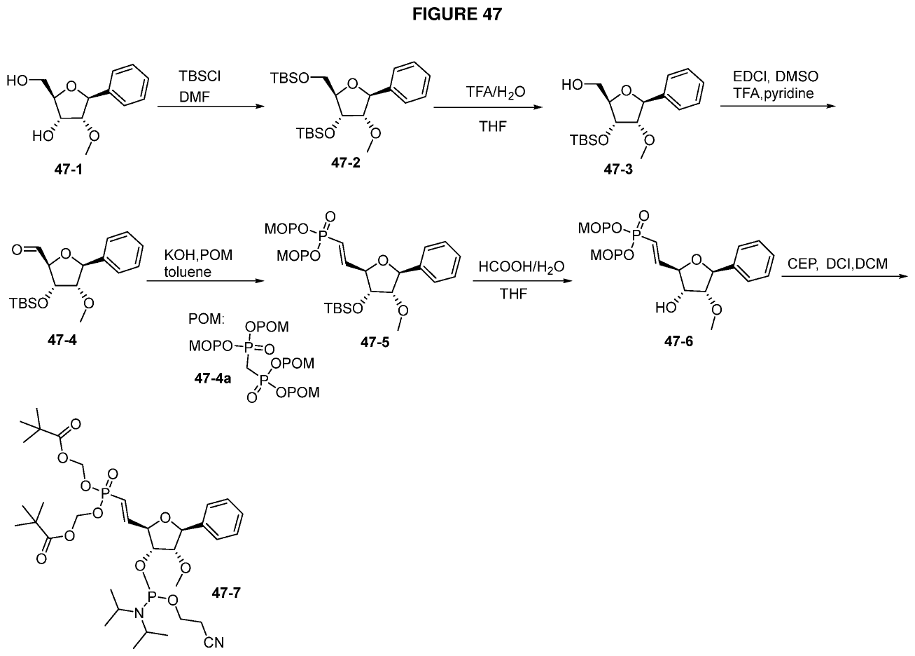

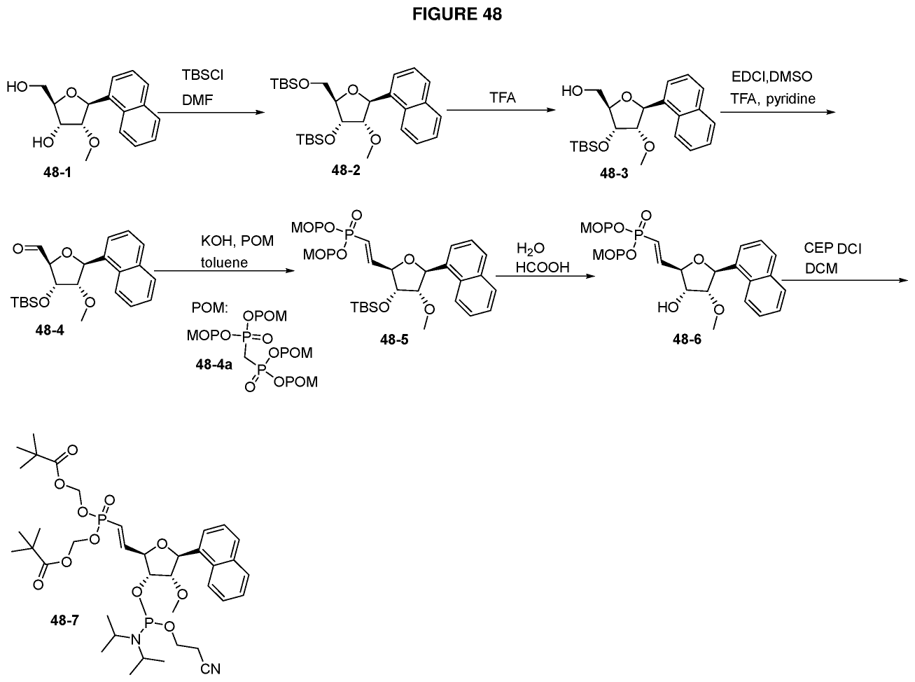

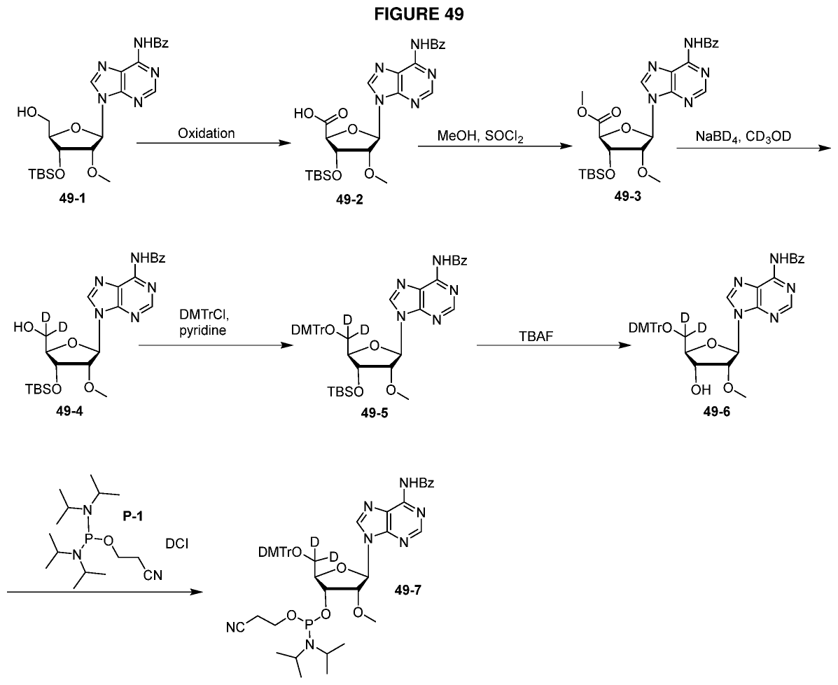

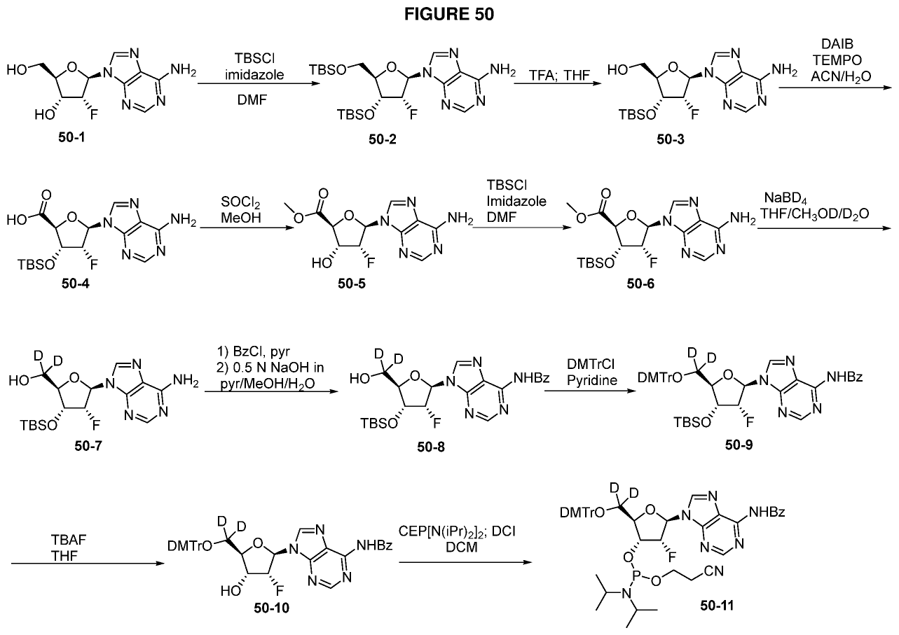

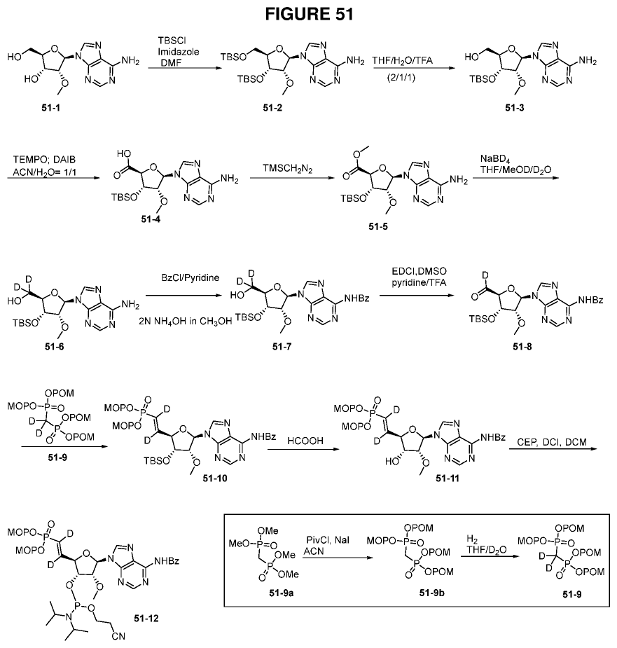

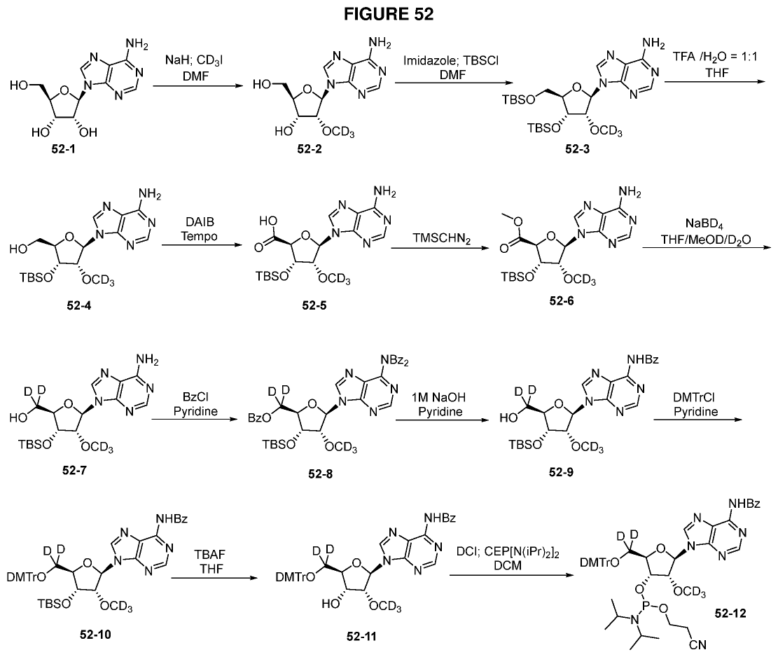

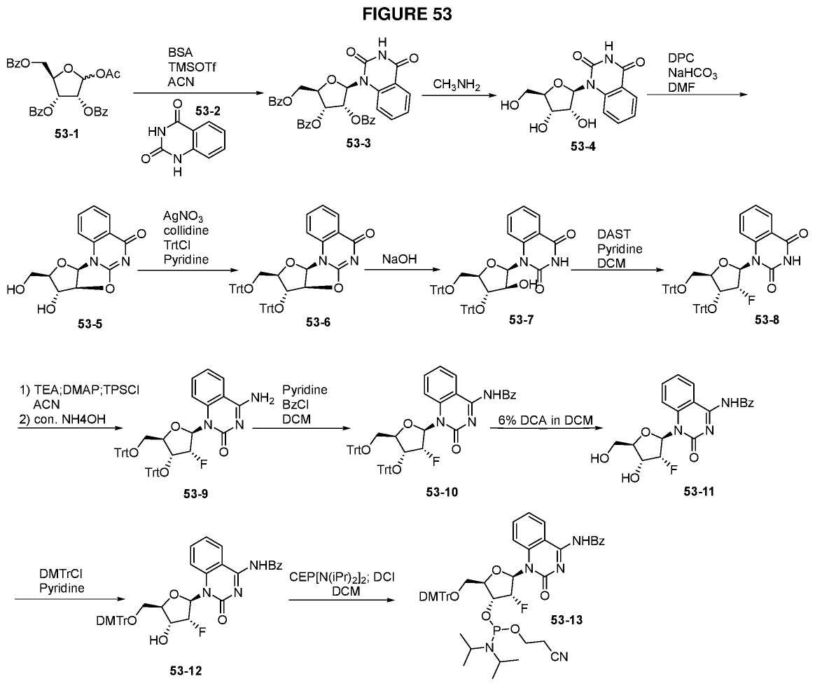

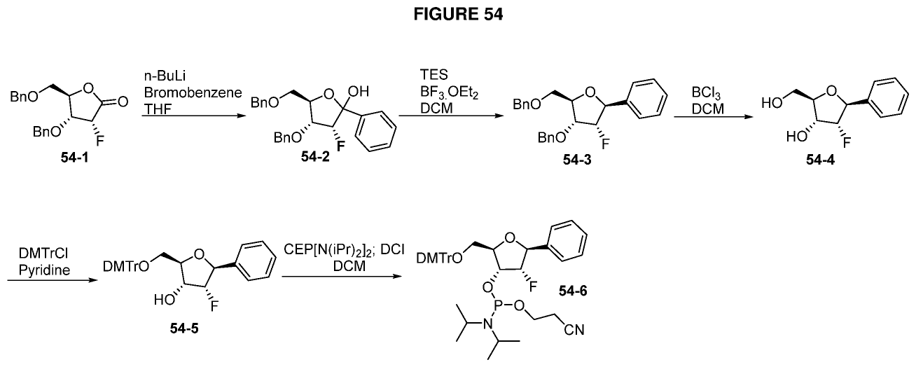

The building block compound 40-9 is useful for making embodiments of modified phosphorothioated oligonucleotides. With reference to FIG. 40, the compound 40-9 was prepared as follows:

Preparation of compound 40-2: To a solution of commercially available glucosamine hydrochloride 40-1 (60 g, 278.25 mmol, 1 eq) in DCM (300 mL) at 0° C. was added Ac₂O (323.83 g, 3.17 mol, 297.09 mL, 11.4 eq) dropwise, followed by pyridine (300 mL) and DMAP (3.40 g, 27.83 mmol, 0.1 eq). The mixture was allowed to gradually warm to 20° C. and stirred at 20° C. for 24 hours. Upon completion as monitored by LCMS, the mixture was concentrated under reduced pressure, diluted with DCM (900 mL), and extracted with NaHCO₃ (sat., aqueous 300 mL*3). The combined organic layers were washed with brine (300 mL), dried over Na₂SO₄, filtered and concentrated under reduced pressure to give compound 40-2 (89.5 g, crude) as a yellow solid. ¹H NMR (400 MHz, CDCl₃) δ=6.16 (d, J=3.8 Hz, 1H), 5.62 (d, J=9.0 Hz, 1H), 5.27-5.16 (m, 2H), 4.54-4.43 (m, 1H), 4.24 (dd, J=4.0, 12.5 Hz, 1H), 4.10-3.94 (m, 2H), 2.18 (s, 3H), 2.08 (s, 3H), 2.04 (d, J=4.0 Hz, 6H), 1.93 (s, 3H; LCMS (ESI): m/z calcd. for C₁₆H₂₃NaNO₁₀ 412.34 [M+Na]⁺, found 412.0).

Preparation of compound 40-3: To a solution of compound 40-2 (40 g, 102.73 mmol, 1 eq) in DCE (320 mL) at 25° C. was added dropwise TMSOTf (23.98 g, 107.87 mmol, 19.49 mL, 1.05 eq), and the mixture was stirred at 60° C. for 4 hours. Upon completion as monitored by LCMS, the mixture was quenched by addition of TEA (60 mL) at 20° C., stirred for 15 min, diluted with DCM (500 mL), and washed with NaHCO₃ (sat., aqueous 300 mL*2). The organic layer was washed with brine (300 mL), dried over Na₂SO₄, filtered, and concentrated under reduced pressure to give compound 40-3 (32.5 g, crude) as a yellow oil. ¹H NMR (400 MHz, CDCl₃) δ=5.96 (d, J=7.3 Hz, 1H), 5.25 (t, J=2.4 Hz, 1H), 4.95-4.88 (m, 1H), 4.19-4.08 (m, 3H), 3.59 (m, 1H), 2.13-2.05 (m, 12H).

Preparation of compound 40-4: To a mixture of compound 40-3 (32.5 g, 98.69 mmol, 1 eq) in DCM (250 mL) was added hex-5-en-1-ol (11.86 g, 118.43 mmol, 13.96 mL, 1.2 eq) and 4A MS (32.5 g). The mixture was stirred at 30° C. for 0.5 h, followed by dropwise addition of TMSOTf (13.16 g, 59.22 mmol, 10.70 mL, 0.6 eq). The mixture was stirred at 30° C. for 16 hours. Upon completion as monitored by LCMS, the reaction mixture was filtered, and the filtrate was diluted with DCM (300 mL) and washed with NaHCO₃ (sat., aqueous 150 mL*2). The organic layer was washed with brine (150 mL), dried over Na₂SO₄, filtered and concentrated under reduced pressure. The residue was purified by flash silica gel chromatography (ISCO®; 220 g SepaFlash® Silica Flash Column, Eluent of 0˜70% PE/EA gradient at 100 mL/min) to give compound 40-4 (12.3 g, 28.64 mmol, 29.02% yield) as a white solid. ¹H NMR (400 MHz, CDCl₃) δ=5.78 (m, 1H), 5.45 (d, J=8.8 Hz, 1H), 5.31 (dd, J=9.4, 10.7 Hz, 1H), 5.06 (t, J=9.5 Hz, 1H), 5.02-4.92 (m, 2H), 4.68 (d, J=8.3 Hz, 1H), 4.30-4.23 (m, 1H), 4.16-4.10 (m, 1H), 3.91-3.76 (m, 2H), 3.73-3.66 (m, 1H), 3.48 (td, J=6.7, 9.5 Hz, 1H), 2.09-2.01 (m, 11H), 1.94 (s, 3H), 1.60-1.36 (m, 4H); LCMS (ESI): m/z calcd. for C₂₀H₃₂NO₉, 430.47 [M+H]⁺, found 430.1.

Preparation of compound 40-5: To a solution of compound 40-4 (12.3 g, 28.64 mmol, 1 eq) in a mixed solvent of DCM (60 mL) and MeCN (60 mL) was added NaIO₄ (2.5 M, 57.28 mL, 5 eq), and the mixture was stirred at 20° C. for 0.5 hours. RuCl₃ (123.00 mg, 592.97 umol, 0.02 eq) was added, and the mixture was stirred at 20° C. for 2 hours. Upon completion as monitored by LCMS, saturated aqueous NaHCO₃ was added to the mixture to adjust pH>7. The mixture was diluted with DCM (300 mL) and subjected to extraction. The aqueous layer was adjusted to pH<7 by citric acid, and the aqueous layer was extracted with DCM (300 mL*3). The combined organic layers were washed with brine (300 mL), dried over Na₂SO₄, filtered, and concentrated under reduced pressure to give compound 40-5 (8.9 g, 19.85 mmol, 69.31% yield, as a brown solid. ¹H NMR (400 MHz, CDCl₃) δ=6.14 (d, J=8.8 Hz, 1H), 5.34-5.20 (m, 1H), 5.08-5.01 (m, 1H), 4.67 (d, J=8.3 Hz, 1H), 4.24 (dd, J=4.8, 12.3 Hz, 1H), 4.17-4.05 (m, 1H), 3.90-3.83 (m, 2H), 3.75-3.62 (m, 2H), 3.50 (d, J=5.9, 9.9 Hz, 1H), 2.44-2.27 (m, 2H), 2.09-1.93 (m, 12H), 1.75-1.53 (m, 4H); LCMS (ESI): m/z calcd. for C₁₉H₃₀NO₁₁, 448.44 [M+H]⁺, found 448.1.

Preparation of compound 40-6: To a solution of compound 40-5 (10 g, 22.35 mmol, 1 eq) and 1-hydroxypyrrolidine-2,5-dione (2.83 g, 24.58 mmol, 1.1 eq) in DCM (100 mL) was added EDCI.HCl (5.57 g, 29.05 mmol, 1.3 eq), and the mixture was stirred at 20° C. for 2 hour. Upon completion as monitored by LCMS, the reaction mixture was diluted with DCM (200 mL) and washed with H₂O (100 mL). The organic layer was washed with NaHCO₃ (sat. aqueous) (100 mL*2) and brine (100 mL), dried over Na₂SO₄, filtered, and concentrated under reduced pressure to give compound 40-6 (10.1 g, 18.47 mmol, 82.66%) as a white solid. ¹H NMR (400 MHz, CDCl₃) δ=5.85 (d, J=8.8 Hz, 1H), 5.31-5.26 (m, 1H), 5.06 (t, J=9.7 Hz, 1H), 4.69 (d, J=8.3 Hz, 1H), 4.25 (dd, J=4.7, 12.2 Hz, 1H), 4.12 (dd, J=2.3, 12.2 Hz, 1H), 3.94-3.79 (m, 2H), 3.75-3.65 (m, 1H), 3.63-3.53 (m, 1H), 2.87 (br d, J=4.3 Hz, 4H), 2.76-2.56 (m, 2H), 2.08 (s, 3H), 2.02 (d, J=1.8 Hz, 6H), 1.92 (s, 3H), 1.86-1.66 (m, 4H); LCMS (ESI): m/z calcd. for C₂₃H₃₃N₂O₁₃, 545.51 [M+H]⁺, found 545.1.

Preparation of compound 40-8: To a solution of compound 40-7 (40-7 prepared by following the general procedure described in WO 2018 013999 A1) (9.8 g, 13.92 mmol, 1 eq) in DCM (100 mL) was added DIEA (3.60 g, 27.84 mmol, 4.85 mL, 2 eq), followed by addition of (2,5-dioxopyrrolidin-1-yl) 5-[3-acetamido-4,5-diacetoxy-6-(acetoxymethyl)tetrahydropyran-2-yl]oxypentanoate (compound 40-6) (9.86 g, 18.10 mmol, 1.3 eq), and the mixture was stirred at 20° C. for 2 hours. Upon completion as monitored by LCMS, the reaction mixture was diluted with water (100 mL), and then extracted with DCM (100 mL*2). The combined organic layers were washed brine (100 mL), dried over anhydrous Na₂SO₄, filtered and the filtrate was concentrated under reduced pressure to give a residue. The residue was purified by flash silica gel chromatography (ISCO®; 120 g SepaFlash® Silica Flash Column, Eluent of 0-6% MeOH/DCM gradient at 80 mL/min) to give compound 40-8 (13.1 g, 11.27 mmol, 80.95% yield, 97.5% purity) as a white solid. ¹H NMR (400 MHz, DMSO-d₆) δ=8.06 (d, J=9.3 Hz, 1H), 7.81 (q, J=5.4 Hz, 2H), 7.21 (d, J=8.8 Hz, 6H), 6.84 (d, J=9.0 Hz, 6H), 5.04 (t, J=10.0 Hz, 1H), 4.78 (t, J=9.7 Hz, 1H), 4.55 (d, J=8.5 Hz, 1H), 4.17 (dd, J=4.5, 12.3 Hz, 1H), 3.97 (d, J=10.0 Hz, 1H), 3.77 (dd, J=2.6, 9.9 Hz, 1H), 3.72-3.64 (m, 11H), 3.46-3.25 (m, 5H), 3.05-2.84 (m, 8H), 2.18 (t, J=7.2 Hz, 2H), 2.05-1.95 (m, 7H), 1.93 (s, 3H), 1.88 (s, 3H), 1.74 (s, 3H), 1.47-1.13 (m, 20H); LCMS (ESI): RT=2.017 min, m/z calcd. for C₆₀H₈₄NaN₄O₁₇, 1156.32[M+Na]⁺, 1155.5.

Preparation of compound 40-9: To a mixture of compound 40-8 (5 g, 4.41 mmol, 1 eq) and 4A MS (5 g) in DCM (50 mL) was added 3-bis(diisopropylamino)phosphanyloxypropanenitrile (1.73 g, 5.74 mmol, 1.82 mL, 1.3 eq) at −10° C., followed by addition of 1H-imidazole-4,5-dicarbonitrile (573.12 mg, 4.85 mmol, 1.1 eq), and the mixture was stirred at 0° C. for 2 hours. Upon completion as monitored by LCMS, the reaction mixture was diluted with DCM (100 mL), washed with NaHCO₃ (sat., aqueous, 50 mL*2), dried over Na₂SO₄, and concentrated under reduced pressure to give a pale yellow foam. The residue was purified by flash silica gel chromatography (ISCO®; 40 g SepaFlash® Silica Flash Column, 0% to 10% i-PrOH in DCM contain 2% TEA) to give compound 40-9 (3.35 g, 2.50 mmol, 56.60% yield, 99.4% purity) as a white solid. ¹H NMR (400 MHz, CD₃CN) δ=7.35-7.25 (m, 6H), 6.88-6.82 (m, 6H), 6.79 (d, J=9.3 Hz, 1H), 6.63-6.46 (m, 2H), 5.17-5.08 (m, 1H), 4.93 (t, J=9.7 Hz, 1H), 4.59 (d, J=8.6 Hz, 1H), 4.22 (dd, J=4.9, 12.2 Hz, 1H), 4.04 (dd, J=2.4, 12.2 Hz, 1H), 3.85-3.32 (m, 22H), 3.15-3.00 (m, 8H), 2.59 (t, J=5.8 Hz, 2H), 2.23 (br t, J=6.6 Hz, 3H), 2.12-2.04 (m, 4H), 2.00 (s, 3H), 1.96 (s, 3H), 1.93 (s, 3H), 1.82 (s, 3H), 1.66-1.45 (m, 12H), 1.42-1.21 (m, 6H), 1.19-1.07 (m, 12H); LCMS (ESI) m/z calcd. for C₆₉H₁₀₁NaN₆O₁₈P 1355.68 [M+Na]⁺, found 1355.7; ³¹P NMR (CD₃CN) δ=147.00.

### Example A31

To the solution of 41-1 (39.2 g, 151.9 mmol) in DMF (390.0 mL), imidazole (33.0 g, 485.3 mmol) and TBSCl (57.2 g, 379.6 mmol) were added at 0° C. The reaction mixture was stirred at room temperature for 15 hours under N₂ atmosphere. After addition of water, the resulting mixture was extracted with EA (500.0 mL). The combined organic layer was washed with water and brine, dried over Na₂SO₄, concentrated to give the crude 41-2 (85.6 g) as a white solid which was used directly for next step. ESI-LCMS: m/z 487.7 [M+H]⁺. ]

Preparation of 41-3: A solution of crude 41-2 (85.6 g) in a mixture solvent of TFA/H₂O=1/1 (400.0 mL) and THF (400.0 mL) was stirred at 0° C. for 30 min. After completion of reaction, the resulting mixture was added con.NH₃*H₂O to pH=7, and then extracted with EA (500.0 mL). The organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 41-3 (36.6 g, 98.4 mmol, 64.7% over two step) as a white solid. ESI-LCMS: m/z 372.5 [M+H]⁺; 1H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 3.68-3.52 (m, 2H), 0.88 (s, 9H), 0.09 (s, 6H).

Preparation of 41-4: To the solution of 41-3 (36.6 g, 98.4 mmol) in dry DCM (200.0 mL) and DMF (50.0 mL) was added PDC (73.9 g, 196.7 mmol), tert-butyl alcohol (188.0 mL) and Ac₂O (93.0 mL) at room temperature under N₂ atmosphere, the reaction mixture was stirred at room temperature for 2 hours. The solvent was removed to give a residue which was purified by silica gel column chromatography (eluent, PE/EA=4:1-2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 41-4 (24.3 g, 54.9 mmol, 55.8%) as a white solid. ESI-LCMS: m/z 443.2 [M+H]⁺; 1H-NMR (400 MHz, DMSO-d₆): δ 11.30 (d, J=1 Hz, 1H), 7.92 (d, J=8 Hz, 1H), 5.86 (d, J=6 Hz, 1H), 5.67-5.65 (m, 1H), 4.33-4.31 (m, 1H), 4.13 (d, J=3 Hz, 1H), 3.73-3.70 (m, 1H), 1.34 (s, 9H), 0.77 (s, 9H), 0.08 (s, 6H).

Preparation of 41-5: To the solution of 41-4 (18.0 g, 40.7 mmol) in dry THF/MeOD/D₂O=10/2/1 (145.0 mL) was added NaBD₄ (5.1 g, 122.1 mmol) three times during an hour at 50° C., the reaction mixture was stirred at room temperature for 2 hours. After completion of reaction, adjusted pH value to 7 with CH₃COOD, after addition of water, the resulting mixture was extracted with EA (300.0 mL). The combined organic layer was washed with water and brine, dried over Na₂SO₄, concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 41-5 (10.4 g, 27.8 mmol, 68.3%) as a white solid. ESI-LCMS: m/z 375.2 [M+H]⁺; 1H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 0.88 (s, 9H), 0.09 (s, 6H).

Preparation of 41-6: To a stirred solution of 41-5 (10.4 g, 27.8 mmol) in pyridine (100.0 mL) was added DMTrCl (12.2 g, 36.1 mmol) at room temperature, The reaction mixture was stirred at room temperature for 2.5 hours, the reaction was quenched with water and extracted with EA (200.0 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 41-6 (13.5 g, 19.9 mmol, 71.6%) as a white solid. ESI-LCMS: m/z 677.8 [M+H]⁺; 1H-NMR (400 MHz, DMSO-d₆): δ 11.39 (d, J=1 Hz, 1H), 7.86 (d, J=4 Hz, 1H), 7.35-7.21 (m, 9H), 6.90-6.88 (m, 4H), 5.78 (d, J=2 Hz, 1H), 5.30-5.27 (m, 1H), 4.33-4.30 (m, 1H), 3.91 (d, J=7 Hz, 1H), 3.85-3.83 (m, 1H), 3.73 (s, 6H), 3.38 (s, 3H), 0.77 (s, 9H), 0.03 (s, 3H), 0.01 (s, 3H).

Preparation of 41-7: To a solution of 41-6 (17 g, 25.1 mmol) in ACN (170 mL) was added DMAP (6.13 g, 50.3 mmol) and TEA (5.1 g, 50.3 mmol, 7.2 mL), Then added TPSCl (11.4 g, 37.7 mmol) at 0° C. under N₂ atmosphere and the mixture was stirred at room temperature for 3 hours under N₂ atmosphere. Then con. NH₃.H₂O (27.3 g, 233.7 mmol) was added at room temperature and the mixture was stirred at room temperature for 16 hours. The reaction was quenched with water and the product was extracted with EA (200 mL). The organic phase was concentrated to give the crude 41-7 (17.0 g) as a white solid which was used directly for next step.

Preparation of 41-8: To a stirred solution of 41-7 (17.0 g, 25.1 mmol) in pyridine (170 mL) were added BzCl (4.3 g, 30.1 mmol) 0° C. under N₂ atmosphere. And the reaction mixture was stirred at room temperature for 2.5 hours. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 41-8 (19.0 g, 24.3 mmol, 95.6% over two step) as a white solid. ESI-LCMS: m/z 780 [M+H]+.

Preparation of 41-9: To a solution of 41-8 (19.0 g, 24.3 mmol) in THF (190 mL) was added 1 M TBAF solution (24 mL). The reaction mixture was stirred at room temperature for 1.0 hours. LC-MS showed 8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 41-9 (15.2 g, 23.1 mmol, 95.5%) as a white solid. ESI-LCMS: m/z 666 [M+H]⁺; 1H-NMR (400 MHz, DMSO-d₆): δ 11.28 (s, 1H), 8.41 (m, 1H), 8.00-7.99 (m, 2H), 7.63-7.15 (m, 13H), 6.93-6.89 (m, 4H), 5.87 (s, 1H), 5.20 (d, J=7.4 Hz, 1H), 4.30 (m, 1H), 4.02 (m, 1H), 3.75 (s, 7H), 3.53 (s, 3H).

Preparation of 41-10: To a suspension of 41-9 (10.0 g, 15.0 mmol) in DCM (100 mL) was added DCI (1.5 g, 12.7 mmol) and CEP[N(iPr)₂]₂ (5.4 g, 18.0 mmol). The mixture was stirred at room temperature for 1 hours. LC-MS showed 41-9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 41-10 (11.5 g, 13.5 mmol, 90.7%) as a white solid. ESI-LCMS: m/z 866 [M+H]⁺; 1H-NMR (400 MHz, DMSO-d₆): δ=11.28 (s, 1H), 8.48-8.41 (m, 1H), 8.00-7.99 (m, 2H), 7.63-7.11 (m, 13H), 6.93-6.89 (m, 4H), 5.92 (m, 1H), 4.55-4.44 (m, 1H), 4.17 (m, 1H), 3.95 (m, 1H), 3.80-3.62 (m, 7H), 3.57-3.46 (m, 5H), 3.32 (s, 1H), 2.78 (m, 1H), 2.62-2.59 (m, 1H), 1.19-0.94 (m, 12H); 31P-NMR (162 MHz, DMSO-d₆): δ=149.52, 148.82.

### Example A32

To a stirred solution of 42-1 (2.0 g, 8.8 mmol) in pyridine (20 mL) were added DMTrCl (3.3 g, 9.7 mmol) at room temperature. The reaction mixture was stirred at room temperature for 2.5 hours. With ice-bath cooling, the reaction was quenched with water and the product was extracted with EA (100 mL). The organic phase was evaporated to dryness under reduced pressure to give a residue which was purified by silica gel column chromatography (eluent, DCM:MeOH=50:1˜20:1) to give 42-2 (3.7 g, 7.2 mmol, 80.1%) as a white solid. ESI-LCMS: m/z 527 [M−H]−.

Preparation of 42-3: To the solution of 42-2 (2.8 g, 5.3 mmol) in dry DMF (56 mL) was added (CD₃O)₂Mg (2.9 g, 31.8 mmol) (Nucleic Acids Research, 2011, Vol. 39, No. 10, 4340-4351) at room temperature under N₂ atmosphere. The reaction mixture was stirred at 100° C. for 15 hours. With ice-bath cooling, the reaction was quenched with saturated aqueous NH₄Cl and extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 42-3 (2.0 g, 3.6 mmol, 67.9%) as a white solid. ESI-LCMS: m/z 562 [M−H]−; 1H-NMR (400 MHz, DMSO-d₆): δ 11.38 (s, 1H), 7.73 (d, J=8 Hz, 1H), 7.46-7.19 (m, 9H), 6.91 (d, J=7.4 Hz, 4H), 5.81-5.76 (AB, J=20 Hz, 1H), 5.30 (d, J=8 Hz, 1H), 5.22 (s, 1H), 4.25-4.15 (m, 1H), 3.99-3.92 (m, 1H), 3.85-3.79 (m, 1H), 3.74 (s, 6H), 3.34-3.18 (m, 31H).

Preparation of 42-4: To the solution of 42-3 (14.3 g, 25.4 mmol, Scheme 2) in pyridine (150 mL) was added imidazole (4.5 g, 66.6 mmol) and TBSCl (6.0 g, 40.0 mmol) at 0° C., and the reaction mixture was stirred at room temperature for 15 hours under N₂ atmosphere. After addition of water, the resulting mixture was extracted with EA (500 mL). The combined organic layer was washed with water and brine, dried over Na₂SO₄, and concentrated to give the crude 42-4 (18.0 g) as a white solid which was used directly for next step. ESI-LCMS: m/z 676 [M−H]−.

Preparation of 42-5: To the solution of 42-4 (18.8 g) in dry ACN (200 mL) was added TPSCl (16.8 g, 65.2 mmol) and TEA (5.6 g, 65.2 mmol) and DMAP (6.8 g, 65.2 mmol), and the reaction mixture was stirred at room temperature for 3.5 hours under N₂ atmosphere. After addition of water, the resulting mixture was extracted with EA (300 mL). The combined organic layer was washed with water and brine, dried over Na₂SO₄, and concentrated to give the crude 42-5 (22.0 g) as a white solid which was used directly for next step. ESI-LCMS: m/z 677 [M−H]+.

Preparation of 42-6: To a solution of 42-5 (22.0 g) in pyridine (150 mL) was added BzCl (6.8 g, 48.9 mmol) under ice bath. The reaction mixture was stirred at room temperature for 2.5 hours. LCMS showed 42-5 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in the crude 42-6 (20.8 g, 26.7 mmol, 82% yield over two steps) as a white solid. ESI-LCMS: m/z 781 [M+H]⁺; 1H-NMR (400 MHz, DMSO-d₆): δ 11.30 (s, 1H), 8.55 (d, J=8.0 Hz, 1H), 8.00-7.98 (m, 2H), 7.74-7.66 (m, 1H), 7.60-7.50 (m, 2H), 7.47-7.31 (m, 4H), 7.30-7.2 (m, 5H), 7.20-7.1 (m, 1H), 6.91 (d, J=7.4 Hz, 4H), 5.91-5.86 (AB, J=20.0 Hz, 1H), 4.30 (d, J=8.0 Hz, 1H), 3.87-3.78 (s, 1H), 3.78-3.70 (m, 6H), 3.62-3.51 (m, 1H), 3.28-3.2 (m, 1H), 2.15-2.05 (m, 3H), 0.73 (s, 9H), 0.00 (m, 6H).

Preparation of 42-7: To a solution of 42-6 (20.8 g, 26.7 mmol) in THF (210 mL) was added 1 M TBAF solution (32 mL). The reaction mixture was stirred at room temperature for 1.5 hours. LCMS showed 42-6 was consumed completely. Water (600 mL) was added. The product was extracted with EA (400 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 42-7 (12.4 g, 18.6 mmol, 70%) as a white solid. ESI-LCMS: m/z 667 [M+H]⁺; 1H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 7H), 2.57-2.42 (m, 2H).

Preparation of 42-8: To a suspension of 42-7 (12.4 g, 18.6 mmol) in DCM (120 mL) was added DCI (1.7 g, 15.8 mmol) and CEP[N(iPr)₂]₂ (7.3 g, 24.2 mmol). The mixture was stirred at room temperature for 2 hours. LC-MS showed 42-7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 42-8 (13.6 g, 15.7 mmol, 84.0%) as a white solid. ESI-LCMS: m/z 867 [M+H]⁺; 1H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 10H), 2.74-2.59 (m, 1H), 2.57-2.43 (m, 1H), 1.27-1.10 (m, 9H), 1.09-0.95 (m, 3H). 31P-NMR (162 MHz, DMSO-d₆): δ 149.59, 148.85.

### Example A33

Preparation of 43-2: To the solution of 43-1 (13.0 g, 52.8 mmol) in DMF (100 mL) was added imidazole (12.6 g, 184.8 mmol) and TBSCl (19.8 g, 132.0 mmol) at 0° C., and the reaction mixture was stirred at room temperature for 15 hours under N₂ atmosphere. After addition of water, the resulting product was extracted with EA (500 mL). The combined organic layer was washed with water and brine, dried over Na₂SO₄, and concentrated to give the crude 43-2 (30.6 g) as a white solid which was used directly for next step. ESI-LCMS: m/z 475 [M+H]⁺. WO2017106710A1

Preparation of 43-3: A solution of crude 43-2 (30.6 g) in a mixture solvent of TFA/H₂O=1/1 (100 mL) and THF (100 mL) was stirred at 0° C. for 30 min. After completion of reaction, the resulting mixture was added con.NH₃*H₂O to pH=7.5, and then the mixture was extracted with EA (500 mL), the organic layer was washed with brine, dried over Na₂SO₄ and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 43-3 (12.0 g, 33.3 mmol, 65.8% over two step) as a white solid. ESI-LCMS: m/z 361 [M+H]⁺; 1H-NMR (400 MHz, DMSO-d₆): δ 11.39 (s, J=1 Hz, 1H, exchanged with D₂O), 7.88 (d, J=8 Hz, 1H), 5.91-5.86 (m, 1H), 5.66-5.62 (m, 1H), 5.21 (t, J=5.2 Hz, 1H, exchanged with D₂O), 5.18-5.03 (m, 1H), 4.37-4.29 (m, 1H), 3.87-3.83 (m, 1H), 3.78-3.73 (m, 1H), 3.56-3.51 (m, 1H), 0.87 (s, 9H), 0.09 (s, 6H).

Preparation of 43-4: To the solution of 43-3 (11.0 g, 30.5 mmol) in dry DCM (60 mL) and DMF (15 mL) was added PDC (21. g, 61.0 mmol), tert-butyl alcohol (45 mL) and Ac₂O (32 mL) at room temperature under N₂ atmosphere. And the reaction mixture was stirred at room temperature for 2 hours. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 43-4 (9.5 g, 22.0 mmol, 72.3%) as a white solid. ESI-LCMS: m/z 431 [M+H]⁺; 1H-NMR (400 MHz, DMSO-d₆): δ 11.45 (s, J=1 Hz, 1H, exchanged with D₂O), 7.93 (d, J=8.5 Hz, 1H), 6.02-5.97 (m, 1H), 5.76-5.74 (m, 1H), 5.29-5.14 (m, 1H), 4.59-4.52 (m, 1H), 4.29-4.27 (m, 1H), 1.46 (s, 9H), 0.89 (s, 9H), 0.12 (s, 6H).

Preparation of 43-5: To the solution of 43-4 (8.5 g, 19.7 mmol) in dry THF/MeOD/D₂O=10/2/1 (80 mL) was added NaBD₄ (2.5 g, 59.1 mmol) three times per an hour at 50° C. And the reaction mixture was stirred at room temperature for 2 hours. After completion of reaction, adjusted pH value to 7 with CH₃COOD, after addition of water, the resulting mixture was extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted 43-5 (3.5 g, 9.7 mmol, 50.3%) as a white solid. ESI-LCMS: m/z 363 [M+H]⁺; 1H-NMR (400 MHz, DMSO-d₆): δ 11.41 (s, J=1 Hz, 1H, exchanged with D₂O), 7.88 (d, J=8 Hz, 1H), 5.91-5.86 (m, 1H), 5.66-5.62 (m, 1H), 5.19 (t, J=5.2 Hz, 1H, exchanged with D₂O), 5.18-5.03 (m, 1H), 4.37-4.29 (m, 1H), 3.87-3.83 (m, 1H), 0.88 (s, 9H), 0.10 (s, 6H). Ref: Painter, George R. et al, PCT Int. Appl., 2019173602, 12 Sep. 2019.

Preparation of 43-6: To a stirred solution of 43-5 (3.4 g, 9.7 mmol) in pyridine (35 mL) were added DMTrCl (3.4 g, 10.1 mmol) at room temperature And the reaction mixture was stirred at room temperature for 2.5 hours. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 43-5 (5.5 g, 8.3 mmol, 85.3%) as a white solid. ESI-LCMS: m/z 665 [M+H]⁺; 1H-NMR (400 MHz, DMSO-d₆): δ 11.50 (d, J=1 Hz, 1H, exchanged with D₂O), 7.92 (d, J=4 Hz, 1H), 7.44-7.27 (m, 9H), 6.96-6.93 (m, 4H), 5.94 (d, J=20.5 Hz, 1H), 5.39-5.37 (m, 1H), 5.32-5.17 (m, 1H), 4.60-4.51 (m, 1H), 4.01 (d, J=8.8 Hz, 1H), 3.80 (s, 6H), 0.80 (s, 9H), 0.09 (s, 3H), −0.05 (s, 3H).

Preparation of 43-7: To a solution of 43-6 (16 g, 24.1 mmol) in ACN (160 mL) was added DMAP (5.9 g, 48.2 mmol) and TEA (4.8 g, 48.2 mmol), then added TPSCl (10.9 g, 36.1 mmol) at 0° C. under N₂ atmosphere and the mixture was stirred at room temperature for 5 hours under N₂ atmosphere. Then con. NH₃.H₂O (30 mL) was added at room temperature and the mixture was stirred at room temperature for 16 hours. The reaction was quenched with water and the product was extracted with EA (200 mL). The organic phase was concentrated to give the crude 43-7 (16.0 g) as a white solid which was used directly for next step.

Preparation of 43-8: To a stirred solution of 43-7 (16.0 g, 24.1 mmol) in pyridine (160 mL) were added BzCl (4.1 g, 28.9 mmol) 0° C. under N₂ atmosphere. And the reaction mixture was stirred at room temperature for 2.5 hours. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 43-8 (18.0 g, 23.4 mmol, 97.0%) as a white solid. ESI-LCMS: m/z 768 [M+H]⁺; 1H-NMR (400 MHz, DMSO-d₆): δ 11.31 (s, 1H), 8.47 (d, J=7.2 Hz, 1H), 7.99 (d, J=7.6 Hz, 2H), 7.65-7.16 (m, 13H), 6.92 (d, J=8.8 Hz, 4H), 6.01 (d, J=18.4 Hz, 1H), 5.18-5.04 (dd, 1H), 4.58-4.52 (m, 1H), 4.07 (d, J=9.6 Hz, 1H), 3.75 (s, 6H), 0.73 (s, 9H), 0.05 (s, 3H), −0.06 (s, 3H).

Preparation of 43-9: To a solution of 43-8 (18.0 g, 23.4 mmol) in THF (180 mL) was added 1 M TBAF solution (23 mL). The reaction mixture was stirred at room temperature for 1.5 hours. LC-MS showed 43-8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 43-9 (13.7 g, 21.1 mmol, 90.5%) as a white solid. ESI-LCMS: m/z 654.2 [M+H]⁺; 1H-NMR (400 MHz, DMSO-d₆): δ 11.31 (s, 1H), 8.35 (d, J=7.4 Hz, 1H), 8.01 (m, 2H), 7.65-7.16 (m, 13H), 6.92 (d, J=8.8 Hz, 4H), 5.94 (d, J=18.0 Hz, 1H), 5.71 (d, J=7.0 Hz, 1H), 5.12-4.98 (dd, 1H), 4.51-4.36 (m, 1H), 4.09 (d, J=9.6 Hz, 1H), 3.75 (s, 6H).

Preparation of 43-10: To a suspension of 43-9 (10.6 g, 16.2 mmol) in DCM (100 mL) was added DCI (1.6 g, 13.7 mmol) and CEP[N(iPr)₂]₂ (5.8 g, 19.4 mmol). The mixture was stirred at room temperature for 1 hours. LC-MS showed 43-9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 43-10 (10.5 g, 14.5 mmol, 75.9%) as a white solid. ESI-LCMS: m/z 854.3 [M+H]⁺; 1H-NMR (400 MHz, DMSO-d₆): δ 11.31 (s, 1H), 8.41-8.37 (m, 1H), 8.01 (d, J=7.7 Hz, 2H), 7.65-7.16 (m, 13H), 6.92-6.88 (m, 4H), 6.06-5.98 (m, 1H), 5.33-5.15 (m, 1H), 4.78-4.58 (m, 1H), 4.23-4.19 (m, 1H), 3.81-3.73 (m, 6H), 3.60-3.50 (m, 3H), 3.32 (s, 1H), 2.76 (t, J=6.0 Hz, 1H), 2.60 (t, J=5.8 Hz, 1H), 1.15-0.94 (m, 12H); 31P-NMR (162 MHz, DMSO d₆): δ 150.23, 150.18, 149.43, 149.38.

### Example A34

Preparation of 44-2: To the solution of 44-1 (14.3 g, 25.4 mmol, Scheme 2) in DMF (150 mL) was added imidazole (4.5 g, 66.6 mmol) and TBSCl (6.0 g, 40.0 mmol) at 0° C., and the reaction mixture was stirred at room temperature for 15 hours under N₂ atmosphere. After addition of water, the resulting mixture was extracted with EA (500 mL). The combined organic layer was washed with water and brine, dried over Na₂SO₄, and concentrated to give the crude 44-2 (18.0 g) as a white solid which was used directly for next step. ESI-LCMS: m/z 676 [M−H]−.

Preparation of 44-3: To the solution of crude 44-2 (18.0 g) in the solution of DCA (6%) in DCM (200 mL) was added triethylsilane (50 mL) at room temperature, and the reaction mixture was stirred at room temperature for 5-10 min. After completion of reaction, the resulting mixture was added pyridine to pH=7, and then the solvent was removed and the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 44-3 (6.5 g, 17.2 mmol, 67.7% for two step) as a white solid. ESI-LCMS: m/z 376 [M+H]⁺; 1H-NMR (400 MHz, DMSO-d₆): δ 7.92 (d, J=8 Hz, 1H), 5.82 (d, J=5.2 Hz, 1H), 5.68-5.63 (m, 1H), 5.20-5.15 (m, 1H), 4.32-4.25 (m, 1H), 3.87-3.80 (m, 2H), 3.69-3.61 (m, 1H), 3.57-3.49 (m, 1H), 0.88 (s, 9H), 0.09 (s, 6H).

Preparation of 44-4: To the solution of 44-3 (6.5 g, 17.2 mmol) in dry DCM (35 mL) and DMF (9 mL) was added PDC (12.9 g, 34.3 mmol), tert-butyl alcohol (34 mL) and (Ac)₂O (17 mL) at room temperature under N₂ atmosphere. And the reaction mixture was stirred at room temperature for 2 hours. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 44-4 (5.5 g, 12.3 mmol, 70.1%) as a white solid. ESI-LCMS: m/z 446 [M+H]⁺; 1H-NMR (400 MHz, DMSO-d₆): δ=11.29 (s, 1H), 7.91 (d, J=8.4 Hz, 1H), 5.85 (d, J=6.4 Hz, 1H), 5.71-5.61 (m, 1H), 4.35-4.28 (m, 1H), 4.12 (d, J=3.2 Hz, 1H), 3.75-3.67 (m, 1H), 1.33 (s, 9H), 0.76 (s, 9H), 0.00 (d, J=1.6 Hz, 6H).

Preparation of 44-5: To the solution of 44-4 (5.4 g, 12.1 mmol) in THF/MeOD/D₂O=10/2/1 (44 mL) was added NaBD₄ (1.5 g, 36.3 mmol) at room temperature and the reaction mixture was stirred at 50° C. for 2 hours. After completion of reaction, adjusted pH value to 7 with CH₃COOD. Water was added, the resulting mixture was extracted with EA (500 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 44-5 (2.6 g, 6.8 mmol, 56.1%) as a white solid. ESI-LCMS: m/z 378 [M+H]⁺; 1H-NMR (400 MHz, DMSO-d₆): δ 11.35 (s, 1H), 7.91 (d, J=8.0 Hz, 1H), 5.82 (d, J=5.2 Hz, 1H), 5.69-5.60 (m, 1H), 5.14 (s, 1H), 4.34-4.20 (m, 1H), 3.88-3.76 (m, 2H), 0.87 (s, 9H), 0.08 (s, 6H).

Preparation of 44-6: To a stirred solution of 44-5 (2.6 g, 6.8 mmol) in pyridine (30 mL) were added DMTrCl (3.5 g, 10.3 mmol) at room temperature And the reaction mixture was stirred at room temperature for 2.5 hours. With ice-bath cooling, the reaction was quenched with water and the product was extracted into EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 44-6 (4.3 g, 6.3 mmol, 90.1%) as a white solid. ESI-LCMS: m/z 678 [M−H]−; 1H-NMR (400 MHz, DMSO-d₆): δ 11.39 (s, 1H), 7.86 (d, J=8.0 Hz, 1H), 7.42-7.17 (m, 9H), 6.96-6.83 (m, 4H), 5.82-5.69 (m, 2H), 5.29 (d, J=8.4 Hz, 1H), 4.36-4.25 (m, 1H), 3.90 (d, J=7.2 Hz, 1H), 3.86-3.80 (m, 1H), 3.73 (s, 6H), 0.75 (s, 9H), 0.02 (s, 3H), −0.04 (s, 3H).

Preparation of 44-7: To a solution of 44-6 (18.8 g, 26.4 mmol) in ACN (200 mL) was added TPSCl (16.8 g, 55.3 mmol) and DMAP (5.6 g, 55.3 mmol) and TEA (6.8 g, 55.3 mmol). The reaction mixture was stirred at room temperature for 3.5 hours. LCMS showed the reaction was consumed. The mixture was diluted with con. NH₄OH (28 mL). The mixture was diluted with water and EA. The product was extracted with EA. The organic layer was washed with brine and dried over Na₂SO₄ and concentrated to give the crude 44-7 (18.5 g) which was used directly for the next step.

Preparation of 44-8: To a solution of 44-7 (18.8 g, 27.69 mmol) in pyridine (200 mL) was added BzCl (5.8 g, 41.5 mmol) under ice bath. The reaction mixture was stirred at room temperature for 2.5 hours. LCMS showed 44-7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃H₃CN/H₂O (0.5% NH₄HCO₃)=1/0 within 25 min, the eluted product was collected at CH₃H₃CN/H₂O (0.5% NH₄HCO₃)=1/0; Detector, UV 254 nm. This resulted in 44-8 (19.8 g, 25.3 mmol, 91% yield) as a white solid. ESI-LCMS: m/z 783 [M−H]−; 1H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of 44-9: To a solution of 44-8 (18.8 g, 26.4 mmol) in THF (190 mL) was added 1 M TBAF solution (28 mL). The reaction mixture was stirred at room temperature for 1.5 hours. LCMS showed 44-8 was consumed completely. Water (200 mL) was added. The product was extracted with EA (200 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in 44-9 (17.1 g, 25.6 mmol, 96%) as a white solid. ESI-LCMS: m/z 669 [M−H]−; 1H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of 44-10: To a suspension of 44-9 (10.8 g, 16.2 mmol) in DCM (100 mL) was added DCI (1.5 g, 13.7 mmol) and CEP[N(iPr)₂]₂ (5.8 g, 19.3 mmol). The mixture was stirred at room temperature for 2 hours. LC-MS showed 44-9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 44-10 (11.3 g, 13 mmol, 80%) as a white solid. ESI-LCMS: m/z 868 [M+H]⁺; 1H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 10H), 2.74-2.59 (m, 1H), 2.57-2.43 (m, 1H), 1.27-1.10 (m, 9H), 1.09-0.95 (m, 3H). 31P-NMR (162 MHz, DMSO-d₆): δ 149.52, 148.81.

### Example A34

Preparation of 45-2: To a solution of 45-1 (40 g, 58.16 mmol) in DMF (60 mL) were added imidazole (11.88 g, 174.48 mmol), NaI (13.08 g, 87.24 mmol), and TBSCl (17.52 g, 116.32 mmol) at 20° C. in one portion. The reaction mixture was stirred at 20° C. for 12 hours. Upon completion, the mixture was diluted with EA (200 mL). The organic layer was washed with brine/water (80 mL/80 mL*4), dried over Na₂SO₄, filtered and evaporated to give 45-2 (50.8 g, crude) as yellow solid. ESI-LCMS: 802.3 [M+H]+

Preparation of 45-3: To a solution of 45-2 (8.4 g, 10.47 mmol) in DCM (120 mL) were added Et₃SiH (3.06 g, 26.3 mmol, 4.2 mL) and TFA (1.29 g, 0.84 mL) dropwise at 0° C. The reaction mixture was stirred at 20° C. for 2 hours. The reaction mixture was washed with saturated aqueous NaHCO₃ (15 mL) and brine (80 mL). The organic layer was dried over Na₂SO₄, filtered and evaporated. The residue was purified by flash silica gel chromatography (ISCO®; 80 g SepaFlash® Silica Flash Column, Eluent of 0˜83% EA/PE gradient at 80 mL/min) to give 3 (2.92 g, 55.8% yield,) as a white solid. ESI-LCMS: 500.2 [M+H]⁺; 1H NMR (400 MHz, CDCl₃) δ=8.79 (s, 1H), 8.14 (s, 1H), 8.02 (d, J=7.6 Hz, 2H), 7.64-7.58 (m, 1H), 7.56-7.49 (m, 2H), 5.98-5.93 (m, 1H), 4.63-4.56 (m, 2H), 4.23 (s, 1H), 3.98 (dd, J=1.5, 13.1 Hz, 1H), 3.75 (dd, J=1.5, 13.1 Hz, 1H), 3.28 (s, 3H), 2.06-1.99 (m, 1H), 1.00-0.90 (m, 9H), 0.15 (d, J=7.0 Hz, 6H)

Preparation of 45-4: 45-3 (6 g, 12.01 mmol) and tert-butyl N-methylsulfonylcarbamate (3.52 g, 18.01 mmol) were co-evaporated with toluene (50 mL), dissolved in dry THF (100 mL), and cooled to 0° C. PPh₃ (9.45 g, 36.03 mmol,) was then added, followed by dropwise addition of DIAD (7.28 g, 36.03 mmol, 7.00 mL) in dry THF (30 mL). The reaction mixture was stirred at 20° C. for 18 hours. Upon completion, the reaction mixture was then diluted with DCM (100 mL) and washed with water (70 mL) and brine (70 mL), dried over Na₂SO₄, filtered and evaporated to give a residue. The residue was purified by flash silica gel chromatography (ISCO®; 80 g SepaFlash® Silica Flash Column, Eluent of 0˜100% Ethyl acetate/Petroleum ether gradient at 60 mL/min) followed by reverse-phase HPLC (0.1% NH₃.H₂O condition, eluent at 74%) to give 45-4 (2.88 g, 25% yield) as a white solid. ESI-LCMS: 677.1 [M+H]⁺; 1H NMR (400 MHz, CDCl₃) δ=9.24 (s, 1H), 8.84 (s, 1H), 8.36 (s, 1H), 8.05 (br d, J=7.3 Hz, 2H), 7.66-7.42 (m, 4H), 6.16 (d, J=5.0 Hz, 1H), 4.52 (br t, J=4.5 Hz, 1H), 4.25-4.10 (m, 1H), 3.97 (br dd, J=8.0, 14.8 Hz, 1H), 3.48 (s, 3H), 3.27 (s, 3H), 1.54 (s, 9H), 0.95 (s, 9H), 0.14 (d, J=0.8 Hz, 6H)

Preparation of 45-5: To a solution of 45-4 (2.8 g, 4.14 mmol) in THF (20 mL) was added TBAF (4 M, 1.03 mL) and the mixture was stirred at 20° C. for 12 hours. The reaction mixture was then evaporated. The residue was purified by flash silica gel chromatography (ISCO®; 12 g SepaFlash® Silica Flash Column, Eluent of 0˜6% MeOH/ethyl acetate gradient at 20 mL/min) to give 45-5 (2.1 g, 83.92% yield) as a white solid. ESI-LCMS: 563.1[M+H]⁺; ¹H NMR (400 MHz, CDCl₃) δ=8.85-8.77 (m, 1H), 8.38 (s, 1H), 8.11-7.99 (m, 2H), 7.64-7.50 (m, 4H), 6.19 (d, J=2.8 Hz, 1H), 4.36-4.33 (m, 1H), 4.29 (br d, J=4.3 Hz, 1H), 4.22-4.02 (m, 2H), 3.65-3.59 (m, 3H), 3.28 (s, 3H), 1.54 (s, 9H)

Preparation of 45-6: To a solution of 45-5 (2.1 g, 3.73 mmol) in DCM (20 mL) was added TFA (7.70 g, 67.53 mmol, 5 mL) at 0° C. The reaction mixture was stirred at 20° C. for 24 hours. Upon completion, the reaction was quenched with saturated aqueous NaHCO₃ to reach pH 7. The organic layer was dried over Na₂SO₄, filtered, and evaporated at low pressure. The residue was purified by flash silica gel chromatography (ISCO®; 12 g SepaFlash® Silica Flash Column, Eluent of 0˜7% DCM/MeOH gradient at 20 mL/min) to give 1.6 g (impure, 75% LCMS purity), followed by prep-HPLC [FA condition, column: Boston Uni C18 40*150*5 um; mobile phase: [water (0.225% FA)-ACN]; B %: 8%-38%, 7.7 min.] to give 45-6 (1.04 g, 63.70% yield) as a white solid. ESI-LCMS: 485.0 [M+Na]+; ¹H NMR (400 MHz, DMSO-d₆) δ=11.27-11.21 (m, 1H), 8.77 (s, 1H), 8.74 (s, 1H), 8.05 (d, J=7.3 Hz, 2H), 7.68-7.62 (m, 1H), 7.59-7.53 (m, 2H), 7.39 (t, J=6.3 Hz, 1H), 6.16 (d, J=6.0 Hz, 1H), 5.48 (d, J=5.5 Hz, 1H), 4.55 (t, J=5.5 Hz, 1H), 4.43-4.37 (m, 1H), 4.08-4.02 (m, 1H), 3.41-3.36 (m, 1H), 3.35 (s, 3H), 3.31-3.22 (m, 1H), 2.91 (s, 3H).

Preparation of 45-7: To a solution of 45-6 (1 g, 2.16 mmol) in DCM (30 mL) was added CEP[N(iPr)₂]₂ (977.58 mg, 3.24 mmol, 1.03 mL), followed by DCI (306.43 mg, 2.59 mmol) at 0° C. in one portion under Ar atmosphere. The mixture was degassed and purged with Ar for 3 times, warmed to 20° C., and stirred for 2 hours under Ar atmosphere. Upon completion as monitored by LCMS and TLC (PE:EtOAc=4:1), the reaction mixture was diluted with sat.aq. NaHCO₃ (30 mL) and extracted with DCM (50 mL*2). The combined organic layers were dried over anhydrous Na₂SO₄, filtered, and the filtrate was concentrated under reduced pressure to give a residue. The crude product was purified by reverse-phase HPLC (40 g C18 column: neutral condition, Eluent of 0˜57% of 0.3% NH₄HCO₃ in H₂O/CH₃CN ether gradient at 35 mL/min) to give 45-7 (0.49 g, 33.7% yield) as a white solid. ESI-LCMS: 663.1[M+H]⁺; 1H NMR (400 MHz, CD₃CN) δ=1.19-1.29 (m, 12H) 2.71 (q, J=5.77 Hz, 2H) 2.94 (d, J=6.27 Hz, 3H) 3.35 (d, J=15.56 Hz, 3H) 3.40-3.52 (m, 2H) 3.61-3.97 (m, 4H) 4.23-4.45 (m, 1H) 4.55-4.74 (m, 2H) 6.02 (dd, J=10.67, 6.40 Hz, 1H) 7.25 (br s, 1H) 7.47-7.57 (m, 2H) 7.59-7.68 (m, 1H) 8.01 (d, J=7.78 Hz, 2H) 8.28 (s, 1H) 8.66 (s, 1H) 9.69 (br s, 1H); 31P NMR (162 MHz, CD₃CN) δ=150.92, 149.78.

### Example A35

Preparation of 46-2: To a solution of 46-1 (11.2 g, 24.7 mmol) in DCM (120 mL), imidazole (4.2 g, 61.9 mmol) and TBSCl (5.6 g, 37.1 mmol) were added at room temperature, mixture was stirred at room temperature for 15 hours, LCMS showed 46-1 was consumed completely. Mixture was added to water (500 mL) and extracted with DCM (50 mL*2). The organic phase was dried over Na₂SO₄ and concentrated to give 46-2 (16.0 g) as an oil for the next step.

Preparation of 46-3: To a solution of 46-2 (16.0 g, 28.4 mmol) was added 6% DCA in DCM (160 mL) and triethylsilane (40 mL) at room temperature The reaction mixture was stirred at room temperature for 2 hours. TLC showed 46-2 was consumed completely. Water (300 mL) was added, mixture was extracted with DCM (50 mL*4), organic phase was dried by Na₂SO₄, concentrated by reduce pressure to give crude which was purified by column chromatography (SiO₂, PE/EA=10:1 to 1:1) to give 46-3 (4.9 g, 65.9% yield) as an oil. ESI-LCMS: m/z 263 [M+H]⁺; 1H-NMR (400 MHz, DMSO-d₆) δ 4.84-4.50 (m, 1H), 4.3-4.09 (m, 1H), 3.90-3.80 (m, 1H), 3.75-3.67 (m, 1H), 3.65-3.57 (m, 2H), 3.50-3.44 (m, 1H), 3.37-3.28 (m, 4H), 0.95-0.78 (s, 9H), 0.13-0.03 (s, 6H).

Preparation of 46-4: To a solution of 46-3 (3.3 g, 12.6 mmol) in DMSO (33 mL) was added EDCI (7.2 g, 37.7 mmol). To the resultant mixture was added pyridine (1.1 g, 13.8 mmol) and TFA (788.6 mg, 6.9 mmol). The reaction mixture was stirred at room temperature for 3 hours. TLC (PE/EA=4:1) showed 46-3 was consumed. The mixture was diluted with EA and water was added. The product was extracted with EA. The organic layer was washed with brine and dried over Na₂SO₄ and concentrated to give the crude. This resulted in 46-4 (3.23 g) as an oil for the next step.

Preparation of 46-5: To a solution of 46-4 (3.3 g, 12.6 mmol) in toluene (30 mL) was added POM ester (7.9 g, 12.6 mmol) and KOH (1.3 g, 22.6 mmol) at room temperature. The reaction mixture was stirred at 40° C. for 8 hours. LCMS showed 46-4 was consumed. The mixture was diluted with water and EA was added. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=91/9 Detector, UV 254 nm. This resulted in 46-5 (5.4 g, 9.5 mmol, 75.9% yield) as an oil. ESI-LCMS: m/z 567.2 [M+H]⁺; 1H-NMR (400 MHz, CDCl₃) δ 6.89-6.77 (m, 1H), 6.07-5.96 (m, 1H), 5.86-5.55 (m, 4H), 4.85-4.73 (m, 1H), 4.36-4.27 (m, 1H), 4.05-3.96 (m, 1H), 3.95-3.85 (m, 1H), 3.73-3.65 (m, 1H), 3.44-3.35 (m, 3H), 1.30-1.25 (s, 18H), 0.94-0.84 (s, 9H), 0.14-0.05 (s, 6H). 31P-NMR (162 MHz, CDCl₃) δ 18.30, 15.11.

Preparation of 46-6: To a solution of 46-5 (5.4 g, 9.5 mmol) in HCOOH (30 mL)/H₂O (30 mL)=1:1 at room temperature. The reaction mixture was stirred at room temperature for 15 hours. LCMS showed the reaction was consumed. The mixture was diluted with con. NH₄OH till pH=7.5. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% HCOOH)=30/70 increasing to CH₃CN/H₂O (0.5% HCOOH)=70/30 within 45 min, the eluted product was collected at CH₃CN/H₂O (0.5% HCOOH)=59/41 Detector, UV 220 nm. This resulted in 46-6 (2.4 g, 5.7 mmol, 59.4% yield) as an oil. ESI-LCMS: m/z 453.2 [M+H]⁺; 1H-NMR (400 MHz, DMSO-d₆) δ 6.84-6.68 (m, 1H), 6.07-5.90 (m, 1H), 5.64-5.55 (m, 4H), 5.32-5.24 (m, 1H), 4.23-4.15 (m, 1H), 4.00-3.90 (m, 1H), 3.89-3.80 (m, 1H), 3.78-3.69 (m, 2H), 3.37-3.30 (s, 3H), 1.30-1.10 (s, 18H). 31P-NMR (DMSO-d₆) δ 18.14.

Preparation of 46-7: To a solution of 46-7 (2.1 g, 4.5 mmol) in DCM (21 mL) were added DCI (452.5 mg, 3.8 mmol) and CEP[N(iPr)₂]₂ (1.8 g, 5.9 mmol) at room temperature The reaction mixture was stirred at room temperature for 15 hours under N₂ atmosphere. LCMS showed 46-6 was consumed. The mixture was diluted with water. The product was extracted with DCM (30 mL). The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 28 min, the eluted product was collected at CH₃CN/H₂O (0.5% NH₄HCO₃)=80/20 Detector, UV 254 nm. This resulted in 46-7 (2.8 g, 4.3 mmol, 95.2% yield) as an oil. ESI-LCMS: m/z 653.2 [M+H]⁺; 1H-NMR (400 MHz, DMSO-d₆) δ 6.89-6.77 (m, 1H), 6.11-5.96 (m, 1H), 5.65-5.50 (m, 4H), 4.39-4.34 (d, J=20 Hz, 1H), 4.18-3.95 (m, 2H), 3.94-3.48 (s, 6H), 3.40-3.28 (m, 4H), 2.84-2.75 (m, 2H), 1.26-1.98 (s, 30H). 31P-NMR (162 MHz, DMSO-d₆) δ 149.018, 148.736, 17.775, 17.508.

### Example A36

Preparation of 47-2: To a solution of 47-1 (10.60 g, 47.32 mmol) in DMF (106 mL), imidazole (11.26 g, 165.59 mmol) and TBSCl (19.88 g, 132.53 mmol) were added. The mixture was stirred at room temperature for 3.5 hours, LCMS showed 47-1 was consumed completely. Water was added and extracted with EA, dried over by Na₂SO₄. The filtrate was evaporated under reduced pressure to give 47-2 (20.80 g, 45.94 mmol, 97.19% yield) for the next step.

Preparation of 47-3: To a solution of 47-2 (20.80 g, 45.94 mmol) in THF (248 mL), TFA (124 mL) and H₂O (124 mL) were added at 0° C., then the reaction mixture was stirred for 30 minutes. LCMS showed 47-2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 47-3 (10.00 g, 29.59 mmol, 64.31% yield). 1H-NMR (400 MHz, DMSO-d₆): δ 7.33-7.18 (m, 5H), 4.83-4.80 (m, 1H), 4.61-4.59 (m, 1H), 4.21-4.19 (m, 1H), 3.75-3.74 (m, 1H), 3.23 (m, 3H), 3.13 (m, 3H), 2.41-2.40 (m, 1H), 0.81 (m, 9H), 0.00 (m, 6H).

Preparation of 47-4: To a solution of 47-3 (3.70 g, 10.95 mmol) in DMSO (37 mL) was added EDCI (6.30 g, 32.84 mmol). Then pyridine (0.95 g, 12.05 mmol) and TFA (0.69 g, 6.02 mmol) was added in N₂ atmosphere. The mixture was stirred for 3 hours at room temperature. LCMS showed 47-3 was consumed completely. Water was poured into and extracted with EA, washed with sat. NaCl (aq.), dried over by Na₂SO₄. The filtrate was evaporated under reduced pressure to give the crude product which was directly used for next step.

Preparation of 47-5: To a solution of 47-4 in toluene (100.00 mL), was added 47-4a (6.93 g, 10.97 mmol) and KOH (1.11 g, 19.78 mmol). It was stirred for 3.5 hours at 40° C. in N₂ atmosphere. TLC and LCMS showed 47-4 was consumed completely. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 47-5 (4.30 g, 6.70 mmol, 61.17% yield). 1H-NMR (400 MHz, CDCl₃): δ 7.27-7.26 (m, 4H), 7.17 (m, 1H), 6.94-6.82 (m, 1H), 6.13-6.02 (m, 1H), 5.63-5.56 (m, 4H), 4.90-4.89 (m, 1H), 4.45-4.41 (m, 1H), 3.98-3.95 (m, 1H), 3.39-3.29 (m, 4H), 1.90 (m, 1H), 1.12-0.83 (m, 29H), 0.00 (m, 7H); 31P-NMR (162 MHz, CDCl₃): δ 18.021, 14.472.

Preparation of 47-6: To a solution of 47-5 (4.30 g, 6.70 mmol) in THF (43.00 mL) was added HCOOH (100 mL) and H₂O (100 mL). It was stirred overnight at room temperature LCMS showed 47-5 was consumed completely. NH₄OH was poured into it and was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 47-6 (2.10 g, 3.98 mmol, 59.32% yield). 1H-NMR (400 MHz, CDCl₃): δ 7.40-7.28 (m, 5H), 7.11-7.00 (m, 1H), 6.19-6.14 (m, 1H), 5.71-5.68 (m, 4H), 4.95-4.94 (m, 1H), 4.48-4.47 (m, 1H), 4.05-4.03 (m, 1H), 3.62-3.61 (m, 1H), 3.46 (m, 3H), 3.00-2.99 (m, 1H), 1.22 (m, 18H); 31P-NMR (162 MHz, CDCl₃): δ 18.134.

Preparation of 47-7: To a solution of 47-6 (2.10 g, 3.98 mmol) in DCM (21 mL) was added DCI (410 mg, 3.47 mmol). CEP[N(iPr)₂]₂ (1.40 g, 4.65 mmol) was added in a N₂ atmosphere. LCMS showed 47-6 was consumed completely. DCM and H₂O was poured, the organic phase was washed with water and sat. NaCl (aq.), dried over by Na₂SO₄. The filtrate was evaporated under reduced pressure at 40° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 47-7 (2.10 g, 2.88 mmol). 1H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H); 31P-NMR (162 MHz, DMSO-d₆): δ 149.588, 148.920, 17.355, 17.010.

### Example A37

Preparation of 48-2: To a solution of 48-1 (5.90 g, 21.50 mmol) in DMF (60.00 mL), imidazole (4.39 g, 64.51 mmol) and TBSCl (7.63 g, 49.56 mmol) were added. The mixture was stirred at room temperature for 3.5 hours, LCMS showed 48-1 was consumed completely. Water was added and extracted with EA, dried over by Na₂SO₄. The filtrate was evaporated under reduced pressure to give 48-2 (11.00 g, 21.91 mmol, 98.19% yield) for the next step. ESI-LCMS: m/z 225.1 [M+H]+.

Preparation of (3): To a solution of 48-2 (11.00 g, 21.91 mmol) in THF (55.00 mL) was added TFA (110.00 mL) and H₂O (55.00 mL) at 0° C., reaction mixture was stirred for 30 min. LCMS showed 48-2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 48-3 (6.20 g, 16.32 mmol, 72.94% yield). ESI-LCMS: m/z 411.2 [M+H]+.

Preparation of 48-4: To a solution of 48-3 (3.50 g, 9.02 mmol) in DMSO (35.00 mL) was added EDCI (5.19 g, 27.06 mmol). Then pyridine (0.78 g, 9.92 mmol) and TFA (0.57 g, 4.96 mmol) was added in N₂ atmosphere. The mixture was stirred for 3 hours at room temperature. Water was poured into it and was extracted with EA, washed with sat. NaCl (aq.), dried over by Na₂SO₄. The filtrate was evaporated under reduced pressure to give the crude product which was directly used for next step. ESI-LCMS: m/z 406.2 [M+H]⁺.

Preparation of 48-5: To a solution of 48-4 in toluene (100.00 mL) was added 48-4a (5.73 g, 9.07 mmol) and KOH (916.3 g, 16.33 mmol). It was stirred for 3.5 h at 40° C. in N₂ atmosphere. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 48-5 (5.02 g, 7.25 mmol, 80.44% yield). ESI-LCMS: m/z 693.2 [M+H]⁺; 31P-NMR (162 MHz, DMSO-d₆): δ 17.811

Preparation of 48-6: To a solution of 48-5 (4.59 g, 6.63 mmol) in THF (46.00 mL) was added HCOOH (92.00 mL) and H₂O (92.00 mL). It was stirred overnight at room temperature NH₄OH was poured into it and extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 48-6 (2.52 g, 4.36 mmol, 65.80% yield).

Preparation of 48-7: To a solution of 48-6 (2.00 g, 3.46 mmol) in DCM (21.00 mL) was added DCI (370.00 mg, 3.11 mmol) and CEP (1.12 g, 4.15 mmol) was added in N₂ atmosphere. DCM and H₂O was poured, the organic phase was washed with water and sat. NaCl (aq.), dried over by Na₂SO₄. The filtrate was evaporated under reduced pressure at 38° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 48-7 (2.10 g, 2.70 mmol, 78.07% yield). 1H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H). 31P-NMR (162 MHz, DMSO-d₆): δ 149.588, 148.920, 17.355, 17.010.

### Example A38

Preparation of 49-2: To a solution of 49-1 (22.6 g, 45.23 mmol) in DCM (500 mL) and H₂O (125 mL) were added TEMPO (6.40 g, 40.71 mmol) and DIB (29.14 g, 90.47 mmol) at 0° C. The mixture was stirred at 20° C. for 20 hours. Upon completion as monitored by LCMS, saturated aqueous NaHCO₃ was added to the mixture to adjust pH>8. The mixture was diluted with H₂O (200 mL) and washed with DCM (100 mL*3). The aqueous layer was collected, adjusted to pH<5 by HCl (4M), and extracted with DCM (200 mL*3). The combined organic layers were washed with brine (300 mL), dried over Na₂SO₄, filtered, and concentrated under reduced pressure to give 49-2 (17.5 g, 68.55% yield) as a yellow solid. ESI-LCMS: 514.2 [M+H]⁺; 1H NMR (400 MHz, DMSO-d₆) δ=11.27 (s, 1H), 8.86 (s, 1H), 8.78 (s, 1H), 8.06 (d, J=7.5 Hz, 2H), 7.68-7.62 (m, 1H), 7.59-7.52 (m, 2H), 6.28 (d, J=6.8 Hz, 1H), 4.82-4.76 (m, 1H), 4.54 (dd, J=4.1, 6.7 Hz, 1H), 4.48 (d, J=1.8 Hz, 1H), 3.32 (s, 3H), 0.94 (s, 9H), 0.18 (d, J=4.8 Hz, 6H)

Preparation of 49-3: To a solution of 49-2 (9.3 g, 18.11 mmol) in MeOH (20 mL) was added SOCl₂ (3.23 g, 27.16 mmol, 1.97 mL) dropwise at 0° C. The mixture was stirred at 20° C. for 0.5 hour. Upon completion as monitored by LCMS, the reaction mixture was quenched by addition of saturated aqueous NaHCO₃ (80 mL) and concentrated under reduced pressure to remove MeOH. The aqueous layer was extracted with DCM (80 mL*3). The combined organic layers were washed with brine (200 mL), dried over Na₂SO₄, filtered and concentrated under reduced pressure to give a residue. The residue was purified by flash silica gel chromatography (ISCO®; 120 g SepaFlash® Silica Flash Column, Eluent of 0˜5%, MeOH/DCM gradient at 85 mL/min) to give 49-3 (5.8 g, 60% yield) as a yellow solid. ESI-LCMS: 528.3 [M+H]⁺; 1H NMR (400 MHz, DMSO-d₆) δ=11.28 (s, 1H), 8.79 (d, J=7.3 Hz, 2H), 8.06 (d, J=7.5 Hz, 2H), 7.68-7.62 (m, 1H), 7.60-7.53 (m, 2H), 6.28 (d, J=6.6 Hz, 1H), 4.87 (dd, J=2.4, 4.0 Hz, 1H), 4.61 (dd, J=4.3, 6.5 Hz, 1H), 4.57 (d, J=2.2 Hz, 1H), 3.75 (s, 3H), 3.32 (s, 3H), 0.94 (s, 9H), 0.17 (d, J=2.2 Hz, 6H)

Preparation of 49-4: To a mixture of 49-3 (5.7 g, 10.80 mmol) in CD₃OD (120 mL) was added NaBD₄ (1.63 g, 43.21 mmol) in portions at 0° C., and the mixture was stirred at 20° C. for 1 hour. Upon completion as monitored by LCMS, the reaction mixture was neutralized by AcOH (˜10 mL) and concentrated under reduced pressure to give a residue. The residue was purified by flash silica gel chromatography (ISCO®; 40 g SepaFlash® Silica Flash Column, Eluent of 0˜5%, MeOH/DCM gradient at 40 mL/min) to give 49-4 (4.15 g, 7.61 mmol, 70.45% yield) as a yellow solid. ESI-LCMS: 502.2 [M+H]⁺; 1H NMR (400 MHz, DMSO-d₆) δ=11.23 (s, 1H), 8.76 (s, 2H), 8.04 (d, J=7.3 Hz, 2H), 7.69-7.62 (m, 1H), 7.60-7.52 (m, 2H), 6.14 (d, J=6.0 Hz, 1H), 5.18 (s, 1H), 4.60-4.51 (m, 2H), 3.98 (d, J=3.0 Hz, 1H), 3.32 (s, 3H), 0.92 (s, 9H), 0.13 (d, J=1.5 Hz, 6H)

Preparation of 49-5: To a solution of 49-4 (4.85 g, 9.67 mmol) in pyridine (50 mL) was added DMTrCl (5.90 g, 17.40 mmol) at 25° C. and the mixture was stirred for 2 hours. Upon completion as monitored by LCMS, the reaction mixture was concentrated under reduced pressure to remove pyridine. The residue was diluted with EtOAc (150 mL) and washed with H₂O (50 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜70%, EA/PE gradient at 60 mL/min) to give 49-5 (6.6 g, 84.06% yield) as a yellow solid. ESI-LCMS: 804.3[M+H]⁺, 1H NMR (400 MHz, DMSO-d₆) δ=11.22 (s, 1H), 8.68 (d, J=11.0 Hz, 2H), 8.03 (d, J=7.3 Hz, 2H), 7.68-7.60 (m, 1H), 7.58-7.49 (m, 2H), 7.37-7.30 (m, 2H), 7.27-7.16 (m, 7H), 6.88-6.79 (m, 4H), 6.17 (d, J=4.2 Hz, 1H), 4.72 (t, J=5.0 Hz, 1H), 4.60 (t, J=4.5 Hz, 1H), 4.03-3.98 (m, 1H), 3.71 (s, 6H), 0.83 (s, 9H), 0.12-0.03 (m, 6H)

Preparation of 49-6: To a solution of 49-5 (6.6 g, 8.21 mmol) in THF (16 mL) was added TBAF (1 M, 8.21 mL,), and the mixture was stirred at 20° C. for 2 hours. Upon completion as monitored by LCMS, the reaction mixture was diluted with EA (150 mL) and washed with H₂O (50 mL*3). The organic layer was washed with brine (150 mL), dried over Na₂SO₄, filtered and concentrated under reduced pressure to give a residue. The residue was purified by flash silica gel chromatography (ISCO®; 80 g SepaFlash® Silica Flash Column, Eluent of 10-100%, EA/PE gradient at 30 mL/min) to give 49-6 (5.4 g, 94.4% yield) as a yellow solid. ESI-LCMS: 690.3 [M+H]+; 1H NMR (400 MHz, DMSO-d₆) δ=11.24 (s, 1H), 8.69 (s, 1H), 8.62 (s, 1H), 8.05 (d, J=7.3 Hz, 2H), 7.69-7.62 (m, 1H), 7.60-7.52 (m, 2H), 7.40-7.33 (m, 2H), 7.30-7.18 (m, 7H), 6.84 (dd, J=5.9, 8.9 Hz, 4H), 6.19 (d, J=4.8 Hz, 1H), 5.36 (d, J=6.0 Hz, 1H), 4.59-4.52 (m, 1H), 4.48 (q, J=5.1 Hz, 1H), 4.11 (d, J=4.8 Hz, 1H), 3.72 (d, J=1.0 Hz, 6H), 3.40 (s, 3H).

Preparation of 49-7: To a solution of 49-6 (8.0 g, 11.60 mmol) in MeCN (150 mL) was added P-1 (4.54 g, 15.08 mmol, 4.79 mL) at 0° C., followed by DCI (1.51 g, 12.76 mmol) in one portion. The mixture was warmed to 20° C. and stirred for 2 hours. Upon completion as monitored by LCMS, the reaction mixture was quenched by addition of saturated aqueous NaHCO₃ (50 mL) and diluted with DCM (250 mL). The organic layer was washed with saturated aqueous NaHCO₃ (50 mL*2), dried over Na₂SO₄, filtered and concentrated under reduced pressure. The residue was purified by a flash silica gel column (0% to 60% EA in PE contain 0.5% TEA) to give 49-7 (5.75 g, 55.37% yield) as a white solid. ESI-LCMS: 890.4 [M+H]⁺; 1H NMR (400 MHz, CD₃CN) δ=9.55 (s, 1H), 8.63-8.51 (m, 1H), 8.34-8.24 (m, 1H), 7.98 (br d, J=7.5 Hz, 2H), 7.65-7.55 (m, 1H), 7.53-7.46 (m, 2H), 7.44-7.37 (m, 2H), 7.32-7.17 (m, 7H), 6.84-6.77 (m, 4H), 6.14 (d, J=4.3 Hz, 1H), 4.84-4.73 (m, 1H), 4.72-4.65 (m, 1H), 4.34-4.27 (m, 1H), 3.91-3.61 (m, 9H), 3.50-3.43 (m, 3H), 2.72-2.61 (m, 1H), 2.50 (t, J=6.0 Hz, 1H), 1.21-1.15 (m, 10H), 1.09 (d, J=6.8 Hz, 2H); 31P NMR (162 MHz, CD₃CN) δ=150.01, 149.65.

### Example A39

Preparation of 50-2: To a solution of 50-1 (35 g, 130.2 mmol) in DMF (350 mL) was added imidazole (26.5 g, 390.0 mmol) then added TBSCl (48.7 g, 325.8 mmol) at 0° C. The mixture was stirred at room temperature for 14 hours. TLC showed 50-1 was consumed completely. Water was added to the reaction. The product was extracted with EA. The organic layer was washed with NaHCO₃ and brine. Then the solution was concentrated under reduced pressure to produce the crude 50-2 (64.6 g) as a white solid which was used directly for next step. ESI-LCMS: m/z 498 [M+H]⁺.

Preparation of 50-3: To a solution of 50-2 (64.6 g, 130.2 mmol) in THF (300 mL) and added TFA/H₂O (1:1, 300 mL) at 0° C. The mixture was stirred at 0° C. for 2 hours. TLC showed 50-2 was consumed completely. NaHCO₃ was added to the reaction. The product was extracted with EA. The organic layer was washed with NaHCO₃ and brine. Then the solution was concentrated under reduced pressure. The residue was purified by silica gel column chromatography (eluent, DCM:MEOH=100:1˜20:1). This resulted in 50-3 (31.3 g, 81.7 mmol, 62.6% over two step) as a white solid. ESI-LCMS: m/z 384 [M+H]⁺.

Preparation of 50-4: To a solution of 50-3 (31.3 g, 81.7 mmol) in ACN/H₂O (1:1, 350 mL) was added DAIB (78.0 g, 244.0 mmol) and Tempo (3.8 g, 24.4 mmol). The mixture was stirred at 40° C. for 2 hours. TLC showed 50-3 was consumed completely. The mixture was then filtered to give 50-4 (22.5 g, 55.5 mmol, 70.9%) as a white solid. ESI-LCMS: m/z 398 [M+H]⁺.

Preparation of 50-5: To a solution of 50-4 (22.5 g, 55.5 mmol) in MeOH (225 mL) held at −15° C. with an ice/MeOH bath was added SOCl₂ (7.6 mL, 94.5 mmol), dropwise at such a rate that the reaction temp did not exceed 7° C. After the addition was complete, cooling was removed, the reaction was allowed to stir at room temp. The mixture was stirred at room temperature for 14 hours. TLC showed 50-4 was consumed completely. Then the solution was concentrated under reduced pressure to get crude 50-5 (23.0 g) as a white solid which was used directly for next step. ESI-LCMS: m/z 298 [M+H]⁺.

Preparation of 50-6: To a solution of 50-5 (23 g, 55.5 mmol) in DMF (220 mL) was added imidazole (11.6 g, 165.0 mmol) then added TBSCl (12.3 g, 82.3 mmol) at 0° C. The mixture was stirred at 20° C. for 14 hours. TLC showed 50-5 was consumed completely. Water was added to the reaction. The product was extracted with EA. The organic layer was washed with NaHCO₃ and brine. Then the solution was concentrated under reduced pressure. The residue was purified by silica gel column chromatography (eluent, DCM:MEOH=100:1˜20:1). This resulted in 50-6 (21.3 g, 51.1 mmol, 90% over two step) as a white solid. ESI-LCMS: m/z 412 [M+H]+.

Preparation of 50-7: To the solution of 50-6 (21.0 g, 51.0 mmol) in dry THF/MeOD/D2O=10/2/1 (260.5 mL) was added NaBD₄ (6.4 g, 153.1 mmol) at room temperature and the reaction mixture was stirred at 50° C. for 2 hours. After completion of reaction, the resulting mixture was added CH₃COOD to pH=7, after addition of water, the resulting mixture was extracted with EA (300 mL). The combined organic layer was washed with water and brine, dried over Na₂SO₄. Then the solution was concentrated under reduced pressure and the residue was used for next step without further purification. ESI-LCMS: m/z 386 [M+H]+.

Preparation of 50-8: To a stirred solution of 50-7 (14.0 g, 35 mmol) in pyridine (50 mL) were added BzCl (17.2 g, 122.5 mmol) at 0° C. under N₂ atmosphere. The mixture was stirred at room temperature for 14 hours. TLC showed 50-7 was consumed completely. Then the solution diluted with EA. The organic layer was washed with NaHCO₃ and brine. Then the solution was concentrated under reduced pressure and the residue was used for next step without further purification. To a solution of the crude in pyridine (300 mL) then added 2M NaOH (MeOH:H₂O=4:1, 60 mL) at 0° C. The mixture was stirred at 0° C. for 10 minutes. Then the solution diluted with EA. The organic layer was washed with NH₄Cl and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in 50-8 (14 g, 28.02 mmol, 69.21% yield) as a white solid. ESI-LCMS: m/z 490 [M+H]⁺; 1H-NMR (400 MHz, DMSO-d₆): δ 11.24 (s, 1H), 8.76 (s, 1H), 8.71 (m, 1H), 8.04 (d, J=7 Hz, 2H), 7.66-7.10 (m, 5H), 6.40-6.35 (dd, 1H), 5.71-5.56 (m, 1H), 5.16 (s, 1H), 4.79-4.72 (m, 1H), 4.01 (m, 1H), 0.91 (s, 9H), 0.14 (m, 6H).

Preparation of 50-9: To a solution of 50-8 (5.1 g, 10.4 mmol) in pyridine (50 mL) was added DMTrCl (5.3 g, 15.6 mmol). The mixture was stirred at room temperature for 1 hour. TLC showed 50-8 was consumed completely. Water was added to the reaction. The product was extracted with EA. The organic layer was washed with NaHCO₃ and brine. Then the solution was concentrated under reduced pressure and the residue was used for next step without further purification. ESI-LCMS: m/z 792 [M+H]⁺.

Preparation of 50-10: To a solution of 50-9 (7.9 g, 10.0 mmol) in THF (80 mL) was added 1M TBAF in THF (12 mL). The mixture was stirred at room temperature for 1 hour. TLC showed 50-9 was consumed completely. Water was added to the reaction. The product was extracted with EA. The organic layer was washed with NaHCO₃ and brine. Then the solution was concentrated under reduced pressure the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in 50-10 as a white solid. ESI-LCMS: m/z 678 [M+H]⁺; 1H-NMR (400 MHz, DMSO-d₆): δ 11.25 (s, 1H), 8.74 (s, 1H), 8.62 (s, 1H), 8.04 (d, J=7 Hz, 2H), 7.66-7.53 (m, 3H), 7.33-7.15 (m, 9H), 6.82-6.78 (m, 4H), 6.43 (d, J=20 Hz, 1H), 5.76-5.60 (m, 1H), 4.88-4.80 (m, 1H), 4.13 (d, J=8 Hz, 1H), 3.71 (m, 6H).

Preparation of 50-11: To a solution of 50-10 (6.2 g, 9.1 mmol) in DCM (60 mL) was added DCI (1.1 g, 9.4 mmol) and CEP (3.3 g, 10.9 mmol) under N₂ pro. The mixture was stirred at 20° C. for 0.5 hours. TLC showed 50-10 was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 50-11 (7.5 g, 8.3 mmol, 90.7%) as a white solid. ESI-LCMS: m/z 878 [M+H]⁺; 1H-NMR (400 MHz, DMSO-d₆): δ 11.25 (s, 1H), 8.68-8.65 (dd, 2H), 8.04 (m, 2H), 7.66-7.53 (m, 3H), 7.33-7.15 (m, 9H), 6.82-6.78 (m, 4H), 6.53-6.43 (m, 1H), 5.96-5.81 (m, 1H), 5.36-5.15 (m, 1H), 4.21 (m, 1H), 3.86-3.52 (m, 10H), 2.79-2.61 (m, 2H), 1.21-0.99 (m, 12H); 31P-NMR (162 MHz, DMSO-d₆): δ 149.60, 149.56, 149.48.

### Example 40

Preparation of 51-2: To a solution of 51-1 (20.0 g, 71.2 mmol) in dry DMF 200.0 mL) was added TBSCl (26.8 g, 177.9 mmol) and imidazole (15.6 g, 227.8 mmol). The mixture was stirred at room temperature for 15 hours. TLC showed 51-1 was consumed completely. The reaction mixture was concentrated to give residue. The residue was quenched with DCM (300.0 mL). The DCM layer was washed with H₂O (100.0 mL*2) and brine. The DCM layer concentrated to give crude 51-2 (45.8 g) as a yellow oil. The crude used to next step directly. ESI-LCMS m/z 510.5 [M+H]⁺.

Preparation of 51-3: To a mixture solution of 51-2 (45.8 g) in THF (300.0 mL) was added mixture of H₂O (100.0 mL) and TFA (100.0 mL) at 0° C. over 30 min. Then the reaction mixture was stirred at 0° C. for 4 hours. TLC showed that 51-2 was consumed completely. The reaction mixture pH was adjusted to 7-8 with NH₃.H₂O (100 mL). Then the mixture was extracted with EA (500.0 mL*2). The combined EA layer was washed with brine and concentrated to give crude which was purified by c.c. (PE:EA=5:1˜1:0) to give compound 51-3 (21.0 g, 53.2 mmol, 74.7% yield over 2 steps) as a white solid. ESI-LCMS m/z 396.2 [M+H]⁺.

Preparation of 51-4: To a solution of 51-3 (21.0 g, 53.2 mmol) in ACN (100.0 mL) and water (100.0 mL) were added (diacetoxyiodo)benzene (51.0 g, 159.5 mmol) and TEMPO (2.5 g, 15.9 mmol), The reaction mixture was stirred at 40° C. for 1 hours. TLC showed that 51-3 was consumed completely. The reaction mixture was cooled down to room temperature and filtered, the filtrate was concentrated to give crude which was purified by crystallization (ACN) to give 51-4 (14.5 g, 35.4 mmol, 66.2% yield). ESI-LCMS m/z 410.1[M+H]⁺.

Preparation of 51-5: To a solution of 51-4 (14.5 g, 35.4 mmol) in toluene (90.0 mL) and MeOH (60.0 mL) was added trimethylsilyldiazomethane (62.5 mL, 2.0 M, 141.8 mmol) at 0° C., then stirred at room temperature for 2 hours. TLC showed that 51-4 was consumed completely. The solvent was removed under reduce pressure, the residue was purified by crystallization (ACN) to give 51-5 (10.0 g, 23.6 mmol, 66.6% yield). ESI-LCMS m/z 424.2 [M+H]+

Preparation of 51-6: To the solution of 51-5 (10.0 g, 23.6 mmol) in dry THF/MeOD/D₂O=10/2/1 (100.0 mL) was added NaBD₄ (2.98 g, 70.9 mmol) three times during an hour at 40° C., the reaction mixture was stirred at room temperature for 2 hours. The resulting mixture was added CH₃COOD change pH=7.5, after addition of water, the resulting mixture was extracted with EA (50.0 mL*3). The combined organic layer was washed with water and brine, dried over Na₂SO₄, concentrated to give a residue which was purified by c.c. (PE/EA=1:1˜1:0). This resulted in 51-6 (6.1 g, 15.4 mmol, 65.3% yield) as a white solid. ESI-LCMS m/z 398.1 [M+H]⁺; 1H-NMR (400 MHz, DMSO-d₆) δ 8.28 (s, 1H), 8.02 (s, 1H), 7.23 (s, 2H), 5.86 (d, J=6.4 Hz, 1H), 5.26 (s, 1H), 4.42-4.41 (m, 1H), 4.35-4.32 (m, 1H), 3.82 (d, J=2.6 Hz, 1H), 3.14 (s, 3H), 0.78 (s, 9H), 0.00 (d, J=0.9 Hz, 6H).

Preparation of 51-7: To a solution of 51-6 (6.1 g, 15.4 mmol) in pyridine (60.0 mL) was added the benzoyl chloride (6.5 g, 46.2 mmol) drop wise at 5° C. The reaction mixture was stirred at room temperature for 2 hours. TLC showed the 51-6 was consumed completely. The reaction mixture was cooled down to 10° C. and quenched with H₂O (20.0 mL), extracted with EA (200.0 mL*2), combined the EA layer. The organic phase was washed with brine and dried over Na₂SO₄, concentrated to give the crude (12.0 g) which was dissolved in pyridine (60.0 mL), cooled to 0° C., 20.0 mL NaOH (2 M in methanol:H₂O=4:1) was added and stirred for 10 min. The reaction was quenched by saturated solution of ammonium chloride, the aqueous layer was extracted with EA (200.0 mL*2), combined the EA layer, washed with brine and dried over Na₂SO₄, concentrated. The residue was purified by c.c. (PE/EA=10:1˜1:1) to give 51-7 (7.0 g, 13.9 mmol, 90.2% yield). ESI-LCMS m/z 502.2 [M+H]⁺; 1H-NMR (400 MHz, DMSO-d₆) δ 11.24 (s, 1H, exchanged with D₂O) 8.77 (s, 2H), 8.04-8.06 (m, 2H), 7.64-7.66 (m, 2H), 7.54-7.58 (m, 2H), 6.14-6.16 (d, J=5.9 Hz, 1H), 5.20-5.23 (m, 1H), 4.58-4.60 (m, 1H), 4.52-4.55 (m, 1H), 3.99-4.01 (m, 1H), 3.34 (s, 4H), 0.93 (s, 9H), 0.14-0.15 (d, J=1.44 Hz, 6H).

Preparation of 51-8: To a stirred solution of 51-7 (5.5 g, 10.9 mmol) in DMSO (55.0 mL) was added EDCI (6.3 g, 32.9 mmol), pyridine (0.9 g, 10.9 mmol) and TFA (0.6 g, 5.5 mmol), the reaction mixture was stirred at room temperature for 15 hours. The reaction was quenched with water and extracted with EA (100.0 mL). The organic phase was washed by brine, dried over Na₂SO₄. The organic phase was evaporated to dryness under reduced pressure to give a residue 51-8 (4.8 g) which was used directly to next step. ESI-LCMS: m/z 517.1 [M+H₂O]+.

Preparation of 51-9b: A solution of 51-9a (35.0 g, 150.8 mmol) and NaI (90.5 g, 603.4 mmol) in dry ACN (180.0 mL) was added chloromethyl pivalate (113.6 g, 754.3 mmol) at room temperature, the reaction was stirred at 80° C. for 4 hours. The reaction was cooled to room temperature and quenched by water, then the mixture was extracted with EA (500.0 mL*3), combined the organic layer was washed with saturated solution of ammonium chloride, followed by with brine and dried over Na₂SO₄. Then the organic layer was concentrated to give a residue which was purified by c.c., this resulted in 51-9b (38.0 g, 60.1 mmol, 39.8% yield) as a white solid. ESI-LCMS m/z 655.2 [M+Na]+; 1H-NMR (400 MHz, CDCl₃): δ 5.74-5.67 (m, 8H), 2.67 (t, J=21.6 Hz, 2H), 1.23 (s, 36H).

Preparation of 51-9: 3.8 g 10% Pd/C was washed with dry THF (30.0 mL) three times. Then transferred into a round-bottom flask charged with 51-9b (38.0 g, 60.1 mmol) and solvent (dry THF:D₂O=5:1, 400.0 mL), the mixture was stirred at 80° C. under 1 L H₂ balloon for 15 hours. The reaction was cooled to room temperature and extracted with EA (500.0 mL*3), combined the organic layer was washed with brine and dried over Na₂SO₄. The residue 51-9 (3.0 g, 3.7 mmol, 38.8% yield) as a white solid was used directly to next step without further purification. ESI-LCMS m/z 657.2 [M+Na]+; 1H-NMR (400 MHz, CDCl₃): δ 5.74-5.67 (m, 8H), 1.23 (s, 36H).

Preparation of 51-10: A solution of 51-8 (4.8 g, 9.6 mmol), 51-9 (7.3 g, 11.5 mmol) and K₂CO₃ (4.0 g, 38.8 mmol) in dry THF (60.0 mL) and D₂O (20.0 mL) was stirred at room temperature 18 h. LC-MS showed 51-8 was consumed completely. The product was extracted with EA (300.0 mL) and the organic layer was washed with brine and dried over Na₂SO₄. Then the organic layer was concentrated to give a residue which was purified by c.c. (PE/EA=5:1˜1:1) and MPLC. This resulted in 10 (3.0 g, 3.7 mmol, 38.8% yield) as a white solid. ESI-LCMS m/z 806.4[M+H]⁺; 1H-NMR (400 MHz, DMSO-d₆): δ 11.25 (s, 1H, exchanged with D₂O) 8.75 (s, 2H), 8.07-8.05 (d, J=8.0 Hz, 2H), 7.67-7.54 (m, 3H), 6.05 (d, J=5.1 Hz, 1H), 5.65-5.58 (m, 4H), 4.80-4.70 (m, 2H), 4.59-4.57 (m, 1H), 3.36 (s, 3H), 1.11 (s, 9H), 1.10 (s, 9H), 0.94 (s, 9H), 0.17-0.16 (m, 6H); 31P NMR (162 MHz, DMSO-d₆) δ 17.02.

Preparation of 51-11: To a round-bottom flask was added 51-10 (3.0 g, 3.7 mmol) in a mixture of H₂O (30.0 mL), HCOOH (30.0 mL). The reaction mixture was stirred at 40° C. for 15 hours. LC-MS showed the 51-10 was consumed completely. The reaction mixture was adjusted the pH=6-7 with con. NH₃.H₂O (100.0 mL). Then the mixture was extracted with DCM (100.0 mL*3). The combined DCM layer was dried over Na₂SO₄. Filtered and filtrate was concentrated to give crude which was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 20 min, the eluted product was collected at CH₃CN/H₂O (0.5% NH₄HCO₃)=3/2; Detector, UV 254 nm. To give product 51-11 (1.8 g, 2.6 mmol, 70.3% yield). ESI-LCMS m/z=692.2[M+H]⁺; 1H-NMR (400 MHz, DMSO-d₆): δ 11.11 (s, 1H, exchanged with D₂O) 8.71-8.75 (d, J=14.4, 2H), 8.04-8.06 (m, 2H), 7.64-7.65 (m, 1H), 7.54-7.58 (m, 2H), 6.20-6.22 (d, J=5.4, 2H), 5.74-5.75 (d, J=5.72, 2H), 5.56-5.64 (m, 4H), 4.64-4.67 (m, 1H), 4.58-4.59 (m, 1H), 4.49-4.52 (m, 1H), 3.37 (s, 3H), 1.09-1.10 (d, J=1.96, 18H); 31P NMR (162 MHz, DMSO-d₆) δ 17.46.

Preparation of 51-12: To a solution of 51-11 (1.8 g, 2.6 mmol) in DCM (18.0 mL) was added the DCI (276.0 mg, 2.3 mmol), then CEP[N(ipr)₂]₂ (939.5 mg, 3.1 mmol) was added. The mixture was stirred at room temperature for 1 hour. TLC showed 51-11 consumed completely. The reaction mixture was washed with H₂O (50.0 mL*2) and brine (50.0 mL*2), dried over Na₂SO₄ and concentrated to give crude which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. The product was concentrated to give 51-12 (2.0 g, 2.2 mmol, 86.2% yield) as a white solid. ESI-LCMS m/z 892.3[M+H]⁺; 1H-NMR (400 MHz, DMSO-d₆): δ 11.27 (s, 1H, exchanged with D₂O) 8.72-8.75 (m, 2H), 8.04-8.06 (m, 2H), 7.54-7.68 (m, 3H), 6.20-6.26 (m, 1H), 5.57-5.64 (m, 4H), 4.70-4.87 (m, 3H), 3.66-3.88 (m, 4H), 3.37-3.41 (m, 3H), 2.82-2.86 (m, 2H), 1.20-1.21 (m, 12H), 1.08-1.09 (m, 18H); 31P-NMR (162 MHz, DMSO-d₆): δ 150.03, 149.19, 17.05, 16.81.

### Example A41

Preparation of 52-2: To a solution of 52-1 (26.7 g*2, 0.1 mol) in DMF (400 mL) was added sodium hydride (4.8 g, 0.1 mol) for 30 min, then was added CD3I (16 g, 0.1 mol) at 0° C. for 2.5 hours. The mixture was stirred at room temperature for another 1 hour. LCMS showed the reaction was consumed. The mixture was filtered and the clear solution was evaporated to dryness and was evaporated with CH₃OH. The crude was purified by silica gel column (SiO₂, DCM/MeOH=50:1˜15:1). This resulted in the product 52-2 (35.5 g, 124.6 mmol, 62% yield) as a solid. ESI-LCMS: m/z 285 [M+H]⁺.

Preparation of 52-3: To a solution of 52-3 (35.5 g, 124.6 mmol) in DMF (360 mL) was added imidazole (29.7 g, 436.1 mmol) and TBSCl (46.9 g, 311.5 mmol). The mixture was stirred at room temperature overnight. LCMS showed 52-2 was consumed completely. The reaction was quenched with water (500 mL). The product was extracted into ethyl acetate (1 L). The organic layer was washed with brine and dried over anhydrous Na₂SO₄. The crude was purified by silica gel column (SiO₂, PE/EA=4:1˜1:1). This resulted in the product 52-3 (20.3 g, 39.6 mmol, 31.8% yield) as a solid. ESI-LCMS: m/z 513 [M+H]⁺; 1H-NMR (400 MHz, DMSO-d₆): δ 8.32 (m, 1H), 8.13 (m, 1H), 7.31 (m, 2H), 6.02-6.01 (d, J=4.0 Hz, 1H), 4.60-4.58 (m, 1H), 4.49-4.47 (m, 1H), 3.96-3.86 (m, 2H), 3.72-3.68 (m, 1H), 0.91-0.85 (m, 18H), 0.13-0.01 (m, 12H).

Preparation of 52-4: To a solution of 52-3 (20.3 g, 39.6 mmol) in THF (80 mL) was added TFA (20 mL) and water (20 mL) at 0° C. The reaction mixture was stirred at 0° C. for 5 hours. LC-MS showed 52-3 was consumed completely. Con. NH₄OH was added to the mixture at 0° C. to quench the reaction until the pH=7.5. The product was extracted into ethyl acetate (200 mL). The organic layer was washed with brine and dried over anhydrous Na₂SO₄. The solution was then concentrated under reduced pressure and the residue was washed by PE/EA=5:1. This resulted in 52-4 (10.5 g, 26.4 mmol, 66.6% yield) as a white solid. ESI-LCMS: m/z 399 [M+H]⁺; 1H-NMR (400 MHz, DMSO-d₆): δ 8.41 (m, 1H), 8.14 (m, 1H), 7.37 (m, 2H), 5.99-5.97 (d, J=8.0 Hz, 1H), 5.43 (m, 1H), 4.54-4.44 (m, 2H), 3.97-3.94 (m, 1H), 3.70-3.53 (m, 2H), 0.91 (m, 9H), 0.13-0.12 (m, 6H).

Preparation of 52-5: To a solution of 52-4 (10.5 g, 26.4 mmol) in ACN/H₂O=1:1 (100 mL) was added DAIB (25.4 g, 79.2 mmol) and TEMPO (1.7 g, 7.9 mmol). The reaction mixture was stirred at 40° C. for 2 hours. LCMS showed 52-4 was consumed. The mixture was diluted with EA and water was added. The product was extracted with EA. The organic layer was washed with brine and dried over anhydrous Na₂SO₄. The solution was then concentrated under reduced pressure and the residue was washed by ACN. This resulted in 52-5 (6.3 g, 15.3 mmol, 57.9% yield) as a white solid. ESI-LCMS: m/z 413 [M+H]⁺; 1H-NMR (400 MHz, DMSO-d₆): δ=8.48 (m, 1H), 8.16 (m, 1H), 7.41 (m, 2H), 6.12-6.10 (d, J=8.0 Hz, 1H), 4.75-4.73 (m, 1H), 4.42-4.36 (m, 2H), 3.17 (m, 6H), 2.07 (m, 2H), 0.93 (m, 9H), 0.17-0.15 (m, 6H).

Preparation of 52-6: To a solution of 52-5 (6.3 g, 15.3 mmol) in toluene (36 mL) and methanol (24 mL) was added (trimethylsilyl)diazomethane (7.0 g, 61.2 mmol) at room temperature for 2 minutes. LCMS showed the reaction was consumed. The solvent was removed to give the cured 52-6 (6.0 g) as a solid witch used for the next step. ESI-LCMS: m/z 427 [M+H]⁺; 1H-NMR (400 MHz, DMSO-d₆): δ 8.45 (m, 1H), 8.15 (m, 1H), 7.35 (m, 2H), 6.12-6.10 (d, J=8.0 Hz, 1H), 4.83-4.81 (m, 1H), 4.50-4.46 (m, 1H), 3.73 (m, 3H), 3.31 (m, 1H), 0.93 (m, 9H), 0.15-0.14 (m, 6H).

Preparation of 52-7: To the solution of 52-6 (6 g) in dry THF/MeOD/D₂O=10/2/1 (78 mL) was added NaBD₄ (2.3 g, 54.8 mmol) at room temperature And the reaction mixture was stirred at room temperature for 2.5 hours. After completion of reaction, adjusted pH value to 7 with CH₃COOD, after addition of water, the resulting mixture was extracted with EA (100 mL). The combined organic layer was washed with water and brine, dried over Na₂SO₄, and concentrated to give 52-7 (5.7 g) which was used for the next step. ESI-LCMS: m/z 401 [M+H]+.

Preparation of 52-8: To a solution of 52-7 (5.7 g) in pyridine (60 mL) was added BzCl (10.0 g, 71.3 mmol) under ice bath. The reaction mixture was stirred at room temperature for 2.5 hours. LCMS showed 52-7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=7/3; Detector, UV 254 nm. This resulted in the crude 52-8 (6.2 g, 8.7 mmol, 57% yield, over two steps) as a white solid. ESI-LCMS: m/z 713 [M+H]⁺.

Preparation of 52-9: To a solution of 52-8 (6.2 g, 8.7 mmol) in pyridine (70 mL) and was added 1M NaOH (MeOH/H₂O=4/1) (24 mL). LCMS showed 52-8 was consumed. The mixture was added saturated NH₄Cl till pH=7.5. The mixture was diluted with water and EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=67/33 Detector, UV 254 nm. This resulted in the product 52-9 (4.3 g, 8.5 mmol, 98% yield) as a white solid. ESI-LCMS: m/z 505 [M+H]⁺; 1H-NMR (400 MHz, DMSO-d₆): δ 11.23 (m, 1H), 8.77 (m, 2H), 8.06-8.04 (m, 2H), 7.66-7.63 (m, 2H), 7.57-7.53 (m, 3H), 6.16-6.14 (d, J=8.0 Hz, 1H), 5.17 (m, 1H), 4.60-4.52 (m, 2H), 3.34 (m, 1H), 0.93 (m, 9H), 0.14 (m, 6H).

Preparation of 52-10: To a stirred solution of 52-9 (4.3 g, 8.5 mmol) in pyridine (45 mL) were added DMTrCl (3.3 g, 9.8 mmol) at room temperature And the reaction mixture was stirred at room temperature for 2.5 hours. With ice-bath cooling, the reaction was quenched with water and the product was extracted into EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=97/3 Detector, UV 254 nm. This resulted in the product 52-10 (6.5 g, 8.1 mmol, 95% yield) as a white solid. ESI-LCMS: m/z 807 [M+H]⁺; 1H-NMR (400 MHz, DMSO-d₆): δ 11.23 (m, 1H), 8.70-8.68 (m, 2H), 8.04-8.02 (m, 2H), 7.66-7.62 (m, 1H), 7.56-7.52 (m, 2H), 7.35-7.26 (m, 2H), 7.25-7.17 (m, 7H), 6.85-6.82 (m, 4H), 6.18-6.16 (d, J=8.0 Hz, 1H), 4.73-4.70 (m, 1H), 4.61-4.58 (m, 1H), 3.71 (m, 6H), 3.32 (m, 1H), 0.83 (m, 9H), 0.09-0.03 (m, 6H).

Preparation of 52-11: To a solution of 52-10 (3.5 g, 4.3 mmol) in THF (35 mL) was added 1 M TBAF solution (5 mL). The reaction mixture was stirred at room temperature for 1.5 hours. LCMS showed 52-10 was consumed completely. Water (100 mL) was added. The product was extracted with EA (100 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 20 min, the eluted product was collected at CH₃CN/H₂O (0.5% NH₄HCO₃)=62/38; Detector, UV 254 nm. This resulted in 52-11 (2.7 g, 3.9 mmol, 90.7%) as a white solid. ESI-LCMS: m/z 693 [M+H]+.

Preparation of 52-12: To a suspension of 52-11 (2.7 g, 3.9 mmol) in DCM (30 mL) was added DCI (0.39 g, 3.3 mmol) and CEP[N(iPr)₂]₂ (1.4 g, 4.7 mmol). The mixture was stirred at room temperature for 2 hours. LC-MS showed 52-11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=73/27; Detector, UV 254 nm. This resulted in 52-12 (3.3 g, 3.7 mmol, 94.9%) as a white solid. ESI-LCMS: m/z 893 [M+H]⁺; 1H-NMR (400 MHz, DMSO-d₆): δ=11.24 (m, 1H), 8.66-8.64 (m, 2H), 8.06-8.03 (m, 2H), 7.65-7.53 (m, 3H), 7.42-7.38 (m, 2H), 7.37-7.34 (m, 2H), 7.25-7.19 (m, 7H), 6.86-6.80 (m, 4H), 6.20-6.19 (d, J=4.0 Hz, 1H), 4.78 (m, 2H), 4.22-4.21 (m, 1H), 3.92-3.83 (m, 1H), 3.72 (m, 6H), 3.62-3.57 (m, 3H), 2.81-2.78 (m, 1H), 2.64-2.61 (m, 1H), 1.17-1.04 (m, 12H); 31P-NMR (162 MHz, DMSO-d₆): δ 149.51, 149.30.

### Example A42

Preparation of 53-3: To the solution of 53-1 (70 g, 138.9 mmol) in dry acetonitrile (700 mL) was added 53-2 (27.0 g, 166.7 mmol), BSA (112.8 g, 555.5 mmol). The mixture was stirred at 50° C. for 1 hour. Then the mixture was cooled to −5° C. and TMSOTf (46.2 g, 208.3 mmol) slowly added to the mixture. Then the reaction mixture was stirred at room temperature for 48 hours. Then the solution was cooled to 0° C. and saturated aqueous NaHCO₃ was added and the resulting mixture was extracted with EA. The combined organic layer was washed with water and brine, dried over Na₂SO₄, and concentrated under reduced pressure to give a residue which was purified by silica gel column chromatography (eluent, PE:EA=3:1˜1:1) to give 53-3 (70 g, 115.3 mmol, 81.6%) as a white solid. ESI-LCMS: m/z 605 [M−H]+.

Preparation of 53-4: To the solution of 54-3 (70.0 g, 115.3 mmol) in methylammonium solution (1 M, 700 mL), and the reaction mixture was stirred at 40° C. for 15 hours. After completion of reaction, the resulting mixture was concentrated. The residue was crystallized from EA. Solid was isolated by filtration, washed with PE and dried overnight at 45° C. in vacuum to give 53-4 (31.0 g, 105.4 mmol, 91.1%) as a white solid. ESI-LCMS: m/z 295 [M+H]+; 1H-NMR (400 MHz, DMSO): δ 11.63 (s, 1H), 8.07-7.99 (m, 1H), 7.81 (d, J=8.4 Hz, 1H), 7.72-7.63 (m, 1H), 7.34-7.26 (m, 1H), 6.18 (d, J=6.4 Hz, 1H), 5.24 (s, 1H), 5.00 (s, 2H), 4.58-4.47 (m, 1H), 4.19-4.10 (m, 1H), 3.85-3.77 (m, 1H), 3.75-3.66 (m, 1H), 3.66-3.57 (m, 1H).

Preparation of 53-5: To the solution of 53-4 (20.0 g, 68.0 mmol) in dry DMF (200 mL) was added DPC (18.9 g, 88.0 mmol) and NaHCO₃ (343 mg, 4 mmol) at room temperature, and the reaction mixture was stirred at 150° C. for 35 min. After completion of reaction, the resulting mixture was poured into tert-butyl methyl ether (4 L). Solid was isolated by filtration, washed with PE and dried under vacuum to give crude 53-5 (21.0 g) as a brown solid which was used directly for next step. ESI-LCMS: m/z 275 [M−H]−. Journal of Organic Chemistry, 1989, vol. 33, p. 1219-1225.

Preparation of 53-6: To the solution of 53-5 (crude, 21.0 g) in pyridine (200 mL) was added AgNO3 (31.0 g, 180.0 mmol) and collidine (88.0 g, 720 mmol) and TrtCl (41.5 g, 181 mmol) at room temperature, and the reaction mixture was stirred at room temperature for 15 hours. After addition of water, the resulting mixture was extracted with EA. The combined organic layer was washed with water and brine, dried over Na₂SO₄, and concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 53-6 (10.0 g, 13.1 mmol, 20% yield over 3 steps) as a white solid. ESI-LCMS: m/z 761 [M+H]⁺.

Preparation of 53-7: To the solution of 53-6 (10.0 g, 13.1 mmol) in THF (100 mL) was added 6 N NaOH (30 mL) at room temperature, and the reaction mixture was stirred at room temperature for 1 hour. After addition of NH₄C1, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in 53-7 (9.3 g, 11.9 mmol, 90%) as a white solid. ESI-LCMS: m/z 777 [M−H]−; 1H-NMR (400 MHz, DMSO-d₆): δ 11.57 (s, 1H), 8.02 (d, J=8.7 Hz, 1H), 7.88-7.81 (m, 1H), 7.39-7.18 (m, 30H), 7.09-6.99 (m, 30H), 6.92-6.84 (m, 30H), 6.44 (d, J=4.0 Hz, 1H), 4.87 (d, J=4.0 Hz, 1H), 4.37-4.29 (m, 1H), 4.00-3.96 (m, 1H), 3.76-3.70 (m, 1H), 3.22-3.13 (m, 1H), 3.13-3.04 (m, 1H).

Preparation of 53-8: To the solution of 53-7 (8.3 g, 10.7 mmol) in dry DCM (80 mL) was added pyridine (5.0 g, 64.2 mmol) and DAST (6.9 g, 42.8 mmol) at 0° C., and the reaction mixture was stirred at room temperature for 15 hour. After addition of NH₄C1, the resulting mixture was extracted with DCM. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 53-8 (6.8 g, 8.7 mmol, 81.2%) as a white solid. ESI-LCMS: m/z 779 [M−H]+; 19F-NMR (376 MHz, DMSO-d₆): δ −183.05.

Preparation of 53-9: To the solution of 53-8 (5.8 g, 7.5 mmol) in dry ACN (60 mL) was added TEA (1.5 g, 15.1 mmol), DMAP (1.84 g, 15.1 mmol) and TPSCl (4.1 g, 13.6 mmol) at room temperature, and the reaction mixture was stirred at room temperature for 3 hours under N₂ atmosphere. After completion of reaction, the mixture was added NH₃.H₂O (12 mL). After addition of water, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 53-9 (5.5 g, 7 mmol, 90.2%) as a white solid. ESI-LCMS: m/z 780 [M+H]⁺.

Preparation of 53-10: To a solution of 53-9 (5.5 g, 7 mmol) in DCM (50 mL) with an inert atmosphere of nitrogen was added pyridine (5.6 g, 70.0 mmol) and BzCl (1.2 g, 8.5 mmol) in order at 0° C. The reaction solution was stirred for 30 minutes at room temperature. The solution was diluted with DCM (100 mL) and the combined organic layer was washed with water and brine, dried over Na₂SO₄, and concentrated under reduced pressure to give a residue which was purified by silica gel column chromatography (eluent, PE:EA=5:1-2:1) to give 53-10 (5.4 g, 6.1 mmol, 90.6%) as a white solid. ESI-LCMS: m/z 884 [M+H]⁺; 19F-NMR (DMSO-d₆): δ −183.64.

Preparation of 53-11: To the solution of 53-10 (5.4 g, 6.1 mmol) in the solution of DCA (6%) in DCM (60 mL) was added TES (15 mL) at room temperature, and the reaction mixture was stirred at room temperature for 5-10 minutes. After completion of reaction, the resulting mixture was added NaHCO₃, the resulting mixture was extracted with DCM. The combined organic layer was washed with water and brine, dried over Na₂SO₄, and concentrated under reduced pressure and the residue was crystallized from EA. Solid was isolated by filtration, washed with PE and dried overnight at 45° C. in vacuum to give 53-11 (2.0 g, 5.0 mmol, 83.2%) as a white solid. ESI-LCMS: m/z 400 [M+H]⁺.

Preparation of 53-12: To a solution of 53-11 (2.0 g, 5.0 mmol) in dry Pyridine (20 mL) was added DMTrCl (2.0 g, 6.0 mmol). The reaction mixture was stirred at room temperature for 2.5 hours. LCMS showed 53-11 was consumed and water (200 mL) was added. The product was extracted with EA (200 mL) and the organic layer was washed with brine and dried over Na₂SO₄ and concentrated to give the crude. The crude was purified by c.c. (PE:EA=4:1˜1:1) to give crude 53-12. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 53-12 (2.1 g, 3 mmol, 60%) as a white solid. ESI-LCMS: m/z 702 [M+H]⁺; 1H-NMR (400 MHz, DMSO-d₆): δ 12.63 (s, 1H), 8.54 (d, J=7.8 Hz, 1H), 8.25 (d, J=7.2 Hz, 2H), 7.82 (d, J=3.6 Hz, 2H), 7.67-7.58 (m, 1H), 7.57-7.49 (m, 2H), 7.49-7.39 (m, 1H), 7.39-7.31 (m, 2H), 7.27-7.09 (m, 7H), 6.82-6.69 (m, 4H), 6.23 (d, J=26.1 Hz, 1H), 5.59-5.49 (m, 1H), 4.83-4.61 (m, 1H), 4.15-4.01 (m, 1H), 3.74-3.59 (m, 6H), 3.33-3.28 (m, 1H), 3.16-3.05 (m, 1H). 19F-NMR (DMSO-d₆): δ −191.66.

Preparation of 53-13: To a suspension of 53-12 (2.1 g, 3.0 mmol) in DCM (20 mL) was added DCI (310 mg, 2.6 mmol) and CEP[N(iPr)₂]₂ (1.1 g, 3.7 mmol). The mixture was stirred at room temperature for 1 hour. LC-MS showed 53-12 was consumed completely. The solution was washed with water twice and washed with brine and dried over Na₂SO₄. Then concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 53-13 (2.1 g, 2.3 mmol, 80.0%) as a white solid. ESI-LCMS: m/z 902 [M+H]⁺; 1H-NMR (400 MHz, DMSO-d₆): δ 12.64 (s, 1H), 8.54 (d, J=7.6 Hz, 1H), 8.24 (d, J=7.7 Hz, 2H), 7.93-7.88 (m, 2H), 7.67-7.58 (m, 1H), 7.56-7.42 (m, 3H), 7.41-7.29 (m, 2H), 7.27-7.08 (m, 7H), 6.82-6.64 (m, 4H), 6.37-6.18 (m, 1H), 6.03-5.72 (m, 1H), 5.26-4.83 (m, 1H), 4.28-4.12 (m, 1H), 3.88-3.72 (m, 1H), 3.71-3.37 (m, 9H), 3.15-3.00 (m, 1H), 2.83-2.75 (m, 1H), 2.66-2.57 (m, 1H), 1.21-0.88 (m, 12H). 19F-NMR (376 MHz, DMSO-d₆): δ −189.71. 31P-NMR (162 MHz, DMSO-d₆): δ 149.48, 149.50, 148.95, 148.88.

### Example A43

Preparation of 54-2: To the solution of bromobenzene (2.1 g, 13.6 mmol) in dry THF (15 mL) was added 1.6 M n-BuLi (7 mL, 11.8 mmol) drop wise at −78° C. The mixture was stirred at −78° C. for 0.5 hours. Then 54-1 (3.0 g, 9.1 mmol, Wang, Guangyi et al., Journal of Medicinal Chemistry, 2016, 59(10), 4611-4624) was dissolved in THF (15 mL) and added to the mixture drop wise with keeping at −78° C. Then the reaction mixture was stirred at −78° C. for 1 hour. LC-MS showed 54-1 was consumed completely. Then the solution was added to saturated aqueous NH₄Cl and the resulting mixture was extracted with EA. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in 54-2 (3.0 g, 7.3 mmol, 80.0%) as a white solid. ESI-LCMS: m/z 391 [M−OH]−.

Preparation of 54-3: To the solution of 54-2 (4.0 g, 9.8 mmol) in DCM (40 mL) was added TES (1.9 g, 11.7 mmol) at −78° C., and the mixture was added BF₃.OEt₂ (2.1 g, 14.7 mmol) drop wise at −78° C. The mixture was stirred at −40° C. for 1 hour. LC-MS showed 54-2 was consumed completely. Then the solution was added to saturated aqueous NaHCO₃ and the resulting mixture was extracted with DCM. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in 54-3 (3.1 g, 5.3 mmol, 54.0%) as a water clear oil. ESI-LCMS: m/z 410 [M+H₂O]+; 1H-NMR (400 MHz, CDCl₃): δ 7.48-7.25 (m, 15H), 5.24-5.13 (m, 1H), 4.93-4.74 (m, 1H), 4.74-4.46 (m, 4H), 4.37-4.25 (m, 1H), 4.19-4.05 (m, 1H), 4.00-3.80 (m, 1H), 3.77-3.63 (m, 1H). 19F-NMR (376 MHz, CDCl₃): δ −196.84.

Preparation of 54-5: To the solution of 54-3 (2.1 g, 5.3 mmol) in dry DCM (20 mL) was added 1 M BCl₃ (25 mL, 25.5 mmol) drop wise at −78° C., and the reaction mixture was stirred at −78° C. for 0.5 hour. LC-MS showed 54-3 was consumed completely. After completion of reaction, the resulting mixture was poured into water (50 mL). The solution was extracted with DCM and the combined organic layer was concentrated under reduced pressure to give a crude. The crude in MeOH (4 mL) was added 1 M NaOH (15 mL), and the mixture was stirred at room temperature for 5-10 min. The mixture was extracted with EA. The combined organic layer was washed with brine, dried over Na₂SO₄, and concentrated under reduced pressure to give a residue which was purified by silica gel column chromatography (eluent, DCM:MeOH=40:1˜15:1) to give 54-4 (1.0 g, 4.7 mmol, 88.6%) as a water clear oil. ESI-LCMS: m/z 211 [M−H]−; 1H-NMR (400 MHz, DMSO-d₆): δ 7.58-7.19 (m, 5H), 5.41 (d, J=6.1 Hz, 1H), 5.09-5.95 (m, 1H), 5.95-4.84 (m, 1H), 4.82-4.59 (m, 1H), 4.14-3.94 (m, 1H), 3.89-3.80 (m, 1H), 3.78-3.67 (m, 1H), 3.65-3.53 (m, 1H). 19F-NMR (376 MHz, DMSO-d₆): δ −196.46.

Preparation of 54-5: To a solution of 54-4 (1.0 g, 4.7 mmol) in pyridine (10 mL) was added DMTrCl (2.0 g, 5.7 mmol). The reaction mixture was stirred at room temperature for 2 hours. LCMS showed 54-4 was consumed and water (100 mL) was added. The product was extracted with EA (100 mL) and the organic layer was washed with brine and dried over Na₂SO₄ and concentrated to give the crude. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in 54-5 (2.1 g, 4.1 mmol, 87.0%) as a red oil. ESI-LCMS: m/z 513 [M−H]−; 1H-NMR (400 MHz, DMSO-d₆): δ 7.56-7.16 (m, 14H), 6.94-9.80 (m, 4H), 5.45 (d, J=6.3 Hz, 1H), 5.21-5.09 (m, 1H), 4.89-4.68 (m, 1H), 4.18-4.03 (m, 2H), 3.74 (s, 6H), 3.33-3.29 (m, 1H), 3.26-3.17 (m, 1H). 19F-NMR (376 MHz, DMSO-d₆): δ −194.08.

Preparation of 54-6: To a suspension of 54-5 (2.1 g, 4.1 mmol) in DCM (20 mL) was added DCI (410 mg, 3.4 mmol) and CEP[N(iPr)₂]₂ (1.5 g, 4.9 mmol). The mixture was stirred at room temperature for 1 hour. LC-MS showed 54-5 was consumed completely. The solution was washed with water twice and washed with brine and dried over Na₂SO₄. Then concentrated to give the crude. The crude was purification by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 54-6 (2.1 g, 2.9 mmol, 70.0%) as a white solid. ESI-LCMS: m/z 715 [M+H]⁺; 1H-NMR (400 MHz, DMSO-d₆): δ 7.59-7.16 (m, 14H), 6.94-9.80 (m, 4H), 5.26-5.12 (m, 1H), 5.06-4.77 (m, 1H), 4.50-4.20 (m, 1H), 4.20-4.10 (m, 1H), 3.83-3.63 (m, 7H), 3.59-3.37 (m, 4H), 3.25-3.13 (m, 1H), 2.80-2.66 (m, 1H), 2.63-2.53 (m, 1H), 1.18-0.78 (m, 12H). 19F-NMR (DMSO-d₆): δ −194.40, −194.42, −194.50, −194.53. 31P-NMR (162 MHz, DMSO-d₆): δ 149.38, 149.30, 149.02, 148.98.

### Examples 1-154

A series of modified oligonucleotides containing phosphorothioated sequences of modified nucleoside units were synthesized on an Expedite or ABI 394 synthesizer using standard phosphoramidite chemistry. The solid support was universal controlled pore glass (CPG, 1000A, Glen Research, Sterling Va. or Chemgenes Corp) and the building block monomers are described in Tables 5-7. The reagent (dimethylamino-methylidene) amino)-3H-1,2,4-dithiazaoline-3-thione (DDTT) was used as the sulfur-transfer agent for the synthesis of oligoribonucleotide phosphorothioates (PS linkages). An extended coupling of 0.1M solution of phosphoramidite in CH₃CN in the presence of 5-(ethylthio)-1H-tetrazole or 0.3 M benzyl-thio-tetrazole (BTT) in acetonitrile as an activator to a solid bound oligonucleotide followed by standard capping, oxidation and deprotection afforded modified oligonucleotides. The stepwise coupling efficiency of all modified phosphoramidites was more than 95%. Several modified oligonucleotides containing sequences of modified nucleoside units but having phosphodiester (PO) linkages instead of phosphorothioate (PS) linkages are also made.

For making modified oligonucleotides containing thiophosphoroamidate modifications, the synthesis was carried out on a 1 μM scale in a 5′ to 3′ (reverse) direction with the 5′-phosphoramidite monomers diluted to a concentration of 0.1 M in anhydrous CH₃CN in the presence of 5-(benzylthio)-1H-tetrazole activator (coupling time 2.0-4.0 min) to a solid bound oligonucleotide followed by standard capping, oxidation and deprotection afforded modified oligonucleotides. The stepwise coupling efficiency of all modified phosphoramidites was more than 97%. The DDTT (dimethylamino-methylidene) amino)-3H-1, 2, 4-dithiazaoline-3-thione was used as the sulfur-transfer agent for the synthesis of oligoribonucleotide phosphorothioates. The reverse 5′-O—[(N,N-diisopropylamino)-2-cyanoethoxyphosphinyl]-2′-O-Me-3*-O-(4,4′-dimethoxytrityl)-N⁶-benzoyladenosine, 5′-O-[(N,N-Diisopropylamino)-2-cyanoethoxyphosphinyl]-2′-O-Me-3′-O-(4,4′-dimethoxytrityl)-N⁴-benzoyl-5-methyl cytosine were purchased from Chemgenes while building blocks 3′-NMMTr-2′-O-Me-A and 3′-NMMTr-2′-O-Me-(5m)C were synthesized internally.

### Cleavage and Deprotection

After completion of synthesis the controlled pore glass (CPG) was transferred to a screw cap vial or screw caps RNase free microfuge tube. The oligonucleotide was cleaved from the support with simultaneous deprotection of base and phosphate groups with 1.0 mL of a mixture of ethanolic ammonia (ammonia:ethanol (3:1)) for 15 hr at 55° C. or AMA (aqu Ammonia:Methylamine 1:1) at 65° C. for 90 min. The vial was cooled briefly on ice and then the ethanolic ammonia mixture was transferred to a new microfuge tube. The CPG was washed with 2×0.1 mL portions of deionized water, put in dry ice for 10 minutes then dried in speed vac.

### Quantitation of Crude Oligomer or Raw Analysis

Samples were dissolved in deionized water (1.0 mL) and quantitated as follows: Blanking was first performed with water alone (1 mL). 20 ul of sample and 980 uL of water were mixed well in a microfuge tube, transferred to cuvette and absorbance reading obtained at 260 nm. The crude material is dried down and stored at −20° C.

### HPLC Purification of Oligomer

The crude oligomers were analyzed and purified by HPLC (Dionex PA 100). The buffer system is A=20 mM Sodium Phosphate, 10% Acetonitrile, pH 8.5; B=20 mM Sodium Phosphate, 10% Acetonitrile and 1.8 M NaBr flow 5.0 mL/min, wavelength 260 nm. First inject a small amount of material (˜5 OD/ml) and analyze by LC-MS. Once the identity of this material is confirmed the crude oligomer can then be purified using a larger amount of material, e.g., 60-100 OD's per run, flow rate of 5 mL/min. Fractions containing the full-length oligonucleotides are then pooled together, evaporated and finally desalted as described below.

### Desalting of Purified Oligomer

The purified dry oligomer was then desalted using Sephadex G-25M (Amersham Biosciences). The cartridge was conditioned with 10 mL of water. The purified oligomer dissolved thoroughly in 2.5 mL RNase free water was applied to the cartridge with very slow dropwise elution. The salt free oligomer was eluted with 3.5 ml water directly into a screw cap vial.

### Electrospray LC/MS Analysis

Approximately 0.2 OD oligomer is first dried down, dissolved in water (50 ul) and then pipetted in special vials for HPLC and LC-MS analysis.

### Final Analytical HPLC Analysis

Approximately 0.2 OD of oligomer was analyzed on analytical HPLC.

Column: Waters XBridge BEH C18, 1.7 μm, 2.1×150 mm

Flow rate: 1.0 ml/min

Temp: 60° C.

UV 250 nm

Buffer A: 100 mM Hexafluoro-isopropanol (HFIP, 16.3 mM Triethylamine (TEA), 1% MeOH

Buffer B: 50% Methanol (MeOH), 50% Acetonitrile

| | Flow | Buffer | Buffer | |
|---|---|---|---|---|
| Time | (ml/min) | A (%) | B (%) | Curve |
| 0 | 1.0 | 92.5 | 7.5 | |
| 1 | 1.0 | 92.5 | 7.5 | Linear |
| 15.5 | 1.0 | 82 | 18 | Linear |
| 15.7 | 1.0 | 0 | 100 | Linear |
| 16.7 | 1.0 | 0 | 100 | Linear |
| 16.9 | 1.0 | 92.5 | 7.5 | Linear |
| 22 | 1.0 | 92.5 | 7.5 | Linear |

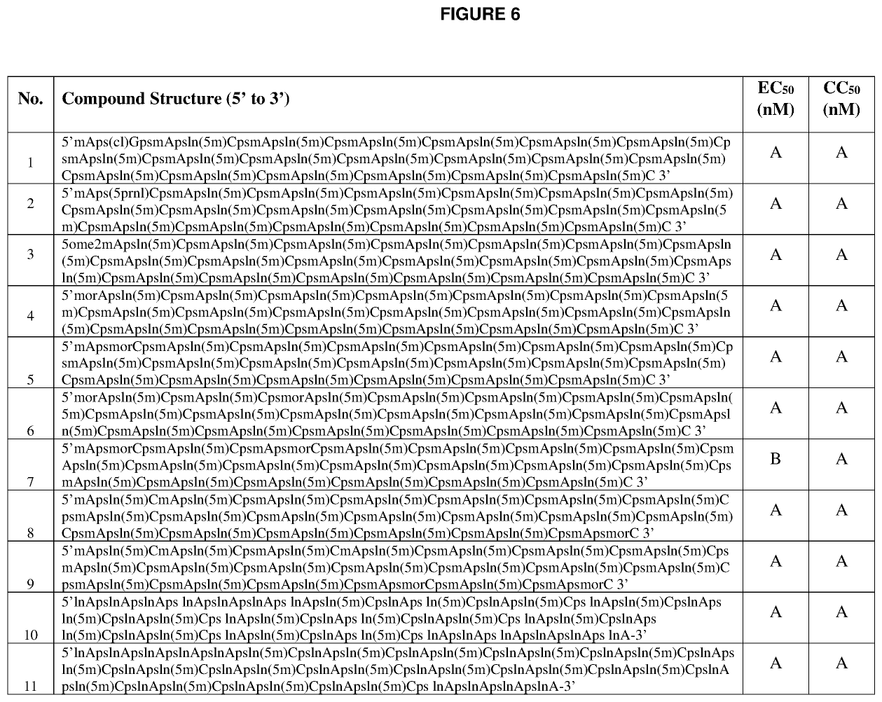

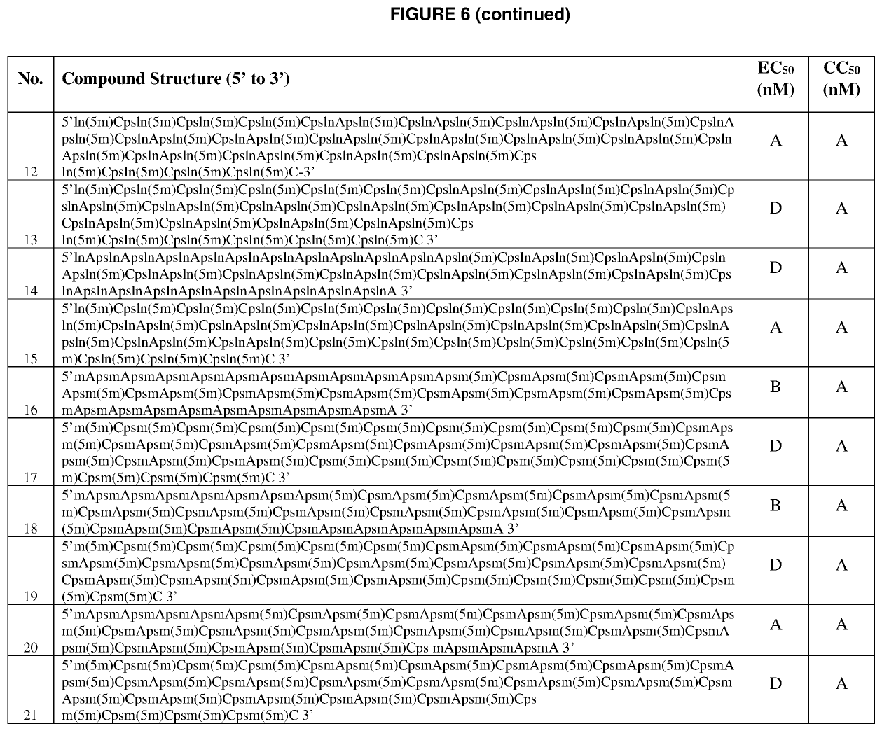

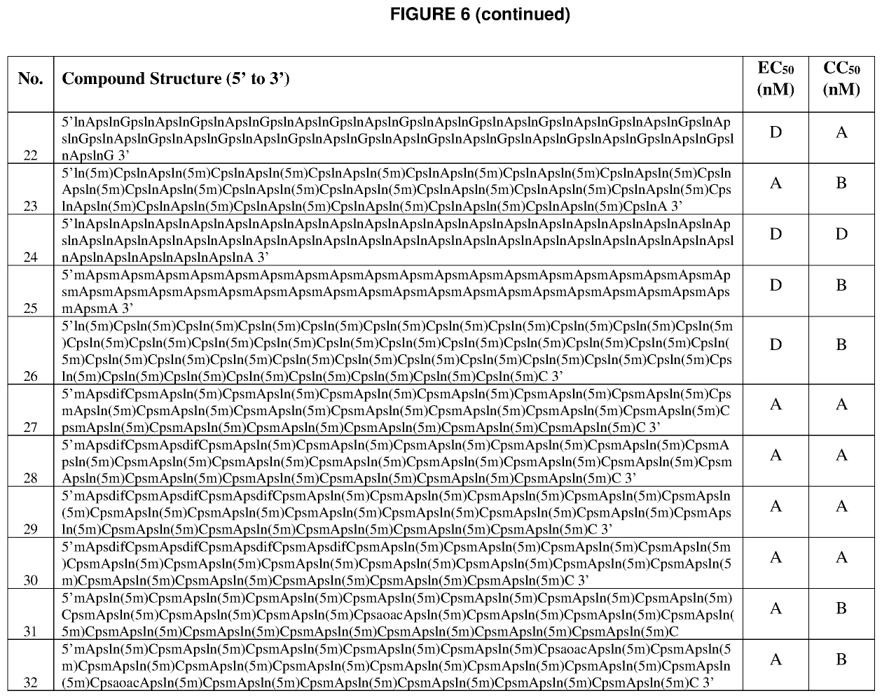

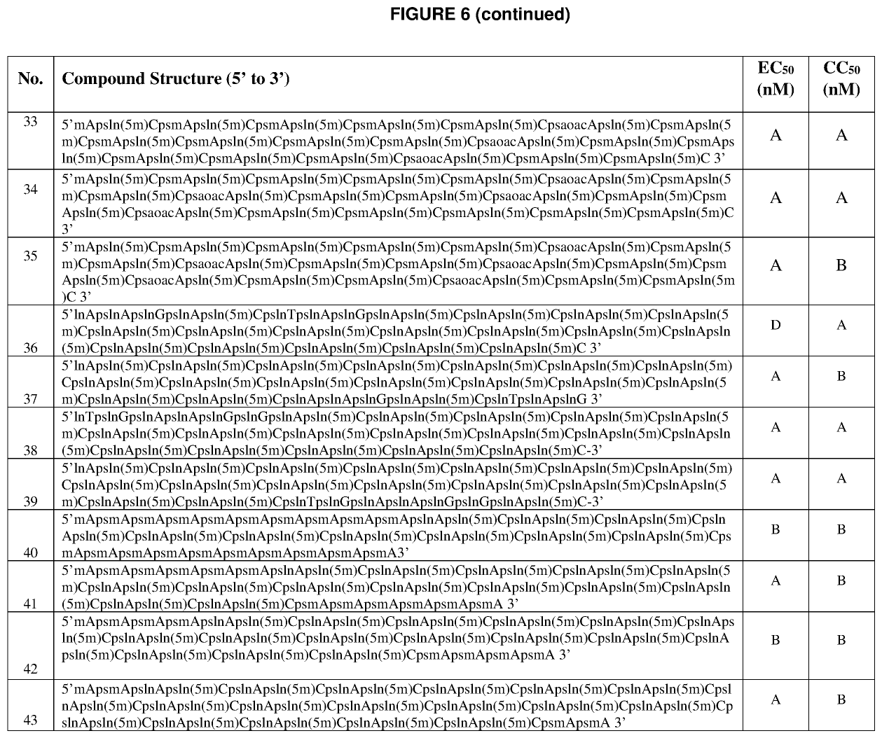

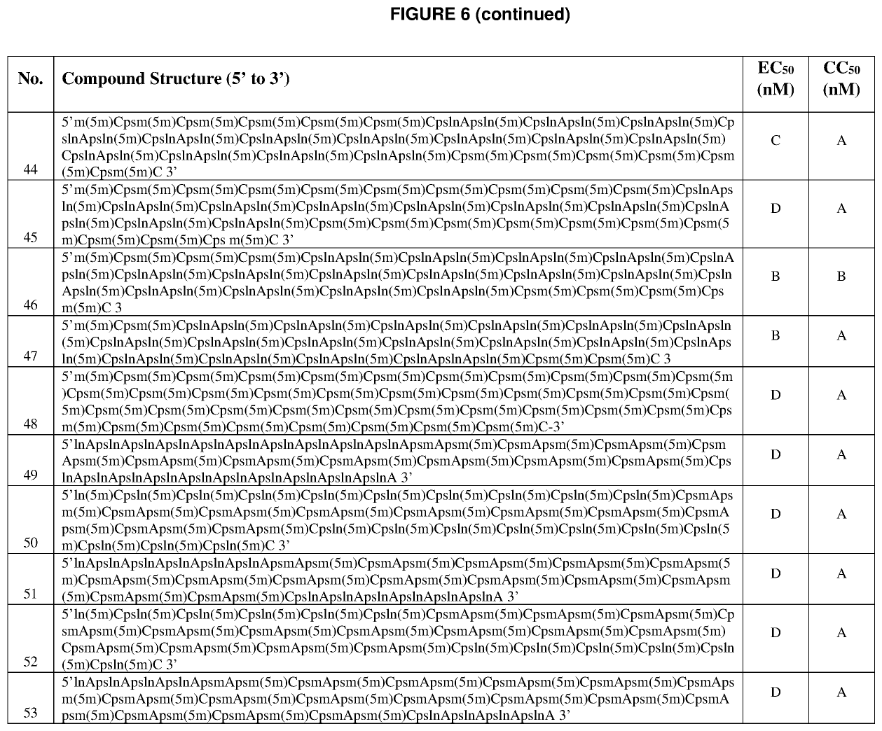

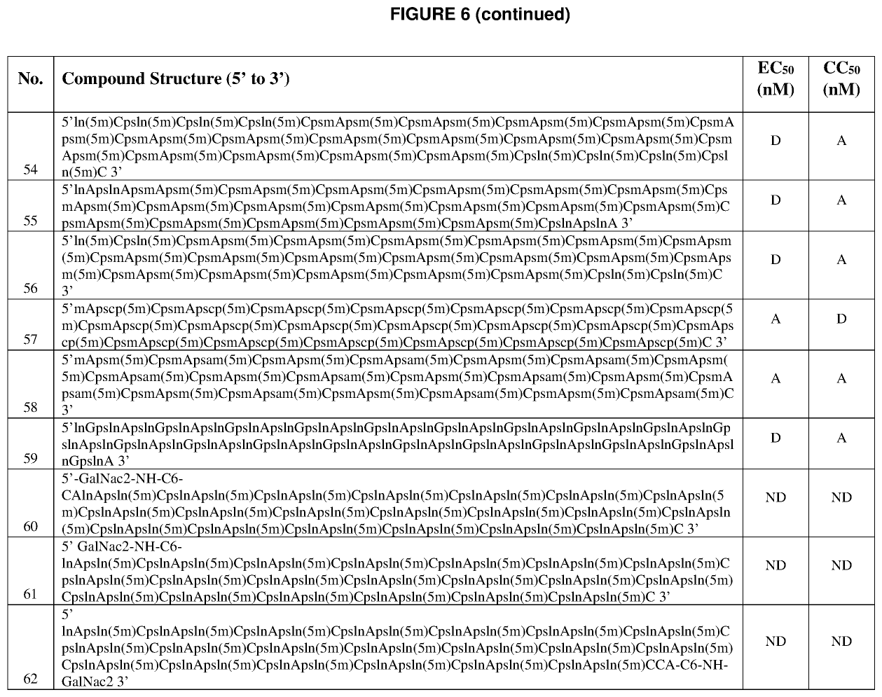

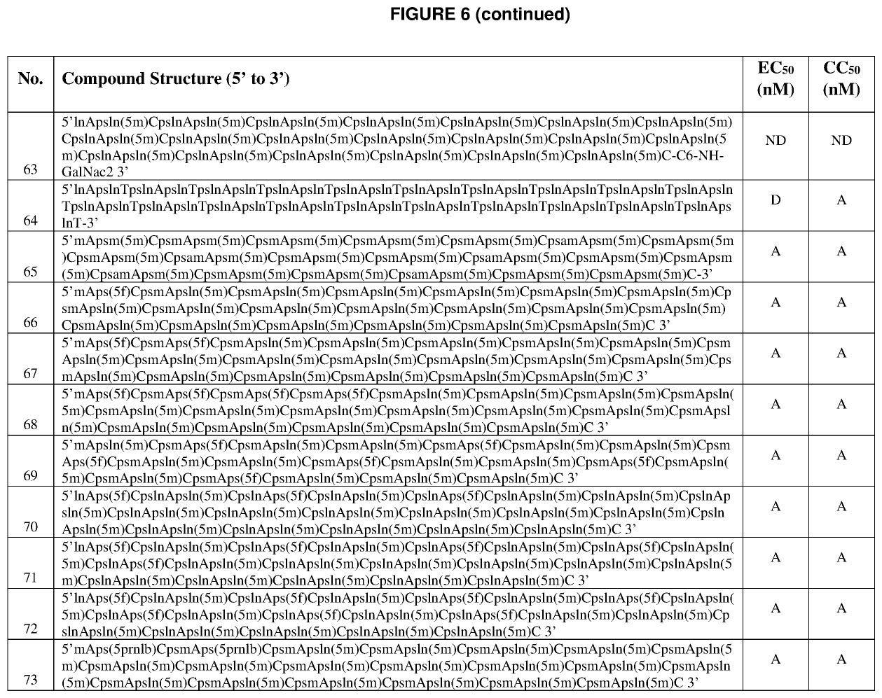

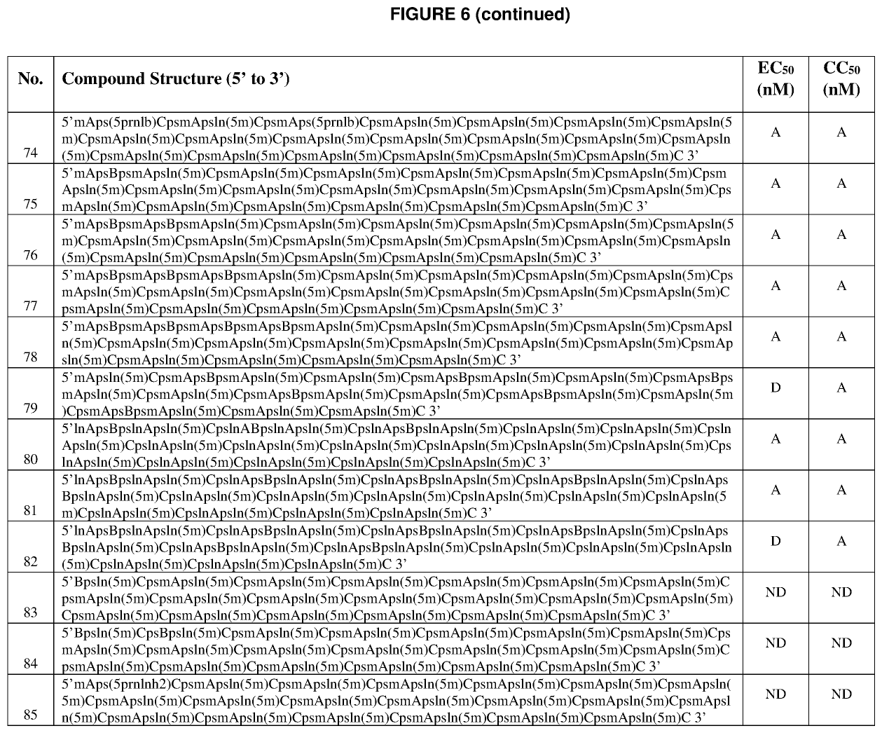

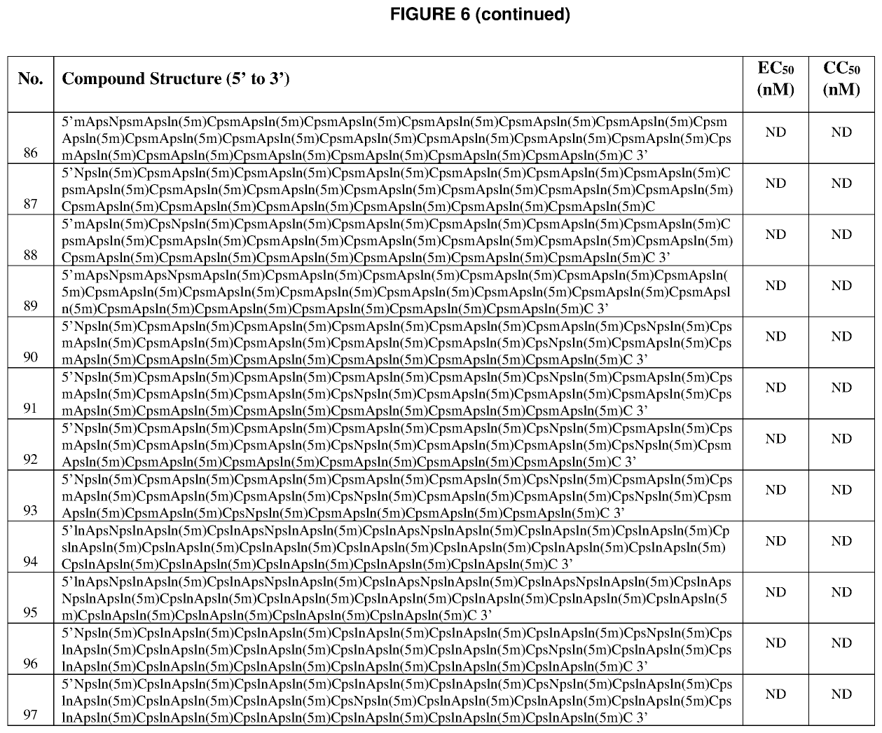

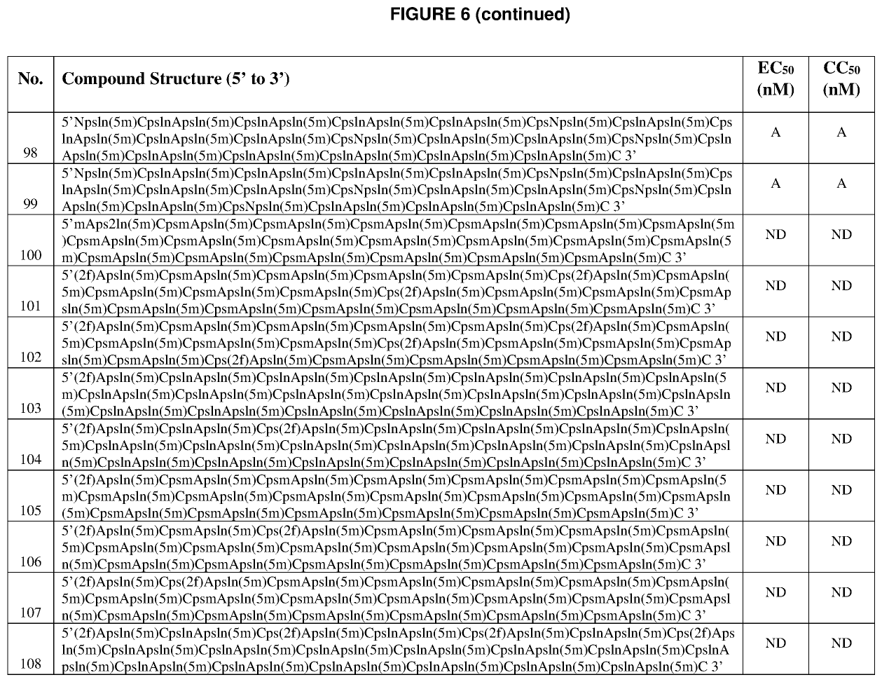

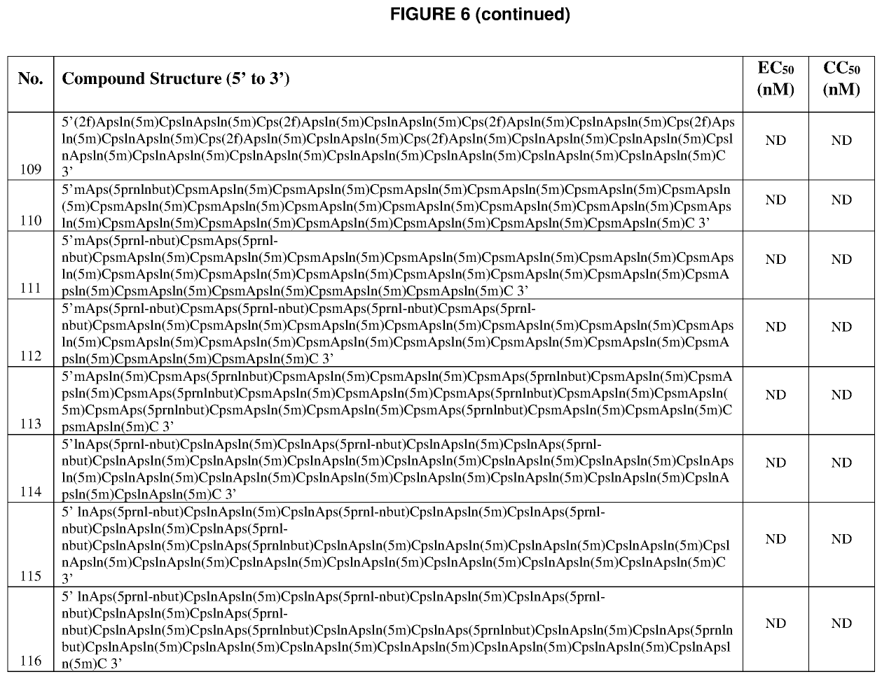

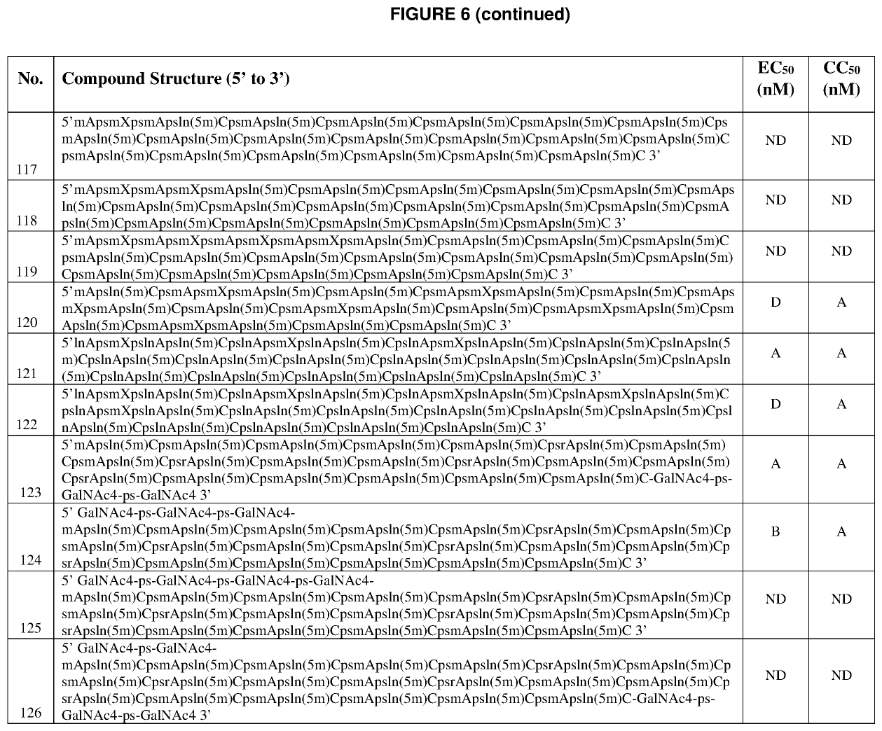

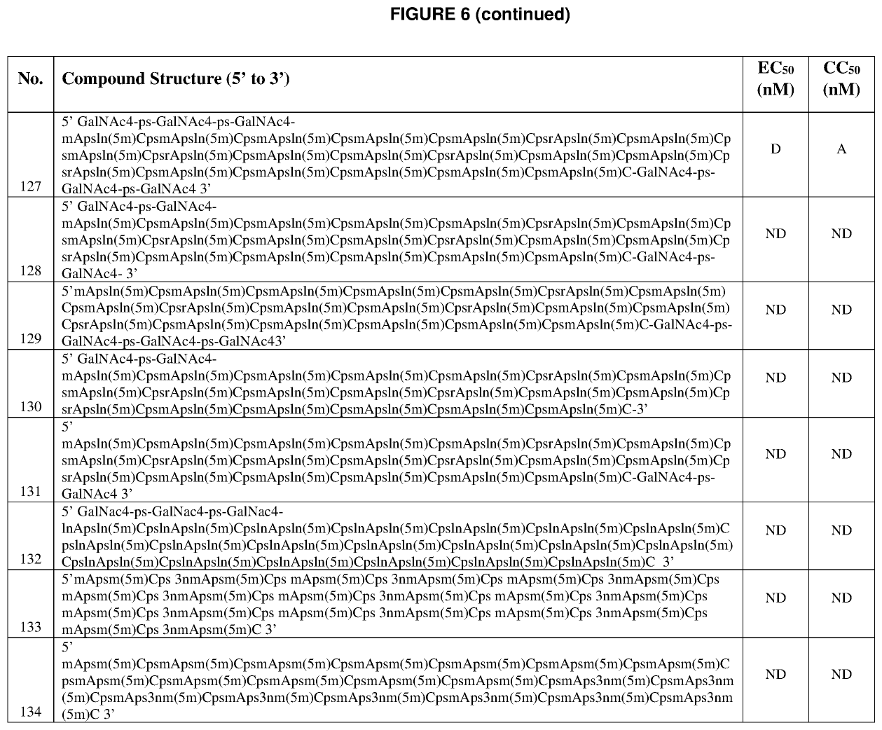

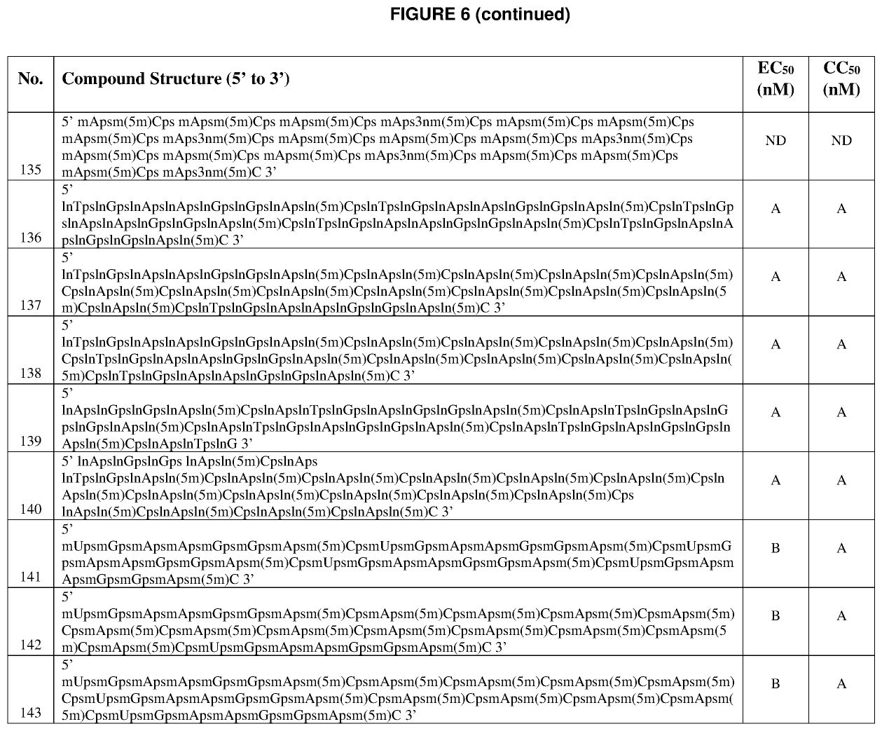

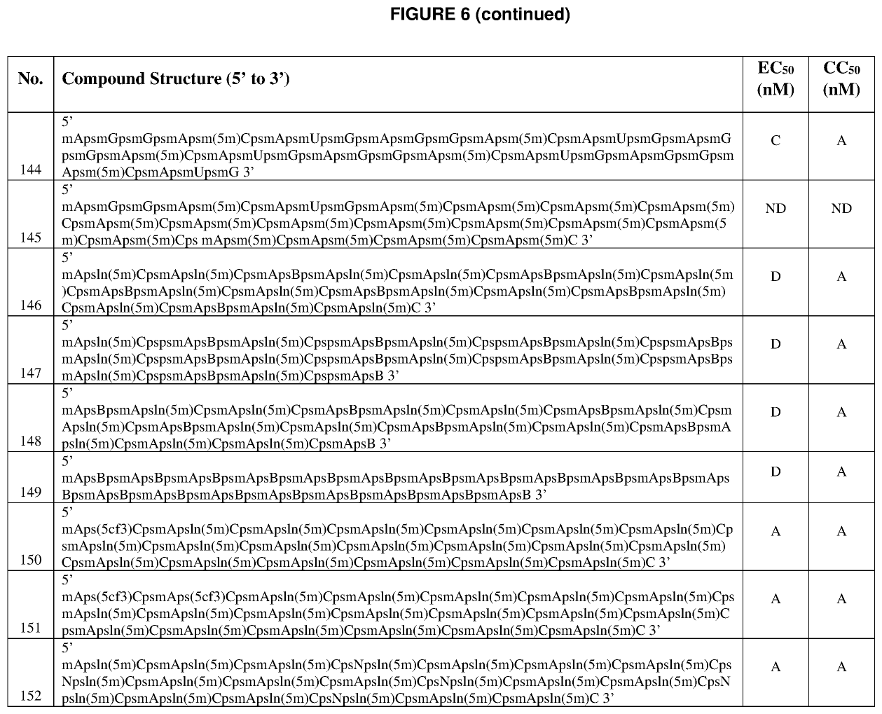

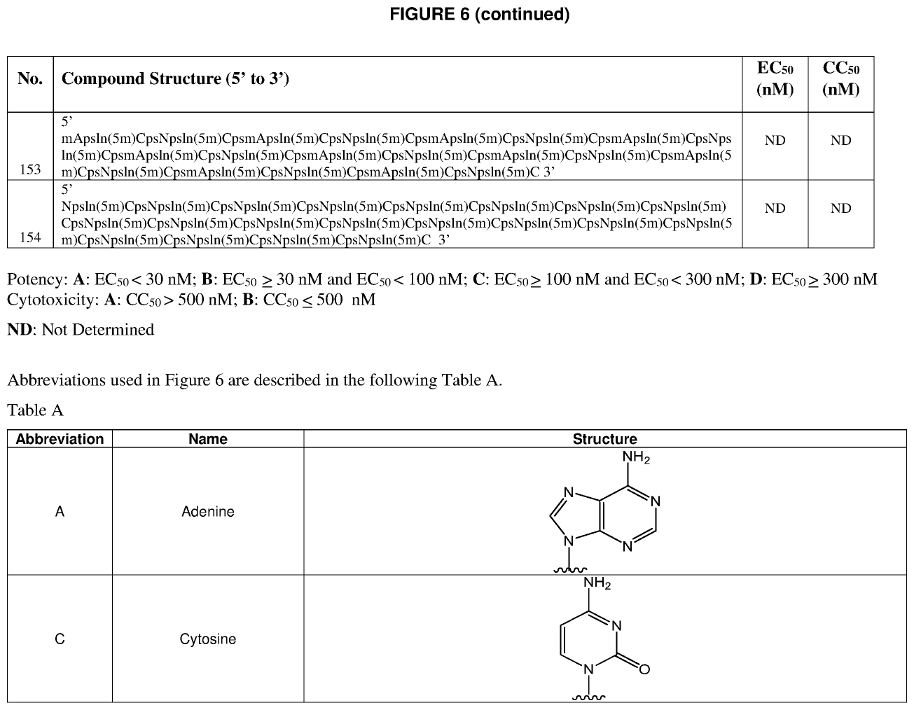

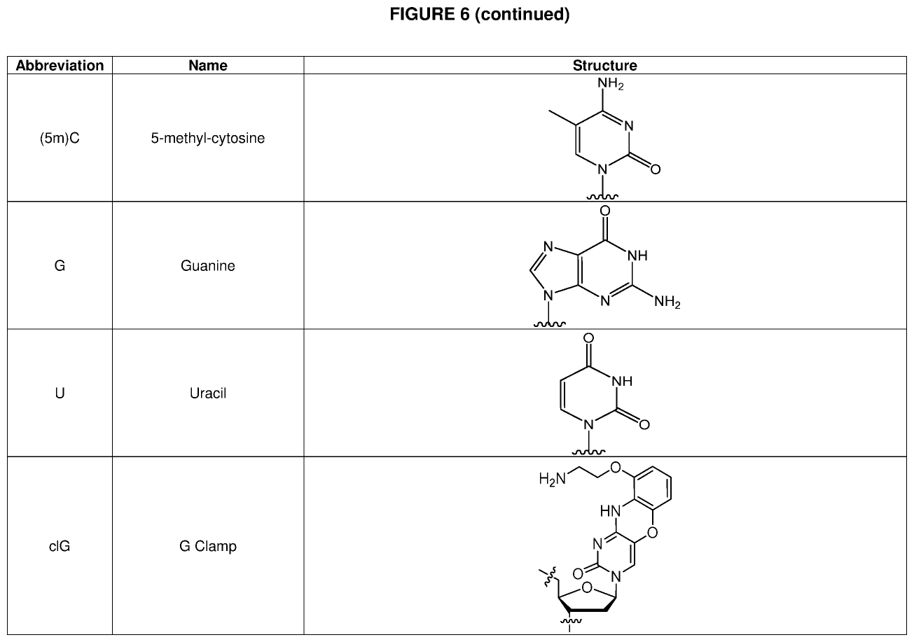

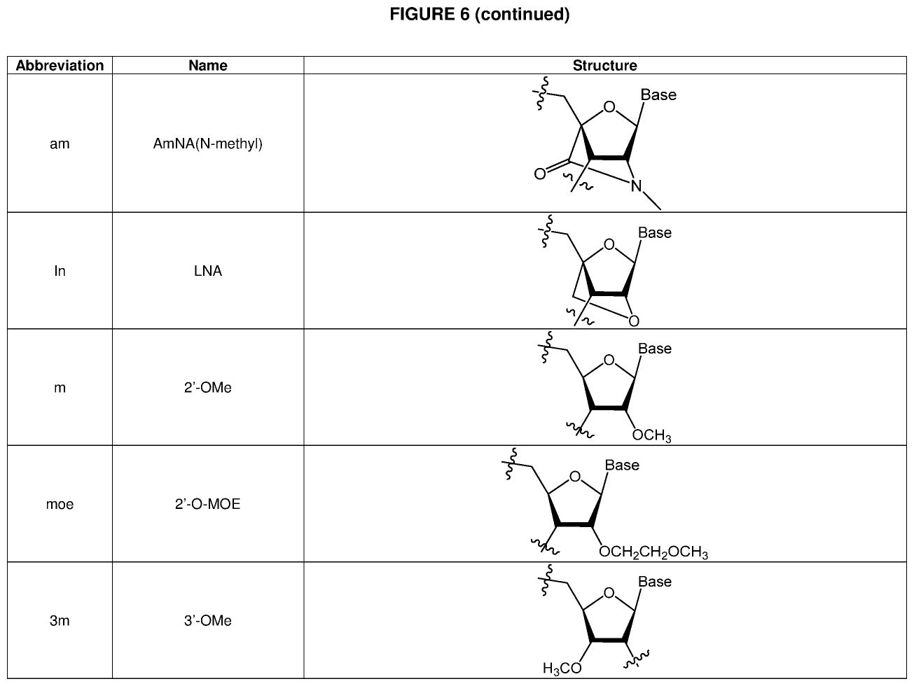

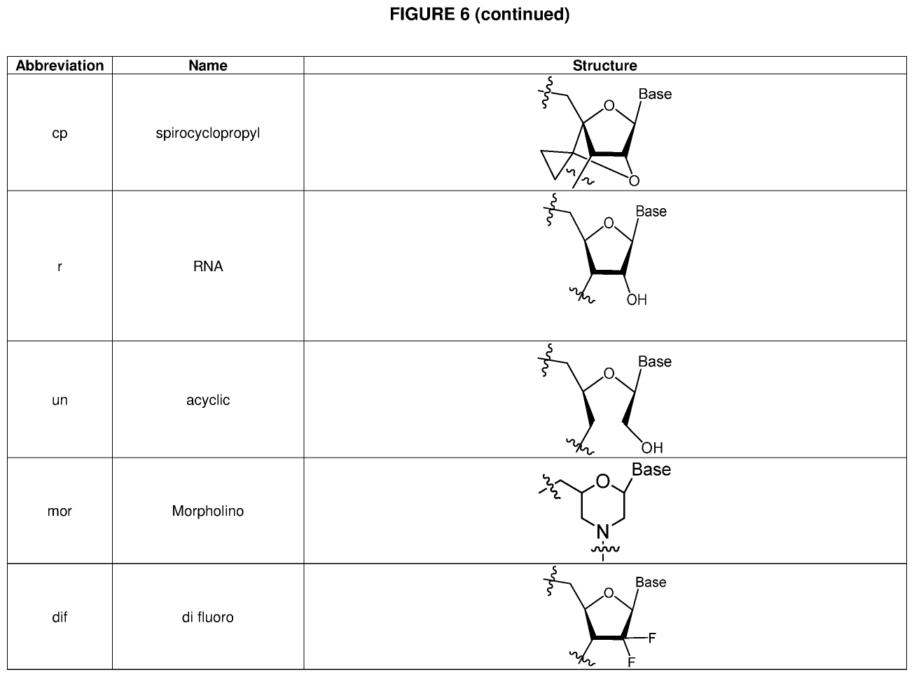

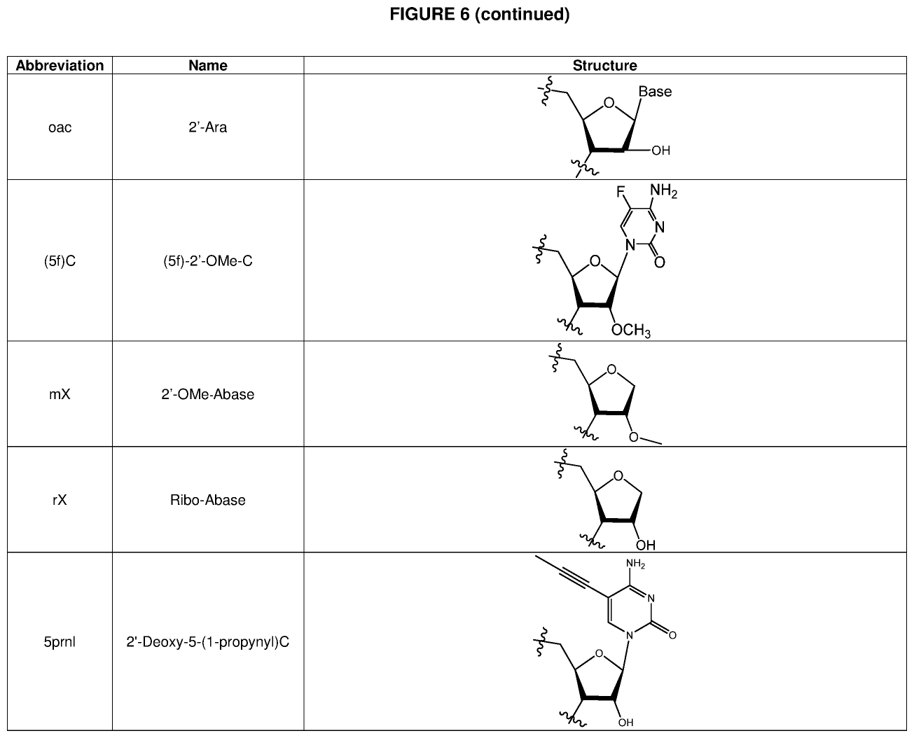

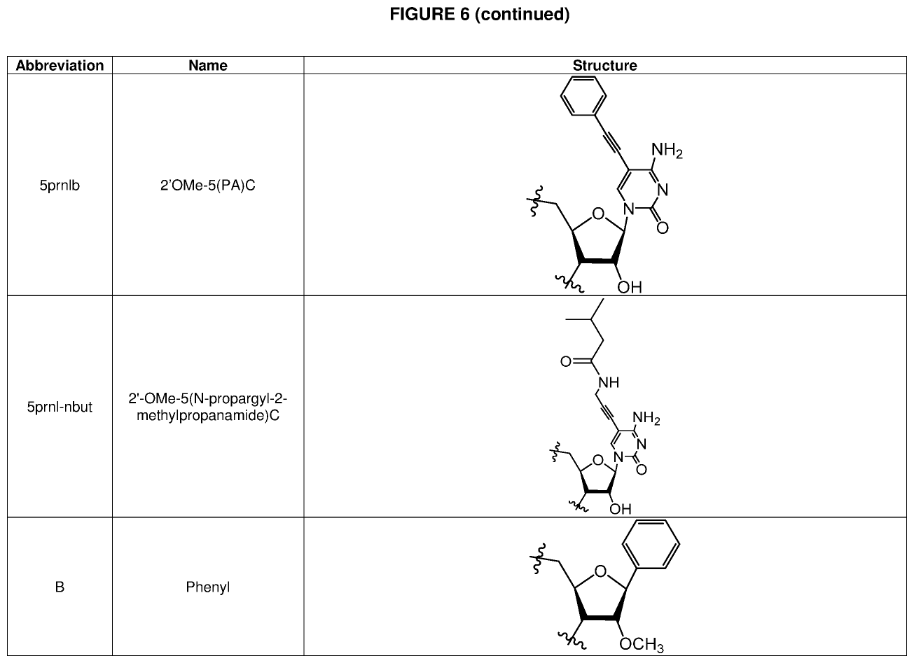

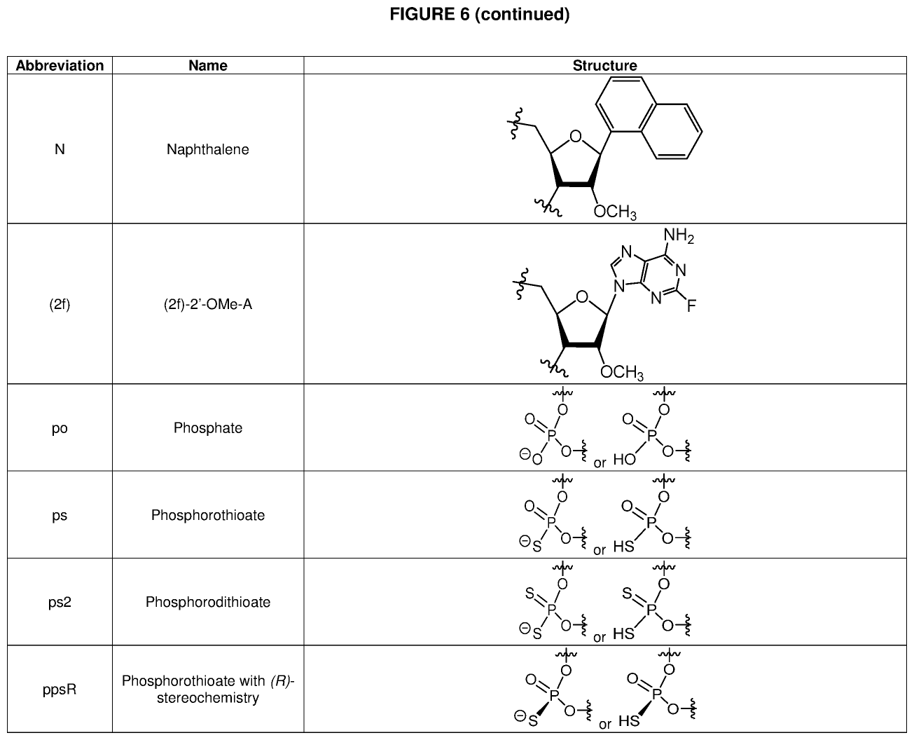

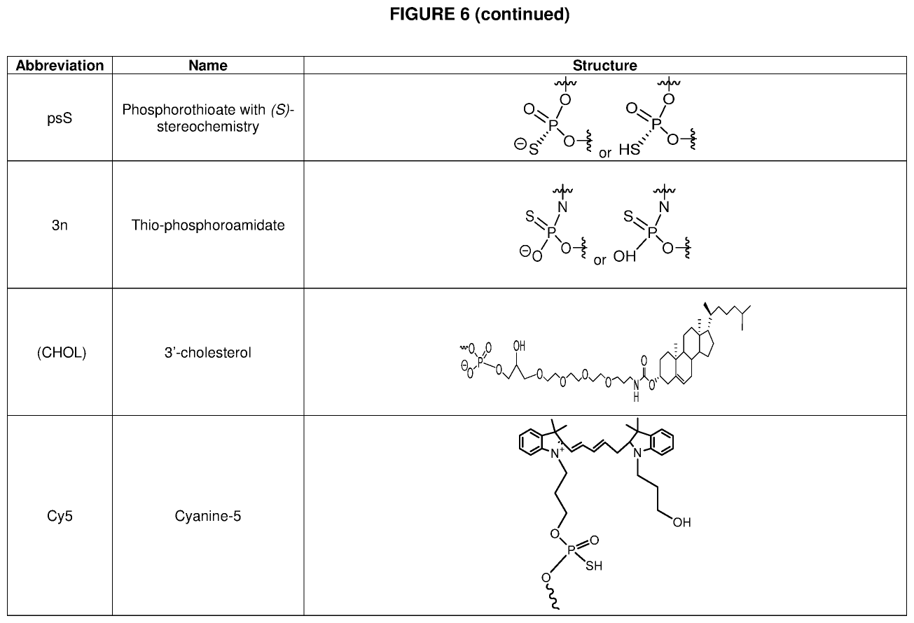

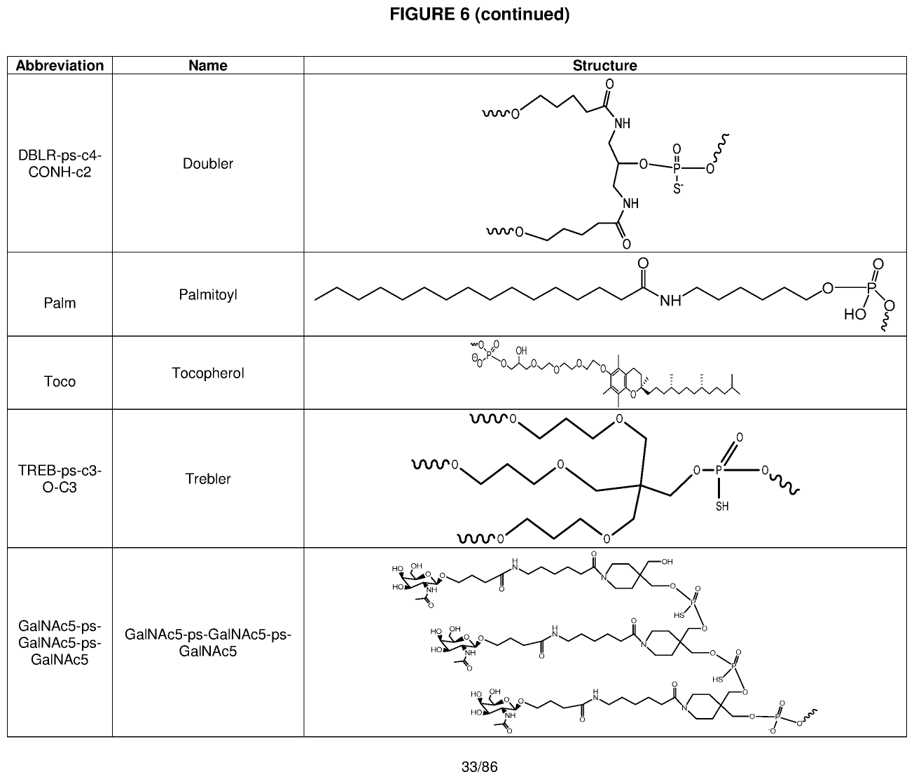

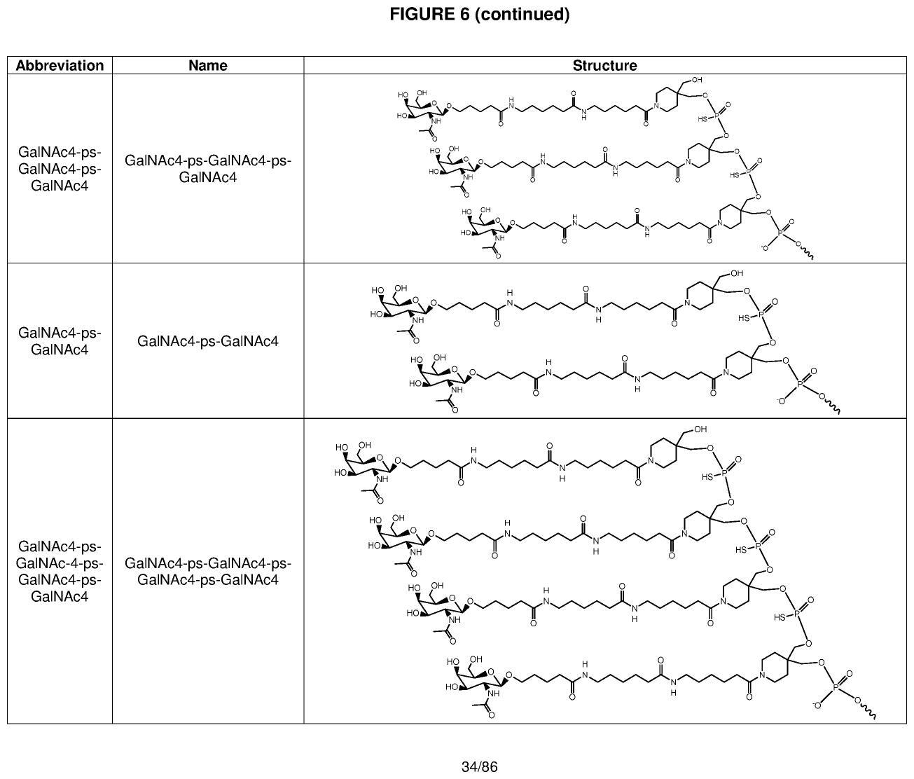

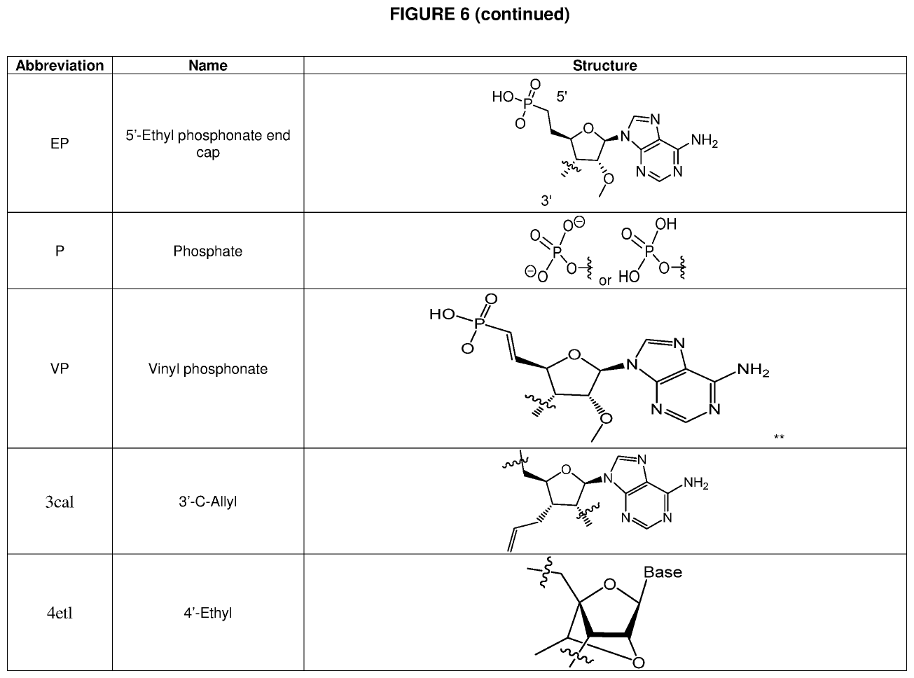

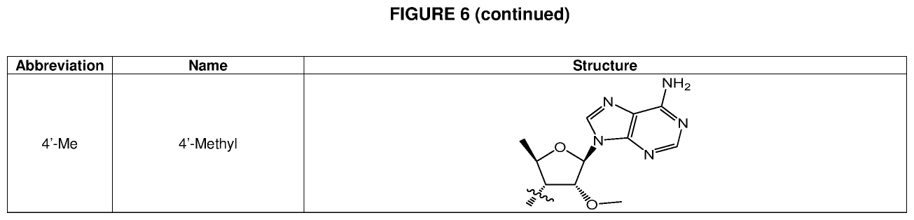

FIG. 6 and Tables 8-26 below summarize aspects of the resulting exemplified modified oligonucleotides. Example 1 illustrates the effect of including a G clamp unit. Examples 2-3 illustrate the effect of including units with 5′-OMe and 5′-(PAC) modifications. Examples 4-9 illustrate the effect of including a morpholino unit. Examples 10-21 illustrate the effect of including A and C blocks. Examples 22-26 (Table 9) illustrate the effect of including other bases. Examples 27-30 (Table 10) illustrate the effect of including 2′-di-fluoro modifications. Examples 31-35 (Table 10) illustrate the effect of including Ara-A modifications.

Examples 36-39 (Table 11) illustrate the effect of including 8-mer blocks (having LNA-G and LNA-T units) at the 3′ and 5′ ends. Examples 40-56 (Table 12) illustrate the effect of including various blocks at both the 3′ and 5′ ends. Examples 57-65 (Table 13) illustrate the effect of including various C, G and T units, as well as linking groups and GalNac2 terminal groups. Examples 66-74 (Table 14) illustrate the effect of including (5f)-2′-OMe-C units, LNA-(5m)C units and 2′-OMe-S(PA)C units (Examples 73-74).

Examples 75-85 (Table 15) illustrate the effect of including 2′-OMe-phenyl units and T-OMe-5(N-propargyl-2-methylpropanamide)C units (Example 85). Examples 86-100 (Table 16) illustrate the effect of including 2′-OMe-Nap units and phosphorodithioate (PS2) linkages (Example 100). Examples 101-109 (Table 17) illustrate the effect of including 2′-OMe-2F-A linkages. Examples 110-116 (Table 18) illustrate the effect of including T-OMe-5(N-propargyl-2-methylpropanamide)C units. Examples 117-122 (Table 19) illustrate the effect of including Abase units.

Examples 123-132 (Table 20) illustrate the effects of including LNA-(5m)C units and targeting ligands that comprise GalNAc. Examples 133-135 (Table 21) illustrate the effects of including 3′-N-2′-OMe-(5m)C units. Examples 136-140 (Table 22) illustrate the effects of including LNA-G and LNA-T units. Examples 141-145 (Table 23) illustrate the effects of including 2′-OMe-G and 2′-OMe-U units. Examples 146-149 (Table 24) illustrate the effect of including 2′-OMe-phenyl units. Examples 150-151 (Table 25) illustrate the effect of including 2′-OMe-5(CF₃) C units. Examples 152-154 (Table 26) illustrate the effect of including 2′-OMe-Nap units.

TABLE 8

| No. | Length | A | C | Comments |
|---|---|---|---|---|
| 1 | (AC)20 | 2′-OMe-A | LNA-(5m)C | 40mer, one G clamp |
| | | | (cl)G | |
| 2 | (AC)20 | 2′-OMe-A | LNA-(5m)C | 40mer, one 2′OMe-5(PA)C |
| | | | 2′OMe-5(PA)C | |
| 3 | (AC)20 | 2′-OMe-A | LNA-(5m)C | 40mer, 5′-OMe-2′-OMe-A |
| | | | 5′-OMe | |
| 4 | (AC)20 | 2′-OMe-A | LNA-(5m)C | 40mer, 1 Morpholino |
| | | mor-A | | |
| 5 | (AC)20 | 2′-OMe-A | LNA-(5m)C | 40mer, 2 Morpholino |
| | | | mor-C | |
| 6 | (AC)20 | 2′-OMe-A | LNA-(5m)C | 40mer, 1 Morpholino |
| | | mor-A | | |
| 7 | (AC)20 | 2′-OMe-A | LNA-(5m)C | 40mer, 2 Morpholino |
| | | | mor-C | |
| 8 | (AC)20 | 2′-OMe-A | LNA-(5m)C | 40mer, 1 Morpholino |
| | | | mor-C | |
| 9 | (AC)20 | 2′-OMe-A | LNA-(5m)C | 40mer, 1 Morpholino |
| | | | mor-C | |
| 10 | (AC)20 | 2′-OMe-A | LNA-(5m)C | 40mer, 2 × 6 A end blocks |
| | | | | (AC)14 repeats |
| 11 | (AC)20 | 2′-OMe-A | LNA-(5m)C | 40mer, 2 × 4 A end blocks |
| | | | | (AC)16 repeats |
| 12 | (AC)20 | 2′-OMe-A | LNA-(5m)C | 40mer, 2 × 4 C end blocks |
| | | | | (AC)16 repeats |
| 13 | (AC)20 | LNA-A | LNA-(5m)C | 40mer, 2 × 6 C end blocks |
| | | | | (AC)14 repeats |
| 14 | (AC)20 | LNA-A | LNA-(5m)C | 40mer, 2 × 10 A end blocks |
| | | | | (AC)16 repeats |
| 15 | (AC)20 | LNA-A | LNA-(5m)C | 40mer, 2 × 10 C end blocks |
| | | | | (AC)16 repeats |
| 16 | (AC)20 | 2′-OMe-A | 2′-OMe-(5m)C | 40mer, 2 × 10 A end blocks |
| | | | | (AC)10 repeats |
| 17 | (AC)20 | 2′-OMe-A | 2′-OMe-(5m)C | 40mer, 2 × 10 C end blocks |
| | | | | (AC)10 repeats |
| 18 | (AC)20 | 2′-OMe-A | 2′-OMe-(5m)C | 40mer, 2 × 6 A end blocks |
| | | | | (AC)14 repeats |
| 19 | (AC)20 | 2′-OMe-A | 2′-OMe-(5m)C | 40mer, 2 × 6 C end blocks |
| | | | | (AC)14 repeats |
| 20 | (AC)20 | 2′-OMe-A | 2′-OMe-(5m)C | 40mer, 2 × 4 A end blocks |
| | | | | (AC)16 repeats |
| 21 | (AC)20 | 2′-OMe-A | 2′-OMe-(5m)C | 40mer, 2 × 4 C end blocks |
| | | | | (AC)16 repeats |

TABLE 9

| No. | Length | Base 1 | Base 2 | Comments |
|---|---|---|---|---|
| 22 | (AG)20 | LNA-A | LNA-G | 40 mer, AG repeat |
| 23 | (CA)20 | LNA-(5m)C | LNA-A | 40 mer, CA repeat |
| 24 | (A)40 | LNA-A | — | 40 mer, A repeat |
| 25 | (A)40 | 2′-OMe-A | — | 40 mer, A repeat |
| 26 | (C)40 | — | LNA-(5m)C | 40 mer, C repeat |

TABLE 10

| No. | Length | A | C | Comments |
|---|---|---|---|---|
| 27 | (AC)20 | 2′-OMe-A | LNA-(5m)C | 40mer, 1 2′-difluoro-C |
| | | | 2′-dif-C | |
| 28 | (AC)20 | 2′-OMe-A | LNA-(5m)C | 40mer, 2 2′-difluoro-C |
| | | | 2′-dif-C | |
| 29 | (AC)20 | 2′-OMe-A | LNA-(5m)C | 40mer, 3 2′-difluoro-C |
| | | | 2′-dif-C | |
| 30 | (AC)20 | 2′-OMe-A | LNA-(5m)C | 40mer, 4 2′-difluoro-C |
| | | | 2′-dif-C | |
| 31 | (AC)20 | 2′-OMe-A | LNA-(5m)C | 40mer, 1 ara-A RNA |
| | | ara-A RNA | | |
| 32 | (AC)20 | 2′-OMe-A | LNA-(5m)C | 40mer, 2 ara-A RNA |
| | | ara-A RNA | | |
| 33 | (AC)20 | 2′-OMe-A | LNA-(5m)C | 40mer, 3 ara-A RNA |
| | | ara-A RNA | | |
| 34 | (AC)20 | 2′-OMe-A | LNA-(5m)C | 40mer, 4 ara-A RNA |
| | | ara-A RNA | | |
| 35 | (AC)20 | 2′-OMe-A | LNA-(5m)C | 40mer, 5 ara-A RNA |
| | | ara-A RNA | | |

TABLE 11

| No. | Length | A | C | G | T | Comments |
|---|---|---|---|---|---|---|
| 36 | AAGAC | LNA-A | LNA-(5m)C | LNA-G | LNA-T | 8mer |
| | TAG | | | | | motif |
| | (AC) | | | | | at |
| | 16 | | | | | 5′end |
| 37 | (AC) | LNA-A | LNA-(5m)C | LNA-G | LNA-T | 8mer |
| | 16- | | | | | motif |
| | AAGA | | | | | at |
| | CTAG | | | | | 3′end |
| 38 | TGAA | LNA-A | LNA-(5m)C | LNA-G | LNA-T | 8mer |
| | GGAC- | | | | | motif |
| | (AC) | | | | | at |
| | 16 | | | | | 5′end |
| 39 | (AC) | LNA-A | LNA-(5m)C | LNA-G | LNA-T | 8mer |
| | 16- | | | | | motif |
| | TGAA | | | | | at |
| | GGAC | | | | | 3′end |

TABLE 12

| No. | Length | A | C | Comments |
|---|---|---|---|---|
| 40 | (A)10(AC)10(A)10 | 2′-OMe-A; | 2′-OMe-(5m)C | 2 × 10 OMe A blocks |
| | | LNA-A | LNA-(5m)C | LNA-(AC)10 |
| 41 | (A)6(AC)14(A)6 | 2′-OMe-A; | 2′-OMe-(5m)C | 2 × 6 OMe A blocks |
| | | LNA-A | LNA-(5m)C | LNA-(AC)14 |
| 42 | (A)4(AC)16(A)4 | 2′-OMe-A; | LNA-(5m)C | 2 × 4 OMe A blocks |
| | | LNA-A | | LNA-(AC)16 |
| 43 | (A)2(AC)18(A)2 | 2′-OMe-A; | 2′-OMe-(5m)C | 2 × 2 OMe A blocks |
| | | LNA-A | LNA-(5m)C | LNA-(AC)18 |
| 44 | (C)6(AC)14(C)6 | 2′-OMe-A; | 2′-OMe-(5m)C | 2 × 6 OMe C blocks |
| | | LNA-A | | LNA-(AC)14 |
| 45 | (C)10(AC)10(C)10 | 2′-OMe-A; | 2′-OMe-(5m)C | 2 × 10 OMe C blocks |
| | | LNA-A | LNA-(5m)C | LNA-(AC)14 |
| 46 | (C)4(AC)16(C)4 | 2′-OMe-A; | 2′-OMe-(5m)C | 2 × 4 OMe C blocks |
| | | LNA-A | LNA-(5m)C | LNA-(AC)16 |
| 47 | (C)2(AC)18(C)2 | 2′-OMe-A; | 2′-OMe-(5m)C | 2 × 2 OMe C blocks |
| | | LNA-A | LNA-(5m)C | LNA-(AC)18 |
| 48 | (C)40 | NA | LNA-(5m)C | 40mer, All C |
| 49 | (A)10(AC)10(A)10 | LNA-A; | LNA-(5m)C | 2 × 10 LNA A blocks |
| | | 2′-OMe-A | 2′-OMe-(5m)C | OMe-(AC)10 |
| 50 | (C)10(AC)10(C)10 | LNA-A; | LNA-(5m)C | 2 × 10 LNA C blocks |
| | | 2′-OMe-A | 2′-OMe-(5m)C | OMe-(AC)10 |
| 51 | (A)6(AC)14(A)6 | LNA-A; | LNA-(5m)C | 2 × 6 LNA A blocks |
| | | 2′-OMe-A | 2′-OMe-(5m)C | OMe-(AC)14 |
| 52 | (C)6(AC)14(C)6 | LNA-A | LNA-(5m)C | 2 × 6 LNA C blocks |
| | | 2′-OMe-A; | 2′-OMe-(Sm) C | OMe-(AC)14 |
| 53 | (A)4(AC)14(A)4 | LNA-A; | LNA-(5m)C | 2 × 4 LNA A blocks |
| | | 2′-OMe-A | 2′-OMe-(5m)C | OMe-(AC)16 |
| 54 | (C)4(AC)14(C)4 | LNA-A; | LNA-(5m)C | 2 × 4 LNA C blocks |
| | | 2′-OMe-A | 2′-OMe-(5m)C | OMe-(AC)16 |
| 55 | (A)2(AC)18(A)2 | LNA-A; | LNA-(5m)C | 2 × 2 LNA A blocks |
| | | 2′-OMe-A | 2′-OMe-(5m)C | OMe-(AC)18 |
| 56 | (C)2(AC)18(C)2 | LNA-A; | LNA-(5m)C | 2 × 2 LNA C blocks |
| | | 2′-OMe-A | 2′-OMe-(5m)C | OMe-(AC)18 |

TABLE 13

| No. | Length | A | C | G | T | Comments |
|---|---|---|---|---|---|---|
| 57 | (AC)20 | 2′-OMe-A | scp-(5m)C | | | 40mer, 20 scp-BNA |
| | | | | | | (50%) |
| 58 | (AC)20 | 2′-OMe-A | 2′-OMe-(5m)C | | | 40mer, 10 AmNA |
| | | | AmNA(5m)C | | | (25%) |
| 59 | (GA)20 | LNA-A | | LNA-G | | 40mer, All LNA |
| | | | | | | GA |
| 60 | GalNac-2-NH-C6-CA(AC)20 | LNA-A | LNA-(5m)C | | | 40mer, All LNA, |
| | | | | | | GalNac2-at 5′-end |
| | | | | | | with CA linker |
| 61 | GalNac-2-NH-C6(AC)20 | LNA-A | LNA-(5m)C | | | 40mer, All LNA, |
| | | | | | | GalNac2-at 5′-end |
| 62 | (AC)20-CA-C6-NH-GalNAc2 | LNA-A | LNA-(5m)C | | | 40mer, All LNA, |
| | | | | | | GalNac2-at 3′-end |
| | | | | | | with CA linker |
| 63 | (AC)20-C6-NH-GalNAc2 | LNA-A | LNA-(5m)C | | | 40mer, All LNA, |
| | | | | | | GalNac2-at 3′-end |
| 64 | (AT)20 | LNA-A | — | — | LNA-T | 40mer, All LNA |
| | | | | | | AT |
| 65 | (AC)20 | 2′-OMe-A | 2′-OMe-(5m)C | | | 40mer, 5 AmNA |
| | | AmNA-A | AmNA(5m)C | | | (12.5%) |

TABLE 14

| No. | Length | A | C | Comments |
|---|---|---|---|---|
| 66 | (AC)20 | 2′-OMe-A | LNA-(5m)C | 40mer, Alternate 2′-OMe/LNA |
| | | | (5f)-2′-OMe—C | with 1 × (50-2′-OMe—C |
| 67 | (AC)20 | 2′-OMe-A | LNA-(5m) C | 40mer, Alternate 2′-OMe/LNA |
| | | | (5f)-2′-OMe—C | with 2 × (50-2′-OMe—C |
| 68 | (AC)20 | 2′-OMe-A | LNA-(5m)—C | 40mer, Alternate 2′-OMe/LNA |
| | | | (5f)-2′-OMeC | with 4 × (50-2′-OMe—C |
| 69 | (AC)20 | 2′-OMe-A | LNA-(5m)C | 40mer, Alternate 2′-OMe/LNA |
| | | | (5f)-2′-OMe—C | with 6 × (50-2′-OMe—C |
| 70 | (AC)20 | LNA-A | LNA-(5m)C | 40mer, All LNA with 3 × (50- |
| | | | (5f)-2′-OMe—C | 2′-OMe—C |
| 71 | (AC)20 | LNA-A | LNA-(5m)C | 40mer, All LNA with 5 × (50- |
| | | | (5f)-2′-OMe—C | 2′-OMe—C |
| 72 | (AC)20 | LNA-A | LNA-(5m)C | 40mer, All LNA with 7 × (50- |
| | | | (5f)-2′-OMe—C | 2′-OMe—C |
| 73 | (AC)20 | 2′-OMe-A | LNA-(5m)C; | 40mer, Alternate 2′-OMe and LNA |
| | | | 2′-OMe-5(PA)C | with 2 × 2′-OMe-5(PA)C on |
| | | | | position 2 and 4 |
| 74 | (AC)20 | 2′-OMe-A | LNA-(5m)C; | 40mer, Alternate 2′-OMe and LNA |
| | | | 2′-OMe-5(PA)C | with 2 × 2′-OMe-5(PA)C on |
| | | | | position 2 and 6 |

TABLE 15

| No. | Length | A | C | Other | Comments |
|---|---|---|---|---|---|
| 75 | (AC)20 | 2′-OMe-A | LNA-(5m)C | 2′-OMe- | 40mer, Alternate 2′-OMe and |
| | | | | phenyl | LNA with 1 Other in place of C |
| 76 | (AC)20 | 2′-OMe-A | LNA-(5m)C | 2′-OMe- | 40mer, Alternate 2′-OMe and |
| | | | | phenyl | LNA with 2 Others in place of |
| | | | | | C |
| 77 | (AC)20 | 2′-OMe-A | LNA-(5m)C | 2′-OMe- | 40mer, Alternate 2′-OMe and |
| | | | | phenyl | LNA with 3 Others in place of |
| | | | | | C |
| 78 | (AC)20 | 2′-OMe-A | LNA-(5m)C | 2′-OMe- | 40mer, Alternate 2′-OMe and |
| | | | | phenyl | LNA with 4 Others in place of |
| | | | | | C |
| 79 | (AC)20 | 2′-OMe-A | LNA-(5m)C | 2′-OMe- | 40mer, Alternate 2′-OMe and |
| | | | | phenyl | LNA with 6 Others in place of |
| | | | | | C |
| 80 | (AC)20 | LNA-A | LNA-(5m)C | 2′-OMe- | 40mer, All LNA with 3 Others |
| | | | | phenyl | in place of C |
| 81 | (AC)20 | LNA-A | LNA-(5m)C | 2′-OMe- | 40mer, All LNA with 5 Others |
| | | | | phenyl | in place of C |
| 82 | (AC)20 | LNA-A | LNA-(5m)C | 2′-OMe- | 40mer, All LNA with 7 Others |
| | | | | phenyl | in place of C |
| 83 | (AC)20 | LNA-A | LNA-(5m)C | 2′-OMe- | 40mer, All LNA with 1 |
| | | | | phenyl | Other in place of A |
| 84 | (AC)20 | LNA-A | LNA-(5m)C | 2′-OMe- | 40mer, All LNA with 2 |
| | | | | phenyl | Others in place of A |
| 85 | (AC)20 | 2′-OMe-A | LNA-(5m)C; | | 40mer, Alternate 2′- |
| | | | 2′-OMe-5(N- | | Ome/LNA with 1 × 2′-OMe- |
| | | | propargyl-2- | | 5(N-propargyl-2- |
| | | | methylpropan | | methylpropanamide)C |
| | | | amide)C | | |

TABLE 16

| No. | Length | A | C | Other | Comments |
|---|---|---|---|---|---|
| 86 | (AC)20 | 2′-OMe-A | LNA-(5m)C | 2′-OMe-Nap | 40mer, Alternate 2′-OMe and |
| | | | | | LNA with 1 Other in place of C |
| 87 | (AC)20 | 2′-OMe-A | LNA-(5m)C | 2′-OMe-Nap | 40mer, Alternate 2′-OMe and |
| | | | | | LNA with 1 Other in place of C |
| 88 | (AC)20 | 2′-OMe-A | LNA-(5m)C | 2′-OMe-Nap | 40mer, Alternate 2′-OMe and |
| | | | | | LNA with 1 Other in place of C |
| 89 | (AC)20 | 2′-OMe-A | LNA-(5m)C | 2′-OMe-Nap | 40mer, Alternate 2′-OMe and |
| | | | | | LNA with 2 Others in place of C |
| 90 | (AC)20 | 2′-OMe-A | LNA-(5m)C | 2′-OMe-Nap | 40mer, Alternate 2′-OMe and |
| | | | | | LNA with 3 Others in place of A |
| 91 | (AC)20 | 2′-OMe-A | LNA-(5m)C | 2′-OMe-Nap | 40mer, Alternate 2′-OMe and |
| | | | | | LNA with 3 Others in place of A |
| 92 | (AC)20 | 2′-OMe-A | LNA-(5m)C | 2′-OMe-Nap | 40mer, Alternate 2′-OMe and |
| | | | | | LNA with 4 Others in place of A |
| 93 | (AC)20 | 2′-OMe-A | LNA-(5m)C | 2′-OMe-Nap | 40mer, Alternate 2′-OMe and |
| | | | | | LNA with 5 Others in place of A |
| 94 | (AC)20 | LNA-A | LNA-(5m)C | 2′-OMe-Nap | 40mer, All LNA with 3 Others in |
| | | | | | place of A |
| 95 | (AC)20 | LNA-A | LNA-(5m)C | 2′-OMe-Nap | 40mer, All LNA with 5 Others |
| | | | | | in place of A |
| 96 | (AC)20 | LNA-A | LNA-(5m)C | 2′-OMe-Nap | 40mer, All LNA with 3 Others in |
| | | | | | place of A |
| 97 | (AC)20 | LNA-A | LNA-(5m)C | 2′-OMe-Nap | 40mer, All LNA with 3 Others |
| | | | | | in place of A |
| 98 | (AC)20 | LNA-A | LNA-(5m)C | 2′-OMe-Nap | 40mer, All LNA with 4 Others in |
| | | | | | place of A |
| 99 | (AC)20 | LNA-A | LNA-(5m)C | 2′-OMe-Nap | 40mer, All LNA with 5 Others in |
| | | | | | place of A |
| 100 | (AC)20 | 2′-OMe-A | LNA-(5m)C | | 40mer, Alternate 2′-OMe and |
| | | | | | LNA with 1 PS2 linkage |

TABLE 17

| No. | Length | A | C | Comments |
|---|---|---|---|---|
| 101 | (AC)20 | 2′-OMe-A; | LNA-(5m) C | 40mer, Alternate 2′-OMe and LNA with |
| | | 2′-OMe—2F-A | | 3 2′-OMe—2F-A in place OMe-A |
| 102 | (AC)20 | 2′-OMe-A; | LNA-(5m) C | 40mer, Alternate 2′-OMe and LNA with |
| | | 2′-OMe—2F-A | | 4 2′-OMe—2F-A in place OMe-A |
| 103 | (AC)20 | 2′-OMe-A; | LNA-(5m) C | 40mer, Alternate 2′-OMe and LNA with |
| | | 2′-OMe—2F-A | | 1 2′-OMe—2F-A in place OMe-A |
| 104 | (AC)20 | 2′-OMe-A; | LNA-(5m) C | 40mer, Alternate 2′-OMe and LNA with |
| | | 2′-OMe—2F-A | | 2 mod (20-2′-OMe-A in place OMe-A |
| 105 | (AC)20 | 2′-OMe-A; | LNA-(5m) C | 40mer, Alternate 2′-OMe and LNA with |
| | | 2′-OMe—2F-A | | 1 2′-OMe—2F-A in place OMe-A |
| 106 | (AC)20 | 2′-OMe-A; | LNA-(5m) C | 40mer, Alternate 2′-OMe and LNA with |
| | | 2′-OMe—2F-A | | 2 2′-OMe—2F-A in place OMe-A |
| 107 | (AC)20 | 2′-OMe-A; | LNA-(5m) C | 40mer, Alternate 2′-OMe and LNA with |
| | | 2′-OMe—2F-A | | 2 2′-OMe—2F-A in place OMe-A |
| 108 | (AC)20 | 2′-OMe-A; | LNA-(5m) C | 40mer, All LNA with 4 2′-OMe—2F-A in |
| | | 2′-OMe—2F-A | | place OMe-A |
| 109 | (AC)20 | 2′-OMe-A; | LNA-(5m) C | 40mer, All LNA with 6 2′-OMe—2F-A in |
| | | 2′-OMe—2F-A | | place OMe-A |

TABLE 18

| No. | Length | A | C | Other | Comments |
|---|---|---|---|---|---|
| 110 | (AC)20 | 2′-OMe-A | LNA-(5m)C | 2′-OMe-5(N- | 40mer, Alternate 2′-OMe and |
| | | | | propargyl-2- | LNA with 1 × 2′-OMe-5(N- |
| | | | | methylpropanamide)C | propargyl-2-methylpropanamide)C |
| 111 | (AC)20 | 2′-OMe-A | LNA-(5m)C | 2′-OMe-5(N- | 40mer, Alternate 2′-OMe and |
| | | | | propargyl-2- | LNA with 2 × 2′-OMe-5(N- |
| | | | | methylpropanamide)C | propargyl-2-methylpropanamide)C |
| 112 | (AC)20 | 2′-OMe-A | LNA-(5m)C | 2′-OMe-5(N- | 40mer, Alternate 2′-OMe and |
| | | | | propargyl-2- | LNA with 4 × 2′-OMe-5(N- |
| | | | | methylpropanamide)C | propargyl-2-methylpropanamide)C |
| 113 | (AC)20 | 2′-OMe-A | LNA-(5m)C | 2′-OMe-5(N- | 40mer, Alternate 2′-OMe and |
| | | | | propargyl-2- | LNA with 6 × 2′-OMe-5(N- |
| | | | | methylpropanamide)C | propargyl-2-methylpropanamide)C |
| 114 | (AC)20 | LNA-A | LNA-(5m)C | 2′-OMe-5(N- | 40mer, All LNA with 3 × 2′-OMe- |
| | | | | propargyl-2- | 5(N-propargyl-2- |
| | | | | methylpropanamide)C | methylpropanamide)C |
| 115 | (AC)20 | LNA-A | LNA-(5m)C | 2′-OMe-5(N- | 40mer, All LNA with 3 × 2′-OMe- |
| | | | | propargyl-2- | 5(N-propargyl-2- |
| | | | | methylpropanamide)C | methylpropanamide)C |
| 116 | (AC)20 | LNA-A | LNA-(5m)C | 2′-OMe-5(N- | 40mer, All LNA with 3 × 2′-OMe- |
| | | | | propargyl-2- | 5(N-propargyl-2- |
| | | | | methylpropanamide)C | methylpropanamide)C |

TABLE 19

| No. | Length | A | C | Comments |
|---|---|---|---|---|
| 117 | (AC)20 | 2′-OMe-A | LNA-(5m)C | 40mer, Alternate 2′-OMe and |
| | | | Abase | LNA with 1 Abase |
| 118 | (AC)20 | 2′-OMe-A | LNA-(5m)C | 40mer, Alternate 2′-OMe and |
| | | | Abase | LNA with 2 Abase |
| 119 | (AC)20 | 2′-OMe-A | LNA-(5m)C | 40mer, Alternate 2′-OMe and |
| | | | Abase | LNA with 4 Abase |
| 120 | (AC)20 | 2′-OMe-A | LNA-(5m)C | 40mer, Alternate 2′-OMe and |
| | | | Abase | LNA with 6 Abase |
| 121 | (AC)20 | LNA-A | LNA-(5m)C | 40mer, Alternate 2′-OMe and |
| | | | Abase | LNA with 3 Abase |
| 122 | (AC)20 | LNA-A | LNA-(5m)C | 40mer, Alternate 2′-OMe and |
| | | | Abase | LNA with 5 Abase |

TABLE 20

| No. | Length | A | C | Comments |
|---|---|---|---|---|
| 123 | (AC)20-GalNAc4-ps- | 2′-OMe-A | LNA-(5m)C | 40mer, alternate 2′-OMe-LNA; 4 |
| | GalNAc4ps-GalNAc4 | Ribo-A | | ribo A with 3 × GalNac4 at 3′-end |
| 124 | GalNAc4-ps-GalNAc4- | 2′-OMe-A | LNA-(5m)C | 40mer, alternate 2′-OMe-LNA; 4 |
| | ps-GalNAc4--(AC)20 | Ribo-A | | ribo A with 3 × GalNac4 at 5′-end |
| 125 | GalNAc4-ps-GalNAc4- | 2′-OMe-A | LNA-(5m)C | 40mer, alternate 2′-OMe-LNA; 4 |
| | ps-GalNAc4-ps- | Ribo-A | | ribo A with 4 × GalNac4 at 5′end |
| | GalNac4--(AC)20 | | | |
| 126 | GalNAc4-ps-GalNAc4ps- | 2′-OMe-A | LNA-(5m)C | 40mer, alternate 2′-OMe-LNA; 4 |
| | (AC)20-GalNAc4ps- | Ribo-A | | ribo A with 2 × GalNac4 at 5′end |
| | GalNAc4ps-GalNAc4 | | | and 3 × GalNAc4 at 3′-end |
| 127 | GalNAc4ps-GalNAc4ps- | 2′-OMe-A | LNA-(5m)C | 40mer, alternate 2′-OMe-LNA; 4 |
| | GalNAc4-ps--(AC)20- | Ribo-A | | ribo A with 3 × GalNac4 at 5′and |
| | GalNAc4ps-GalNAc4ps- | | | 3′-end |
| | GalNAc4 | | | |
| 128 | GalNAc4ps-GalNAc4ps- | 2′-OMe-A | LNA-(5m)C | 40mer, alternate 2′-OMe-LNA; 4 |
| | (AC)20-GalNAc4ps- | Ribo-A | | ribo A with 2 × GalNac4 at 5′and |
| | GalNAc4 | | | 3′end |
| 129 | (AC)20-po-GalNAc4ps- | 2′-OMe-A | LNA-(5m)C | 40mer, alternate 2′-OMe-LNA; 4 |
| | GalNAc4ps-GalNAc4-ps- | Ribo-A | | ribo A with 4 × GalNac4 at 3′end |
| | GalNac4 | | | |
| 130 | GalNAc4ps-GalNAc4ps- | 2′-OMe-A | LNA-(5m)C | 40mer, alternate 2′-OMe-LNA; 4 |
| | (AC)20 | Ribo-A | | ribo A with 2 × GalNac4 at 5′end |
| 131 | (AC)20-GalNAc4-ps- | 2′-OMe-A | LNA-(5m)C | 40mer, alternate 2′-OMe-LNA; 4 |
| | GalNAc4 | Ribo-A | | ribo A with 4 × GalNac4 at 3′end |
| 132 | GalNAc4ps-GalNAc4-ps- | LNA-A | LNA-(5m)C | 40mer, All-LNA; with 3 × GalNac4 |
| | GalNac4-(AC)20 | | | at 3′end |

TABLE 21

| No. | Length | A | C | Other | Comments |
|---|---|---|---|---|---|
| 133 | (AC)20 | 2′-OMe-A | 2′-OMe-(5m)C | 3′-N-T-OMe- | 40mer, All 2′-OMe with |
| | | | | (5m)C | 10 × 3′-N-2′-OMe-(5m) C |
| 134 | (AC)20 | 2′-OMe-A | 2′-OMe-(5m)C | 3′-N-T-OMe- | 40mer, All 2′-OMe with |
| | | | | (5m)C | 8 × 3′-N-2′-OMe-(5m) C |
| 135 | (AC)20 | 2′-OMe-A | 2′-OMe-(5m)C | 3′-N-2′-OMe- | 40mer, All 2′-OMe with |
| | | | | (5m)C | 5 × 3′-N-2′-OMe-(5m)C |

TABLE 22

| No. | Length | A | C | G | T | Comments |
|---|---|---|---|---|---|---|
| 136 | AAGA | LNA-A | LNA-(5m)C | LNA-G | LNA-T | AH LNA; |
| | CTAG- | | | | | Alternate |
| | AAGA | | | | | 8mer |
| | CTAG | | | | | motif |
| | AAGA | | | | | |
| | CTAG | | | | | |
| | AAGA | | | | | |
| | CTAG | | | | | |
| | AAGA | | | | | |
| | CTAG | | | | | |
| 137 | AAGA | LNA-A | LNA-(5m)C | LNA-G | LNA-T | All LNA; |
| | CTAG | | | | | 8mer |
| | (AC)12- | | | | | motif |
| | AAGA | | | | | at |
| | CTAG | | | | | 5′and |
| | | | | | | 3′end |
| 138 | TGAA | LNA-A | LNA-(5m)C | LNA-G | LNA-T | All LNA; |
| | GGAC- | | | | | 3× 8mer |
| | (AC)4- | | | | | motif |
| | TGAAG | | | | | |
| | GAC- | | | | | |
| | (AC)4 | | | | | |
| | TGAAG | | | | | |
| | GAC | | | | | |
| 139 | (AGGA | LNA-A | LNA-(5m)C | LNA-G | LNA-T | All LNA; |
| | CATG) | | | | | All |
| | 5 | | | | | Scramble |
| 140 | (AGGA | LNA-A | LNA-(5m)C | LNA-G | LNA-T | All LNA; |
| | CATG) | | | | | Scramble, |
| | (AC) | | | | | 8mer motif |
| | 16- | | | | | at 5′end |

TABLE 23

| No. | Length | A | C | G | T/U | Comments |
|---|---|---|---|---|---|---|
| 141 | AAGA | 2′- | 2′-OMe- | 2′- | 2′- | All |
| | CUAG- | OMe-A | (5m)C | OMe-G | OMe-U | 2′-O-Me: |
| | AAGA | | | | | Alternate |
| | CUAG | | | | | 8mer |
| | AAGA | | | | | motif |
| | CUAG | | | | | |
| | AAGA | | | | | |
| | CUAG | | | | | |
| | AAGA | | | | | |
| | CUAG | | | | | |
| 142 | AAGA | 2′- | 2′-OMe- | 2′- | 2′- | All |
| | CUAG | OMe-A | (5m)C | OMe-G | OMe-U | 2′-O-Me: |
| | (AC)12- | | | | | 8mer |
| | AAGA | | | | | motif at |
| | CTAG | | | | | 5′ |
| | | | | | | and |
| | | | | | | 3′end |
| 143 | UGAA | 2′- | 2′-OMe- | 2′- | 2′- | All |
| | GGAC- | OMe-A | (5m)C | OMe-G | OMe-U | 2′-O-Me: |
| | (AC)4- | | | | | 3× 8mer |
| | UGAA | | | | | motif |
| | GGAC- | | | | | |
| | (AC)4 | | | | | |
| | UGAA | | | | | |
| | GGAC | | | | | |
| 144 | (AGGA | 2′- | 2′-OMe- | 2′- | 2′- | All |
| | CAUG)5 | OMe-A | (5m)C | OMe-G | OMe-U | 2′-O-Me; |
| | | | | | | All |
| | | | | | | Scramble |
| 145 | (AGGA | 2′- | 2′-OMe- | 2′- | 2′- | All |
| | CAUG) | OMe-A | (5m)C | OMe-G | OMe-U | 2′-O-Me; |
| | (AC)16- | | | | | Scramble |
| | | | | | | 8mer |
| | | | | | | motif |
| | | | | | | at |
| | | | | | | 5′end |

TABLE 24

| No. | Length | A | C | Other | Comments |
|---|---|---|---|---|---|
| 146 | (AC)20 | 2′-OMe-A | LNA-(5m)C | 2′-OMe-phenyl | 40mer, Alternate 2′-OMe and |
| | | | | | LNA with 6 2′-OMe-phenyl |
| | | | | | in place of C |
| 147 | (AC)20 | 2′-OMe-A | LNA-(5m)C | 2′-OMe-phenyl | 40mer, Alternate 2′-OMe and |
| | | | | | LNA with 10 2′-OMe-phenyl |
| | | | | | in place of C |
| 148 | (AC)20 | 2′-OMe-A | LNA-(5m)C | 2′-OMe-phenyl | 40mer, Alternate 2′-OMe and |
| | | | | | LNA with 6 2′-OMe-phenyl |
| | | | | | in place of C |
| 149 | (AC)20 | 2′-OMe-A | LNA-(5m)C | 2′-OMe-phenyl | 40mer, Alternate 2′-OMe and |
| | | | | | LNA with 20 2′-OMe-phenyl |
| | | | | | in place of C |

TABLE 25

| No. | Length | A | C | Other | Comments |
|---|---|---|---|---|---|
| 150 | (AC)20 | 2′-OMe-A | LNA-(5m)C | 2′-OMe-5(CF₃) C | 40mer, Alternate 2′-OMe and |
| | | | | | LNA with 1 × 2′-OMe-5(CF₃) C |
| 151 | (AC)20 | 2′-OMe-A | LNA-(5m)C | 2′-OMe-5(CF₃) C | 40mer, Alternate 2′-OMe and |
| | | | | | LNA with 2 × 2′-OMe-5(CF₃) C |

TABLE 26

| No. | Length | A | C | Other | Comments |
|---|---|---|---|---|---|
| 152 | (AC)20 | 2′-OMe-A | LNA-(5m)C | 2′-OMe-Nap | 40mer, Alternate 2′-OMe and LNA |
| | | | | | with 5 × 2′-OMe-Nap in place of C |
| 153 | (AC)20 | 2′-OMe-A | LNA-(5m)C | 2′-OMe-Nap | 40mer, Alternate 2′-OMe and LNA |
| | | | | | with 10 × 2′-OMe-Nap in place of C |
| 154 | (AC)20 | 2′-OMe-A | LNA-(5m)C | 2′-OMe-Nap | 40mer, Alternate 2′-OMe and LNA |
| | | | | | with 20 × 2′-OMe-Nap in place of C |

### Example B1

### HBsAg Secretion Assay and Cytotoxicty Assay

The sequence independent antiviral activity against hepatitis B (as determined by HBsAg Secretion Assay) and the cytotoxicity of a number of exemplified modified oligonucleotide compounds was determined as described below and summarized in FIG. 6.

### HBsAg Release Assay Protocol

### Cell Culture

HepG2.2.15 cells were maintained in DMEM medium with 10% fetal bovine serum (FBS) and 1% penicillin/streptomycin, 1% Glutamine, 1% non-essential amino acids, 1% Sodium Pyruvate and 380 ug/ml G418. Cells were maintained at 37° C. in a 5% CO₂ atmosphere.

### HBsAg Secretion Assay

HepG2.2.15 cells were grown in DMEM medium as described above. Cells were plated at a concentration of 45,000 cells/well in collagen-I coated 96 well plates. Immediately after addition of the cells, test compounds are added. A DNA 20mer ((AC)10, alternating deoxy A/deoxy C) and a DNA 40mer ((AC)20, alternating deoxy A/deoxy C) were used as controls.

Selected compounds may also be tested following Lipofectamine® RNAiMAX transfection. Lipofectamine® RNAiMAX Transfection Reagent (Thermo Fisher) is used following the manufacturer's instructions.

The 50% inhibitory concentration (EC₅₀) and 50% cytotoxic concentration (CC₅₀; below) were assessed by solubilizing in 1×PBS to 100× the desired final testing concentration. Each compound was then serially diluted (1:3) up to 8 distinct concentrations to 10× the desired final testing concentration in DMEM medium with 10% FBS. A 10 μL sample of the 10× compounds in cell culture media was used to treat the HepG2.2.15 cells in a 96-well format. Cells were initially incubated with compounds for 3 days at 37° C. in a 5% CO₂ atmosphere.

Three days post compound addition/transfection replace media and compound with fresh media/compound with RNAiMax and incubate for a further 3 days for a total incubation time of 6 days. Collect both the cellular supernatant and cell lysate (see below) for quantification of HBsAg.

Secreted HBsAg was measured quantitatively using HBsAg ELISA kit (Autobio-CL0310).

The EC₅₀, the concentration of the drug required for reducing HBsAg secretion by 50% in relation to the untreated cell control value was calculated from the plot of the percentage reduction of the HBsAg level against the drug concentrations using Microsoft Excel (forecast function).

Set up a parallel set of plates that are to be used for testing compound induced cellular cytotoxicity (see below).

### Cytotoxicity Assay

HepG2.2.15 cells were cultured and treated as above. At Day 6, cellular cytotoxicity was assessed using a cell proliferation assay (CellTiter-Glo Luminescent Cell Viability Assay; Promega) according to the manufacturer's instructions or a suitable alternative.

The CC₅₀, the concentration of the drug required for reducing cell viability by 50% in relation to the untreated cell control value was calculated from the plot of the percentage reduction of viable cells against the drug concentrations using Microsoft Excel (forecast function).

### Example B2

### Cross-Genotype Activates of Modified Oligonucleotides in HBV

HBsAg Release Inhibition

A modified oligonucleotide (compound B2) having the following structure was prepared as described in WO 2020/097342:
5′ mApsln(5m)CpsmApsln(5m)CpsmApsln(5m)CpsmApsln(5m)CpsmApsln(5m)CpsrApsln(5m)CpsmApsln(5m)CpsmApsln(5m)CpsrApsln(5m)CpsmApsln(5m)CpsmApsln(5m)CpsrApsln(5m)CpsmApsln(5m)CpsmApsln(5m)CpsrApsln(5m)CpsmApsln(5m)CpsmApsln(5m)CpsrApsln(5m)CpsmApsln(5m)CpsmApsln(5m)C 3′

The cross-genotype activities of compound B2 and REP-2139 were evaluated as described below and summarized in Table 27.

### Testing in Stable Cell Line

For HBV Genotype D: The HepG2.2.15 cell line (Sells M A, Chen M, Acs G. Production of hepatitis B virus particles in Hep G2 cells transfected with cloned hepatitis B virus DNA. Proc Nat Acad Sci USA 1987; 84:1005-1009), which contains 2 head-to-tail integrated copies of the HBV genome and produces infectious HBV particles, was used as an in vitro model of HBV infection. The cell line was licensed and obtained from the Fox Chase

Cancer Center (Philadelphia, Pa.). HepG2.2.15 cells were maintained in DMEM/F12 (Catalog 10-092-CM, Corning) with 10% fetal bovine serum, 1% penicillin and streptomycin, 2 mM glutamine, 1% non-essential amino acids, and 1% sodium pyruvate. To prepare the HepG2.2.15 for assay, the cells were trypsinized at 37° C. and diluted to 0.35×10⁶/mL with maintenance medium.

The transfection mixture was prepared as follows: First, a master mixture was prepared by combining RNAiMAX (Catalog 13778-150, Thermo Fisher; 0.3 μL/well for a 96-well plate) with Opti-MEM I (5.2 μL/well), which was then vortexed and incubated for 5 minutes at room temperature. At least a 20% excess volume of the master mixture was prepared. Next, serial dilutions of the test compounds (3-fold) were made with Opti-MEM I at 20× of final concentration (8-point dose response), which was mixed with equal volumes of master mixture and then incubated for another 5-10 minutes.

The resulting test compound/RNAiMAX mixtures were added to 96-well plates at a volume of 11 μL per well and then 100 μL of HepG2.2.15 cells per well were added. The plates were swirled for 10 seconds by hand then incubated at 37° C. for 3 days. After 3 days, the medium was refreshed, and the same test compound and RNAiMAX transfection mixture was added a second time, swirled for 10 seconds by hand, then incubated at 37° C. for another 3 days.

On Day 6, the supernatant was harvested for HBsAg quantitation and the cells were assayed for viability. The HBsAg was measured with an ELISA kit (Catalog DS187701, Diasino) and cell viability was measured with the CellTiter-Glo (Promega) assay kit according to the manufacturer's instructions.

The HepG2-GtA and HepG2-GtB cell lines were established at Aligos Therapeutics, Inc. These cell lines contain 1.3×HBV genomes (AB246338.1 genotype A and AB246341 genotype B) and produce HBV viral products continuously. Otherwise the protocol was the same as HepG2.2.15.

Live HBV-Infected HepG2-NTCP

HepG2-NTCP cells contain an over-expressed NTCP receptor in the HepG2 cell line and have been shown to be a robust cell culture system supporting the complete life cycle of HBV (see Michailidis E, Pabon J, Xiang K, et al. A robust cell culture system supporting the complete life cycle of hepatitis B virus. Sci Rep 2017; 7(1): 16616. doi: 10.1038/s41598-017-16882-5). The cell line was licensed and obtained from the Fox Chase Cancer Center (Philadelphia, Pa.). HepG2-NTCP cells were maintained in DMEM/F12 (Catalog 10-092-CM, Corning) with 10% fetal bovine serum, 1% penicillin and streptomycin, 2 mM glutamine, 1% non-essential amino acids and 1% sodium pyruvate. The cells were trypsinized at 37° C. and diluted to 0.15×10⁶/mL with maintenance medium. Briefly, the cells were seeded at 20,000/well in a 96-well plate on Day −2 and infected with HBV at a multiplicity of infection of 500 on Day 0. Infectious HBV particles (Genotype D) were harvested and concentrated from the supernatant of HepG2.2.15 cells (see Sells et. al. 1987), also licensed from the Fox Chase Cancer Center. Genotype B HBV (GenBank Accession No. JN406371) and Genotype C HBV (GenBank Accession No. AB246345) clinical isolates were purified from the supernatant of cultured HepG2 cells that had been transfected with plasmid DNA containing the corresponding HBV genomes.

The infected cells were transfected with test compounds on Days 5 and 8. The HBsAg was measured in the supernatant on Day 11 and the remaining cells were measured for cell viability.

The transfection mixture was prepared as follows: First, a master mixture was prepared by combining RNAiMAX (Catalog 13778-150, Thermo Fisher; 0.3 μL/well for a 96-well plate) with Opti-MEM I (5.2 μL/well), which was then vortexed and incubated for 5 minutes at room temperature. At least a 20% excess volume of master mixture was prepared. Next, serial dilutions of the test compounds (3-fold) were made with Opti-MEM I at 20× of final concentration (8-point dose response), which was then mixed with equal volumes of the master mixture and then incubated for another 5-10 minutes.

The above test compound/RNAiMAX mixtures were added to 96-well plates containing HBV-infected HepG2-NTCP cells in a volume of 11 μL per well. The plates were swirled for 10 seconds by hand and then incubated at 37° C. for 3 days. After 3 days, the medium was refreshed, and the same test compound and RNAiMAX transfection mixture was added for a second time, swirled for 10 seconds by hand, then incubated at 37° C. for an additional 3 days.

Three days after the second compound treatment, the supernatant was harvested for HBsAg detection. HBsAg was measured with an ELISA kit (Catalog DS187701, Diasino) and cell viability was measured with the CellTiter-Glo (Promega) assay kit according to the manufacturer's instructions.

The results are summarized in Table 27 below and demonstrate that the modified oligonucleotide (compound B2) demonstrated greater potency than REP-2139. Compound B2 also demonstrated enhanced cross genotypic activity, inhibiting the HBsAg release in cells containing HBV genotype A, B, C and D viruses with EC₅₀ values of 7.9, 9.25, 0.72 and 3.9 nM, respectively.

TABLE 27

| | | Activity (EC₅₀) | Activity (EC₅₀) in Live |
|---|---|---|---|
| HBV | | in Stable | HBV-Infected |
| Genotype | Compound | Cell Lines (nM) | HepG2-NTCP (nM) |
| A | B2 | 7.6 ± 2.0 | — |
| | REP-2139 | 71.7 ± 15.6 | — |
| B | B2 | 18.24 ± 8.1 | 9.25 ± 1.26 |
| | REP-2139 | >10,000 | 71.89 ± 10.74 |
| C | B2 | — | 0.72 ± 0.03 |
| | REP-2139 | — | 71.04 ± 15.69 |
| D | B2 | 4.8 ± 1.1 | 2.7 ± 0.9 |
| | REP-2139 | 260.3 ± 98 | 320.7 ± 110 |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A modified oligonucleotide or complex thereof having sequence independent antiviral activity against hepatitis B, comprising an at least partially phosphorothioated sequence of modified nucleoside units that comprise modified A units, modified C units and/or other modified nucleoside units, wherein:
the modified A units comprise one or more selected from: 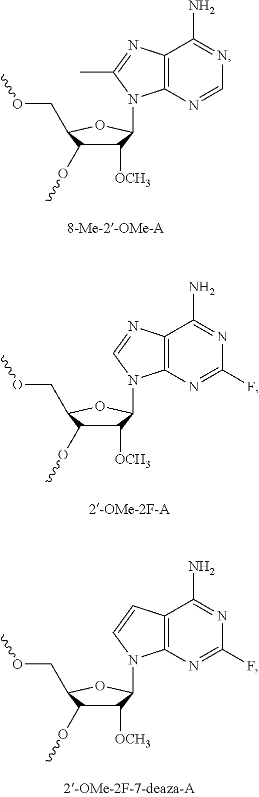 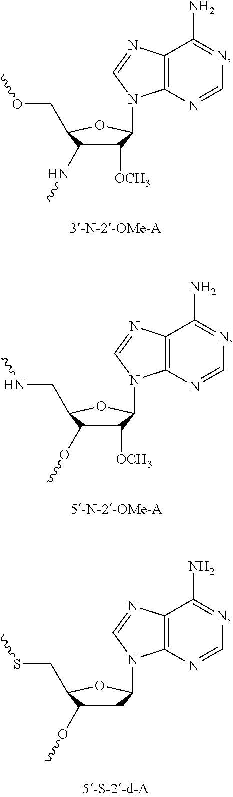 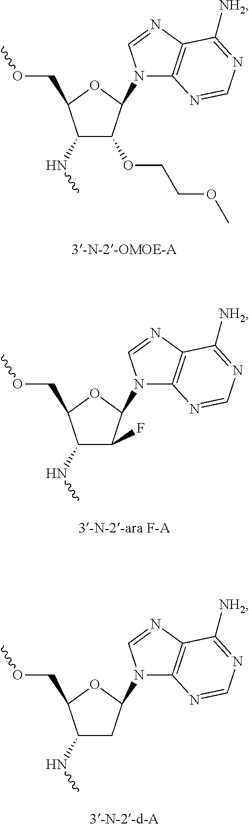 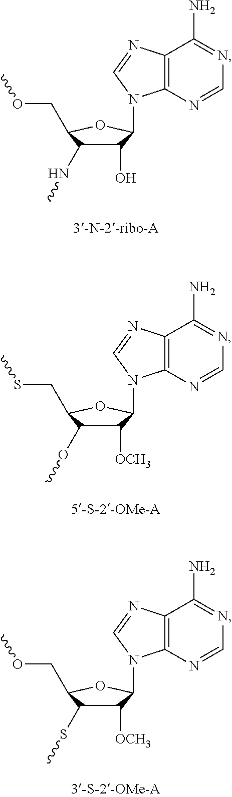 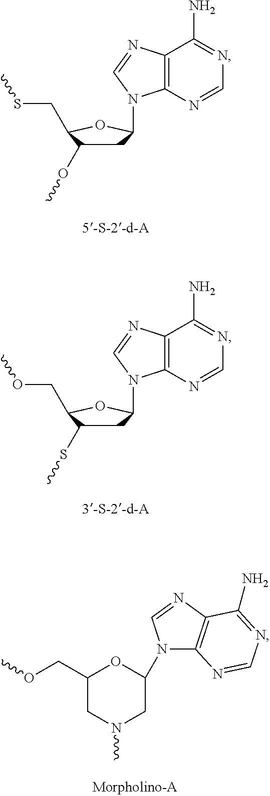 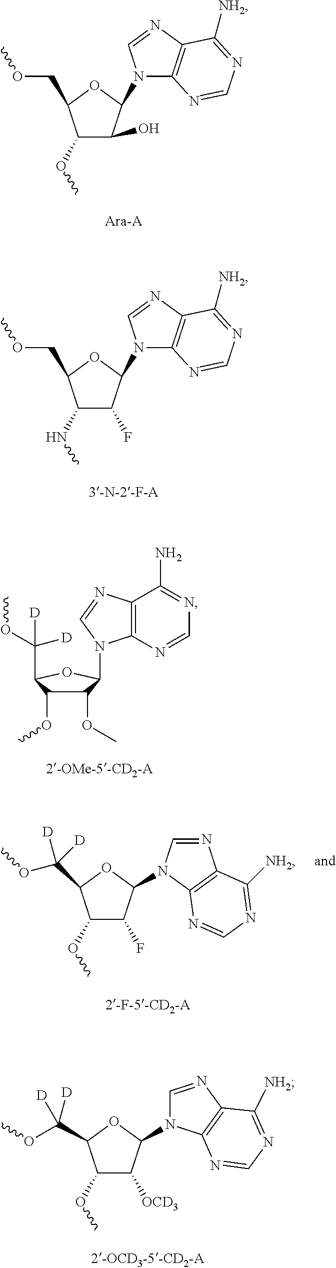
the modified C units comprise one or more selected from: 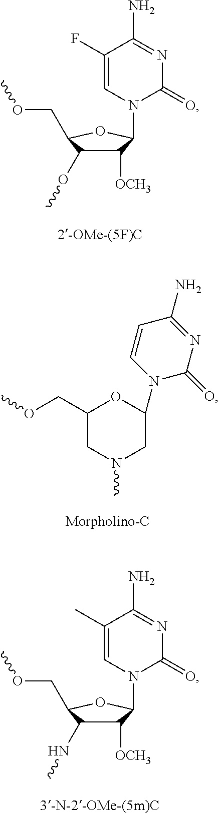 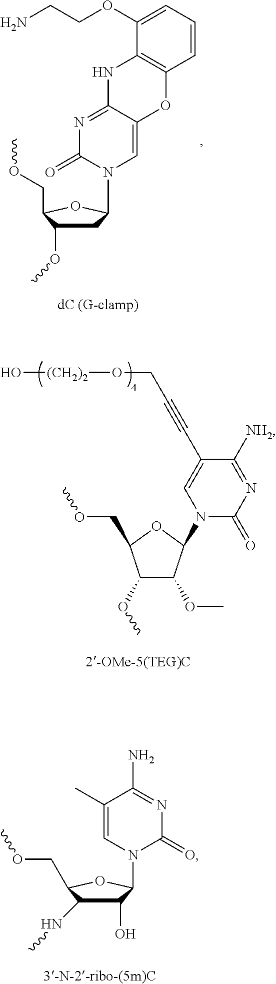 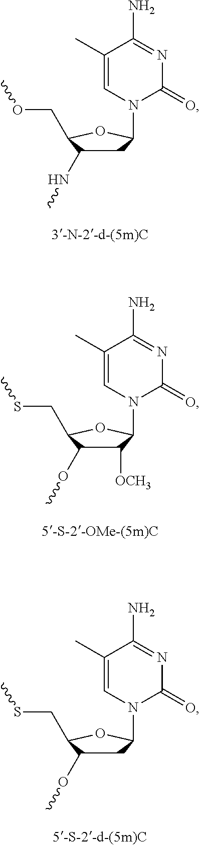 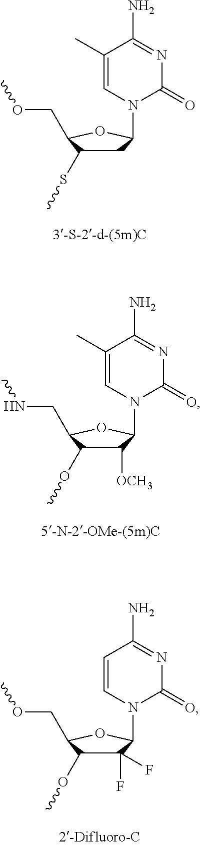 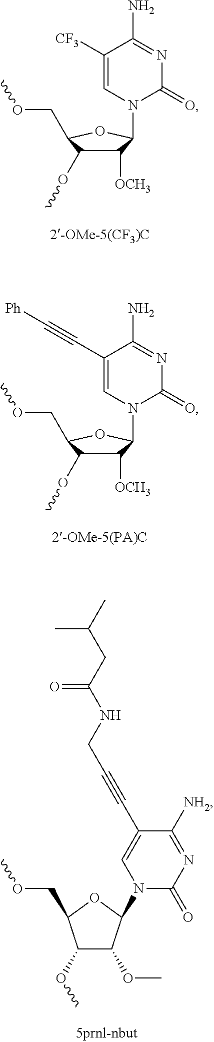 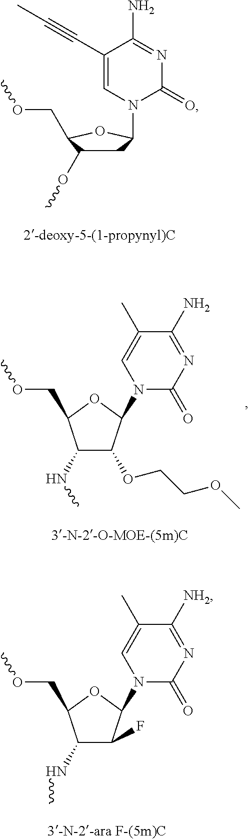 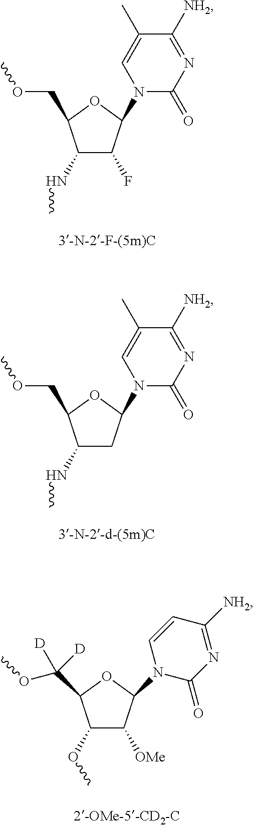 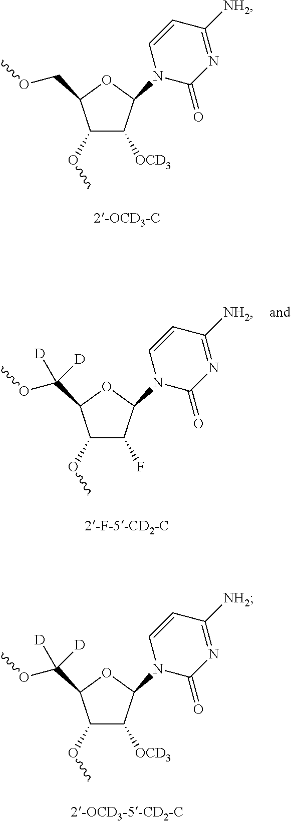
the other modified nucleoside units comprise one or more selected from: 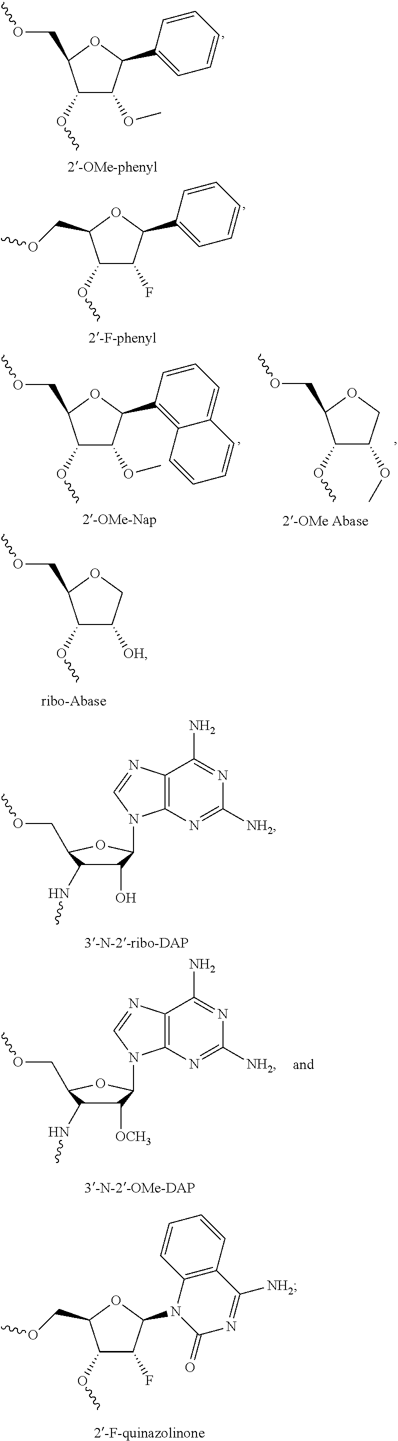
each terminal 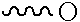
is independently hydroxyl, an O,O-dihydrogen phosphorothioate, a dihydrogen phosphate, an endcap or a linking group;
each terminal 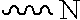
is independently an amine, a C₁₋₆ alkylamine, a di-C₁₋₆alkylamine, an endcap or a linking group;
each terminal 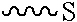
is independently a thiol, an O,O-dihydrogen phosphorothioate, a dihydrogen phosphate, an endcap or a linking group;
each internal 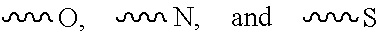
is joined together with me internal 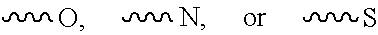
of a neighboring nucleoside unit to form a phosphorus-containing internucleoside linkage of the formula 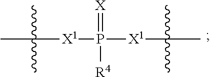
each X is individually S or O, with the proviso that at least one X is S;
each X¹ is individually O, NR^{b}, or S;
each R⁴ is individually OH, SH, optionally substituted C₁₋₆ alkyl, optionally substituted C₁₋₆ alkoxy, or optionally substituted amino;
each R^{b} is individually hours or C₁₋₆ alkyl; and
the sequence independent antiviral activity against hepatitis B, as determined by HBsAg Secretion Assay, is an EC₅₀ that is less than 100 nM.

2. The modified oligonucleotide or complex thereof of claim 1, wherein the modified A unit is

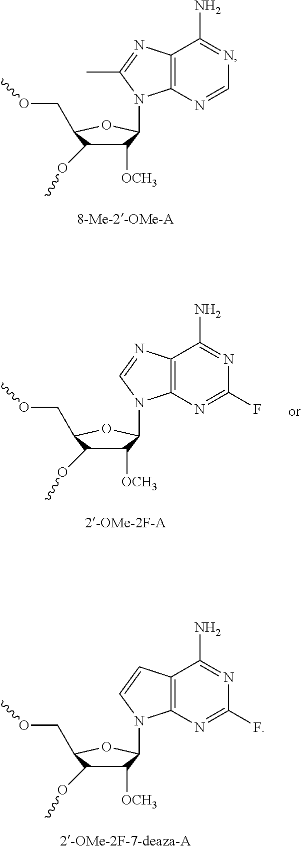

3. The modified oligonucleotide or complex thereof of claim 1, wherein the modified A unit is

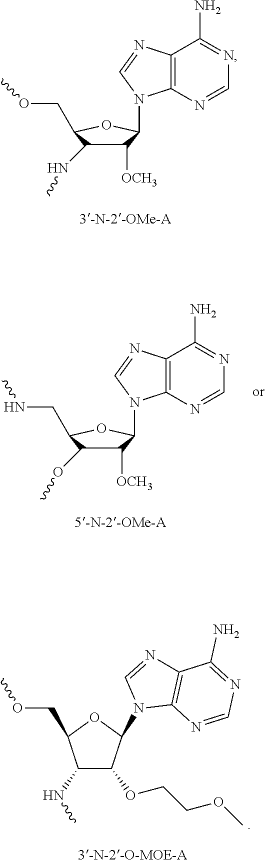

4. The modified oligonucleotide or complex thereof of claim 1, wherein the modified A unit is

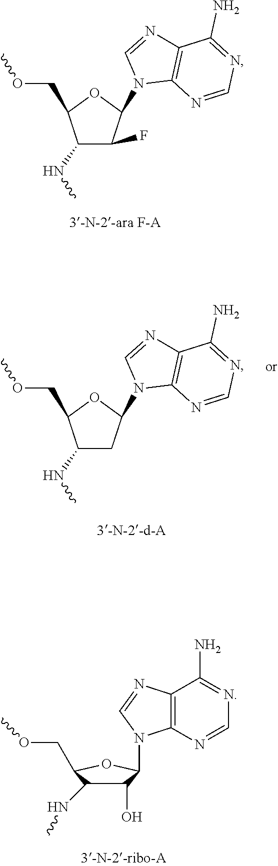

5. The modified oligonucleotide or complex thereof of claim 1, wherein the modified A unit is

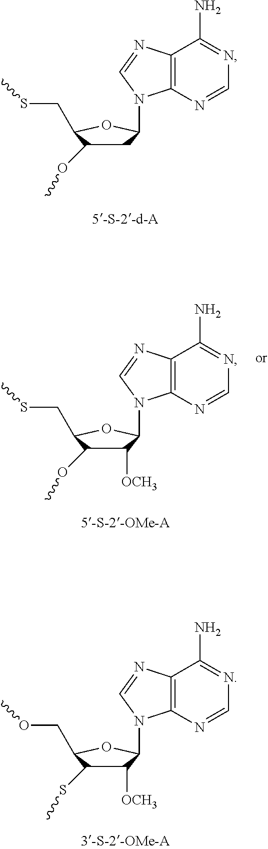

6. The modified oligonucleotide or complex thereof of claim 1, wherein the modified A unit is

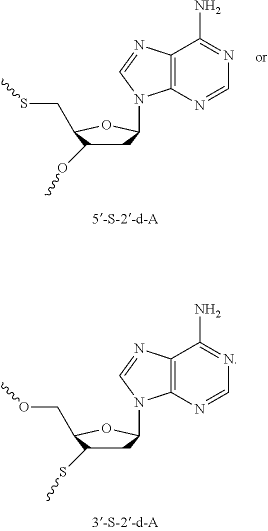

7. The modified oligonucleotide or complex thereof of claim 1, wherein 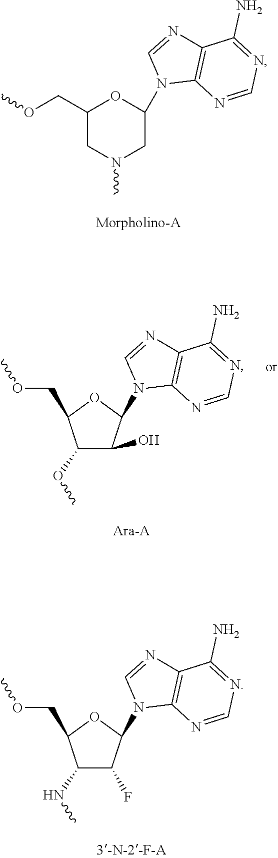
the modified A unit is

8. The modified oligonucleotide or complex thereof of claim 1, wherein the modified A unit is

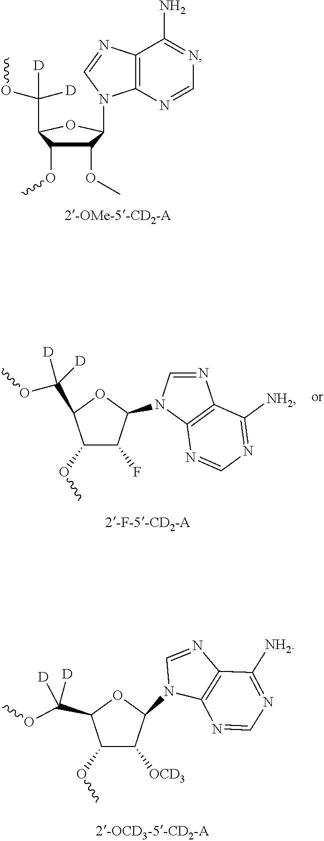

9. The modified oligonucleotide or complex thereof of claim 1, wherein the other modified nucleoside unit is

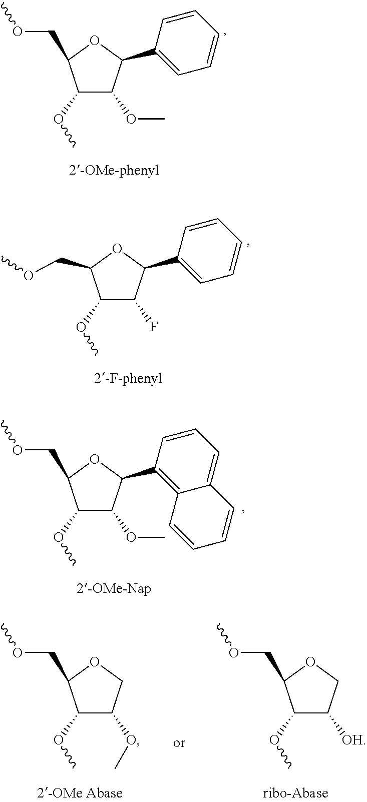

10. The modified oligonucleotide or complex thereof of claim 1, wherein the at least partially phosphorothioated sequence of modified nucleoside units further comprises an A unit of Table 4.

11. The modified oligonucleotide or complex thereof of claim 1, wherein the modified C unit is

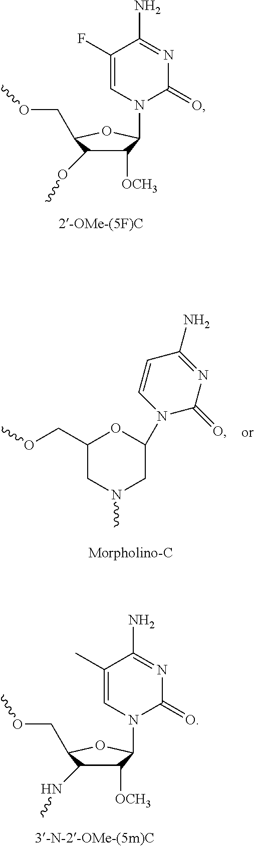

12. The modified oligonucleotide or complex thereof of claim 1, wherein the modified C unit is

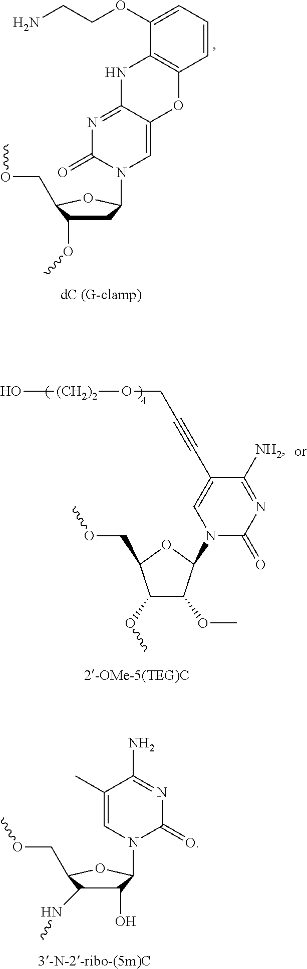

13. The modified oligonucleotide or complex thereof of any claim 1, wherein the modified C unit is

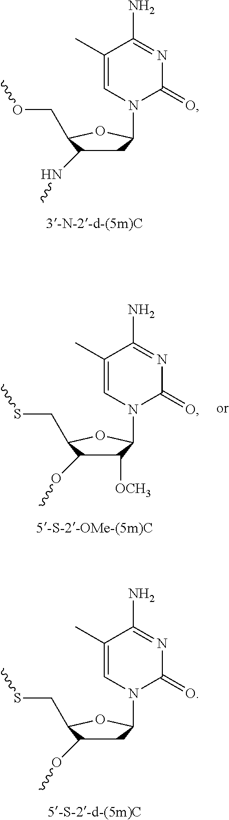

14. The modified oligonucleotide or complex thereof of claim 1, wherein the modified C unit is

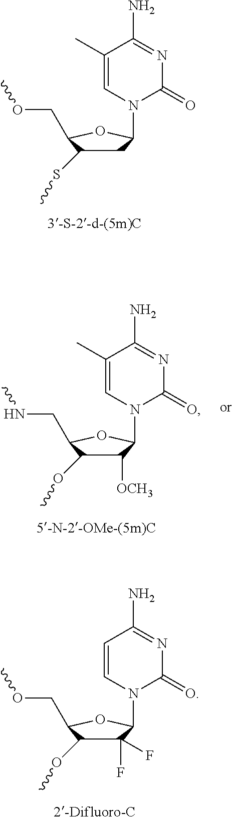

15. The modified oligonucleotide or complex thereof of claim 1, wherein the modified C unit is

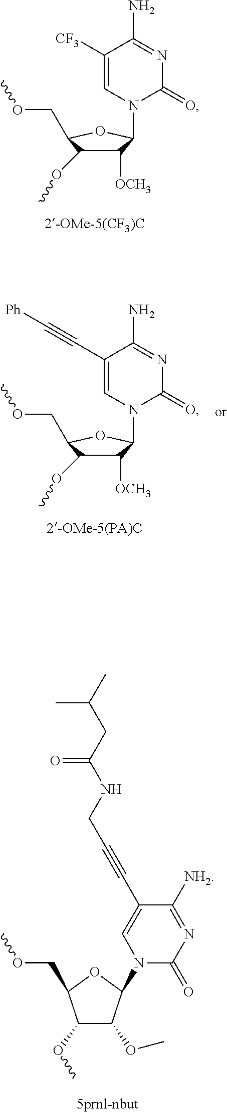

16. The modified oligonucleotide or complex thereof of claim 1, wherein the modified C unit is

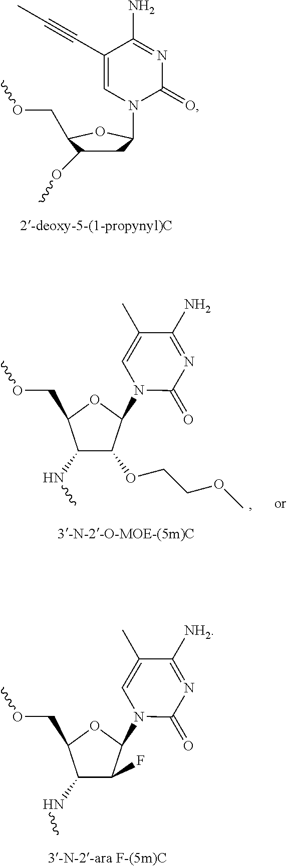

17. The modified oligonucleotide or complex thereof of claim 1, wherein the modified C unit is

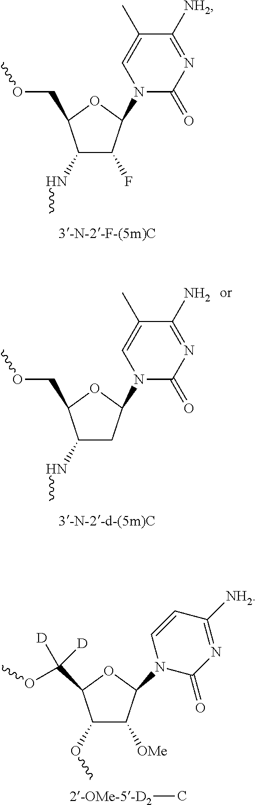

18. The modified oligonucleotide or complex thereof of claim 1, wherein the modified C unit is

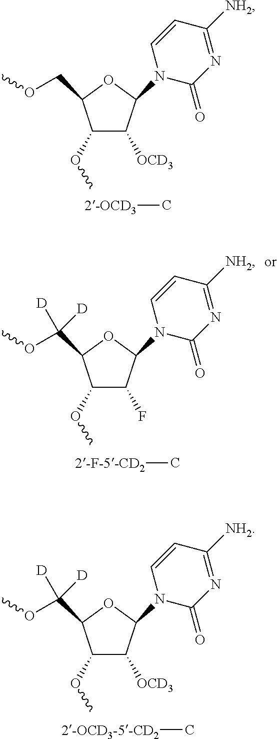

19. The modified oligonucleotide or complex thereof of claim 1, wherein the other modified nucleoside unit is

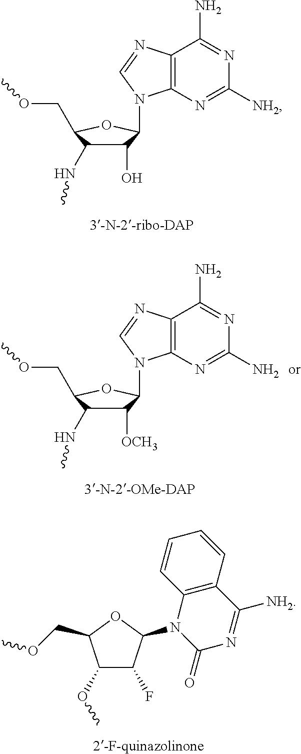

20. The modified oligonucleotide or complex thereof of claim 1, further comprising a unit selected from

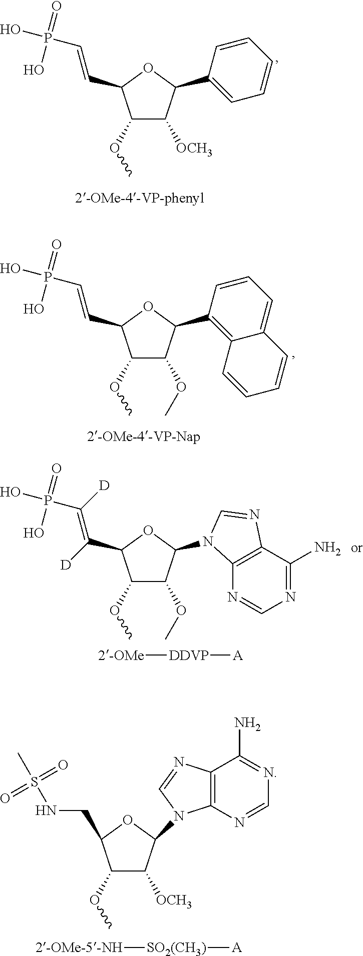

21. The modified oligonucleotide or complex thereof of claim 1, wherein the at least partially phosphorothioated sequence of modified nucleoside units further comprises a C unit of Table 4.

22. The modified oligonucleotide or complex thereof of claim 1, further comprising an A unit and a C unit of Table 4.

23. The modified oligonucleotide or complex thereof of claim 1 that is partially phosphorothioated.

24. The modified oligonucleotide or complex thereof of claim 23 that is at least 85% phosphorothioated.

25. The modified oligonucleotide or complex thereof of claim 1 that is fully phosphorothioated.

26. The modified oligonucleotide or complex thereof of claim 23, comprising at least one stereochemically defined phosphorothioate linkage.

27. The modified oligonucleotide or complex thereof of claim 26, comprising at least 6 stereochemically defined phosphorothioate linkages.

28. The modified oligonucleotide or complex thereof of claim 26, wherein the at least one stereochemically defined phosphorothioate linkage has an R configuration.

29. The modified oligonucleotide or complex thereof of claim 26, wherein the at least one stereochemically defined phosphorothioate linkage has an S configuration.

30. The modified oligonucleotide or complex thereof of claim 1, comprising a 5′ endcap.

31. The modified oligonucleotide or complex thereof of claim 30, wherein the 5′ endcap is selected from 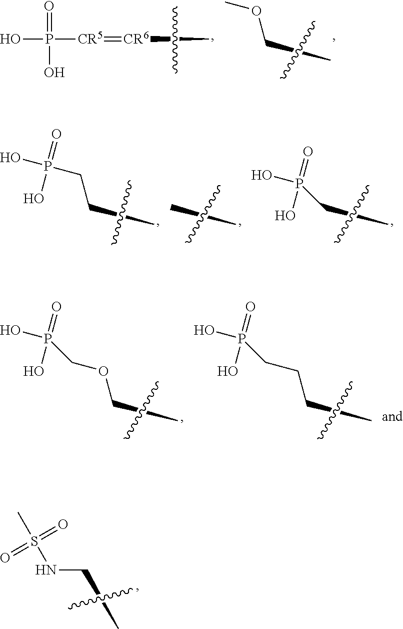
wherein R⁵ and R⁶ are each individually selected from hydrogen, deuterium, phosphate, thioC₁₋₆ alkyl, and cyano.

32. The modified oligonucleotide or complex thereof of claim 31, wherein R⁵ and R⁶ are both hydrogen.

33. The modified oligonucleotide or complex thereof of claim 31, wherein R⁵ and R⁶ are not both hydrogen.

34. The modified oligonucleotide or complex thereof of claim 31, wherein the 5′ endcap is selected from

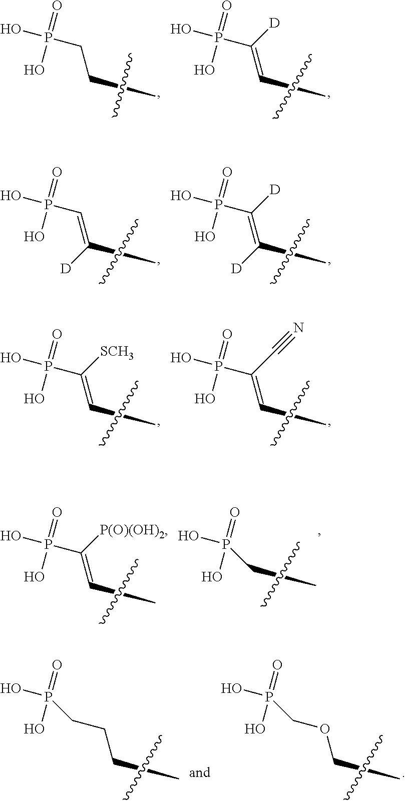

35. The modified oligonucleotide or complex thereof of claim 31, wherein the 5′ endcap is

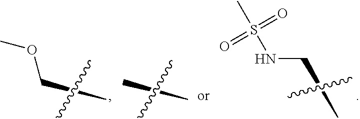

36. The modified oligonucleotide or complex thereof of claim 1, wherein the at least partially phosphorothioated sequence of modified nucleoside units has a sequence length in the range of about 8 units to about 200 units.

37. The modified oligonucleotide or complex thereof of claim 1, wherein the at least partially phosphorothioated sequence of modified nucleoside units has a sequence length in the range of 18 units to 60 units.

38. The modified oligonucleotide or complex thereof of claim 1, wherein the at least partially phosphorothioated sequence of modified nucleoside units has a sequence length in the range of 30 units to 50 units.

39. The modified oligonucleotide or complex thereof of claim 1, wherein the at least partially phosphorothioated sequence of modified nucleoside units has a sequence length in the range of 34 units to 46 units.

40. The modified oligonucleotide or complex thereof of claim 1, wherein the at least partially phosphorothioated sequence of modified nucleoside units has a sequence length in the range of 36 units to 44 units.

41. The modified oligonucleotide or complex thereof of claim 1, wherein at least one terminal 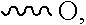
at least one terminal 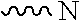
or at least one terminal 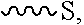
is a linking group.

42. The modified oligonucleotide or complex thereof of claim 41, further comprising at least one second oligonucleotide that is attached to the modified oligonucleotide via the linking group.

43. The modified oligonucleotide or complex thereof of claim 41, further comprising a targeting ligand that is attached to the modified oligonucleotide via the linking group.

44. The modified oligonucleotide or complex thereof of claim 43, wherein the targeting ligand comprises N-acetylgalactosamine (GalNAc), triantennary-GalNAc, a tocopherol or cholesterol.

45. The modified oligonucleotide or complex thereof of claim 1, wherein at least some of the modified A units are not 2′-O-methylated on the ribose ring.

46. The modified oligonucleotide or complex thereof of claim 1, wherein the phosphorus-containing internucleoside linkage is

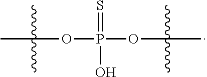 [SMILES: *OP(O)(=S)O*]

47. The modified oligonucleotide or complex thereof of claim 1, wherein the at least partially phosphorothioated sequence of A and C units further comprises one or more modifications to a phosphorothioate linkage.

48. The modified oligonucleotide or complex thereof of claim 47, wherein the modification to the phosphorothioate linkage is a modified linkage selected from phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate, diphosphodiester or 3′-S-phosphorothiolate.

49. The modified oligonucleotide or complex thereof of claim 48, wherein the modified linkage is a phosphodiester linkage.

50. The modified oligonucleotide or complex thereof of claim 1, further comprising at least two partially phosphorothioated sequences of modified nucleoside units linked together to form a concatemer.

51. The modified oligonucleotide or complex thereof of claim 1, wherein the modified oligonucleotide has an EC₅₀ value, as determined by HBsAg Secretion Assay, that is in the range of 30 nM to less than 100 nM.

52. The modified oligonucleotide or complex thereof of claim 1, wherein the modified oligonucleotide has an EC₅₀ value, as determined by HBsAg Secretion Assay, that is less than 30 nM.

53. The modified oligonucleotide or complex thereof of claim 1, wherein the at least partially phosphorothioated sequence has a sequence length and modified nucleoside units as set forth in FIG. 6.

54. A modified oligonucleotide or complex thereof having sequence independent antiviral activity against hepatitis B, comprising an at least partially phosphorothioated sequence of modified nucleoside units, wherein the sequence of modified nucleoside units comprises an A block that consists of 4-10 consecutive modified A units selected from Table 1, a C block that consists of 4-10 consecutive C units selected from Table 2, and/or an other block that consists of 4-10 consecutive other modified units selected from Table 3.

55. The modified oligonucleotide or complex thereof of claim 54, wherein the A block is at a first end position at a 3′ or 5′ end of the sequence of modified nucleoside units.

56. The modified oligonucleotide or complex thereof of claim 55, further comprising a second A block that is at a second end position at the opposite end of the sequence of modified nucleoside units from the first end position.

57. The modified oligonucleotide or complex thereof of claim 54, wherein the C block is at a first end position at a 3′ or 5′ end of the sequence of modified nucleoside units.

58. The modified oligonucleotide or complex thereof of claim 57, further comprising a second C block that is at a second end position at the opposite end of the sequence of modified nucleoside units from the first end position.

59. The complex of the modified oligonucleotide of claim 1, wherein the complex is a chelate complex.

60. The complex of claim 59, wherein the complex is a calcium,
magnesium or zinc chelate complex of the modified oligonucleotide.

61. The complex of the modified oligonucleotide of claim 1, wherein the complex is a monovalent counterion complex.

62. The complex of claim 61, wherein the complex is a lithium, sodium or potassium complex of the modified oligonucleotide.

63. The complex of the modified oligonucleotide of claim 62, wherein the complex is a monovalent counterion complex that comprises a sodium or potassium complex of the modified oligonucleotide.

64. A pharmaceutical composition, comprising an amount of the modified oligonucleotide or complex thereof of claim 1, that is effective for treating a subject infected with hepatitis B; and a pharmaceutically acceptable carrier.

65. A pharmaceutical composition, comprising an amount of the modified oligonucleotide or complex thereof of claim 1, that is effective for treating a subject infected with hepatitis D; and a pharmaceutically acceptable carrier.

66. A treatment for hepatitis B, hepatitis D or both, comprising an effective amount of the modified oligonucleotide or complex thereof of claim 1.

67. A cross genotypic treatment for hepatitis B, hepatitis D or both, comprising an effective amount of the modified oligonucleotide or complex thereof of claim 1.

68. (canceled)

69. (canceled)

70. (canceled)

71. A method of treating hepatitis B, comprising administering an effective amount of the modified oligonucleotide or complex thereof of claim 1 to a subject in need thereof.

72. (canceled)

73. (canceled)

74. (canceled)

75. (canceled)

76. (canceled)

77. (canceled)

78. (canceled)

79. A method of treating hepatitis D, comprising administering an effective amount of the modified oligonucleotide or complex thereof of claim 1 to a subject in need thereof.

80. (canceled)

81. (canceled)

82. (canceled)

83. (canceled)

84. (canceled)

85. (canceled)

86. (canceled)

87. A method of treating hepatitis B or hepatitis D, comprising subcutaneously administering a safe and effective amount of the modified oligonucleotide or complex thereof of claim 1, to a subject in need thereof.

88. (canceled)

89. (canceled)

90. (canceled)

91. (canceled)

92. (canceled)

93. (canceled)

94. A dinucleotide consisting of two modified nucleoside units connected by a phosphorus-containing stereochemically defined linkage, wherein the two modified nucleoside units are each individually selected from a modified A unit, a modified C units and an other modified nucleoside unit, wherein:
the modified A unit is selected from: 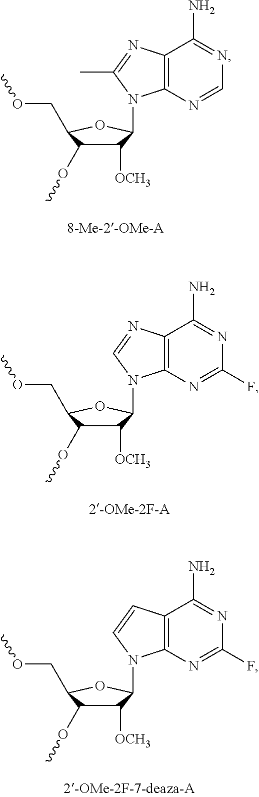 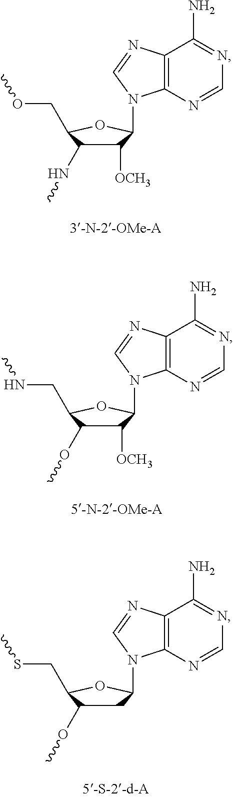 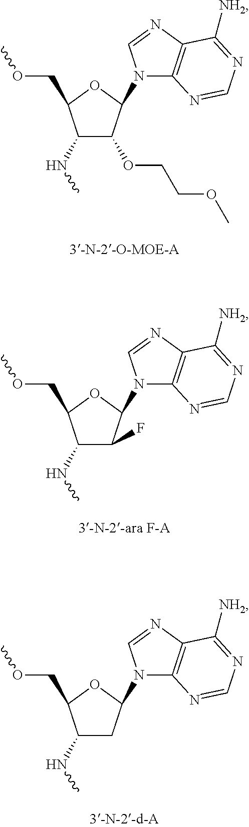 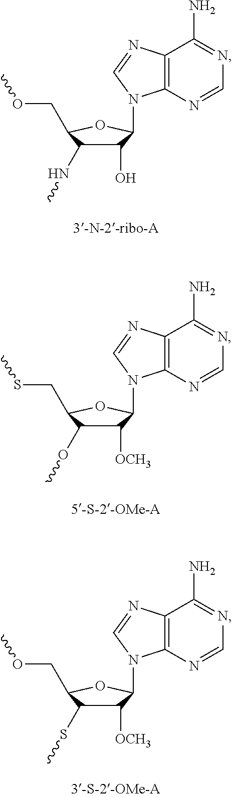 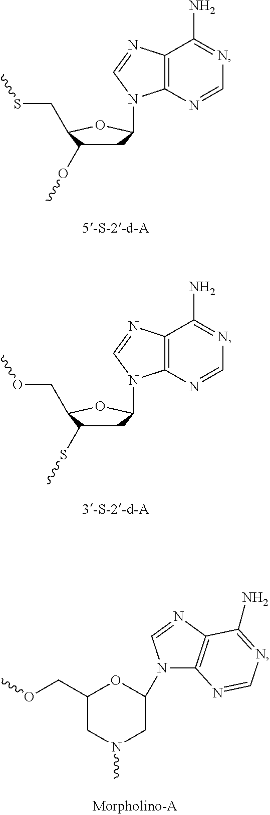 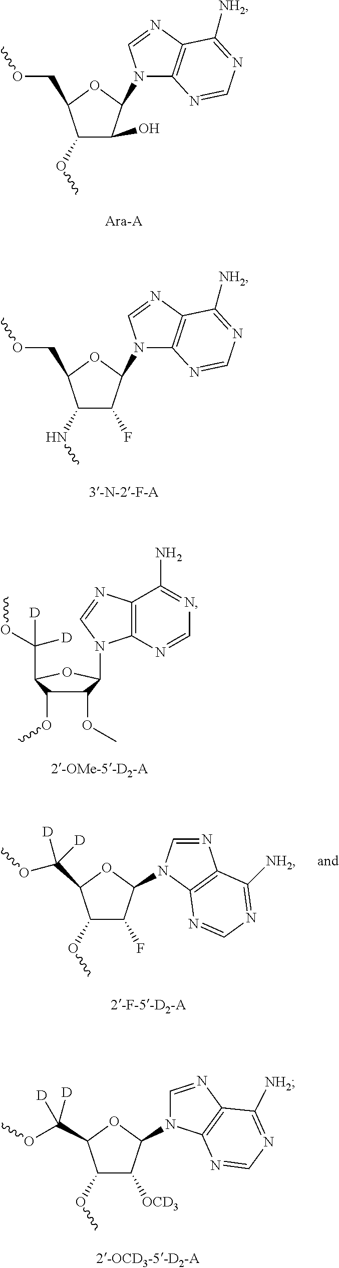 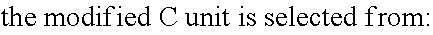 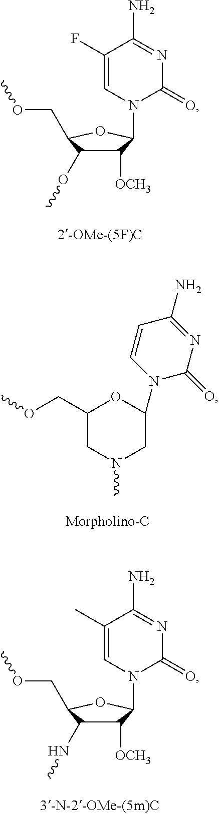 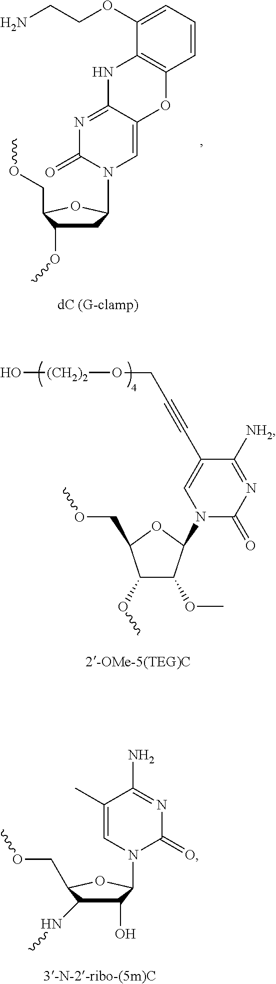 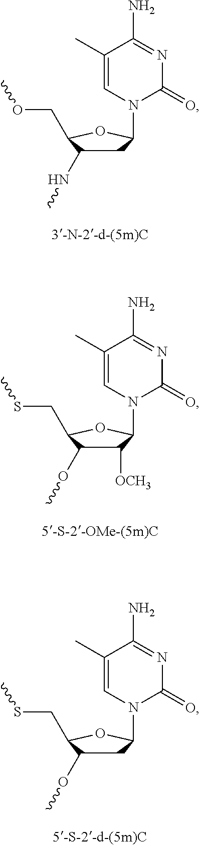 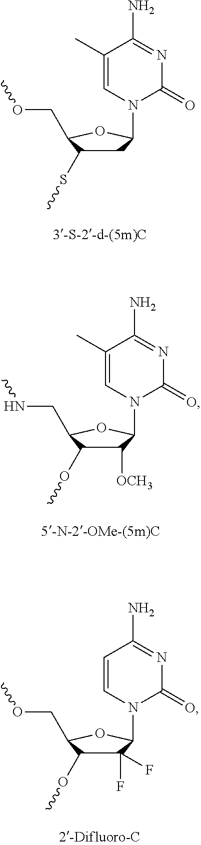 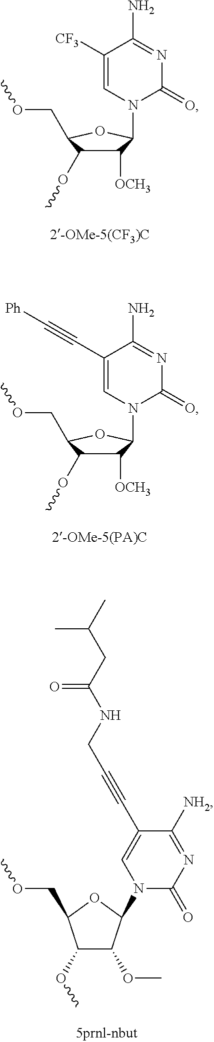 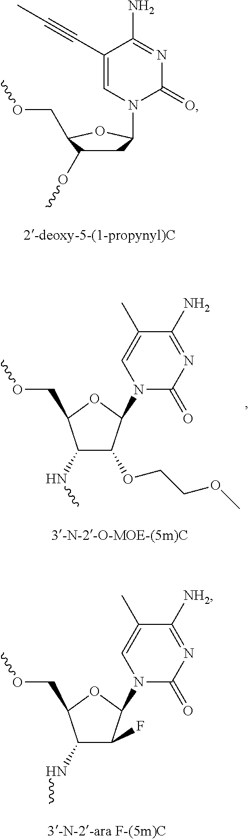 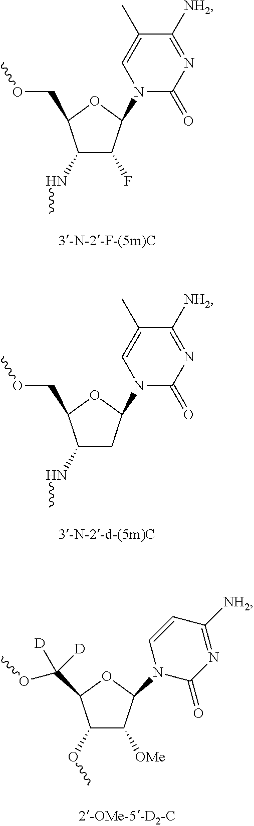 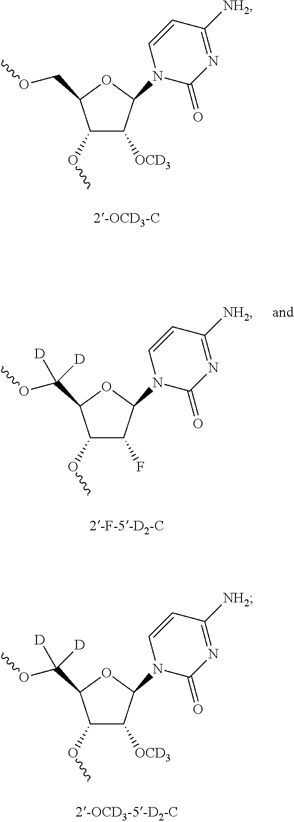
the other modified nucleoside unit is selected from: 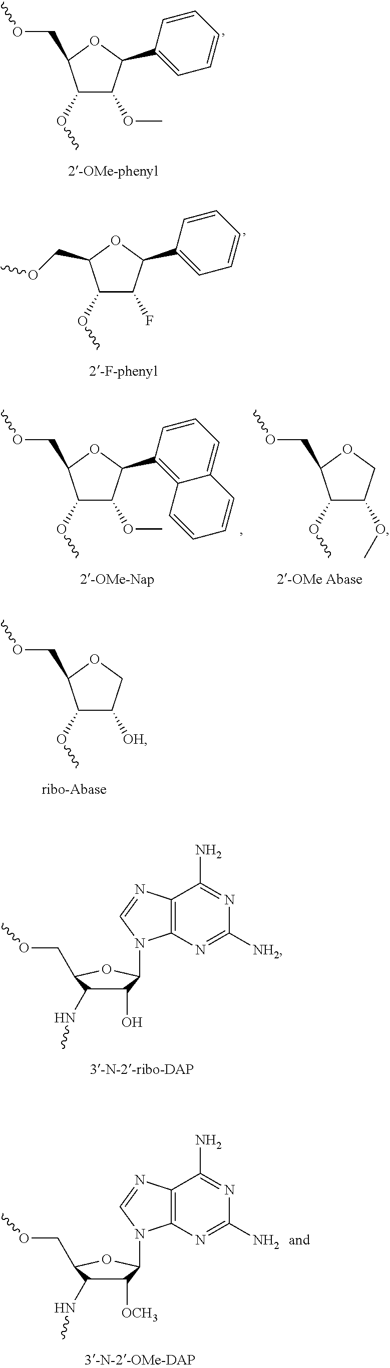 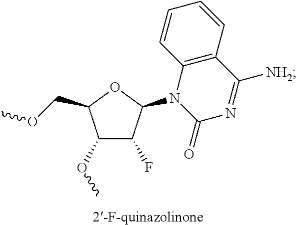
two internal 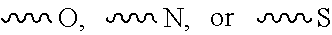
groups are together joined to form the phosphorus-containing stereochemically defined linkage;
each terminal 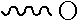
is independently hydroxyl, an O,O-dihydrogen phosphorothioate, an O,O-dihydrogen phosphate, a phosphoramidite, a trityl ether (TrO), a methoxytrityl ether (MMTrO), or a dimethoxytrityl ether (DMTO or DMTrO);
each terminal 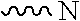
is independently an amine, a phosphoramidate, a thiophosphoramdiate, a phosphorodiamidate, a phosphorothiodiamidate, a tritylamino (TrNH), a methoxytritylamino (MMTrNH), or a dimethoxytrityl amino (DMTNH or DMTrNH); and
each terminal 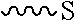
is independently a phosphoramidate, a S-phosphoramidite, a thiol, a thiolate, a phosphothioate, a phosphodithiolate, a trityl thioether (TrS), a methoxytrityl thioether (MMTrS), or a dimethoxytrityl thioether (DMTS or DMTrS).

95. (canceled)

96. (canceled)

97. (canceled)

98. (canceled)

99. (canceled)

100. (canceled)

101. (canceled)

102. A method for making the modified oligonucleotide or complex thereof of claim 1, comprising coupling the corresponding dinucleotide.
